SEQUENCE LISTING (108 sequences)

SEQ ID NO: 1 - DNA, 20 bp, Artificial sequence
  Synthetic polynucleotide
  misc_feature: TRAC gRNA spacer, TRAC 2
agagcaacagtgctgtggcc

SEQ ID NO: 2 - DNA, 20 bp, Artificial sequence
  Synthetic polynucleotide
  misc_feature: TRAC gRNA spacer, TRAC 3
tctctcagctggtacacggc

SEQ ID NO: 3 - DNA, 20 bp, Artificial sequence
  Synthetic polynucleotide
  misc_feature: TRAC gRNA spacer, TRAC 1
acaaaactgtgctagacatg

SEQ ID NO: 4 - DNA, 20 bp, Artificial sequence
  Synthetic polynucleotide
  misc_feature: IL2Rg gRNA spacer, GC1
accagtgcctggcatgtagt

SEQ ID NO: 5 - DNA, 20 bp, Artificial sequence
  Synthetic polynucleotide
  misc_feature: IL2Rg gRNA spacer, GC2
ccagtgcctggcatgtagta

SEQ ID NO: 6 - DNA, 20 bp, Artificial sequence
  Synthetic polynucleotide
  misc_feature: IL2Rg gRNA spacer, GC3
cagtgcctggcatgtagtag

SEQ ID NO: 7 - DNA, 20 bp, Artificial sequence
  Synthetic polynucleotide
  misc_feature: IL2Rg gRNA spacer, GC4
gtaggggcacaacaaatata

SEQ ID NO: 8 - DNA, 20 bp, Artificial sequence
  Synthetic polynucleotide
  misc_feature: IL2Rg gRNA spacer, GC5
gaatcctttcctgtttgcat

SEQ ID NO: 9 - DNA, 20 bp, Artificial sequence
  Synthetic polynucleotide
  misc_feature: IL2Rg gRNA spacer, GC6
cctgtttgcattggaagccg

SEQ ID NO: 10 - DNA, 20 bp, Artificial sequence
  Synthetic polynucleotide
  misc_feature: IL2Rg gRNA spacer, GC7
gaagccgtggttatctctgt

SEQ ID NO: 11 - DNA, 20 bp, Artificial sequence
  Synthetic polynucleotide
  misc_feature: IL2Rg gRNA spacer, GC8
ggttatctctgttggctcca

SEQ ID NO: 12 - DNA, 20 bp, Artificial sequence
  Synthetic polynucleotide
  misc_feature: IL2Rg gRNA spacer, GC9
gttatctctgttggctccat

SEQ ID NO: 13 - DNA, 20 bp, Artificial sequence
  Synthetic polynucleotide
  misc_feature: IL2Rg gRNA spacer, GC10
aaggctgataatcaatccca

SEQ ID NO: 14 - DNA, 20 bp, Artificial sequence
  Synthetic polynucleotide
  misc_feature: IL2Rg gRNA spacer, GC11
ggagccaacagagataacca

SEQ ID NO: 15 - DNA, 20 bp, Artificial sequence
  Synthetic polynucleotide
  misc_feature: IL2Rg gRNA spacer, GC12
ccacggcttccaatgcaaac

SEQ ID NO: 16 - DNA, 20 bp, Artificial sequence
  Synthetic polynucleotide
  misc_feature: IL2Rg gRNA spacer, GC13
gcttccaatgcaaacaggaa

SEQ ID NO: 17 - DNA, 20 bp, Artificial sequence
  Synthetic polynucleotide
  misc_feature: IL2Rg gRNA spacer, GC14
tagaaaaaagaaaagcaaag

SEQ ID NO: 18 - DNA, 20 bp, Artificial sequence
  Synthetic polynucleotide
  misc_feature: IL2Rg gRNA spacer, GC15
ttgtgcccctactacatgcc

SEQ ID NO: 19 - DNA, 3469 bp, Artificial sequence
  Synthetic polynucleotide
  misc_feature: TRAC AAV 1 TRAC 2 HA TRAC 2-C11D5.3-CD8-CD28- CD3z-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtgtgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 3469 bp, Artificial sequence
  Synthetic polynucleotide
  misc_feature: TRAC AAV 1 TRAC 3 HA TRAC 3-C11D5.3-CD8-CD28- CD3z-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcctgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 3469 bp, Artificial sequence
  Synthetic polynucleotide
  misc_feature: TRAC AAV 1 TRAC 1 HA TRAC 1-C11D5.3-CD8-CD28- CD3z-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagactgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 3054 bp, Artificial sequence
  Synthetic polynucleotide
  misc_feature: TRAC AAV 2 TRAC 2 HA TRAC 2-2A-C11D5.3-CD8- CD28-CD3z-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtgggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 3054 bp, Artificial sequence
  Synthetic polynucleotide
  misc_feature: TRAC AAV 2 TRAC 3 HA TRAC 3-2A-C11D5.3-CD8- CD28-CD3z-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 3054 bp, Artificial sequence
  Synthetic polynucleotide
  misc_feature: TRAC AAV 2 TRAC 1 HA TRAC 1-2A-C11D5.3-CD8- CD28-CD3z-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 3633 bp, Artificial sequence
  Synthetic polynucleotide
  misc_feature: TRAC AAV 3 TRAC 2 HA TRAC 2-2A-C11D5.3-CD8- CD28-CD3z-CNb30-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtgggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggcagcggcgaaggcagaggatccctgcttacatgtggcgacgtggaagagaaccctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 26 - DNA, 3633 bp, Artificial sequence
  Synthetic polynucleotide
  misc_feature: TRAC AAV 3 TRAC 3 HA TRAC 3-2A-C11D5.3-CD8- CD28-CD3z-CNb30-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggcagcggcgaaggcagaggatccctgcttacatgtggcgacgtggaagagaaccctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27 - DNA, 3633 bp, Artificial sequence
  Synthetic polynucleotide
  misc_feature: TRAC AAV 3 TRAC 1 HA TRAC 1-2A-C11D5.3-CD8- CD28-CD3z-CNb30-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggcagcggcgaaggcagaggatccctgcttacatgtggcgacgtggaagagaaccctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 4840 bp, Artificial sequence
  Synthetic polynucleotide
  misc_feature: TRAC AAV 4 TRAC 2 HA TRAC 2-C11D5.3-CD8-CD28- CD3z-P2A-CISCb-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtgtgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 29 - DNA, 4840 bp, Artificial sequence
  Synthetic polynucleotide
  misc_feature: TRAC AAV 4 TRAC 3 HA TRAC 3-C11D5.3-CD8-CD28- CD3z-P2A-CISCb-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcctgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - DNA, 4840 bp, Artificial sequence
  Synthetic polynucleotide
  misc_feature: TRAC AAV 4 TRAC 1 HA TRAC 1-C11D5.3-CD8-CD28- CD3z-P2A-CISCb-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagactgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 31 - DNA, 4425 bp, Artificial sequence
  Synthetic polynucleotide
  misc_feature: TRAC AAV 5 TRAC 2 HA TRAC 2-2A-C11D5.3-CD8- CD28-CD3z-CISCb-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtgggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - DNA, 4425 bp, Artificial sequence
  Synthetic polynucleotide
  misc_feature: TRAC AAV 5 TRAC 3 HA TRAC 3-2A-C11D5.3-CD8- CD28-CD3z-CISCb-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 4425 bp, Artificial sequence
  Synthetic polynucleotide
  misc_feature: TRAC AAV 5 TRAC 1 HA TRAC 1-2A-C11D5.3-CD8- CD28-CD3z-CISCb-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - DNA, 4867 bp, Artificial sequence
  Synthetic polynucleotide
  misc_feature: TRAC AAV 6 TRAC 2 HA TRAC 2-C11D5.3-CD8-41BB- CD3z-P2A-CISCb-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtgtgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 35 - DNA, 4867 bp, Artificial sequence
  Synthetic polynucleotide
  misc_feature: TRAC AAV 6 TRAC 3 HA TRAC 3-C11D5.3-CD8-41BB- CD3z-P2A-CISCb-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcctgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 36 - DNA, 4867 bp, Artificial sequence
  Synthetic polynucleotide
  misc_feature: TRAC AAV 6 TRAC 1 HA TRAC 1-C11D5.3-CD8-41BB- CD3z-P2A-CISCb-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagactgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 37 - DNA, 4452 bp, Artificial sequence
  Synthetic polynucleotide
  misc_feature: TRAC AAV 7 TRAC 2 HA TRAC 2-2A-C11D5.3-AAA-CD8-41BB-CD3z-P2A-CISCb-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtgggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 38 - DNA, 4452 bp, Artificial sequence
  Synthetic polynucleotide
  misc_feature: TRAC AAV 7 TRAC 3 HA TRAC 3-2A-C11D5.3-AAA-CD8-41BB-CD3z-P2A-CISCb-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgagtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 39 - DNA, 4452 bp, Artificial sequence
  Synthetic polynucleotide
  misc_feature: TRAC AAV 7 TRAC 1 HA TRAC 1-2A-C11D5.3-AAA-CD8-41BB-CD3z-P2A-CISCb-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 40 - DNA, 3718 bp, Artificial sequence
  Synthetic polynucleotide
  misc_feature: IL2RG AAV 8 HA-MND-nakedFRB-tLNGFR-CNb30-CISCg HA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagttaattaagtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggtgctggcgcaactggacgcgctatggatggacctcgcttgctgcttcttctgcttctcggggtctcattgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcatgtcaggataagcagaacaccgtctgcgaagagtgccccgatggcacctacagcgatgaagcgaaccatgtagacccctgcctgccttgcaccgtttgtgaagacacggaacgacagttgcgggagtgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcctacatcgctttcaaaagaggttccggggagggccgagggtcattgctgacgtgtggagacgtggaggagaatcctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaagagaatcctttcctgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcatcaattgtctacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 41 - DNA, 3718 bp, Artificial sequence
  Synthetic polynucleotide
  misc_feature: IL2RG AAV 8 GC8 HA-MND-nakedFRB-tLNGFR-CNb30- CISCg HA for GC8
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagttaattaagtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggtgctggcgcaactggacgcgctatggatggacctcgcttgctgcttcttctgcttctcggggtctcattgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcatgtcaggataagcagaacaccgtctgcgaagagtgccccgatggcacctacagcgatgaagcgaaccatgtagacccctgcctgccttgcaccgtttgtgaagacacggaacgacagttgcgggagtgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcctacatcgctttcaaaagaggttccggggagggccgagggtcattgctgacgtgtggagacgtggaggagaatcctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaagaaaacccctttttgttcgctcttgaggctgtcgtgattagcgtcggatccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcatcaattgtctacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - DNA, 3718 bp, Artificial sequence
  Synthetic polynucleotide
  misc_feature: IL2RG AAV 8 GC10 HA-MND-nakedFRB-tLNGFR-CNb30- CISCg HA for GC10
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagttaattaagtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggtgctggcgcaactggacgcgctatggatggacctcgcttgctgcttcttctgcttctcggggtctcattgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcatgtcaggataagcagaacaccgtctgcgaagagtgccccgatggcacctacagcgatgaagcgaaccatgtagacccctgcctgccttgcaccgtttgtgaagacacggaacgacagttgcgggagtgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcctacatcgctttcaaaagaggttccggggagggccgagggtcattgctgacgtgtggagacgtggaggagaatcctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaagaaaacccctttttgttcgctcttgaggctgtcgtgattagcgtcggaagtatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcatcaattgtctacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 43 - DNA, 3718 bp, Artificial sequence
  Synthetic polynucleotide
  misc_feature: IL2RG AAV 8 GC12 HA-MND-nakedFRB-tLNGFR-CNb30- CISCg HA for GC12
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagttaattaagtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggtgctggcgcaactggacgcgctatggatggacctcgcttgctgcttcttctgcttctcggggtctcattgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcatgtcaggataagcagaacaccgtctgcgaagagtgccccgatggcacctacagcgatgaagcgaaccatgtagacccctgcctgccttgcaccgtttgtgaagacacggaacgacagttgcgggagtgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcctacatcgctttcaaaagaggttccggggagggccgagggtcattgctgacgtgtggagacgtggaggagaatcctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaagaaaacccctttttgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcatcaattgtctacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 4678 bp, Artificial sequence
  Synthetic polynucleotide
  misc_feature: IL2RG AAV 9 GC10 HA-MND-B2MCAR-nakedFRB-tLNGFR- CNb30-CISCg HA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagttaattaagtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatgagcaggtcagtggcgttggcggttctggcgcttttgagtttgagcggactggaagccatccaacgaacgcctaagatccaggtatattcacgccacccggcggaaaacggcaaaagtaacttccttaattgttatgtgtctggcttccacccgtctgatattgaggtggacctccttaaaaacggtgaacggatcgagaaagtggagcattccgatcttagtttcagtaaggattggagcttttaccttctctattacactgagttcactccgactgaaaaggatgagtacgcctgtcgggtcaaccacgtcaccctgtctcaaccaaaaatagtcaaatgggacagagatatgtcagatatttacatatgggcaccacttgcgggcacgtgtggcgtcctgcttctgagtctcgtcattacgctttattgtaaacggggtagaaaaaaactcctttatatatttaaacagccatttatgcggccagttcaaacgacgcaggaagaagacggctgtagttgcagatttccagaggaagaggaaggtggatgcgagcttcgggtcaagtttagtaggtctgcagacgctcccgcctatcaacagggtcagaatcagctttataacgaactcaacctcggtcgccgagaagagtacgacgtactcgataaaagaaggggtagagacccggaaatggggggcaaaccgcgccgcaaaaatccacaagaggggctttataatgagcttcaaaaagacaaaatggccgaagcatacagtgagattgggatgaaaggtgaacgcagaagaggtaagggtcacgacgggctgtaccagggtttgtcaactgccacaaaggatacttatgacgctctgcatatgcaagctcttcccccacgcggcagcggcgaaggcagaggatccctgcttacatgtggcgacgtggaagagaaccctggccccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggtgctggcgcaactggacgcgctatggatggacctcgcttgctgcttcttctgcttctcggggtctcattgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcatgtcaggataagcagaacaccgtctgcgaagagtgccccgatggcacctacagcgatgaagcgaaccatgtagacccctgcctgccttgcaccgtttgtgaagacacggaacgacagttgcgggagtgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcctacatcgctttcaaaagaggttccggggagggccgagggtcattgctgacgtgtggagacgtggaggagaatcctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaagagaatcctttcctgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcatcaattgtctacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 45 - DNA, 4201 bp, Artificial sequence
  Synthetic polynucleotide
  misc_feature: IL2RG AAV 10 HA-MND-nakedFRB-CNb30-CISCb-CISCg HA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagttaattaagtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggcagcggcgaaggcagaggatccctgcttacatgtggcgacgtggaagagaaccctggccccatgccacttggcctgctctggctgggcttggcattgctcggcgcgctccacgcccaggctgaactgatccgcgtggccatattgtggcacgagatgtggcacgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaagagaatcctttcctgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcatcaattgtctacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 46 - DNA, 4582 bp, Artificial sequence
  Synthetic polynucleotide
  misc_feature: IL2RG AAV 11 HA-MND-B2MCAR-nakedFRB-CISCb-CISCg HA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagttaattaagtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatgagcaggtcagtggcgttggcggttctggcgcttttgagtttgagcggactggaagccatccaacgaacgcctaagatccaggtatattcacgccacccggcggaaaacggcaaaagtaacttccttaattgttatgtgtctggcttccacccgtctgatattgaggtggacctccttaaaaacggtgaacggatcgagaaagtggagcattccgatcttagtttcagtaaggattggagcttttaccttctctattacactgagttcactccgactgaaaaggatgagtacgcctgtcgggtcaaccacgtcaccctgtctcaaccaaaaatagtcaaatgggacagagatatgtcagatatttacatatgggcaccacttgcgggcacgtgtggcgtcctgcttctgagtctcgtcattacgctttattgtaaacggggtagaaaaaaactcctttatatatttaaacagccatttatgcggccagttcaaacgacgcaggaagaagacggctgtagttgcagatttccagaggaagaggaaggtggatgcgagcttcgggtcaagtttagtaggtctgcagacgctcccgcctatcaacagggtcagaatcagctttataacgaactcaacctcggtcgccgagaagagtacgacgtactcgataaaagaaggggtagagacccggaaatggggggcaaaccgcgccgcaaaaatccacaagaggggctttataatgagcttcaaaaagacaaaatggccgaagcatacagtgagattgggatgaaaggtgaacgcagaagaggtaagggtcacgacgggctgtaccagggtttgtcaactgccacaaaggatacttatgacgctctgcatatgcaagctcttcccccacgcggcagcggcgaaggcagaggatccctgcttacatgtggcgacgtggaagagaaccctggccccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgccacttggcctgctctggctgggcttggcattgctcggcgcgctccacgcccaggctgaactgatccgcgtggccatattgtggcacgagatgtggcacgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaagagaatcctttcctgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcatcaattgtctacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 47 - protein, 108 aa, Artificial sequence
  Synthetic polypeptide
  misc_feature: FKBP CISC domain
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 48 - protein, 100 aa, Artificial sequence
  Synthetic polypeptide
  misc_feature: FRB CISC domain
ELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQ

SEQ ID NO: 49 - protein, 39 aa, Artificial sequence
  Synthetic polypeptide
  misc_feature: ILR2g CISC fragment
GSNTSKENPFLFALEAVVISVGSMGLIISLLCVYFWLER

SEQ ID NO: 50 - protein, 123 aa, Artificial sequence
  Synthetic polypeptide
  misc_feature: ILR2g CISC domain
GSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 51 - protein, 315 aa, Artificial sequence
  Synthetic polypeptide
  misc_feature: ILR2b CISC domain
GKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 52 - protein, 147 aa, Artificial sequence
  Synthetic polypeptide
  misc_feature: CISCg fragment
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLER

SEQ ID NO: 53 - protein, 231 aa, Artificial sequence
  Synthetic polypeptide
  misc_feature: CISCg component
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 54 - protein, 415 aa, Artificial sequence
  Synthetic polypeptide
  misc_feature: CISCb component
ELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 55 - protein, 246 aa, Artificial sequence
  Synthetic polypeptide
  misc_feature: anti-BCMA scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 56 - protein, 83 aa, Artificial sequence
  Synthetic polypeptide
  misc_feature: CD8 transmembrane domain
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 57 - protein, 41 aa, Artificial sequence
  Synthetic polypeptide
  misc_feature: CD28 co-stimulatory domain
RSKRSRLLHSDYMNMTPRRPGPTRKHYQPYAPPRDFAAYRS

SEQ ID NO: 58 - protein, 47 aa, Artificial sequence
  Synthetic polypeptide
  misc_feature: 4-1BB co-stimulatory domain
RFSVVKRGRKKLLYIFKQPFMRPVQTTQEEDGCSCRFPEEEEGGCEL

SEQ ID NO: 59 - protein, 112 aa, Artificial sequence
  Synthetic polypeptide
  misc_feature: CD3zeta activation domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 482 aa, Artificial sequence
  Synthetic polypeptide
  misc_feature: anti-BCMA CAR, CD28
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 491 aa, Artificial sequence
  Synthetic polypeptide
  misc_feature: anti-BCMA CAR, 4-1BB
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 119 aa, Artificial sequence
  Synthetic polypeptide
  misc_feature: beta-2-microglobulin domain
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 63 - protein, 26 aa, Artificial sequence
  Synthetic polypeptide
  misc_feature: CD8 transmembrane domain
SDIYIWAPLAGTCGVLLLSLVITLYC

SEQ ID NO: 64 - protein, 42 aa, Artificial sequence
  Synthetic polypeptide
  misc_feature: 4-1BB co-stimulatory domain
KRGRKKLLYIFKQPFMRPVQTTQEEDGCSCRFPEEEEGGCEL

SEQ ID NO: 65 - protein, 299 aa, Artificial sequence
  Synthetic polypeptide
  misc_feature: beta-2-microglobulin chimeric receptor
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMSDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 274 aa, Artificial sequence
  Synthetic polypeptide
  misc_feature: tLNGFR polypeptide
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 67 - protein, 172 aa, Artificial sequence
  Synthetic polypeptide
  misc_feature: CNb30 polypeptide
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNTKLADTQLQQIVDKTIINADKDGDGRISFEEFCAVVGGLDIHKKMVVDV

SEQ ID NO: 68 - protein, 90 aa, Artificial sequence
  Synthetic polypeptide
  misc_feature: naked FRB wild-type polypeptide
MEMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 69 - protein, 90 aa, Artificial sequence
  Synthetic polypeptide
  misc_feature: naked FRB mutant polypeptide
MEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 70 - protein, 21 aa, Artificial sequence
  Synthetic polypeptide
  misc_feature: CD8 signal
MALPVTALLLPLALLLHAARP

SEQ ID NO: 71 - protein, 20 aa, Artificial sequence
  Synthetic polypeptide
  misc_feature: ER signal
MPLGLLWLGLALLGALHAQA

SEQ ID NO: 72 - protein, 21 aa, Artificial sequence
  Synthetic polypeptide
  misc_feature: T2A
GSGEGRGSLLTCGDVEENPGP

SEQ ID NO: 73 - protein, 22 aa, Artificial sequence
  Synthetic polypeptide
  misc_feature: P2A
GSGATNFSLLKQAGDVEENPGP

SEQ ID NO: 74 - DNA, 367 bp, Artificial sequence
  Synthetic polynucleotide
  misc_feature: MND promoter
acgtaagcttgtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcaaagcttacgt

SEQ ID NO: 75 - DNA, 373 bp, Artificial sequence
  Synthetic polynucleotide
  misc_feature: MSCV promoter
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggc

SEQ ID NO: 76 - protein, 131 aa, Artificial sequence
  Synthetic polypeptide
  misc_feature: truncated IL2Rbeta domain
PAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQ

SEQ ID NO: 77 - protein, 245 aa, Artificial sequence
  Synthetic polypeptide
  misc_feature: CISCb component, truncated
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQ

SEQ ID NO: 78 - DNA, 20 bp, Artificial sequence
  Synthetic polynucleotide
  misc_feature: BCMA target sequence
tattaagctcagtcccaaac

SEQ ID NO: 79 - DNA, 20 bp, Artificial sequence
  Synthetic polynucleotide
  misc_feature: AAVS1  target sequence
ggggccactagggacaggat

SEQ ID NO: 80 - DNA, 400 bp, Artificial sequence
  Synthetic polynucleotide
  misc_feature: TRAC 1 5′ homology arm
tggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagac

SEQ ID NO: 81 - DNA, 400 bp, Artificial sequence
  Synthetic polynucleotide
  misc_feature: TRAC 1 3′ homology arm
atgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgc

SEQ ID NO: 82 - DNA, 400 bp, Artificial sequence
  Synthetic polynucleotide
  misc_feature: TRAC 2 5′ homology arm
ccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtg

SEQ ID NO: 83 - DNA, 400 bp, Artificial sequence
  Synthetic polynucleotide
  misc_feature: TRAC 2 3′ homology arm
gcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctc

SEQ ID NO: 84 - DNA, 400 bp, Artificial sequence
  Synthetic polynucleotide
  misc_feature: TRAC 3 5′ homology arm
cccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcc

SEQ ID NO: 85 - DNA, 400 bp, Artificial sequence
  Synthetic polynucleotide
  misc_feature: TRAC 3 3′ homology arm
gtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaaga

SEQ ID NO: 86 - DNA, 400 bp, Artificial sequence
  Synthetic polynucleotide
  misc_feature: GC8 5′ homology arm
caacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatag

SEQ ID NO: 87 - DNA, 436 bp, Artificial sequence
  Synthetic polynucleotide
  misc_feature: GC8 3′ homology arm
gaaaacccctttttgttcgctcttgaggctgtcgtgattagcgtcggatccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcat

SEQ ID NO: 88 - DNA, 400 bp, Artificial sequence; identical to SEQ ID NO: 86
  Synthetic polynucleotide
  misc_feature: GC10 5′ homology arm

SEQ ID NO: 89 - DNA, 436 bp, Artificial sequence
  Synthetic polynucleotide
  misc_feature: GC10 3′ homology arm
gaaaacccctttttgttcgctcttgaggctgtcgtgattagcgtcggaagtatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcat

SEQ ID NO: 90 - DNA, 400 bp, Artificial sequence; identical to SEQ ID NO: 86
  Synthetic polynucleotide
  misc_feature: GC12 5′ homology arm

SEQ ID NO: 91 - DNA, 436 bp, Artificial sequence
  Synthetic polynucleotide
  misc_feature: GC12 3′ homology arm
gaaaacccctttttgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcat

SEQ ID NO: 92 - DNA, 4541 bp, Artificial sequence
  Synthetic polynucleotide
  misc_feature: AAV HA TRAC 1-TNP-AAA-CD8-CD28-CD3z-P2A-CISCb- HA TRAC 1
tggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagactgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacattgtgatgacccagtctcaaaaattcatgtccacatcagtaggagacagggtcagcatcacctgcaaggccagtcagaatgtgggtactgctgtagcctggtatcaacagaaaccaggacaatctcctaaactactgatttactcggcatccaatcggtacactggagtccctgatcgcttcacaggcagtggatctgggacagatttcactctcaccatcagcaatatgcagtctgaagacctggcagattatttctgccagcaatatagcagctatcctctcacgttcggtgctgggaccaagctggagctgaaaggcagcaccagcggctccggcaagcctggctctggcgagggcagcacaaagggacaggtccagctgcagcagtctggacctgagctggtgaagcctggggcttcagtgaggatatcctgcaaggcttctggctacaccttcacaagctactatatacactgggtgaagcagaggcctggacagggacttgagtggattggatggatttatcctggaaatgttaatactaagtacaatgagaagttcaagggcaaggccacactgactgcagacaaatcctccagcacagcctacatgcagctcagcagcctgacctctgaggactctgcggtctatttctgtgcaagaaactacggtagtagctacgggcttgcttactggggccaagggactctggtcactgtctctgcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgc

SEQ ID NO: 93 - DNA, 4568 bp, Artificial sequence
  Synthetic polynucleotide
  misc_feature: AAV HA TRAC 1-TNP-AAA-CD8-41BB-CD3z-P2A-CISCb- HA TRAC 1
tggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagactgaatgaatgattaattaataaaagatctttattttcattagatctgtgtgttggttttttgtgtgatcctcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgcgccagtccggtaccagtcgccaccatggccctgcctgtgacagctctgctcctccctctggccctgctgctccatgccgccagacccgacattgtgatgacccagtctcaaaaattcatgtccacatcagtaggagacagggtcagcatcacctgcaaggccagtcagaatgtgggtactgctgtagcctggtatcaacagaaaccaggacaatctcctaaactactgatttactcggcatccaatcggtacactggagtccctgatcgcttcacaggcagtggatctgggacagatttcactctcaccatcagcaatatgcagtctgaagacctggcagattatttctgccagcaatatagcagctatcctctcacgttcggtgctgggaccaagctggagctgaaaggcagcaccagcggctccggcaagcctggctctggcgagggcagcacaaagggacaggtccagctgcagcagtctggacctgagctggtgaagcctggggcttcagtgaggatatcctgcaaggcttctggctacaccttcacaagctactatatacactgggtgaagcagaggcctggacagggacttgagtggattggatggatttatcctggaaatgttaatactaagtacaatgagaagttcaagggcaaggccacactgactgcagacaaatcctccagcacagcctacatgcagctcagcagcctgacctctgaggactctgcggtctatttctgtgcaagaaactacggtagtagctacgggcttgcttactggggccaagggactctggtcactgtctctgcagccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaacttgtttattgcagcttataatggttacaaataaagcaatagcatcacaaatttcacaaataaagcatttttttcactgcattctagttgtggtttgtccaaactcatcaatgtatcttacgccggcgtgaatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgc

SEQ ID NO: 94 - DNA, 5203 bp, Artificial sequence
  Synthetic polynucleotide
  misc_feature: TRAC AAV TRAC 2 HA TRAC 2-synpA-MND-Kozak-ER-FKBP-IL2RG-P2A-ER-FRB-IL2RB-P2A-mCherry-WPRE 3-BGHpA-HA TRAC 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccgcggcggcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtttaattaaatgaaataaaagatctttattttcattagatctgtgtgttggttttttgtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgccacttggcctgctctggctgggcttggcattgctcggcgcgctccacgcccaggctggcgttcaagttgaaaccattagtcccggagacggtcgaacatttcccaaacggggccagacgtgcgtggtacactacaccggaatgctggaggatggaaaaaaatttgacagcagccgggacagaaacaaaccattcaagttcatgcttggtaaacaagaggtaatacggggttgggaagagggtgtggcccagatgtcagtagggcaacgcgcgaagttgaccataagccccgactatgcctatggggcgacaggccatcccggtataattcctccgcacgctacactggtgtttgatgttgagttgctgaagctggagcaaaatcttgttattccgtgggctcccgagaacctcacattgcacaaattgtccgaatcacaattggagcttaattggaacaatagattcctgaatcactgccttgagcacctcgtacaataccggacagactgggatcactcttggacggagcagtccgtggactaccgacataaattctcactcccctcagtggatggccagaaacgctatacctttagagtccggtcccgcttcaacccgttgtgcggcagcgcacagcactggagtgaatggagtcatccgatacactggggaagcaatacgtcaaaagagaacccgttcctttttgcgctggaagcagtcgtgatcagcgttggatctatggggctgatcatctcccttctctgcgtctatttctggctcgaaagaactatgccacgcatccctacgctgaaaaatctggaggatcttgtgacggaatatcatggaaatttttccgcctggagtggagtttccaaaggtctcgctgaatctctgcagccagactatagtgagcggctctgcttggtctctgagattccacctaaggggggggcgctcggggaaggcccgggcgcaagtccgtgtaatcaacacagtccgtactgggctccaccatgctataccctcaagccggaaactggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggaagcggagctactaacttcagcctgctgaagcaggctggagacgtggaggagaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggtaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacgccggcgagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctcctagggaattgccttaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 95 - DNA, 5203 bp, Artificial sequence
  Synthetic polynucleotide
  misc_feature: TRAC AAV TRAC 3 HA TRAC 3-synpA-MND-Kozak-ER-FKBP-IL2RG-P2A-ER-FRB-IL2RB-P2A-mCherry-WPRE 3-BGHpA-HA TRAC 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccgcggcggtgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacttaattaaatgaaataaaagatctttattttcattagatctgtgtgttggttttttgtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgccacttggcctgctctggctgggcttggcattgctcggcgcgctccacgcccaggctggcgttcaagttgaaaccattagtcccggagacggtcgaacatttcccaaacggggccagacgtgcgtggtacactacaccggaatgctggaggatggaaaaaaatttgacagcagccgggacagaaacaaaccattcaagttcatgcttggtaaacaagaggtaatacggggttgggaagagggtgtggcccagatgtcagtagggcaacgcgcgaagttgaccataagccccgactatgcctatggggcgacaggccatcccggtataattcctccgcacgctacactggtgtttgatgttgagttgctgaagctggagcaaaatcttgttattccgtgggctcccgagaacctcacattgcacaaattgtccgaatcacaattggagcttaattggaacaatagattcctgaatcactgccttgagcacctcgtacaataccggacagactgggatcactcttggacggagcagtccgtggactaccgacataaattctcactcccctcagtggatggccagaaacgctatacctttagagtccggtcccgcttcaacccgttgtgcggcagcgcacagcactggagtgaatggagtcatccgatacactggggaagcaatacgtcaaaagagaacccgttcctttttgcgctggaagcagtcgtgatcagcgttggatctatggggctgatcatctcccttctctgcgtctatttctggctcgaaagaactatgccacgcatccctacgctgaaaaatctggaggatcttgtgacggaatatcatggaaatttttccgcctggagtggagtttccaaaggtctcgctgaatctctgcagccagactatagtgagcggctctgcttggtctctgagattccacctaaggggggggcgctcggggaaggcccgggcgcaagtccgtgtaatcaacacagtccgtactgggctccaccatgctataccctcaagccggaaactggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggaagcggagctactaacttcagcctgctgaagcaggctggagacgtggaggagaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggtaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacgccggcgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtcctagggaattgccttaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 96 - DNA, 5217 bp, Artificial sequence
  Synthetic polynucleotide
  misc_feature: TRAC AAV TRAC 1 HA AS-synpA-MND-Kozak-ER-FKBP-IL2RG-P2A-ER-FRB-IL2RB-P2A-mCherry-WPR E3-BGHpA-HA TRAC 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccgcggcggccgcgccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacatgttaattaaatgaaataaaagatctttattttcattagatctgtgtgttggttttttgtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgccacttggcctgctctggctgggcttggcattgctcggcgcgctccacgcccaggctggcgttcaagttgaaaccattagtcccggagacggtcgaacatttcccaaacggggccagacgtgcgtggtacactacaccggaatgctggaggatggaaaaaaatttgacagcagccgggacagaaacaaaccattcaagttcatgcttggtaaacaagaggtaatacggggttgggaagagggtgtggcccagatgtcagtagggcaacgcgcgaagttgaccataagccccgactatgcctatggggcgacaggccatcccggtataattcctccgcacgctacactggtgtttgatgttgagttgctgaagctggagcaaaatcttgttattccgtgggctcccgagaacctcacattgcacaaattgtccgaatcacaattggagcttaattggaacaatagattcctgaatcactgccttgagcacctcgtacaataccggacagactgggatcactcttggacggagcagtccgtggactaccgacataaattctcactcccctcagtggatggccagaaacgctatacctttagagtccggtcccgcttcaacccgttgtgcggcagcgcacagcactggagtgaatggagtcatccgatacactggggaagcaatacgtcaaaagagaacccgttcctttttgcgctggaagcagtcgtgatcagcgttggatctatggggctgatcatctcccttctctgcgtctatttctggctcgaaagaactatgccacgcatccctacgctgaaaaatctggaggatcttgtgacggaatatcatggaaatttttccgcctggagtggagtttccaaaggtctcgctgaatctctgcagccagactatagtgagcggctctgcttggtctctgagattccacctaaggggggggcgctcggggaaggcccgggcgcaagtccgtgtaatcaacacagtccgtactgggctccaccatgctataccctcaagccggaaactggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggaagcggagctactaacttcagcctgctgaagcaggctggagacgtggaggagaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggtaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacgccggcgtggcggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctccctagggaattgccttaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 97 - DNA, 3069 bp, Artificial sequence
  Synthetic polynucleotide
  misc_feature: IL2RG cassette  CISC-tLNGFR
atgaaataaaagatctttattttcattagatctgtgtgttggttttttgtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgggtgctggcgcaactggacgcgctatggatggacctcgcttgctgcttcttctgcttctcggggtctctttgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcctgtcaggataagcagaacaccgtctgcgaagagtgccccgatggtacctacagcgatgaagcgaaccatgtagacccatgcctgccttgcaccgtttgtgaagacacggaacgacagttgcgggaatgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcgtacatcgctttcaaaagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggatccggcgctacaaatttttcactgctgaaacaggcgggtgatgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaa

SEQ ID NO: 98 - DNA, 1509 bp, Artificial sequence
  Synthetic polynucleotide
  misc_feature: TRAC 1 cassette C11D5.3-CD8-CD28-CD3z
atggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 99 - DNA, 1572 bp, Artificial sequence
  Synthetic polynucleotide
  misc_feature: TRAC 2 cassette 2A-C11D5.3-CD8-CD28-CD3z
ggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 100 - DNA, 2151 bp, Artificial sequence
  Synthetic polynucleotide
  misc_feature: TRAC 3 cassette 2A-C11D5.3-CD8-CD28-CD3z-CNb30
ggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggcagcggcgaaggcagaggatccctgcttacatgtggcgacgtggaagagaaccctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtg

SEQ ID NO: 101 - DNA, 2880 bp, Artificial sequence
  Synthetic polynucleotide
  misc_feature: TRAC 4 cassette C11D5.3-CD8-CD28-CD3z-P2A-CISCb
atggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtg

SEQ ID NO: 102 - DNA, 2943 bp, Artificial sequence
  Synthetic polynucleotide
  misc_feature: TRAC 5 cassette 2A-C11D5.3-CD8-CD28-CD3z-CISCb
ggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtg

SEQ ID NO: 103 - DNA, 2907 bp, Artificial sequence
  Synthetic polynucleotide
  misc_feature: TRAC 6 cassette C11D5.3-CD8-41BB-CD3z-P2A-CISCb
atggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtg

SEQ ID NO: 104 - DNA, 2970 bp, Artificial sequence
  Synthetic polynucleotide
  misc_feature: TRAC 7 cassette 2A-C11D5.3-AAA-CD8-41BB-CD3z- P2A-CISCb
ggttccggggagggccgagggtcattgctgacgtgtggagacgtggaggagaatcctggccccatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagcgccgccgccttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacagattcagcgtcgtgaagcggggcagaaagaagctgctgtacatcttcaagcagcccttcatgcggcccgtgcagaccacacaagaggaagatggctgctcctgcagattccctgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagaggatccggcgctacaaatttttcactgctgaaacaggcgggtgatgtggaggagaaccctggacccatgccacttggcctgctctggctgggcttggcattgctcggcgcgctccacgcccaggctgaactgatccgcgtggccatattgtggcatgagatgtggcatgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtg

SEQ ID NO: 105 - DNA, 2571 bp, Artificial sequence
  Synthetic polynucleotide
  misc_feature: IL2RG 8 cassette MND-nakedFRB-tLNGFR-CNb30- CISCg
gtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggtgctggcgcaactggacgcgctatggatggacctcgcttgctgcttcttctgcttctcggggtctcattgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcatgtcaggataagcagaacaccgtctgcgaagagtgccccgatggcacctacagcgatgaagcgaaccatgtagacccctgcctgccttgcaccgtttgtgaagacacggaacgacagttgcgggagtgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcctacatcgctttcaaaagaggttccggggagggccgagggtcattgctgacgtgtggagacgtggaggagaatcctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaa

SEQ ID NO: 106 - DNA, 3531 bp, Artificial sequence
  Synthetic polynucleotide
  misc_feature: IL2RG 9 cassette MND-B2MCAR-nakedFRB-tLNGFR- CNb30-CISCg
gtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatgagcaggtcagtggcgttggcggttctggcgcttttgagtttgagcggactggaagccatccaacgaacgcctaagatccaggtatattcacgccacccggcggaaaacggcaaaagtaacttccttaattgttatgtgtctggcttccacccgtctgatattgaggtggacctccttaaaaacggtgaacggatcgagaaagtggagcattccgatcttagtttcagtaaggattggagcttttaccttctctattacactgagttcactccgactgaaaaggatgagtacgcctgtcgggtcaaccacgtcaccctgtctcaaccaaaaatagtcaaatgggacagagatatgtcagatatttacatatgggcaccacttgcgggcacgtgtggcgtcctgcttctgagtctcgtcattacgctttattgtaaacggggtagaaaaaaactcctttatatatttaaacagccatttatgcggccagttcaaacgacgcaggaagaagacggctgtagttgcagatttccagaggaagaggaaggtggatgcgagcttcgggtcaagtttagtaggtctgcagacgctcccgcctatcaacagggtcagaatcagctttataacgaactcaacctcggtcgccgagaagagtacgacgtactcgataaaagaaggggtagagacccggaaatggggggcaaaccgcgccgcaaaaatccacaagaggggctttataatgagcttcaaaaagacaaaatggccgaagcatacagtgagattgggatgaaaggtgaacgcagaagaggtaagggtcacgacgggctgtaccagggtttgtcaactgccacaaaggatacttatgacgctctgcatatgcaagctcttcccccacgcggcagcggcgaaggcagaggatccctgcttacatgtggcgacgtggaagagaaccctggccccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggtgctggcgcaactggacgcgctatggatggacctcgcttgctgcttcttctgcttctcggggtctcattgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcatgtcaggataagcagaacaccgtctgcgaagagtgccccgatggcacctacagcgatgaagcgaaccatgtagacccctgcctgccttgcaccgtttgtgaagacacggaacgacagttgcgggagtgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcctacatcgctttcaaaagaggttccggggagggccgagggtcattgctgacgtgtggagacgtggaggagaatcctggccccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaa

SEQ ID NO: 107 - DNA, 3054 bp, Artificial sequence
  Synthetic polynucleotide
  misc_feature: IL2RG 10 cassette MND-nakedFRB-CNb30-CISCb- CISCg
gtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgggcagcggcgaaggcagaggatccctgcttacatgtggcgacgtggaagagaaccctggccccatgccacttggcctgctctggctgggcttggcattgctcggcgcgctccacgcccaggctgaactgatccgcgtggccatattgtggcacgagatgtggcacgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaa

SEQ ID NO: 108 - DNA, 3435 bp, Artificial sequence
  Synthetic polynucleotide
  misc_feature: IL2RG 11 cassette MND-B2MCAR-nakedFRB-CISCb- CISCg
gtgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggtaccgccgccaccatgagcaggtcagtggcgttggcggttctggcgcttttgagtttgagcggactggaagccatccaacgaacgcctaagatccaggtatattcacgccacccggcggaaaacggcaaaagtaacttccttaattgttatgtgtctggcttccacccgtctgatattgaggtggacctccttaaaaacggtgaacggatcgagaaagtggagcattccgatcttagtttcagtaaggattggagcttttaccttctctattacactgagttcactccgactgaaaaggatgagtacgcctgtcgggtcaaccacgtcaccctgtctcaaccaaaaatagtcaaatgggacagagatatgtcagatatttacatatgggcaccacttgcgggcacgtgtggcgtcctgcttctgagtctcgtcattacgctttattgtaaacggggtagaaaaaaactcctttatatatttaaacagccatttatgcggccagttcaaacgacgcaggaagaagacggctgtagttgcagatttccagaggaagaggaaggtggatgcgagcttcgggtcaagtttagtaggtctgcagacgctcccgcctatcaacagggtcagaatcagctttataacgaactcaacctcggtcgccgagaagagtacgacgtactcgataaaagaaggggtagagacccggaaatggggggcaaaccgcgccgcaaaaatccacaagaggggctttataatgagcttcaaaaagacaaaatggccgaagcatacagtgagattgggatgaaaggtgaacgcagaagaggtaagggtcacgacgggctgtaccagggtttgtcaactgccacaaaggatacttatgacgctctgcatatgcaagctcttcccccacgcggcagcggcgaaggcagaggatccctgcttacatgtggcgacgtggaagagaaccctggccccatggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcacgtctttaggcgcattagtaagggcagcggcgccacaaatttcagcctgctgaaacaggccggcgacgtggaagagaaccctggacccatgccacttggcctgctctggctgggcttggcattgctcggcgcgctccacgcccaggctgaactgatccgcgtggccatattgtggcacgagatgtggcacgagggattggaggaggcgagtaggctgtactttggggaaaggaatgttaaagggatgtttgaggtccttgaacccctccacgctatgatggaaagaggacctcaaacgcttaaagagacgtcattcaatcaagcctatggacgggatcttatggaagctcaagaatggtgtcgaaaatacatgaaaagcgggaatgttaaggacctcacgcaagcctgggatctgtattaccatgttttccgacgcatttctaaacaaggaaaagatactatcccatggttggggcacttgctcgttgggctcagtggggcgtttggattcatcatcctcgtatatctgttgattaattgtcggaacacaggtccctggcttaaaaaagttttgaagtgtaacaccccggatccttctaaattttttagtcaacttagttcagaacacgggggcgatgttcaaaagtggctgagttccccgtttcccagttcaagtttctcccctgggggtctcgcccccgagatatcacctcttgaagtgctcgagcgggacaaagttacacagcttcttttgcaacaggataaggttccggagccggcgtctctcagctctaaccattcactcacttcttgtttcaccaaccaagggtattttttcttccatctgcctgatgccttggagattgaggcttgtcaggtgtactttacctatgacccctatagtgaggaagaccctgacgaaggcgtagctggcgcccccactggctccagtccacagcctcttcagcctctgtcaggggaggacgacgcatattgtacgttcccctcacgggacgaccttctgctgttttcaccctcactgctcggcggaccctccccgccaagcacggcacctggggggagtggggcaggagaagaaaggatgcctcctagtttgcaggagcgggttcctcgcgactgggatccgcaacccctcggaccacccacccctggcgtacctgatctggtcgacttccaaccacctccggagcttgtcctcagagaggccggagaggaagtcccagacgcggggccaagagagggtgtgtcatttccctggtcccgccctccgggacagggtgagtttcgggcgctgaatgcgaggctcccccttaataccgatgcgtacctgtcattgcaggaacttcagggccaggatcctacccacctggtgggatccggcgctacaaatttttcactgctgaaacaggcgggtgacgtggaggagaaccctggacccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcagcaacaccagcaaa

(19) United States
(12) Patent Application Publication
(10) Pub. No.: US 2021/0228629 A1
(43) Pub. Date: Jul. 29, 2021

(54) ANTI-BCMA CAR-T-CELLS FOR PLASMA CELL DEPLETION

(71) Applicants: CRISPR THERAPEUTICS AG, Zug (CH); BAYER HEALTHCARE, LLC, Whippany, NJ (US)

(72) Inventor: Gregory COST, Cambridge, MA (US)

(21) Appl. No.: 17/050,694

(22) PCT Filed: Apr. 26, 2019
(86) PCT No.: PCT/US2019/029503

§ 371 Date: Oct. 26, 2020

Related U.S. Application Data
(60) Provisional application No. 62/773,058, filed on Nov. 29, 2018.
(60) Provisional application No. 62/663,974, filed on Apr. 27, 2018.

(51) Int. Cl. A61K 35/17; C12N 15/113; C07K 14/715; A61P 43/00; C12N 15/90

(52) U.S. Cl. CPC A61K 35/17; C12N 15/113; C12N 15/907; A61P 43/00; C07K 14/7155

(57) ABSTRACT

The present application relates to compositions and methods for controlled plasma cell depletion, such as for the treatment of various diseases and conditions associated with plasma cells.

### CROSS-REFERENCE TO RELATED APPLICATIONS

This application claims the benefit of priority to U.S. Provisional Patent Application No. 62/663,974, filed Apr. 27, 2018, and U.S. Provisional Patent Application No. 62/773,058, filed Nov. 29, 2018, the disclosures of each of which are incorporated herein by reference in their entireties.

### SEQUENCE LISTING

This application contains a Sequence Listing which has been submitted electronically in ASCII format and is hereby incorporated by reference in its entirety. The ASCII copy, created on Apr. 26, 2019, is named 052984-515001WO_SL_ST25.txt, and is 295,038 bytes in size.

### FIELD

The present disclosure relates to compositions and methods for controlled plasma cell depletion in an individual. In particular, the compositions include a general architecture for generating physiologically functional synthetic chemical-induced signaling complexes (CISCs) that allow for controlling the survival and/or proliferation of T cells. Further provided are methods of using such compositions, such as for the treatment of various diseases and conditions.

### BACKGROUND

Chimeric antigen receptors (CARs) are engineered receptors used to genetically engineer T cells for use in adoptive cellular immunotherapy (see Pule, M. et al. (2003). Cytother., 5(3):211-226; Restifo, N. P. et al. (2012). Nat. Rev. Immunol. 12(4):269-281). Antigen binding stimulates the signaling domains on the intracellular segment of the CAR, thereby activating signaling pathways. CAR-based adoptive cellular immunotherapy has been used to treat cancer patients with tumors refractory to conventional standard-of-care treatments (see Grupp, S. A. et al. (2013). N. Engl. J. Med. 368(16):1509-1518; Kalos, M. et al. (2011). Sci. Transl. Med. 3(95):95ra73).

CAR-based adoptive cellular immunotherapy can also be used to target host cells involved in a disease or condition. For example, CAR T cells specific for antibody-producing plasma cells could potentially be used to treat diseases or conditions characterized by an adverse antibody-mediated immune response, such as autoimmunity or organ graft rejection. However, administration of conventional CAR T cells targeting plasma cells in an individual would lead to uncontrolled depletion of plasma cells in the individual, which could result in severe adverse effects, such as inability to respond to pathogenic infections. There remains a need for new compositions and methods that allow for controlling the depletion of plasma cells to allow for viable treatments for diseases and conditions characterized by adverse antibody production.

### SUMMARY

Described herein are engineered T cells cytotoxic towards plasma cells, wherein the engineered T cells comprise a chemical-induced signaling complex (CISC) allowing for controlled survival and/or proliferation of the engineered T cells, methods of making and using the engineered T cells, and compositions useful for the methods.

In one aspect, provided herein is an engineered T cell comprising a) an endogenous T cell receptor alpha (TRA) gene modified to encode a non-functional T cell receptor alpha constant (TRAC) domain; and b) a nucleic acid encoding a chimeric antigen receptor (CAR) that can recognize B-cell maturation antigen (BCMA). In some embodiments, the survival and/or proliferation of the engineered T cell can be controlled by modulating the amount of a ligand in contact with the engineered T cell.

In some embodiments, the CAR that can recognize BCMA comprises an extracellular BCMA recognition domain, a transmembrane domain, a co-stimulatory domain, and a cytoplasmic signaling domain.

In some embodiments, the extracellular BCMA recognition domain is an antibody moiety that can specifically bind to BCMA.

In some embodiments, the antibody moiety comprises a heavy chain variable domain (V_{H}) and a light chain variable domain (V_{L}) comprising heavy chain complementarity-determining region (HC-CDR)1, HC-CDR2, HC-CDR3, light chain complementarity-determining region (LC-CDR)1, LC-CDR2, and LC-CDR3 from SEQ ID NO: 55.

In some embodiments, the antibody moiety is an scFv.

In some embodiments, the CAR transmembrane domain comprises a CD8 transmembrane domain, the CAR co-stimulatory domain comprises a 4-1BB and/or a CD28 co-stimulatory domain, and/or the CAR cytoplasmic signaling domain comprises a CD3-ζ cytoplasmic signaling domain.

In some embodiments, the b) nucleic acid encoding a CAR that can recognize BCMA is inserted into the region of the endogenous TRA gene encoding the TRAC domain or the b) nucleic acid encoding a CAR that can recognize BCMA is inserted into an endogenous IL2RG gene.

In some embodiments, the polypeptide components of the CISC comprise i) a first CISC component comprising a first extracellular binding domain or portion thereof, a hinge domain, a transmembrane domain, and a signaling domain or portion thereof; and ii) a second CISC component comprising a second extracellular binding domain or portion thereof, a hinge domain, a transmembrane domain, and a signaling domain or portion thereof; wherein the first CISC component and the second CISC component are configured such that when expressed, they dimerize in the presence of the ligand to create a signaling-competent CISC.

In some embodiments, the signaling domain of the first CISC component comprises an IL-2 receptor subunit gamma (IL2Rγ) cytoplasmic signaling domain.

In some embodiments, the IL2Rγ cytoplasmic signaling domain comprises the amino acid sequence of SEQ ID NO: 50 or a variant thereof having at least 85% homology to the amino acid sequence of SEQ ID NO: 50.

In some embodiments, the first extracellular binding domain or portion thereof comprises an FK506 binding protein (FKBP) domain or a portion thereof.

In some embodiments, the FKBP domain comprises the amino acid sequence of SEQ ID NO: 47 or a variant thereof having at least 85% homology to the amino acid sequence of SEQ ID NO: 47.

In some embodiments, the signaling domain of the second CISC component comprises an IL-2 receptor subunit beta (IL2Rβ) cytoplasmic signaling domain.

In some embodiments, the IL2Rβ cytoplasmic signaling domain comprises the amino acid sequence of SEQ ID NO: 51 or a variant thereof having at least 85% homology to the amino acid sequence of SEQ ID NO: 51.

In some embodiments, the second extracellular binding domain or portion thereof comprises an FKBP rapamycin binding (FRB) domain or a portion thereof.

In some embodiments, the FRB comprises the amino acid sequence of SEQ ID NO: 48 or a variant thereof having at least 85% homology to the amino acid sequence of SEQ ID NO: 48.

In some embodiments, the transmembrane domain of the first and second CISC components comprises, independently, an IL-2 receptor transmembrane domain.

In some embodiments, 1) the one or more nucleic acids encoding the first CISC component are inserted into an endogenous IL2RG gene and the one or more nucleic acids encoding the second CISC component are inserted into the region of the endogenous TRA gene encoding the TRAC domain; or 2) the one or more nucleic acids encoding the first CISC component are inserted into the region of the endogenous TRA gene encoding the TRAC domain and the one or more nucleic acids encoding the second CISC component are inserted into the endogenous IL2RG gene.

In some embodiments, the ligand is rapamycin or a rapamycin analog (rapalog).

In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

In some embodiments, the ligand is present or provided in an amount from 0.05 nM to 100 nM.

In some embodiments, the cell further comprises d) one or more nucleic acids encoding a chimeric receptor comprising an extracellular β2-microglobulin domain, a transmembrane domain, a co-stimulatory domain, and a cytoplasmic signaling domain.

In some embodiments, the chimeric receptor transmembrane domain comprises a CD8 transmembrane domain, the chimeric receptor co-stimulatory domain comprises a 4-1BB co-stimulatory domain, and/or the chimeric receptor cytoplasmic signaling domain comprises a CD3-ζ cytoplasmic signaling domain.

In some embodiments, the chimeric receptor comprises the amino acid sequence of SEQ ID NO: 65 or a variant thereof having at least 85% homology to the amino acid sequence of SEQ ID NO: 65

In some embodiments, the d) one or more nucleic acids encoding the chimeric receptor are inserted into the region of the endogenous TRA gene encoding the TRAC domain or the d) one or more nucleic acids encoding the chimeric receptor are inserted into an endogenous IL2RG gene.

In some embodiments, the cell further comprises g) a nucleic acid encoding a selectable marker.

In some embodiments, the selectable marker is a truncated low-affinity nerve growth factor receptor (tLNGFR) polypeptide.

In some embodiments, the tLNGFR polypeptide comprises the amino acid sequence of SEQ ID NO: 66.

In some embodiments, the nucleic acid encoding the selectable marker is inserted into the region of the endogenous TRA gene encoding the TRAC domain or the nucleic acid encoding the selectable marker is inserted into an endogenous IL2RG gene.

In some embodiments, the cell further comprises e) a nucleic acid encoding a polypeptide that confers resistance to one or more calcineurin inhibitors.

In some embodiments, the polypeptide that confers resistance to one or more calcineurin inhibitors confers resistance to tacrolimus (FK506) and/or cyclosporin A (CsA).

In some embodiments, the polypeptide that confers resistance to one or more calcineurin inhibitors is a mutant calcineurin (CN) polypeptide.

In some embodiments, the mutant CN polypeptide confers resistance to tacrolimus (FK506) and cyclosporin A (CsA).

In some embodiments, the mutant CN polypeptide is CNb30 (SEQ ID NO: 67).

In some embodiments, the nucleic acid encoding the polypeptide that confers resistance to one or more calcineurin inhibitors is inserted into the region of the endogenous TIM gene encoding the TRAC domain or the nucleic acid encoding the polypeptide that confers resistance to one or more calcineurin inhibitors is inserted into an endogenous IL2RG gene.

In some embodiments, the cell further comprises f) a nucleic acid encoding a FKBP-rapamycin binding (FRB) domain polypeptide of the mammalian target of rapamycin (mTOR) kinase.

In some embodiments, the FRB domain polypeptide is expressed intracellularly.

In some embodiments, the FRB domain polypeptide comprises the amino acid of SEQ ID NO: 68 or 69 or a variant having at least 90% sequence homology to the amino acid of SEQ ID NO: 68 or 69.

In some embodiments, the nucleic acid encoding the FRB domain polypeptide is inserted into the region of the endogenous TRA gene encoding the TRAC domain or the nucleic acid encoding the FRB domain polypeptide is inserted into an endogenous IL2RG gene.

In another aspect, provided herein is a guide RNA (gRNA) comprising a sequence that is complementary to a sequence in an endogenous TRA gene within or near a region encoding the TRAC domain.

In some embodiments, the gRNA comprises the polynucleotide sequence of any one of SEQ ID NOs: 1-3, or a variant thereof having at least 85% homology to any one of SEQ ID NOs: 1-3.

In another aspect, provided herein is a guide RNA (gRNA) comprising a sequence that is complementary to a sequence within or near an endogenous IL2RG gene.

In some embodiments, the gRNA comprises the polynucleotide sequence of any one of SEQ ID NOs: 4-18, or a variant thereof having at least 85% homology to any one of SEQ ID NOs: 4-18.

In another aspect, provided herein is a system comprising a) a first gRNA and/or a second gRNA, wherein the first gRNA is a gRNA according to any of the embodiments described above and the second gRNA is a gRNA according to any of the embodiments described above; and b) an RNA-guided endonuclease (RGEN) or a nucleic acid encoding the RGEN.

In some embodiments, the system further comprises c) one or more donor templates comprising nucleic acid encoding: i) a CAR that can recognize a B-cell maturation antigen (BCMA) polypeptide; ii) a first CISC component comprising a first extracellular binding domain or portion thereof, a hinge domain, a transmembrane domain, and a signaling domain or portion thereof or functional derivative thereof; and iii) a second CISC component comprising a second extracellular binding domain or portion thereof, a hinge domain, a transmembrane domain, and a signaling domain or portion thereof, wherein the first CISC component and the second CISC component are configured such that when expressed by a T cell, they dimerize in the presence of a ligand to create a signaling competent CISC capable of promoting the survival and/or proliferation of the T cell.

In some embodiments, the CAR that can recognize BCMA comprises an extracellular BCMA recognition domain, a transmembrane domain, a co-stimulatory domain, and a cytoplasmic signaling domain.

In some embodiments, the extracellular BCMA recognition domain is an antibody moiety that can specifically bind to BCMA.

In some embodiments, the antibody moiety comprises a heavy chain variable domain (V_{H}) and a light chain variable domain (V_{L}) comprising heavy chain complementarity-determining region (HC-CDR)1, HC-CDR2, HC-CDR3, light chain complementarity-determining region (LC-CDR)1, LC-CDR2, and LC-CDR3 from SEQ ID NO: 55.

In some embodiments, the antibody moiety is an scFv.

In some embodiments, the CAR transmembrane domain comprises a CD8 transmembrane domain, the CAR co-stimulatory domain comprises a 4-1BB and/or a CD28 co-stimulatory domain, and/or the CAR cytoplasmic signaling domain comprises a CD3-cytoplasmic signaling domain.

In some embodiments, the signaling domain of the first CISC component comprises an IL-2 receptor subunit gamma (IL2Rγ) domain.

In some embodiments, the IL2Rγ cytoplasmic signaling domain comprises the amino acid sequence of SEQ ID NO: 50 or a variant thereof having at least 85% homology to the amino acid sequence of SEQ ID NO: 50.

In some embodiments, the first extracellular binding domain or portion thereof comprises an FK506 binding protein (FKBP) domain or a portion thereof.

In some embodiments, the FKBP domain comprises the amino acid sequence of SEQ ID NO: 47 or a variant thereof having at least 85% homology to the amino acid sequence of SEQ ID NO: 47.

In some embodiments, the signaling domain of the second CISC component comprises an IL-2 receptor subunit beta (IL2Rβ) domain.

In some embodiments, the IL2Rβ cytoplasmic signaling domain comprises the amino acid sequence of SEQ ID NO: 51 or a variant thereof having at least 85% homology to the amino acid sequence of SEQ ID NO: 51.

In some embodiments, the second extracellular binding domain or portion thereof comprises an FKBP rapamycin binding (FRB) domain or a portion thereof.

In some embodiments, the FRB comprises the amino acid sequence of SEQ ID NO: 48 or a variant thereof having at least 85% homology to the amino acid sequence of SEQ ID NO: 48.

In some embodiments, the transmembrane domain of the first and second CISC components comprises, independently, an IL-2 receptor transmembrane domain.

In some embodiments, the ligand is rapamycin or a rapalog.

In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

In some embodiments, the c) one or more donor templates further comprise nucleic acid encoding one or more of: iv) a chimeric receptor comprising an extracellular β2-microglobulin domain, a transmembrane domain, a co-stimulatory domain, and a cytoplasmic signaling domain; v) a selectable marker; vi) a polypeptide that confers resistance to one or more calcineurin inhibitors; or vii) an FKBP-rapamycin binding (FRB) domain polypeptide of the mammalian target of rapamycin (mTOR) kinase.

In some embodiments, the chimeric receptor transmembrane domain comprises a CD8 transmembrane domain polypeptide, the chimeric receptor co-stimulatory domain comprises a 4-1BB co-stimulatory domain, and/or the chimeric receptor cytoplasmic signaling domain comprises a CD3-ζ cytoplasmic signaling domain.

In some embodiments, the chimeric receptor comprises the amino acid sequence of SEQ ID NO: 65 or a variant thereof having at least 85% homology to the amino acid sequence of SEQ ID NO: 65.

In some embodiments, the selectable marker is a truncated low-affinity nerve growth factor receptor (tLNGFR) polypeptide.

In some embodiments, the tLNGFR polypeptide comprises the amino acid sequence of SEQ ID NO: 66.

In some embodiments, the polypeptide that confers resistance to one or more calcineurin inhibitors is a mutant calcineurin (CN) polypeptide.

In some embodiments, the mutant CN polypeptide is CNb30 (SEQ ID NO: 67).

In some embodiments, the FRB domain polypeptide comprises the amino acid of SEQ ID NO: 68 or 69 or a variant having at least 90% sequence homology to the amino acid of SEQ ID NO: 68 or 69.

In some embodiments, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

In some embodiments, the RGEN is Cas9.

In some embodiments, the nucleic acid encoding the RGEN is a ribonucleic acid (RNA) sequence.

In some embodiments, the RNA sequence encoding the RGEN is linked to the first gRNA or the second gRNA via a covalent bond.

In some embodiments, the system comprises an Adeno-Associated Virus (AAV) vector comprising one of the one or more donor templates.

In some embodiments, the AAV vector comprises the polynucleotide sequence of any one of SEQ ID NOs: 19-46 and variants thereof having at least 85% homology to the polynucleotide sequence of any one of SEQ ID NOs: 19-46.

In some embodiments, the system comprises the first gRNA and a first AAV vector and the second gRNA and a second AAV vector, wherein (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of any one of SEQ ID NOs: 28, 31, 34, and 37 and variants thereof having at least 85% homology to the polynucleotide sequence of any one of SEQ ID NOs: 28, 31, 34, and 37, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of any one of SEQ ID NOs: 40-44 or a variant thereof having at least 85% homology to the polynucleotide sequence of any one of SEQ ID NOs: 40-44; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of any one of SEQ ID NOs: 29, 32, 35, and 38 and variants thereof having at least 85% homology to the polynucleotide sequence of any one of SEQ ID NOs: 29, 32, 35, and 38, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of any one of SEQ ID NOs: 40-44 or a variant thereof having at least 85% homology to the polynucleotide sequence of any one of SEQ ID NOs: 40-44; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of any one of SEQ ID NOs: 30, 33, 36, and 39 and variants thereof having at least 85% homology to the polynucleotide sequence of any one of SEQ ID NOs: 30, 33, 36, and 39, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of any one of SEQ ID NOs: 40-44 or a variant thereof having at least 85% homology to the polynucleotide sequence of any one of SEQ ID NOs: 40-44.

In some embodiments, the system comprises: (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 19 or 22 or a variant thereof having at least 85% homology to the polynucleotide sequence of SEQ ID NO: 19 or 22, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 20 or 23 or a variant thereof having at least 85% homology to the polynucleotide sequence of SEQ ID NO: 20 or 23, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 21 or 24 or a variant thereof having at least 85% homology to the polynucleotide sequence of SEQ ID NO: 21 or 24, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45.

In some embodiments, the system comprises: (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 25 or a variant thereof having at least 85% homology to the polynucleotide sequence of SEQ ID NO: 25, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 26 or a variant thereof having at least 85% homology to the polynucleotide sequence of SEQ ID NO: 26, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 27 or a variant thereof having at least 85% homology to the polynucleotide sequence of SEQ ID NO: 27, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46.

In some embodiments, the system comprises a ribonucleoprotein (RNP) complex comprising the RGEN and the first gRNA and/or the second gRNA.

In some embodiments, the RGEN is precomplexed with the first gRNA and/or the second gRNA at a molar ratio of gRNA to RGEN between 1:1 to 20:1, respectively, to form the RNP.

In another aspect, provided herein is a vector comprising the nucleic acid sequence of any one of SEQ ID NOs: 19-46, or a variant thereof having at least 85% homology to any one of SEQ ID NOs: 19-46.

In some embodiments, the vector is an Adeno Associated Virus (AAV) vector.

In another aspect, provided herein is a method of editing the genome of a cell, the method comprising providing to the cell: a) a first gRNA and/or a second gRNA, wherein the first gRNA is a gRNA according to any of the embodiments described above and the second gRNA is a gRNA according to any of the embodiments described above; b) an RGEN or a nucleic acid encoding the RGEN; and c) one or more donor templates comprising nucleic acid encoding: i) a CAR that can recognize a BCMA polypeptide; ii) a first CISC component comprising a first extracellular binding domain or portion thereof, a hinge domain, a transmembrane domain, and a signaling domain or portion thereof or functional derivative thereof; and iii) a second CISC component comprising a second extracellular binding domain or portion thereof, a hinge domain, a transmembrane domain, and a signaling domain or portion thereof, wherein the first CISC component and the second CISC component are configured such that when expressed by a T cell, they dimerize in the presence of a ligand to create a signaling competent CISC capable of promoting the survival and/or proliferation of the T cell.

In some embodiments, the CAR that can recognize BCMA comprises an extracellular BCMA recognition domain, a transmembrane domain, a co-stimulatory domain, and a cytoplasmic signaling domain.

In some embodiments, the extracellular BCMA recognition domain is an antibody moiety that can specifically bind to BCMA.

In some embodiments, the antibody moiety comprises a heavy chain variable domain (V_{H}) and a light chain variable domain (V_{L}) comprising heavy chain complementarity-determining region (HC-CDR)1, HC-CDR2, HC-CDR3, light chain complementarity-determining region (LC-CDR)1, LC-CDR2, and LC-CDR3 from SEQ ID NO: 55.

In some embodiments, the antibody moiety is an scFv.

In some embodiments, the CAR transmembrane domain comprises a CD8 transmembrane domain, the CAR co-stimulatory domain comprises a 4-1BB and/or a CD28 co-stimulatory domain, and/or the CAR cytoplasmic signaling domain comprises a CD3-cytoplasmic signaling domain.

In some embodiments, the signaling domain of the first CISC component comprises an IL-2 receptor subunit gamma (IL2Rγ) cytoplasmic signaling domain.

In some embodiments, the IL2Rγ cytoplasmic signaling domain comprises the amino acid sequence of SEQ ID NO: 50 or a variant thereof having at least 85% homology to the amino acid sequence of SEQ ID NO: 50.

In some embodiments, the first extracellular binding domain or portion thereof comprises an FK506 binding protein (FKBP) domain or a portion thereof.

In some embodiments, the FKBP domain comprises the amino acid sequence of SEQ ID NO: 47 or a variant thereof having at least 85% homology to the amino acid sequence of SEQ ID NO: 47.

In some embodiments, the signaling domain of the second CISC component comprises an IL-2 receptor subunit beta (IL2Rβ) cytoplasmic signaling domain.

In some embodiments, the IL2Rβ cytoplasmic signaling domain comprises the amino acid sequence of SEQ ID NO: 51 or a variant thereof having at least 85% homology to the amino acid sequence of SEQ ID NO: 51.

In some embodiments, the second extracellular binding domain or portion thereof comprises an FKBP rapamycin binding (FRB) domain or a portion thereof.

In some embodiments, the FRB domain comprises the amino acid sequence of SEQ ID NO: 48 or a variant thereof having at least 85% homology to the amino acid sequence of SEQ ID NO: 48.

In some embodiments, the transmembrane domain of the first and second CISC components comprises, independently, an IL-2 receptor transmembrane domain.

In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

In some embodiments, the c) one or more donor templates further comprise nucleic acid encoding one or more of: iv) a chimeric receptor comprising an extracellular β2-microglobulin domain, a transmembrane domain, a co-stimulatory domain, and a cytoplasmic signaling domain; v) a selectable marker; vi) a polypeptide that confers resistance to one or more calcineurin inhibitors; or vii) an FKBP-rapamycin binding (FRB) domain polypeptide of the mammalian target of rapamycin (mTOR) kinase.

In some embodiments, the chimeric receptor transmembrane domain comprises a CD8 transmembrane domain polypeptide, the chimeric receptor co-stimulatory domain comprises a 4-1BB co-stimulatory domain, and/or the chimeric receptor cytoplasmic signaling domain comprises a CD3-ζ cytoplasmic signaling domain.

In some embodiments, the chimeric receptor comprises the amino acid sequence of SEQ ID NO: 65 or a variant thereof having at least 85% homology to the amino acid sequence of SEQ ID NO: 65

In some embodiments, the selectable marker is a truncated low-affinity nerve growth factor receptor (tLNGFR) polypeptide.

In some embodiments, the tLNGFR polypeptide comprises the amino acid sequence of SEQ ID NO: 66.

In some embodiments, the polypeptide that confers resistance to one or more calcineurin inhibitors is a mutant calcineurin (CN) polypeptide.

In some embodiments, the mutant CN polypeptide is CNb30 (SEQ ID NO: 67).

In some embodiments, the FRB domain polypeptide comprises the amino acid of SEQ ID NO: 68 or 69 or a variant having at least 90% sequence homology to the amino acid of SEQ ID NO: 68 or 69.

In another aspect, provided herein is a method of editing the genome of a cell, the method comprising providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first vector comprises the polynucleotide sequence of any one of SEQ ID NOs: 28, 31, 34, and 37 and variants thereof having at least 85% homology to the polynucleotide sequence of any one of SEQ ID NOs: 28, 31, 34, and 37, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44; (B) the first gRNA comprises the polynucleotide sequence of SEQ ID NO: 2 or a variant thereof having at least 85% homology to the polynucleotide sequence of SEQ ID NO: 2, the first vector comprises the polynucleotide sequence of any one of SEQ ID NOs: 29, 32, 35, and 38 and variants thereof having at least 85% homology to the polynucleotide sequence of any one of SEQ ID NOs: 29, 32, 35, and 38, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44; or (C) the first gRNA comprises the polynucleotide sequence of SEQ ID NO: 3 or a variant thereof having at least 85% homology to the polynucleotide sequence of SEQ ID NO: 3, the first vector comprises the polynucleotide sequence of any one of SEQ ID NOs: 30, 33, 36, and 39 and variants thereof having at least 85% homology to the polynucleotide sequence of any one of SEQ ID NOs: 30, 33, 36, and 39, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44.

In another aspect, provided herein is a method of editing the genome of a cell, the method comprising providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 19 or 22 or a variant thereof having at least 85% homology to the polynucleotide sequence of SEQ ID NO: 19 or 22, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 20 or 23 or a variant thereof having at least 85% homology to the polynucleotide sequence of SEQ ID NO: 20 or 23, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 21 or 24 or a variant thereof having at least 85% homology to the polynucleotide sequence of SEQ ID NO: 21 or 24, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45.

In another aspect, provided herein is a method of editing the genome of a cell, the method comprising providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 25 or a variant thereof having at least 85% homology to the polynucleotide sequence of SEQ ID NO: 25, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 26 or a variant thereof having at least 85% homology to the polynucleotide sequence of SEQ ID NO: 26, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 27 or a variant thereof having at least 85% homology to the polynucleotide sequence of SEQ ID NO: 27, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46.

In some embodiments, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

In some embodiments, the RGEN is Cas9.

In some embodiments, the nucleic acid encoding the RGEN is a ribonucleic acid (RNA) sequence.

In some embodiments, the RNA sequence encoding the RGEN is linked to the first gRNA or the second gRNA via a covalent bond.

In some embodiments, the donor template is contained in an AAV vector.

In some embodiments, the RGEN is precomplexed with the first gRNA and/or the second gRNA, forming an RNP complex, prior to the provision to the cell.

In some embodiments, the RGEN is precomplexed with the first gRNA and/or the second gRNA at a molar ratio of gRNA to RGEN between 1:1 to 20:1, respectively.

In some embodiments, the one or more donor templates are, independently, inserted into the genome of the cell.

In some embodiments, a first donor template is inserted at, within, or near a TRA gene or gene regulatory element and/or a second donor template is inserted at, within, or near an IL2RG gene or gene regulatory element.

In some embodiments, nucleic acid encoding i) the first CISC component is inserted into an endogenous IL2RG gene, and/or nucleic acid encoding ii) the second CISC component is inserted into the region of the endogenous TRA gene encoding the TRAC domain; or nucleic acid encoding i) the first CISC component is inserted into the region of the endogenous TRA gene encoding the TRAC domain, and/or nucleic acid encoding ii) the second CISC component is inserted into the endogenous IL2RG gene.

In some embodiments, the cell is a T cell.

In some embodiments, the T cell is a CD8+ cytotoxic T lymphocyte or a CD3+ pan T cell.

In some embodiments, the T cell is a member of a pool of T cells derived from multiple donors.

In some embodiments, the multiple donors are human donors.

In some embodiments, the cell is cytotoxic to plasma cells.

In another aspect, provided herein is an engineered cell produced by a method according to any of the embodiments described above.

In some embodiments, the engineered cell is cytotoxic to plasma cells.

In another aspect, provided herein is a method of treating graft vs host disease (GvHD) or an autoimmune disease in a subject in need thereof, the method comprising: administering an engineered cell according to any of the embodiments described above to the subject.

In another aspect, provided herein is a method of treating a disease or condition in a subject in need thereof, wherein the disease or condition is characterized by adverse antibody production, the method comprising: a) editing the genome of T cells according to a method according to any of the embodiments described above, thereby producing engineered T cells; and b) administering the engineered T cells to the subject.

In some embodiments, the T cells are autologous to the subject.

In some embodiments, the T cells are allogenic to the subject.

In some embodiments, the T cells comprise a pool of T cells derived from multiple donors.

In some embodiments, the multiple donors are human donors.

In another aspect, provided herein is a method of treating a disease or condition in a subject in need thereof, wherein the disease or condition is characterized by adverse antibody production, the method comprising editing the genome of a T cell in the subject according to a method according to any of the embodiments described above.

In some embodiments, the T cells comprise CD8+ cytotoxic T cells or CD3+ pan T cells.

In some embodiments, the subject is human.

In some embodiments, the method further comprises administering rapamycin or a rapalog to the subject.

In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

In some embodiments, the rapamycin or the rapalog is administered in a concentration from 0.05 nM to 100 nM.

In some embodiments, the disease or condition is graft-versus-host disease (GvHD), antibody-mediated autoimmunity, or light-chain amyloidosis.

In some embodiments, the disease or condition is GvHD, and the subject has previously received an allogeneic transplant.

In another aspect, provided herein is a kit comprising instructions for use and a) an engineered cell according to any of the embodiments described above and/or one or more components of a system according to any of the embodiments described above; and/or b) rapamycin or a rapalog.

In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

In another aspect, provided herein is a syringe comprising an engineered cell according to any of the embodiments described above or a composition comprising one or more components of a system according to any of the embodiments described above.

In another aspect, provided herein is a catheter comprising an engineered cell according to any of the embodiments described above or a composition comprising one or more components of a system according to any of the embodiments described above.

In another aspect, provided herein is an engineered T cell according to any of the embodiments described above for use in the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is an engineered T cell according to any of the embodiments described above for use in the manufacture of a medicament for the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is a system according to any of the embodiments described above for use in the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is a system according to any of the embodiments described above for use in the manufacture of a medicament for the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is one or more gRNAs, one or more donor templates, a kit, a syringe, and/or a catheter according to any of the embodiments described above for use in the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is one or more gRNAs, one or more donor templates, a kit, a syringe, and/or a catheter according to any of the embodiments described above for use in the manufacture of a medicament for the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

### BRIEF DESCRIPTION OF THE DRAWINGS

FIG. 1 shows schematics for donor template constructs #1-#11, depicting elements present in the donor template constructs (not shown to scale) and their relative positions. 5′ HA: 5′ homology arm; 3′ HA: 3′ homology arm; s pA: synthetic polyA signal; SV40 pA: SV40 polyA signal; pMSCV: murine stem cell virus (MSCV) promoter; CD8 sp: CD8 signal peptide; CD8 tm: CD8 transmembrane domain; CD28: CD28 co-stimulatory domain; 4-1BB: 4-1BB co-stimulatory domain; CD3z: CD3-ζ cytoplasmic signaling domain; WPRE3: Woodchuck Hepatitis Virus (WHP) Posttranscriptional Regulatory Element (WPRE) 3; CISCβ: CISC subunit with FRB domain and IL2Rβ domain; tCISCγ: CISC subunit with FKBP domain and fragment of IL2Rγ domain; β2M CR: β2-microglobulin chimeric receptor; FRB: naked FRB domain polypeptide; P2A, T2A: self-cleaving peptide; ER: endoplasmic reticulum signal sequence; CNb30: mutant calcineurin polypeptide; tLNGFR: truncated low-affinity nerve growth factor receptor.

### DETAILED DESCRIPTION

Described herein are engineered T cells cytotoxic towards plasma cells, wherein the engineered T cells comprise a chemical-induced signaling complex (CISC) allowing for controlled survival and/or proliferation of the engineered T cells, such as engineered T cells expressing an anti-BCMA chimeric antigen receptor (CAR) that confers cytotoxicity towards BCMA-expressing cells, methods of making and using the engineered T cells, and compositions useful for the methods.

The Applicant has developed a series of novel CRISPR/Cas systems for targeted integration of heterologous nucleic acid sequences encoding an anti-BCMA CAR and/or a CISC into a TIM gene and/or an IL2RG gene in a cell genome, where the CISC is capable of IL2R-like signaling upon binding of rapamycin or rapamycin analogs, taking advantage of integration of the heterologous nucleic acid sequences functionally repressing endogenous TCR and/or IL2RG expression in edited cells. Guide RNAs (gRNAs) with spacer sequences targeting TIM or IL2RG were analyzed for on-target and off-target cleavage and found to have favorable profiles, making them candidates for downstream uses, such as in cell-based therapies. Primary human CD3+ T cells were successfully edited to express an anti-BCMA CAR and/or a CISC, and edited cells showed decreased expression of endogenous TCR and/or IL2RG. These findings indicate that the CRISPR/Cas systems described herein are useful for treating diseases, for example, diseases associated with BCMA-expressing cells.

### Definitions

Unless defined otherwise, all technical and scientific terms used herein have the same meaning as commonly understood by one of ordinary skill in the art to which the disclosure pertains. All patents, applications, published applications and other publications referenced herein are expressly incorporated by reference in their entireties unless stated otherwise. In the event that there are a plurality of definitions for a term herein, those in this section prevail unless stated otherwise.

As used herein, “a” or “an” may mean one or more than one.

“About” has its plain and ordinary meaning when read in light of the specification, and may be used, for example, when referring to a measurable value and may be meant to encompass variations of ±20% or ±10%, ±5%, ±1%, or ±0.1% from the specified value.

As used herein, “protein sequence” refers to a polypeptide sequence of amino acids that is the primary structure of a protein. As used herein “upstream” refers to positions 5′ of a location on a polynucleotide, and positions toward the N-terminus of a location on a polypeptide. As used herein “downstream” refers to positions 3′ of a location on nucleotide, and positions toward the C-terminus of a location on a polypeptide. Thus, the term “N-terminal” refers to the position of an element or location on a polynucleotide toward the N-terminus of a location on a polypeptide.

“Nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also comprises so-called “peptide nucleic acids,” which comprise naturally-occurring or modified nucleic acid bases attached to a polyamide backbone. Nucleic acids can be either single-stranded or double-stranded. In some embodiments, a nucleic acid sequence encoding a fusion protein is provided. In some embodiments, the nucleic acid is RNA or DNA.

“Coding for” or “encoding” are used herein, and refers to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

A “nucleic acid sequence coding for a polypeptide” comprises all nucleotide sequences that are degenerate versions of each other and that code for the same amino acid sequence. In some embodiments, a nucleic acid is provided, wherein the nucleic acid encodes a fusion protein.

“Vector,” “expression vector,” or “construct” is a nucleic acid used to introduce heterologous nucleic acids into a cell that has regulatory elements to provide expression of the heterologous nucleic acids in the cell. Vectors include but are not limited to plasmid, minicircles, yeast, and viral genomes. In some embodiments, the vectors are plasmid, minicircles, yeast, or viral genomes. In some embodiments, the vector is a viral vector. In some embodiments, the viral vector is a lentivirus. In some embodiments, the vector is an adeno-associated viral (AAV) vector. In some embodiments, the vector is for protein expression in a bacterial system such as E. coli. As used herein, the term “expression,” or “protein expression” refers to refers to the translation of a transcribed RNA molecule into a protein molecule. Protein expression may be characterized by its temporal, spatial, developmental, or morphological qualities as well as by quantitative or qualitative indications. In some embodiments, the protein or proteins are expressed such that the proteins are positioned for dimerization in the presence of a ligand.

As used herein, “fusion proteins” or “chimeric proteins” are proteins created through the joining of two or more genes that originally coded for separate proteins or portions of proteins. The fusion proteins can also be made up of specific protein domains from two or more separate proteins. Translation of this fusion gene can result in a single or multiple polypeptides with functional properties derived from each of the original proteins.

Recombinant fusion proteins can be created artificially by recombinant DNA technology for use in biological research or therapeutics. Such methods for creating fusion proteins are known to those skilled in the art. Some fusion proteins combine whole peptides and therefore can contain all domains, especially functional domains, of the original proteins. However, other fusion proteins, especially those that are non-naturally occurring, combine only portions of coding sequences and therefore do not maintain the original functions of the parental genes that formed them.

As used herein, the term “regulatory element” refers to a DNA molecule having gene regulatory activity, e.g., one that has the ability to affect the transcription and/or translation of an operably linked transcribable DNA molecule. Regulatory elements such as promoters, leaders, introns, and transcription termination regions are DNA molecules that have gene regulatory activity and play an integral part in the overall expression of genes in living cells. Isolated regulatory elements, such as promoters, that function in plants are therefore useful for modifying plant phenotypes through the methods of genetic engineering.

As used herein, the term “operably linked” refers to a first molecule joined to a second molecule, wherein the molecules are so arranged that the first molecule affects the function of the second molecule. The two molecules may be part of a single contiguous molecule and may be adjacent. For example, a promoter is operably linked to a transcribable DNA molecule if the promoter modulates transcription of the transcribable DNA molecule of interest in a cell.

A “promoter” is a region of DNA that initiates transcription of a specific gene. The promoters can be located near the transcription start site of a gene, on the same strand and upstream on the DNA (the 5′-region of the sense strand). The promoter can be a conditional, inducible or a constitutive promoter. The promoter can be specific for bacterial, mammalian or insect cell protein expression. In some embodiments, wherein a nucleic acid encoding a fusion protein is provided, the nucleic acid further comprises a promoter sequence. In some embodiments, the promoter is specific for bacterial, mammalian or insect cell protein expression. In some embodiments, the promoter is a conditional, inducible or a constitutive promoter. In other embodiments, the promoter is an MND promoter.

“Dimeric chemical-induced signaling complex,” “dimeric CISC,” or “dimer” as used herein refers to two components of a CISC, which may or may not be fusion protein complexes that join together. “Dimerization” refers to the process of the joining together of two separate entities into a single entity. In some embodiments, a ligand or agent stimulates dimerization. In some embodiments, dimerization refers to homodimerization, or the joining of two identical entities, such as two identical CISC components. In some embodiments, dimerization refers to heterodimerization, of the joining of two different entities, such as two different and distinct CISC components. In some embodiments, the dimerization of the CISC components results in a cellular signaling pathway. In some embodiments, the dimerization of the CISC components allows for the selective expansion of a cell or a population of cells. Additional CISC systems can include a CISC gibberellin CISC dimerization system, or a SLF-TMP CISC dimerization system. Other chemically inducible dimerization (CID) systems and component parts may be used.

As used herein, “chemical-induced signaling complex” or “CISC” refers to an engineered complex that initiates a signal into the interior of a cell as a direct outcome of ligand-induced dimerization. A CISC may be a homodimer (dimerization of two identical components) or a heterodimer (dimerization of two distinct components). Thus, as used herein the term “homodimer” refers to a dimer of two protein components described herein with identical amino acid sequences. The term “heterodimer” refers to a dimer of two protein components described herein with non-identical amino acid sequences.

The CISC may be a synthetic complex as described herein in greater detail. “Synthetic” as used herein refers to a complex, protein, dimer, or composition, as described herein, which is not natural, or that is not found in nature. In some embodiments, an IL2R-CISC refers to a signaling complex that involves interleukin-2 receptor components. In some embodiments, an IL2/15-CISC refers to a signaling complex that involves receptor signaling subunits that are shared by interleukin-2 and interleukin-15. In some embodiments, an IL7-CISC refers to a signaling complex that involves an interleukin-7 receptor components. A CISC may thus be termed according to the component parts that make up the components of a given CISC. One of skill in the art will recognize that the component parts of the chemical-induced signaling complex may be composed of a natural or a synthetic component useful for incorporation into a CISC. Thus, the examples provided herein are not intended to be limiting.

As used herein, “cytokine receptor” refers to receptor molecules that recognize and bind to cytokines. In some embodiments, cytokine receptor encompasses modified cytokine receptor molecules (e.g., “variant cytokine receptors”), comprising those with substitutions, deletions, and/or additions to the cytokine receptor amino acid and/or nucleic acid sequence. Thus, it is intended that the term encompass wild-type, as well as, recombinant, synthetically-produced, and variant cytokine receptors. In some embodiments, the cytokine receptor is a fusion protein, comprising an extracellular binding domain, a hinge domain, a transmembrane domain, and a signaling domain. In some embodiments, the components of the receptor (that is, the domains of the receptor) are natural or synthetic. In some embodiments, the domains are human derived domains.

“FKBP” as used herein, is a FK506 binding protein domain. FKBP refers to a family of proteins that have prolyl isomerase activity and are related to the cyclophilins in function, though not in amino acid sequence. FKBPs have been identified in many eukaryotes from yeast to humans and function as protein folding chaperones for proteins containing proline residues. Along with cyclophilin, FKBPs belong to the immunophilin family. The term FKBP comprises, for example, FKBP12 as well as, proteins encoded by the genes AIP; AIPL1; FKBP1A; FKBP1B; FKBP2; FKBP3; FKBP5; FKBP6; FKBP7; FKBP8; FKBP9; FKBP9L; FKBP10; FKBP11; FKBP14; FKBP15; FKBP52; and/or L00541473; comprising homologs thereof and functional protein fragments thereof.

“FRB” as used herein, as a FKBP rapamycin binding domain. FRB domains are polypeptide regions (protein “domains”) that are configured to form a tripartite complex with an FKBP protein and rapamycin or rapalog thereof. FRB domains are present in a number of naturally occurring proteins, comprising mTOR proteins (also referred to in the literature as FRAP, RAPT 1, or RAFT) from human and other species; yeast proteins comprising Tor1 and/or Tor2; and a Candida FRAP homolog. Both FKBP and FRB are major constituents in the mammalian target of rapamycin (mTOR) signaling.

A “naked FKBP rapamycin binding domain polypeptide” or a “naked FRB domain polypeptide” (which can also be referred to as an “FKBP rapamycin binding domain polypeptide” or an “FRB domain polypeptide”) refers to a polypeptide comprising only the amino acids of an FRB domain or a protein wherein about 90%, 91%, 92%, 93%, 94%, 95%, 96%, 97%, 98%, 99%, or 100% of the amino acids of the protein are amino acids of an FRB domain. The FRB domain can be expressed as a 12 kDa soluble protein (Chen, J. et al. (1995). Proc. Natl. Acad. Sci. U.S.A., 92(11):4947-4951). The FRB domain forms a four helix bundle, a common structural motif in globular proteins. Its overall dimensions are 30 Å by 45 Å by 30 Å, and all four helices have short underhand connections similar to the cytochrome b562 fold (Choi, J. et al. (1996). Science, 273(5272):239-242). In some embodiments, the naked FRB domain comprises the amino acid sequence of SEQ ID NO: 68 or 69.

In some embodiments, the immunomodulatory imide drug used in the approaches described herein may comprise: thalidomide (including analogues, derivatives, and including pharmaceutically acceptable salts thereof. Thalidomide may include Immunoprin, Thalomid, Talidex, Talizer, Neurosedyn, α-(N-Phthalimido)glutarimide, 2-(2,6-dioxopiperidin-3-yl)-2,3-dihydro-1H-isoindole-1,3-dione); pomalidomide (including analogues, derivatives, and including pharmaceutically acceptable salts thereof. Pomalidomide may include Pomalyst, Imnovid, (RS)-4-Amino-2-(2,6-dioxopiperidin-3-yl)isoindole-1,3-dione); lenalidomide (including analogues, derivatives, and including pharmaceutically acceptable salts thereof. Lenalidomide may include Revlimid, (RS)-3-(4-Amino-1-oxo-1,3-dihydro-2H-isoindol-2-yl)piperidine-2,6-dione); or apremilast (including analogues, derivatives, and including pharmaceutically acceptable salts thereof. Apremilast may include Otezla, CC-10004, N-{2-[(1 S)-1-(3-Ethoxy-4-methoxyphenyl)-2-(methyl sulfonyl)ethyl]-1,3-dioxo-2,3-dihydro-1H-isoindol-4-yl}acetamide); or any combinations thereof.

As used herein, the term “extracellular binding domain” refers to a domain of a complex that is outside of the cell, and which is configured to bind to a specific atom or molecule. In some embodiments, the extracellular binding domain of a CISC is a FKBP domain or a portion thereof. In some embodiments, the extracellular binding domain is an FRB domain or a portion thereof. In some embodiments, the extracellular binding domain is configured to bind a ligand or agent, thereby stimulating dimerization of two CISC components. In some embodiments, the extracellular binding domain is configured to bind to a cytokine receptor modulator.

As used herein, the term “cytokine receptor modulator” refers to an agent, which modulates the phosphorylation of a downstream target of a cytokine receptor, the activation of a signal transduction pathway associated with a cytokine receptor, and/or the expression of a particular protein such as a cytokine. Such an agent may directly or indirectly modulate the phosphorylation of a downstream target of a cytokine receptor, the activation of a signal transduction pathway associated with a cytokine receptor, and/or the expression of a particular protein such as a cytokine. Thus, examples of cytokine receptor modulators include, but are not limited to, cytokines, fragments of cytokines, fusion proteins and/or antibodies or binding portions thereof that immunospecifically bind to a cytokine receptor or a fragment thereof. Further, examples of cytokine receptor modulators include, but are not limited to, peptides, polypeptides (e.g., soluble cytokine receptors), fusion proteins and/or antibodies or binding portions thereof that immunospecifically bind to a cytokine or a fragment thereof.

As used herein, the term “activate” refers to an increase in at least one biological activity of a protein of interest. Similarly, the term “activation” refers to a state of a protein of interest being in a state of increased activity. The term “activatable” refers to the ability of a protein of interest to become activated in the presence of a signal, an agent, a ligand, a compound, or a stimulus. In some embodiments, a dimer, as described herein, is activated in the presence of a signal, an agent, a ligand, a compound, or a stimulus, and becomes a signaling competent dimer. As used herein, the term “signaling competent” refers to the ability or configuration of the dimer so as to be capable of initiating or sustaining a downstream signaling pathway.

As used herein, the term “hinge domain” refers to a domain that links the extracellular binding domain to the transmembrane domain, and may confer flexibility to the extracellular binding domain. In some embodiments, the hinge domain positions the extracellular domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

As used herein, the term “transmembrane domain” or “TM domain” refers to a domain that is stable in a membrane, such as in a cell membrane. The terms “transmembrane span,” “integral protein,” and “integral domain” are also used herein. In some embodiments, the hinge domain and the extracellular domain is located N-terminal to the transmembrane domain. In some embodiments, the transmembrane domain is a natural or a synthetic domain. In some embodiments, the transmembrane domain is an IL-2 receptor transmembrane domain.

As used herein, the term “signaling domain” refers to a domain of the fusion protein or CISC component that is involved in a signaling cascade inside the cell, such as a mammalian cell. A signaling domain refers to a signaling moiety that provides to cells, such as T-cells, a signal which, in addition to the primary signal provided by for instance the CD3 zeta chain of the TCR/CD3 complex, mediates a cellular response, such as a T-cell response, comprising, but not limited to, activation, proliferation, differentiation, and/or cytokine secretion. In some embodiments, the signaling domain is N-terminal to the transmembrane domain, the hinge domain, and the extracellular domain. In some embodiments, the signaling domain is a synthetic or a natural domain. In some embodiments, the signaling domain is a concatenated cytoplasmic signaling domain. In some embodiments, the signaling domain is a cytokine signaling domain. In some embodiments, the signaling domain is an antigen signaling domain. In some embodiments, the signaling domain is an interleukin-2 receptor subunit gamma (IL2Rγ or IL2RG) domain. In some embodiments, the signaling domain is an interleukin-2 receptor subunit beta (IL2Rβ or IL2RB) domain. In some embodiments, binding of an agent or ligand to the extracellular binding domain causes a signal transduction through the signaling domain by the activation of a signaling pathway, as a result of dimerization of the CISC components. As used herein, the term “signal transduction” refers to the activation of a signaling pathway by a ligand or an agent binding to the extracellular domain. Activation of a signal is a result of the binding of the extracellular domain to the ligand or agent, resulting in CISC dimerization.

As used herein, the term “IL2RB” or “IL2Rβ” refers to an interleukin-2 receptor subunit beta. Similarly, the term “IL2RG” or IL2Rγ″ refers to an interleukin-2 receptor subunit gamma, and the term “IL2RA” or “IL2Ra” refers to an interleukin-2 receptor subunit alpha. The IL-2 receptor has three forms, or chains, alpha, beta, and gamma, which are also subunits for receptors for other cytokines. IL2Rβ and IL2Rγ are members of the type I cytokine receptor family. “IL2R” as used herein refers to interleukin-2 receptor, which is involved in T cell-mediated immune responses. IL2R is involved in receptor-mediated endocytosis and transduction of mitogenic signals from interleukin 2. Similarly, the term “IL-2/15R” refers to a receptor signaling subunit that is shared by IL-2 and IL-15, and may include a subunit alpha (IL2/15Ra or IL2/15Rα), beta (IL2/15Rb or IL2/15Rβ, or gamma (IL2/15Rg or IL2/15Rγ).

In some embodiments, a chemical-induced signaling complex is a heterodimerization activated signaling complex comprising two components. In some embodiments, the first component comprises an extracellular binding domain that is one part of a heterodimerization pair, an optional hinge domain, a transmembrane domain, and one or more concatenated cytoplasmic signaling domains. In some embodiments, the second component comprises an extracellular binding domain that is the other part of a heterodimizeration pair, an optional hinge domain, a transmembrane domain, and one or more concatenated cytoplasmic signaling domains. Thus, in some embodiments, there are two distinct modification events. In some embodiments, the two CISC components are expressed in a cell, such as a mammalian cell. In some embodiments, the cell, such as a mammalian cell, or a population of cells, such as a population of mammalian cells, is contacted with a ligand or agent that causes heterodimerization, thereby initiating a signal. In some embodiments, a homodimerization pair dimerize, whereby a single CISC component is expressed in a cell, such as a mammalian cell, and the CISC components homodimerize to initiate a signal.

As used herein, the term “ligand” or “agent” refers to a molecule that has a desired biological effect. In some embodiments, a ligand is recognized by and bound by an extracellular binding domain, forming a tripartite complex comprising the ligand and two binding CISC components. Ligands include, but are not limited to, proteinaceous molecules, comprising, but not limited to, peptides, polypeptides, proteins, post-translationally modified proteins, antibodies, binding portions thereof; small molecules (less than 1000 Daltons), inorganic or organic compounds; and nucleic acid molecules comprising, but not limited to, double-stranded or single-stranded DNA, or double-stranded or single-stranded RNA (e.g., antisense, RNAi, etc.), aptamers, as well as, triple helix nucleic acid molecules. Ligands can be derived or obtained from any known organism (comprising, but not limited to, animals (e.g., mammals (human and non-human mammals)), plants, bacteria, fungi, and protista, or viruses) or from a library of synthetic molecules. In some embodiments, the ligand is a protein, an antibody or portion thereof, a small molecule, or a drug. In some embodiments, the ligand is rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Thus, in some embodiments, the rapalog is everolimus, merilimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophenolic acid, benidipine hydrochloride, AP23573, or AP1903, or metabolites, derivatives, and/or combinations thereof. In some embodiments, the ligand is an IMID-class drug (e.g. thalidomide, pomalidomide, lenalidomide or related analogues).

Accordingly, in some embodiments, the ligand or agent used in the approaches described herein for chemical induction of the signaling complex may comprise: rapamycin (including analogues, derivatives, and including pharmaceutically acceptable salts thereof. Rapamycin may include Sirolimus, Rapamune, (3 S,6R,7E,9R,10R,12R,14S,15E,17E,19E,21S,23 S,26R,27R,34aS)-9,10,12,13,14,21,22,23,24,25,26,27,32,33,34,34a-hexadecahydro-9,27-dihydroxy-3-[(1R)-2-[(1 S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4] oxaazacyclohentriacontine-1,5,11,28,29 (4H,6H,31H)-pentone); everolimus (including analogues, derivatives, and including pharmaceutically acceptable salts thereof. Everolimus may include RAD001, Zortress, Certican, Afinitor, Votubia, 42-O-(2-hydroxyethyl)rapamycin, (1R,9S,12S,15R,16E,18R,19R,21R,23 S,24E,26E,28E,30S,32S,35R)-1,18-dihydroxy-12-[(2R)-1-[(1 S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone); merilimus (including analogues, derivatives, and including pharmaceutically acceptable salts thereof. Merilimus may include SAR943, 42-O-(tetrahydrofuran-3-yl)rapamycin (Merilimus-1); 42-O-(oxetan-3-yl)rapamycin (Merilimus-2), 42-O-(tetrahydropyran-3-yl)rapamycin (Merilimus-3), 42-O-(4-methyl, tetrahydrofuran-3-yl)rapamycin, 42-O-(2,5,5-trimethyl, tetrahydrofuran-3-yl) rapamycin, 42-O-(2,5-diethyl-2-methyl, tetrahydrofuran-3-yl)rapamycin, 42-O-(2H-Pyran-3-yl, tetrahydro-6-methoxy-2-methyl)rapamycin, or 42-O-(2H-Pyran-3-yl, tetrahydro-2,2-dimethyl-6-phenyl)rapamycin); novolimus (including analogues, derivatives, and including pharmaceutically acceptable salts thereof. Novolimus may include 16-O-Demethyl Rapamycin); pimecrolimus (including analogues, derivatives, and including pharmaceutically acceptable salts thereof. Pimecrolimus may include Elidel, (3 S,4R,5S,8R,9E,12S,14S,15R,16S,18R,19R,26aS)-3-((E)-2-((1R,3R,4S)-4-chloro-3 methoxycyclohexyl)-1-methylvinyl)-8-ethyl 5,6,8,11,12,13,14,15,16,17,18,19,24,26,26ahexadecahydro-5,19-epoxy-3H-pyrido(2,1-c)(1,4)oxaazacyclotricosine-1,17,20,21(4H,23H)-tetrone 33-epi-Chloro-33-desoxyascomycin); ridaforolimus (including analogues, derivatives, and including pharmaceutically acceptable salts thereof. Ridaforolimus may include AP23573, MK-8669, deforolimus, (1R,9S,12S,15R,16E,18R,19R,21R,23 S,24E,26E,28E,30S,32S,35R)-12-((1R)-2-((1 S,3R,4R)-4-((Dimethylphosphinoyl)oxy)-3-methoxycyclohexyl)-1-methylethyl)-1,18-dihydroxy-19,30-dimethoxy15,17,21,23,29,35-hexamethyl-11,36-dioxa-4-azatricyclo(30.3.1.04,9)hexatriaconta-16,24,26,28-tetraene-2,3,10,14,20-pentone); tacrolimus (including analogues, derivatives, and including pharmaceutically acceptable salts thereof. Tacrolimus may include FK-506, fujimycin, Prograf, Advagraf, protopic, 3S-[3R*[E(1S*,3S*,4S*)],4S*,5R*,8S*,9E,12R*,14R*,15S*,16R*,18S*,19S*,26aR*5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4] oxaazacyclotricosine-1,7,20,21(4H,23H)-tetrone, monohydrate); temsirolimus (including analogues, derivatives, and including pharmaceutically acceptable salts thereof. Temsirolimus may include CCI-779, CCL-779, Torisel, (1R,2R,4S)-4-{(2R)-2-[(3 S,6R,7E,9R,10R,12R,14S,15E,17E,19E,21S,23 S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4] oxazacyclohentriacontin-3-yl]propyl}-2-methoxycyclohexyl 3-hydroxy-2-(hydroxymethyl)-2-methylpropanoate); umirolimus (including analogues, derivatives, and including pharmaceutically acceptable salts thereof. Umirolimus may include Biolimus, Biolimus A9, BA9, TRM-986, 42-O-(2-ethoxyethyl)Rapamycin); zotarolimus (including analogues, derivatives, and including pharmaceutically acceptable salts thereof. Zotarolimus may include ABT-578, (42S)-42-Deoxy-42-(1H-tetrazol-1-yl)-rapamycin); C20-methallylrapamycin (including analogues, derivatives, and including pharmaceutically acceptable salts thereof. C20-methallylrapamycin may include C20-Marap); C16-(S)-3-methylindolerapamycin (including analogues, derivatives, and including pharmaceutically acceptable salts thereof. C16-(S)-3-methylindolerapamycin may include C16-iRap); AP21967 (including analogues, derivatives, and including pharmaceutically acceptable salts thereof. AP21967 may include C-16-(S)-7-methylindolerapamycin); sodium mycophenolic acid (including analogues, derivatives, and including pharmaceutically acceptable salts thereof. Sodium mycophenolic acid may include CellCept, Myfortic, (4E)-6-(4-Hydroxy-6-methoxy-7-methyl-3-oxo-1,3-dihydro-2-benzofuran-5-yl)-4-methylhex-4-enoi c acid); benidipine hydrochloride (including analogues, derivatives, and including pharmaceutically acceptable salts thereof. Benidipine hydrochloride may include Benidipinum, Coniel); or AP1903 (including analogues, derivatives, and including pharmaceutically acceptable salts thereof. AP1903 may include Rimiducid, [(1R)-3-(3,4-dimethoxyphenyl)-1-[3-[2-[2-[[2-[3-[(1R)-3-(3,4-dimethoxyphenyl)-1-[(2S)-1-[(2 S)-2-(3,4,5-trimethoxyphenyl)butanoyl]piperidine-2-carbonyl]oxypropyl]phenoxy]acetyl]amino]ethylamino]-2-oxoethoxy]phenyl]propyl] (2S)-1-[(2 S)-2-(3,4,5-trimethoxyphenyl)butanoyl]piperidine-2-carboxylate); or any combinations thereof.

As used herein, the term “gibberellin” refers to a synthetic or naturally occurring form of the diterpenoid acids that are synthesized by the terpenoid pathway in plastids and then modified in the endoplasmic reticulum and cytosol until they reach their biologically-active form. Gibberellin may be a natural gibberellin or an analogue thereof, including, for example, gibberellins derived from the ent-gibberellane skeleton, or synthesized via ent-kauren, including gibberelling 1 (GA1), GA2, GA3 . . . GA136, and analogues and derivatives thereof. In some embodiments, gibberellin or an analogue or derivative thereof is utilized for CISC dimerization.

As used herein, “SLF-TMP” or “synthetic ligand of FKBP linked to trimethoprim” refers to a dimerizer for CISC dimerization. In some embodiments, the SLF moiety binds to a first CISC component and the TMP moiety binds to a second CISC component, causing CISC dimerization. In some embodiments, SLF can bind, for example, to FKBP and TMP can bind to E. coli dihydrofolate reductase (eDHFR).

As used herein, the term “simultaneous binding” refers to the binding of the ligand by two or more CISC components at the same time or, in some cases, at substantially the same time, to form a multicomponent complex, comprising the CISC components and the ligand component, and resulting in subsequent signal activation. Simultaneous binding requires that the CISC components are configured spatially to bind a single ligand, and also that both CISC components are configured to bind to the same ligand, including to different moieties on the same ligand.

As used herein, the term “selective expansion” refers to an ability of a desired cell, such as a mammalian cell, or a desired population of cells, such as a population of mammalian cells, to expand. In some embodiments, selective expansion refers to the generation or expansion of a pure population of cells, such as mammalian cells, that have undergone two genetic modification events. One component of a dimerization CISC is part of one modification and the other component is the other modification. Thus, one component of the heterodimerizing CISC is associated with each genetic modification. Exposure of the cells to a ligand allows for selective expansion of only the cells, such as mammalian cells, having both desired modifications. Thus, in some embodiments, the only cells, such as mammalian cells, that will be able to respond to contact with a ligand are those that express both components of the heterodimerization CISC.

As used herein, “host cell” comprises any cell type, such as a mammalian cell, that is susceptible to transformation, transfection, or transduction, with a nucleic acid construct or vector. In some embodiments, the host cell, such as a mammalian cell, is a T cell or a T regulatory cell (Treg). In some embodiments, the host cell, such as a mammalian cell, is a hematopoietic stem cell. In some embodiments, the host cell is a CD3+, CD8+, or a CD4+ cell. In some embodiments, the host cell is a CD8+T cytotoxic lymphocyte cell selected from the group consisting of naïve CD8+ T cells, central memory CD8+ T cells, effector memory CD8+ T cells, and bulk CD8+ T cells. In some embodiments, the host cell is a CD4+T helper lymphocyte cell selected from the group consisting of naïve CD4+ T cells, central memory CD4+ T cells, effector memory CD4+ T cells, and bulk CD4+ T cells. As used herein, the term “population of cells” refers to a group of cells, such as mammalian cells, comprising more than one cell. In some embodiments, a cell, such as a mammalian cell, is manufactured, wherein the cell comprises the protein sequence as described herein or an expression vector that encodes the protein sequence as described herein.

As used herein, the term “transformed” or “transfected” refers to a cell, such as a mammalian cell, tissue, organ, or organism into which a foreign polynucleotide molecule, such as a construct, has been introduced. The introduced polynucleotide molecule may be integrated into the genomic DNA of the recipient cell, such as a mammalian cell, tissue, organ, or organism such that the introduced polynucleotide molecule is inherited by subsequent progeny. A “transgenic” or “transfected” cell, such as a mammalian cell, or organism also comprises progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules. “Transduction” refers to virus-mediated gene transfer into cells, such as mammalian cells.

The term “engineered cell” refers to a cell comprising the construct(s) of the invention, regardless of whether the cell was “directly” engineered (for example, the cell was physically altered from an original or wild type condition), or descended from a cell that was so modified. Thus, “engineered cell” includes the directly modified cells and their progeny.

As used herein, a “subject” refers to an animal that is the object of treatment, observation or experiment. “Animal” comprises cold- and warm-blooded vertebrates and invertebrates such as fish, shellfish, reptiles and, in particular, mammals. “Mammal” comprises, without limitation, mice, rats, rabbits, guinea pigs, dogs, cats, sheep, goats, cows, horses, primates, such as monkeys, chimpanzees, and apes, and, in particular, humans. In some alternative, the subject is human.

In some embodiments, an effective amount of a ligand used for inducing dimerization is an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values.

A “marker sequence,” as described herein, encodes a protein that is used for selecting or tracking a protein or cell, such as a mammalian cell, that has a protein of interest. In the embodiments described herein, the fusion protein provided can comprise a marker sequence that can be selected in experiments, such as flow cytometry.

“Chimeric receptor” or “chimeric antigen receptor,” as used herein refers to a synthetically designed receptor comprising a ligand binding domain of an antibody or other protein sequence that binds to a molecule associated with the disease or disorder and is linked via a spacer domain to one or more intracellular signaling domains of a T-cell or other receptors, such as a costimulatory domain. In some embodiments, a cell, such as a mammalian cell, is manufactured wherein the cell comprises a nucleic acid encoding a fusion protein and wherein the cell comprises a chimeric antigen receptor.

“Cytotoxic T lymphocyte” (CTL), as used herein, refers to a T lymphocyte that expresses CD8 on the surface thereof (e.g., a CD8⁺ T-cell). In some embodiments, such cells are “memory” T-cells (T_{M} cells) that are antigen-experienced. In some embodiments, a cell for fusion protein secretion is provided. In some embodiments, the cell is a cytotoxic T lymphocyte. “Central memory” T-cell (or “T_{CM}”) as used herein, refers to an antigen experienced CTL that expresses CD62L, CCR-7 and/or CD45RO on the surface thereof, and does not express or has decreased expression of CD45RA, as compared to naive cells. In some embodiments, a cell for fusion protein secretion is provided. In some embodiments, the cell is a central memory T-cell (T_{CM}). In some embodiments, the central memory cells are positive for expression of CD62L, CCR7, CD28, CD127, CD45RO, and/or CD95, and may have decreased expression of CD54RA, as compared to naïve cells. “Effector memory” T-cell (or “T_{EM}”) as used herein refers to an antigen experienced T-cell that does not express or has decreased expression of CD62L on the surface thereof, as compared to central memory cells, and does not express or has a decreased expression of CD45RA, as compared to naïve cell. In some embodiments, a cell for fusion protein secretion is provided. In some embodiments, the cell is an effector memory T-cell. In some embodiments, effector memory cells are negative for expression of CD62L and/or CCR7, as compared to naïve cells or central memory cells, and may have variable expression of CD28 and/or CD45RA.

“Naïve T-cells” as used herein, refers to a non-antigen experienced T lymphocyte that expresses CD62L and/or CD45RA, and does not express CD45RO−, as compared to central or effector memory cells. In some embodiments, a cell, such as a mammalian cell, for fusion protein secretion is provided. In some embodiments, the cell, such as a mammalian cell, is a naïve T-cell. In some embodiments, naïve CD8+T lymphocytes are characterized by the expression of phenotypic markers of naïve T-cells comprising CD62L, CCR7, CD28, CD127, and/or CD45RA.

“Effector” T-cells as used herein, refers to antigen experienced cytotoxic T lymphocyte cells that do not express or have decreased expression of CD62L, CCR7, and/or CD28, and are positive for granzyme B and/or perforin, as compared to central memory or naïve T-cells. In some embodiments, a cell, such as a mammalian cell, for fusion protein secretion is provided. In some embodiments, the cell, such as a mammalian cell, is an effector T-cell. In some embodiments, the cell, such as a mammalian cell, does not express or have decreased expression of CD62L, CCR7, and/or CD28, and are positive for granzyme B and/or perforin, as compared to central memory or naïve T-cells.

“Epitope” as used herein, refers to a part of an antigen or molecule that is recognized by the immune system comprising antibodies, T-cells, and/or B-cells. Epitopes usually have at least 7 amino acids and can be a linear or a conformational epitope. In some embodiments, a cell, such as a mammalian cell, expressing a fusion protein is provided, wherein the cell further comprises a chimeric antigen receptor. In some embodiments, the chimeric antigen receptor comprises a scFv that can recognize an epitope on a cancer cell. “Isolating,” or “purifying” when used to describe the various polypeptides or nucleic acids disclosed herein, refers to a polypeptide or nucleic acid that has been identified and separated and/or recovered from a component of its natural environment. In some embodiments, the isolated polypeptide or nucleic acid is free of association with all components with which it is naturally associated. Contaminant components of its natural environment are materials that would typically interfere with diagnostic or therapeutic uses for the polypeptide or nucleic acid, and can include enzymes, hormones, and other proteinaceous or non-proteinaceous solutes. In some embodiments, a method is provided wherein the method comprises delivering the nucleic acid of anyone of the embodiments described herein or the expression vector of anyone of the embodiments described herein to a bacterial cell, mammalian cell or insect cell, growing the cell up in a culture, inducing expression of the fusion protein and purifying the fusion protein for treatment.

“Percent (%) amino acid sequence identity” with respect to the CISC sequences identified herein is defined as the percentage of amino acid residues in a candidate sequence that are identical with the amino acid residues in the reference sequence for each of the extracellular binding domain, hinge domain, transmembrane domain, and/or the signaling domain, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, comprising any algorithms needed to achieve maximal alignment over the full-length of the sequences being compared. For example, % amino acid sequence identity values generated using the WU-BLAST-2 computer program (Altschul, S. F. et al. (1996). Methods in Enzymol., 266:460-480) uses several search parameters, most of which are set to the default values. Those that are not set to default values (e.g., the adjustable parameters) are set with the following values: overlap span=1, overlap fraction=0.125, word threshold (T)=11 and scoring matrix=BLOSUM62. In some embodiments of the CISC, the CISC comprises an extracellular binding domain, a hinge domain, a transmembrane domain, and a signaling domain, wherein each domain comprises a natural, synthetic, or a mutated or truncated form (such as a truncated form of an ILRβ signaling domain) of the native domain. In some embodiments, a mutated or truncated form of any given domain comprises an amino acid sequence with 100%, 95%, 90%, 85% sequence identity, or a percent sequence identity that is within a range defined by any two of the aforementioned percentages to a sequence set forth in a sequence provided herein.

“CISC variant polypeptide sequence” or “CISC variant amino acid sequence” as used herein refers to a protein sequence as defined below having at least 80%, 85%, 90%, 95%, 98% or 99% amino acid sequence identity (or a percentage amino acid sequence identity within a range defined by any two of the aforementioned percentages) with the protein sequences provided herein, or a specifically derived fragment thereof, such as protein sequence for an extracellular binding domain, a hinge domain, a transmembrane domain and/or a signaling domain. Ordinarily, a CISC variant polypeptide or fragment thereof will have at least 80% amino acid sequence identity, at least 81% amino acid sequence identity, at least 82% amino acid sequence identity, at least 83% amino acid sequence identity, at least 84% amino acid sequence identity, at least 85% amino acid sequence identity, at least 86% amino acid sequence identity, at least 87% amino acid sequence identity, at least 88% amino acid sequence identity, at least 89% amino acid sequence identity, at least 90% amino acid sequence identity, at least 91% amino acid sequence identity, at least 92% amino acid sequence identity, at least 93% amino acid sequence identity, at least 94% amino acid sequence identity, at least 95% amino acid sequence identity, at least 96% amino acid sequence identity, at least 97% amino acid sequence identity, at least 98% amino acid sequence identity, or at least 99% amino acid sequence identity with the amino acid sequence or a derived fragment thereof. Variants do not encompass the native protein sequence.

“T-cells” or “T lymphocytes” as used herein can be from any mammalian, species, including without limitation monkeys, dogs, primates, and humans. In some embodiments, the T-cells are allogeneic (from the same species but different donor) as the recipient subject; in some embodiments the T-cells are autologous (the donor and the recipient are the same); in some embodiments the T-cells are syngeneic (the donor and the recipients are different but are identical twins).

“RNA-guided endonuclease,” “RGEN,” “Cas endonuclease,” or “Cas nuclease” as used herein includes, but is not limited to, for example, an RNA-guided DNA endonuclease enzyme associated with the CRISPR (Clustered Regularly Interspaced Short Palindromic Repeats) adaptive immunity system. Herein, “RGEN” or “Cas endonuclease” refers to both naturally-occurring and recombinant Cas endonucleases.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “comprising at least.” When used in the context of a process, the term “comprising” means that the process comprises at least the recited steps, but may include additional steps. When used in the context of a compound, composition or device, the term “comprising” means that the compound, composition or device comprises at least the recited features or components, but may also include additional features or components.

### Systems for Controlled Plasma Cell Depletion

In one aspect, provided herein is a system for generating engineered cells (e.g., engineered T cells) for controlled depletion of plasma cells in an individual. The system comprises a) nucleic acid for integration into the genome of a cell (e.g., a T cell) encoding i) an anti-plasma cell construct capable of conferring to the cell cytotoxicity towards a plasma cell, and ii) polypeptide components of a dimerization activatable chemical-induced signaling complex (CISC), wherein the signaling-competent CISC is capable of producing a stimulatory signal in a signaling pathway that promotes survival and/or proliferation of the cell, and b) genome editing elements for integrating the nucleic acid into the genome of the cell to produce an engineered cell expressing the anti-plasma cell construct and the CISC. The CISC allows for controlling the survival and/or proliferation of the engineered cell by modulating the amount of a ligand required for CISC dimerization in contact with the engineered cell. In some embodiments, the CISC comprises a first CISC component and a second CISC component, wherein the first CISC component and the second CISC component are configured such that when expressed by the engineered cell, they dimerize in the presence of the ligand to create the signaling-competent CISC. In some embodiments, the engineered cell is unable to survive and/or proliferate in the absence of the ligand. In some embodiments, the engineered cell is defective in an endogenous signaling pathway involved in survival and/or proliferation of the cell, and the signaling-competent CISC is capable of supplementing the defective endogenous signaling pathway such that the engineered cell can survive and/or proliferate.

### Anti Plasma Cell Construct

In some embodiments, the systems described herein comprise nucleic acid encoding an anti-plasma cell construct. In some embodiments, the anti-plasma cell construct is an anti-plasma cell chimeric antigen receptor (CAR). The anti-plasma cell CAR recognizes an antigen present on the surface of a plasma cell. In some embodiments, the anti-plasma cell CAR recognizes an antigen selectively expressed on the surface of a plasma cell. In some embodiments, the plasma cell is a non-malignant plasma cell. In some embodiments, the anti-plasma cell CAR recognizes CD27 (Tumor Necrosis Factor Receptor Superfamily, Member 7, TNFRSF7), CD126 (interleukin-6 receptor, IL6R), CD138 (syndecan 1), CD269 (B-cell maturation antigen, BCMA), or CD319 (SLAM family member 7, SLAMF7). In some embodiments, the anti-plasma cell CAR is an anti-BCMA CAR. In some embodiments, the anti-BCMA CAR recognizes wild-type BCMA. Antibody moieties specific for BCMA are known in the art, and the anti-BCMA CAR may comprise any of these anti-BCMA antibody moieties. For example, in some embodiments, the anti-BCMA CAR comprises an antibody moiety derived from the anti-BCMA antibody C11D5.3. In some embodiments, the anti-BCMA CAR comprises an anti-BCMA scFv comprising heavy chain and light chain CDR3s derived from the anti-BCMA antibody C11D5.3. In some embodiments, the anti-BCMA CAR comprises an anti-BCMA scFv, wherein each of the anti-BCMA scFv CDRs are derived from the anti-BCMA antibody C11D5.3. In some embodiments, the anti-BCMA scFv comprises the amino acid sequence of SEQ ID NO: 55 or a variant thereof having at least 85% homology to the amino acid sequence of SEQ ID NO: 55.

In some embodiments, the systems described herein comprise nucleic acid encoding an anti-BCMA CAR. In some embodiments, the anti-BCMA CAR comprises an extracellular BCMA recognition domain, a transmembrane domain, a co-stimulatory domain, and a cytoplasmic signaling domain. In some embodiments, the extracellular BCMA recognition domain is an antibody moiety that can specifically bind to BCMA. In some embodiments, the antibody moiety is an anti-BCMA scFv. In some embodiments, the anti-BCMA scFv comprises a heavy chain variable domain (V_{H}) comprising heavy chain complementarity-determining region (HC-CDR)1, HC-CDR2, and HC-CDR3, and a light chain variable domain (V_{L}) comprising light chain complementarity-determining region (LC-CDR)1, LC-CDR2, and LC-CDR3, wherein some of the CDRs are derived from an anti-BCMA antibody. In some embodiments, the HC-CDR3 and the LC-CD3 are derived from the anti-BCMA antibody. In some embodiments, the HC-CDR1, the HC-CDR2, the HC-CDR3, the LC-CDR1, the LC-CDR2, and the LC-CDR3 are derived from the anti-BCMA antibody. In some embodiments, the anti-BCMA antibody is C11D5.3. In some embodiments, the anti-BCMA scFv comprises the amino acid sequence of SEQ ID NO: 55 or a variant thereof having at least 85% homology to the amino acid sequence of SEQ ID NO: 55. In some embodiments, the anti-BCMA CAR transmembrane domain comprises a CD8 transmembrane domain. In some embodiments, the CD8 transmembrane domain comprises the amino acid sequence of SEQ ID NO: 56 or a variant thereof having at least 85% homology to the amino acid sequence of SEQ ID NO: 56. In some embodiments, the anti-BCMA CAR co-stimulatory domain comprises a 4-1BB and/or a CD28 co-stimulatory domain. In some embodiments, the CD28 co-stimulatory domain comprises the amino acid sequence of SEQ ID NO: 57 or a variant thereof having at least 85% homology to the amino acid sequence of SEQ ID NO: 57. In some embodiments, the 4-1BB co-stimulatory transmembrane domain comprises the amino acid sequence of SEQ ID NO: 58 or a variant thereof having at least 85% homology to the amino acid sequence of SEQ ID NO: 58. In some embodiments, the anti-BCMA CAR cytoplasmic signaling domain comprises a CD3-t cytoplasmic signaling domain. In some embodiments, the CD3-ζ cytoplasmic signaling domain comprises the amino acid sequence of SEQ ID NO: 59 or a variant thereof having at least 85% homology to the amino acid sequence of SEQ ID NO: 59. In some embodiments, the anti-BCMA CAR comprises the amino acid sequence of SEQ ID NO: 60 or 61 or a variant thereof having at least 85% homology to the amino acid sequence of SEQ ID NO: 60 or 61.

### CISC

In some embodiments, the systems described herein comprise nucleic acid encoding a dimeric CISC comprising a first CISC component and a second CISC component. In some embodiments, the first CISC component comprises a first extracellular binding domain or portion thereof, a first transmembrane domain, and a first signaling domain or portion thereof. In some embodiments, the first CISC component further comprises a first hinge domain. In some embodiments, the second CISC component comprises a second extracellular binding domain or portion thereof, a second transmembrane domain, and a second signaling domain or portion thereof. In some embodiments, the second CISC component further comprises a second hinge domain. In some embodiments, the first and second CISC components may be configured such that when expressed, they dimerize in the presence of a ligand. In some embodiments, the first extracellular binding domain or portion thereof comprises an FK506 binding protein (FKBP) domain or a portion thereof, and the second extracellular binding domain or portion thereof comprises an FKBP rapamycin binding (FRB) domain or a portion thereof. In some embodiments, the second extracellular binding domain or portion thereof comprises an FK506 binding protein (FKBP) domain or a portion thereof, and the first extracellular binding domain or portion thereof comprises an FKBP rapamycin binding (FRB) domain or a portion thereof. In some embodiments, the ligand is rapamycin or a rapalog. In some embodiments, the first signaling domain is a signaling domain derived from IL2Rγ and/or the first transmembrane domain is a transmembrane domain derived from IL2Rγ, and the second signaling domain is a signaling domain derived from IL2Rβ and/or the second transmembrane domain is a transmembrane domain derived from IL2Rβ. In some embodiments, the second signaling domain is a signaling domain derived from IL2Rγ and/or the second transmembrane domain is a transmembrane domain derived from IL2Rγ, and the first signaling domain is a signaling domain derived from IL2Rβ and/or the first transmembrane domain is a transmembrane domain derived from IL2R3.

In some embodiments, the systems described herein comprise nucleic acid encoding a dimeric CISC comprising a first CISC component and a second CISC component, wherein the CISC comprises IL2Rγ and IL2Rβ signaling domains. In some embodiments, the first CISC component comprises a portion of IL2Rγ including a signaling domain and the second CISC component comprises a portion of IL2Rβ including a signaling domain, or the second CISC component comprises a portion of IL2Rγ including a signaling domain and the first CISC component comprises a portion of IL2Rβ including a signaling domain. In some embodiments, the first CISC component comprises a portion of IL2Rγ comprising the amino acid sequence of SEQ ID NO: 50 or a variant thereof having at least 85% homology to the amino acid sequence of SEQ ID NO: 50 and the second CISC component comprises a portion of IL2Rβ comprising the amino acid sequence of SEQ ID NO: 51 or a variant thereof having at least 85% homology to the amino acid sequence of SEQ ID NO: 51, or the second CISC component comprises a portion of IL2Rγ comprising the amino acid sequence of SEQ ID NO: 50 or a variant thereof having at least 85% homology to the amino acid sequence of SEQ ID NO: 50 and the first CISC component comprises a portion of IL2Rβ comprising the amino acid sequence of SEQ ID NO: 51 or a variant thereof having at least 85% homology to the amino acid sequence of SEQ ID NO: 51. In some embodiments, the first extracellular binding domain or portion thereof comprises an FK506 binding protein (FKBP) domain or a portion thereof, and the second extracellular binding domain or portion thereof comprises an FKBP rapamycin binding (FRB) domain or a portion thereof. In some embodiments, the second extracellular binding domain or portion thereof comprises an FK506 binding protein (FKBP) domain or a portion thereof, and the first extracellular binding domain or portion thereof comprises an FKBP rapamycin binding (FRB) domain or a portion thereof. In some embodiments, the FKBP domain comprises the amino acid sequence of SEQ ID NO: 47 or a variant thereof having at least 85% homology to the amino acid sequence of SEQ ID NO: 47. In some embodiments, the FRB comprises the amino acid sequence of SEQ ID NO: 48 or a variant thereof having at least 85% homology to the amino acid sequence of SEQ ID NO: 48. In some embodiments, the first and second CISC components dimerize in the presence of rapamycin or a rapalog to form a signaling competent CISC. In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

In other embodiments, the CISC component comprising an IL2Rβ signaling domain comprises a truncated intracellular IL2Rβ domain. The truncated IL2Rβ domain retains the ability to activate downstream IL2 signaling upon heterodimerization with the CISC component comprising an IL2Rγ signaling domain. In some embodiments, the truncated IL2Rβ comprises an amino acid sequence as set forth in SEQ ID NO: 76. In some embodiments, the truncated IL2Rβ domain of SEQ ID NO: 76 lacks any of 1, 2, 3, 4, 5, 6, 7, 8, 9, or 10 N-terminal amino acids. In some embodiments, the CISC component comprising a truncated intracellular IL2Rβ domain comprises the amino acid sequence of SEQ ID NO: 77. In some embodiments, according to any of the CISC components comprising an IL2Rβ signaling domain described herein, the CISC component can be substituted with a CISC component comprising a truncated intracellular IL2Rβ domain. For example, in some embodiments, a CISC component comprising an IL2Rβ signaling domain described herein is substituted with a CISC component comprising the amino acid sequence of SEQ ID NO: 77.

### Anti-Cytotoxic T Cell Construct

In some embodiments, the systems described herein further comprise nucleic acid encoding an anti-cytotoxic T cell construct. In some embodiments, the anti-cytotoxic T cell construct is capable of conferring to an edited cell expressing the construct cytotoxicity towards a cytotoxic T cell that recognizes the edited cell as foreign, while the edited T cell is non-cytotoxic towards cytotoxic T cells that do not recognize the edited cell as foreign. In some embodiments, the anti-cytotoxic T cell construct is a chimeric receptor comprising an extracellular β2-microglobulin domain, a transmembrane domain, a co-stimulatory domain, and a cytoplasmic signaling domain. In some embodiments, the extracellular β2-microglobulin domain comprises the amino acid sequence of SEQ ID NO: 62 or a variant thereof having at least 85% homology to the amino acid sequence of SEQ ID NO: 62. In some embodiments, the chimeric receptor transmembrane domain comprises a CD8 transmembrane domain polypeptide. In some embodiments, the chimeric receptor CD8 transmembrane domain comprises the amino acid sequence of SEQ ID NO: 63 or a variant thereof having at least 85% homology to the amino acid sequence of SEQ ID NO: 63. In some embodiments, the chimeric receptor co-stimulatory domain comprises a 4-1BB co-stimulatory domain. In some embodiments, the chimeric receptor 4-1BB co-stimulatory domain comprises the amino acid sequence of SEQ ID NO: 64 or a variant thereof having at least 85% homology to the amino acid sequence of SEQ ID NO: 64. In some embodiments, the chimeric receptor cytoplasmic signaling domain comprises a CD3-ζ cytoplasmic signaling domain. In some embodiments, the chimeric receptor CD3-ζ cytoplasmic signaling domain comprises the amino acid sequence of SEQ ID NO: 59 or a variant thereof having at least 85% homology to the amino acid sequence of SEQ ID NO: 59. In some embodiments, the chimeric receptor comprises the amino acid sequence of SEQ ID NO: 65 or a variant thereof having at least 85% homology to the amino acid sequence of SEQ ID NO: 65.

### Selectable Marker

In some embodiments, the systems described herein further comprise nucleic acid encoding a selectable marker. In some embodiments, the selectable marker is capable of conferring to an edited cell expressing the selectable marker the ability to survive in a selective condition, such as in the presence of a toxin or in the absence of a nutrient. In some embodiments, the selectable marker is a surface marker that allows for selection of cells expressing the selectable marker. In some embodiments, the selectable marker is a truncated low-affinity nerve growth factor receptor (tLNGFR) polypeptide. In some embodiments, the tLNGFR polypeptide comprises the amino acid sequence of SEQ ID NO: 66 or a variant thereof having at least 85% homology to the amino acid sequence of SEQ ID NO: 66.

### Calcineurin Inhibitor Resistance

In some embodiments, the systems described herein further comprise nucleic acid encoding a polypeptide that confers resistance to one or more calcineurin inhibitors. In some embodiments, the polypeptide is capable of conferring to an edited cell expressing the polypeptide resistance to the one or more calcineurin inhibitors. In some embodiments, the polypeptide that confers resistance to one or more calcineurin inhibitors confers resistance to tacrolimus (FK506) and/or cyclosporin A (CsA). In some embodiments, the polypeptide that confers resistance to one or more calcineurin inhibitors is a mutant calcineurin (CN) polypeptide. In some embodiments, the mutant CN polypeptide confers resistance to tacrolimus (FK506) and cyclosporin A (CsA). In some embodiments, the mutant CN polypeptide is CNb30 (SEQ ID NO: 67).

### Rapamycin Resistance

While useful, CISC-expressing cells exposed to rapamycin have been observed to undergo less proliferation compared to the amount of proliferation achieved using the rapalog AP21967. The mammalian target of rapamycin (mTOR) is a kinase that in humans is encoded by the MTOR gene. mTOR is a member of the phosphatidylinositol 3-kinase-related kinase family of protein kinases. This protein is a growth regulator that stimulates cellular growth by phosphorylating substrates that govern anabolic processes such as lipid synthesis and mRNA translation, as well as retarding catabolic processes such as autophagy. Without being bound to theory, it is believed that the binding of a rapamycin/FKBP complex to the FRB domain of mTOR blocks or decreases mTOR-mediated intracellular signaling leading to decreased mRNA translation and cellular growth.

In some embodiments, the systems described herein further comprise nucleic acid encoding a polypeptide that confers resistance to rapamycin. In some embodiments, the polypeptide is capable of conferring to an edited cell expressing the polypeptide resistance to rapamycin. In some embodiments, the polypeptide is an FKBP-rapamycin binding (FRB) domain polypeptide of the mammalian target of rapamycin (mTOR) kinase. In some embodiments, the polypeptide that confers resistance rapamycin comprises the amino acid sequence of SEQ ID NO: 68 or 69 or a variant thereof having at least 85% homology to the amino acid sequence of SEQ ID NO: 68 or 69.

### Genome Editing Elements

In some embodiments, the systems described herein comprise genome-editing elements for integrating nucleic acid into the genome of a cell to produce an engineered cell expressing an anti-plasma cell construct and CISC described herein. In some embodiments, the genome editing elements are capable of inserting nucleic acid encoding the various polypeptides described herein into an endogenous TRA gene and/or an endogenous IL2RG gene. In some embodiments, the genome editing elements comprise a CRISPR system comprising a) a first gRNA targeting an endogenous TRA gene and/or a second gRNA targeting an endogenous IL2RG gene; and b) an RNA-guided endonuclease (RGEN) or a nucleic acid encoding the RGEN. In some embodiments, the first gRNA targets an endogenous TRA gene within or near a region encoding the TRAC domain. A gRNA target site is “near” a region encoding the TRAC domain if integration at that target site is capable of disrupting the TRAC domain expression and/or function, typically in a flanking or an adjacent sequence. In some embodiments, the first gRNA comprises the polynucleotide sequence of any one of SEQ ID NOs: 1-3, or a variant thereof having at least 85% homology to any one of SEQ ID NOs: 1-3. In some embodiments, the second gRNA comprises the polynucleotide sequence of any one of SEQ ID NOs: 4-18, or a variant thereof having at least 85% homology to any one of SEQ ID NOs: 4-18. In some embodiments, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

In some embodiments, the systems described herein comprise genome-editing elements comprising a) a first gRNA targeting an endogenous TIM gene and/or a second gRNA targeting an endogenous IL2RG gene; and b) an RNA-guided endonuclease (RGEN) or a nucleic acid encoding the RGEN. In some embodiments, the first gRNA targets an endogenous TIM gene within or near a region encoding the TRAC domain. In some embodiments, the first gRNA comprises the polynucleotide sequence of any one of SEQ ID NOs: 1-3, or a variant thereof having at least 85% homology to any one of SEQ ID NOs: 1-3. In some embodiments, the second gRNA comprises the polynucleotide sequence of any one of SEQ ID NOs: 4-18, or a variant thereof having at least 85% homology to any one of SEQ ID NOs: 4-18. In some embodiments, the RGEN is Cas9. In some embodiments, the nucleic acid encoding the RGEN is a ribonucleic acid (RNA) sequence. In some embodiments, the RNA sequence encoding the RGEN is linked to the first gRNA or the second gRNA via a covalent bond. In some embodiments, the system comprises one or more donor templates comprising nucleic acid encoding an anti-plasma cell construct and CISC described herein. In some embodiments, the anti-plasma cell construct is an anti-BCMA CAR according to any of the embodiments described herein. In some embodiments, the one or more donor templates further comprise nucleic acid encoding one or more of an anti-cytotoxic T cell construct, a selectable marker, a polypeptide that confers calcineurin inhibitor resistance, and a polypeptide that confers resistance to rapamycin according to any of the embodiments described herein. In some embodiments, the anti-cytotoxic T cell construct is a chimeric receptor comprising an extracellular β2-microglobulin domain, a transmembrane domain, a co-stimulatory domain, and a cytoplasmic signaling domain. In some embodiments, the system comprises a first donor template for insertion into the endogenous TRA gene and/or a second donor template for insertion into the endogenous IL2RG gene.

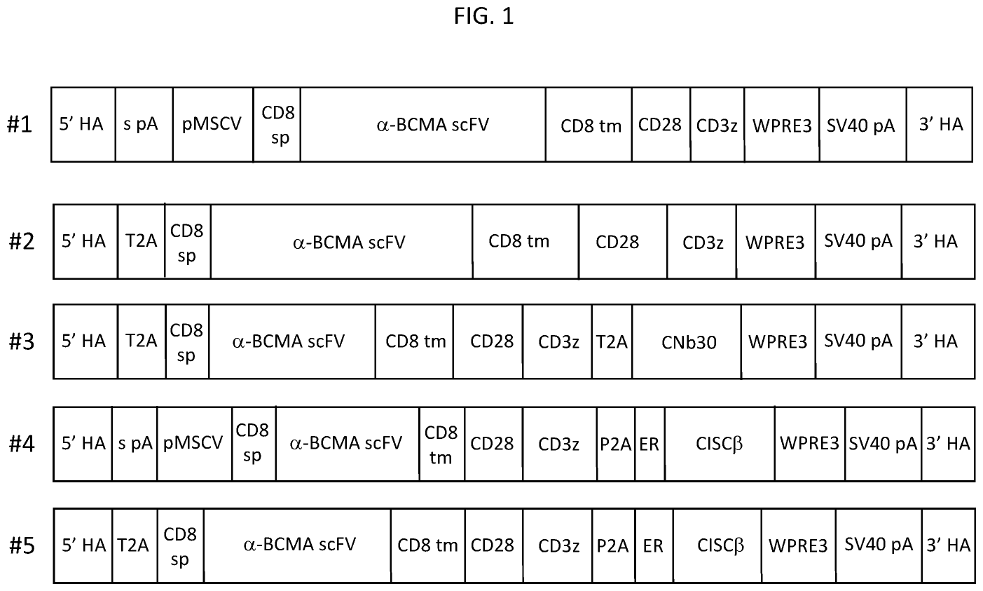

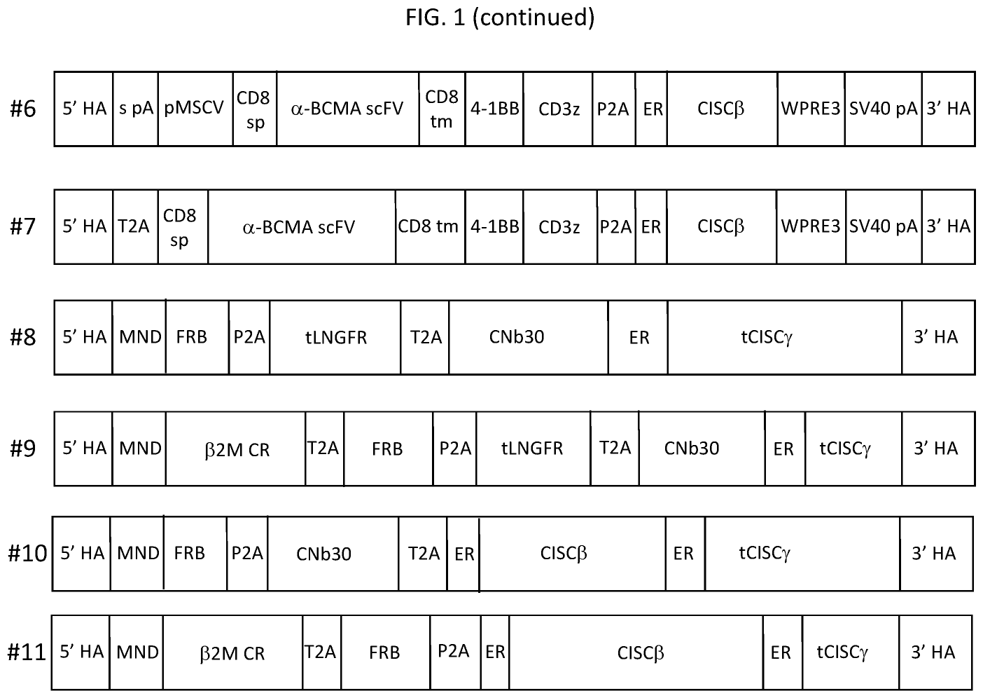

In some embodiments, the systems described herein comprise one or more donor templates comprising nucleic acid encoding the following system components: i) an anti-plasma cell construct; ii) a first CISC component comprising an IL2Rβ signaling domain; iii) a polypeptide that confers resistance to rapamycin; iv) a selectable marker; v) a polypeptide that confers resistance to one or more calcineurin inhibitors; and vi) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. The TRAC domain in a cell is non-functional if the cell is unable to express a functional native (unmodified) T cell receptor. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #4-#7. In some embodiments, the first donor template comprises a sequence of contiguous nucleotides from any one of SEQ ID NOs: 28-39. For example, in some embodiments, the first donor template comprises the nucleotide sequence of any one of SEQ ID NOs: 101-104. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #8. In some embodiments, the second donor template comprises a sequence of contiguous nucleotides from any one of SEQ ID NOs: 40-43. For example, in some embodiments, the second donor template comprises the nucleotide sequence of SEQ ID NO: 105. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 28-39 and variants thereof having at least 85% homology to the polynucleotide sequence of any one of SEQ ID NOs: 28-39. In some embodiments, the first AAV vector comprises the polynucleotide sequence of any one of SEQ ID NOs: 101-104 and variants thereof having at least 85% homology to the polynucleotide sequence of any one of SEQ ID NOs: 101-104. In some embodiments, the second AAV vector comprises the polynucleotide sequence of any one of SEQ ID NOs: 40-43 or a variant thereof having at least 85% homology to the polynucleotide sequence of any one of SEQ ID NOs: 40-43. In some embodiments, the second AAV vector comprises the polynucleotide sequence of SEQ ID NO: 105 or a variant thereof having at least 85% homology to the polynucleotide sequence of SEQ ID NO: 105.

In some embodiments, according to any of the donor templates described herein, the donor template comprises nucleic acid encoding an anti-plasma cell construct. In some embodiments, the anti-plasma cell construct is an anti-BCMA CAR. In some embodiments, the anti-BCMA CAR comprises an extracellular BCMA recognition domain, a transmembrane domain, a co-stimulatory domain, and a cytoplasmic signaling domain. In some embodiments, the extracellular BCMA recognition domain is an antibody moiety that can specifically bind to BCMA. In some embodiments, the antibody moiety is an anti-BCMA scFv. In some embodiments, the anti-BCMA scFv comprises a heavy chain variable domain (V_{H}) comprising heavy chain complementarity-determining region (HC-CDR)1, HC-CDR2, and HC-CDR3, and a light chain variable domain (V_{L}) comprising light chain complementarity-determining region (LC-CDR)1, LC-CDR2, and LC-CDR3, wherein some of the CDRs are derived from an anti-BCMA antibody. In some embodiments, the HC-CDR3 and the LC-CD3 are derived from the anti-BCMA antibody. In some embodiments, the HC-CDR1, the HC-CDR2, the HC-CDR3, the LC-CDR1, the LC-CDR2, and the LC-CDR3 are derived from the anti-BCMA antibody. In some embodiments, the anti-BCMA antibody is C11D5.3. In some embodiments, the anti-BCMA scFv comprises the amino acid sequence of SEQ ID NO: 55 or a variant thereof having at least 85% homology to the amino acid sequence of SEQ ID NO: 55. In some embodiments, the anti-BCMA CAR transmembrane domain comprises a CD8 transmembrane domain. In some embodiments, the CD8 transmembrane domain comprises the amino acid sequence of SEQ ID NO: 56 or a variant thereof having at least 85% homology to the amino acid sequence of SEQ ID NO: 56. In some embodiments, the anti-BCMA CAR co-stimulatory domain comprises a 4-1BB and/or a CD28 co-stimulatory domain. In some embodiments, the CD28 co-stimulatory domain comprises the amino acid sequence of SEQ ID NO: 57 or a variant thereof having at least 85% homology to the amino acid sequence of SEQ ID NO: 57. In some embodiments, the 4-1BB co-stimulatory transmembrane domain comprises the amino acid sequence of SEQ ID NO: 58 or a variant thereof having at least 85% homology to the amino acid sequence of SEQ ID NO: 58. In some embodiments, the anti-BCMA CAR cytoplasmic signaling domain comprises a CD3-ζ cytoplasmic signaling domain. In some embodiments, the CD3-t cytoplasmic signaling domain comprises the amino acid sequence of SEQ ID NO: 59 or a variant thereof having at least 85% homology to the amino acid sequence of SEQ ID NO: 59. In some embodiments, the anti-BCMA CAR comprises the amino acid sequence of SEQ ID NO: 60 or 61 or a variant thereof having at least 85% homology to the amino acid sequence of SEQ ID NO: 60 or 61.

In some embodiments, according to any of the donor templates described herein, the donor template comprises nucleic acid encoding a first CISC component comprising an IL2Rβ signaling domain. In some embodiments, the first extracellular binding domain of the first CISC component comprises an FRB domain. In some embodiments, the first CISC component comprises the amino acid sequence of SEQ ID NO: 54 or a variant thereof having at least 85% homology to the amino acid sequence of SEQ ID NO: 54.

In some embodiments, according to any of the donor templates described herein, the donor template comprises nucleic acid encoding a polypeptide that confers resistance to rapamycin. In some embodiments, the polypeptide that confers resistance to rapamycin is an FRB domain polypeptide. In some embodiments, the FRB domain polypeptide comprises the amino acid sequence of SEQ ID NO: 68 or 69 or a variant thereof having at least 85% homology to the amino acid sequence of SEQ ID NO: 68 or 69.

In some embodiments, according to any of the donor templates described herein, the donor template comprises nucleic acid encoding a selectable marker. In some embodiments, the selectable marker is a tLNGFR polypeptide. In some embodiments, the tLNGFR polypeptide comprises the amino acid sequence of SEQ ID NO: 66 or a variant thereof having at least 85% homology to the amino acid sequence of SEQ ID NO: 66.

In some embodiments, according to any of the donor templates described herein, the donor template comprises nucleic acid encoding a polypeptide that confers resistance to one or more calcineurin inhibitors. In some embodiments, the polypeptide that confers resistance to one or more calcineurin inhibitors is a mutant CN polypeptide. In some embodiments, the mutant CN polypeptide is CNb30 (SEQ ID NO: 67).

In some embodiments, according to any of the donor templates described herein, the donor template comprises nucleic acid encoding a second CISC component comprising an IL2Rγ signaling domain or fragment thereof. In some embodiments, the second extracellular binding domain of the second CISC component comprises an FKBP domain. In some embodiments, the second CISC component comprises the amino acid sequence of SEQ ID NO: 53 or a variant thereof having at least 85% homology to the amino acid sequence of SEQ ID NO: 53. In some embodiments, the donor template comprise nucleic acid encoding a fragment of the second CISC component comprising the amino acid sequence of SEQ ID NO: 52 or a variant thereof having at least 85% homology to the amino acid sequence of SEQ ID NO: 52.

In some embodiments, according to any of the donor templates described herein, the donor template comprises an MSCV promoter. In some embodiments, the MSCV promoter comprises the polynucleotide sequence of SEQ ID NO: 75 or a variant thereof having at least 85% homology to the polynucleotide sequence of SEQ ID NO: 75.

In some embodiments, according to any of the donor templates described herein, the donor template comprises an MND promoter. In some embodiments, the MND promoter comprises the polynucleotide sequence of SEQ ID NO: 74 or a variant thereof having at least 85% homology to the polynucleotide sequence of SEQ ID NO: 74.

In some embodiments, according to any of the donor templates described herein, the donor template comprises nucleic acid encoding a 2A self-cleaving peptide between adjacent system component-encoding nucleic acids. In some embodiments, the donor template comprise nucleic acid encoding a 2A self-cleaving peptide between each of the adjacent system component-encoding nucleic acids. For example, in some embodiments, the donor template comprises, in order from 5′ to 3′, nucleic acid encoding a polypeptide that confers resistance to rapamycin, nucleic acid encoding a 2A self-cleaving peptide, nucleic acid encoding a selectable marker, nucleic acid encoding a 2A self-cleaving peptide, nucleic acid encoding a polypeptide that confers resistance to one or more calcineurin inhibitors, nucleic acid encoding a 2A self-cleaving peptide, and nucleic acid encoding a second CISC component or a fragment thereof. In some embodiments, each of the 2A self-cleaving peptides is, independently, a T2A self-cleaving peptide or a P2A self-cleaving peptide. In some embodiments, the T2A self-cleaving peptide comprises the amino acid sequence of SEQ ID NO: 72 or a variant thereof having at least 85% homology to the amino acid sequence of SEQ ID NO: 72. In some embodiments, the P2A self-cleaving peptide comprises the amino acid sequence of SEQ ID NO: 73 or a variant thereof having at least 85% homology to the amino acid sequence of SEQ ID NO: 73.

In some embodiments, the systems described herein comprise one or more donor templates comprising nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a selectable marker; vi) a polypeptide that confers resistance to one or more calcineurin inhibitors; and vii) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the anti-cytotoxic T cell construct, the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #4-#7. In some embodiments, the first donor template comprises a sequence of contiguous nucleotides from any one of SEQ ID NOs: 28-39. For example, in some embodiments, the first donor template comprises the nucleotide sequence of any one of SEQ ID NOs: 101-104. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #9. In some embodiments, the second donor template comprises a sequence of contiguous nucleotides from SEQ ID NO: 44. For example, in some embodiments, the second donor template comprises the nucleotide sequence of SEQ ID NO: 106. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 28-39 and variants thereof having at least 85% homology to the polynucleotide sequence of any one of SEQ ID NOs: 28-39. In some embodiments, the first AAV vector comprises the polynucleotide sequence of any one of SEQ ID NOs: 101-104 and variants thereof having at least 85% homology to the polynucleotide sequence of any one of SEQ ID NOs: 101-104. In some embodiments, the second AAV vector comprises the polynucleotide sequence of SEQ ID NO: 44 or a variant thereof having at least 85% homology to the polynucleotide sequence of SEQ ID NO: 44. In some embodiments, the second AAV vector comprises the polynucleotide sequence of SEQ ID NO: 106 or a variant thereof having at least 85% homology to the polynucleotide sequence of SEQ ID NO: 106.

In some embodiments, according to any of the donor templates described herein, the donor template comprises nucleic acid encoding an anti-cytotoxic T cell construct. In some embodiments, the anti-cytotoxic T cell construct is capable of conferring to an edited T cell expressing the construct cytotoxicity towards a cytotoxic T cell that recognizes the edited T cell as foreign, while the edited T cell is non-cytotoxic towards cytotoxic T cells that do not recognize the edited T cell as foreign. In some embodiments, the anti-cytotoxic T cell construct is a chimeric receptor comprising an extracellular β2-microglobulin domain, a transmembrane domain, a co-stimulatory domain, and a cytoplasmic signaling domain. In some embodiments, the extracellular β2-microglobulin domain comprises the amino acid sequence of SEQ ID NO: 62 or a variant thereof having at least 85% homology to the amino acid sequence of SEQ ID NO: 62. In some embodiments, the chimeric receptor transmembrane domain comprises a CD8 transmembrane domain polypeptide. In some embodiments, the chimeric receptor CD8 transmembrane domain comprises the amino acid sequence of SEQ ID NO: 63 or a variant thereof having at least 85% homology to the amino acid sequence of SEQ ID NO: 63. In some embodiments, the chimeric receptor co-stimulatory domain comprises a 4-1BB co-stimulatory domain. In some embodiments, the chimeric receptor 4-1BB co-stimulatory domain comprises the amino acid sequence of SEQ ID NO: 64 or a variant thereof having at least 85% homology to the amino acid sequence of SEQ ID NO: 64. In some embodiments, the chimeric receptor cytoplasmic signaling domain comprises a CD3-ζ cytoplasmic signaling domain. In some embodiments, the chimeric receptor CD3-t cytoplasmic signaling domain comprises the amino acid sequence of SEQ ID NO: 59 or a variant thereof having at least 85% homology to the amino acid sequence of SEQ ID NO: 59. In some embodiments, the chimeric receptor comprises the amino acid sequence of SEQ ID NO: 65 or a variant thereof having at least 85% homology to the amino acid sequence of SEQ ID NO: 65.

In some embodiments, the systems described herein comprise one or more donor templates comprising nucleic acid encoding the following system components: i) an anti-plasma cell construct; ii) a first CISC component comprising an IL2Rβ signaling domain; iii) a polypeptide that confers resistance to rapamycin; iv) a polypeptide that confers resistance to one or more calcineurin inhibitors; and v) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct. In some embodiments, the second coding cassette comprises the nucleic acid encoding the polypeptide that confers resistance to rapamycin, the nucleic acid encoding the polypeptide that confers resistance to one or more calcineurin inhibitors, the nucleic acid encoding the first CISC component, and the nucleic acid encoding the second CISC component or a fragment thereof. In some embodiments, the first donor template comprises a synthetic polyA sequence upstream of a first promoter operably linked to the first coding cassette, such that expression of the nucleic acid encoding the anti-plasma cell construct is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding sequence, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #1 and #2. In some embodiments, the first donor template comprises a sequence of contiguous nucleotides from any one of SEQ ID NOs: 19-24. For example, in some embodiments, the first donor template comprises the nucleotide sequence of any one of SEQ ID NOs: 98-99. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #10. In some embodiments, the second donor template comprises a sequence of contiguous nucleotides from SEQ ID NO: 45. For example, in some embodiments, the second donor template comprises the nucleotide sequence of SEQ ID NO: 107. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 19-24 and variants thereof having at least 85% homology to the polynucleotide sequence of any one of SEQ ID NOs: 19-24. In some embodiments, the first AAV vector comprises the polynucleotide sequence of any one of SEQ ID NOs: 98-99 and variants thereof having at least 85% homology to the polynucleotide sequence of any one of SEQ ID NOs: 98-99. In some embodiments, the second AAV vector comprises the polynucleotide sequence of SEQ ID NO: 45 or a variant thereof having at least 85% homology to the polynucleotide sequence of SEQ ID NO: 45. In some embodiments, the second AAV vector comprises the polynucleotide sequence of SEQ ID NO: 107 or a variant thereof having at least 85% homology to the polynucleotide sequence of SEQ ID NO: 107.

In some embodiments, the systems described herein comprise one or more donor templates comprising nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a polypeptide that confers resistance to one or more calcineurin inhibitors; and vi) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the polypeptide that confers resistance to one or more calcineurin inhibitors. In some embodiments, the second coding cassette comprises the nucleic acid encoding the polypeptide that confers resistance to rapamycin, the nucleic acid encoding the first CISC component, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template construct #3. In some embodiments, the first donor template comprises a sequence of contiguous nucleotides from any one of SEQ ID NOs: 25-27. For example, in some embodiments, the first donor template comprises the nucleotide sequence of SEQ ID NO: 100. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #11. In some embodiments, the second donor template comprises a sequence of contiguous nucleotides from SEQ ID NO: 46. For example, in some embodiments, the second donor template comprises the nucleotide sequence of SEQ ID NO: 108. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 25-27 and variants thereof having at least 85% homology to the polynucleotide sequence of any one of SEQ ID NOs: 25-27. In some embodiments, the first AAV vector comprises the polynucleotide sequence of SEQ ID NO: 100 and variants thereof having at least 85% homology to the polynucleotide sequence of SEQ ID NO: 100. In some embodiments, the second AAV vector comprises the polynucleotide sequence of SEQ ID NO: 46 or a variant thereof having at least 85% homology to the polynucleotide sequence of SEQ ID NO: 46. In some embodiments, the second AAV vector comprises the polynucleotide sequence of SEQ ID NO: 108 or a variant thereof having at least 85% homology to the polynucleotide sequence of SEQ ID NO: 108.

In some embodiments, the systems described herein comprise one or more donor templates and one or more gRNAs. In some embodiments, the one or more donor templates comprise a first donor template and a second donor template and the one or more gRNAs comprise a first gRNA and a second gRNA. In some embodiments, the first donor template is a first AAV vector and/or the second donor template is a second AAV vector. In some embodiments, the first AAV vector comprises the polynucleotide sequence of any one of SEQ ID NOs: 28, 31, 34, and 37 and variants thereof having at least 85% homology to the polynucleotide sequence of any one of SEQ ID NOs: 28, 31, 34, and 37, and the first gRNA comprises the polynucleotide sequence of SEQ ID NO: 1 or a variant thereof having at least 85% homology to the polynucleotide sequence of SEQ ID NO: 1, and the second AAV vector comprises the polynucleotide sequence of any one of SEQ ID NOs: 40-44 or a variant thereof having at least 85% homology to the polynucleotide sequence of any one of SEQ ID NOs: 40-44, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of any one of SEQ ID NOs: 29, 32, 35, and 38 and variants thereof having at least 85% homology to the polynucleotide sequence of any one of SEQ ID NOs: 29, 32, 35, and 38, and the first gRNA comprises the polynucleotide sequence of SEQ ID NO: 2 or a variant thereof having at least 85% homology to the polynucleotide sequence of SEQ ID NO: 2, and the second AAV vector comprises the polynucleotide sequence of any one of SEQ ID NOs: 40-44 or a variant thereof having at least 85% homology to the polynucleotide sequence of any one of SEQ ID NOs: 40-44, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of any one of SEQ ID NOs: 30, 33, 36, and 39 and variants thereof having at least 85% homology to the polynucleotide sequence of any one of SEQ ID NOs: 30, 33, 36, and 39, and the first gRNA comprises the polynucleotide sequence of SEQ ID NO: 3 or a variant thereof having at least 85% homology to the polynucleotide sequence of SEQ ID NO: 3, and the second AAV vector comprises the polynucleotide sequence of any one of SEQ ID NOs: 40-44 or a variant thereof having at least 85% homology to the polynucleotide sequence of any one of SEQ ID NOs: 40-44, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of SEQ ID NO: 19 or 22 or a variant thereof having at least 85% homology to the polynucleotide sequence of SEQ ID NO: 19 or 22, and the first gRNA comprises the polynucleotide sequence of SEQ ID NO: 1 or a variant thereof having at least 85% homology to the polynucleotide sequence of SEQ ID NO: 1, and the second AAV vector comprises the polynucleotide sequence of SEQ ID NO: 45 or a variant thereof having at least 85% homology to the polynucleotide sequence of SEQ ID NO: 45, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of SEQ ID NO: 20 or 23 or a variant thereof having at least 85% homology to the polynucleotide sequence of SEQ ID NO: 20 or 23, and the first gRNA comprises the polynucleotide sequence of SEQ ID NO: 2 or a variant thereof having at least 85% homology to the polynucleotide sequence of SEQ ID NO: 2, and the second AAV vector comprises the polynucleotide sequence of SEQ ID NO: 45 or a variant thereof having at least 85% homology to the polynucleotide sequence of SEQ ID NO: 45, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of SEQ ID NO: 21 or 24 or a variant thereof having at least 85% homology to the polynucleotide sequence of SEQ ID NO: 21 or 24, and the first gRNA comprises the polynucleotide sequence of SEQ ID NO: 3 or a variant thereof having at least 85% homology to the polynucleotide sequence of SEQ ID NO: 3, and the second AAV vector comprises the polynucleotide sequence of SEQ ID NO: 45 or a variant thereof having at least 85% homology to the polynucleotide sequence of SEQ ID NO: 45, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of SEQ ID NO: 25 or a variant thereof having at least 85% homology to the polynucleotide sequence of SEQ ID NO: 25, and the first gRNA comprises the polynucleotide sequence of SEQ ID NO: 1 or a variant thereof having at least 85% homology to the polynucleotide sequence of SEQ ID NO: 1, and the second AAV vector comprises the polynucleotide sequence of SEQ ID NO: 46 or a variant thereof having at least 85% homology to the polynucleotide sequence of SEQ ID NO: 46, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of SEQ ID NO: 26 or a variant thereof having at least 85% homology to the polynucleotide sequence of SEQ ID NO: 26, and the first gRNA comprises the polynucleotide sequence of SEQ ID NO: 2 or a variant thereof having at least 85% homology to the polynucleotide sequence of SEQ ID NO: 2, and the second AAV vector comprises the polynucleotide sequence of SEQ ID NO: 46 or a variant thereof having at least 85% homology to the polynucleotide sequence of SEQ ID NO: 46, and the second gRNA comprises the polynucleotide sequence of any one of SEQ ID NOs: 4-18 and variants thereof having at least 85% homology to the polynucleotide sequence of any one of SEQ ID NOs: 4-18. In some embodiments, the first AAV vector comprises the polynucleotide sequence of SEQ ID NO: 27 or a variant thereof having at least 85% homology to the polynucleotide sequence of SEQ ID NO: 27, and the first gRNA comprises the polynucleotide sequence of SEQ ID NO: 3 or a variant thereof having at least 85% homology to the polynucleotide sequence of SEQ ID NO: 3, and the second AAV vector comprises the polynucleotide sequence of SEQ ID NO: 46 or a variant thereof having at least 85% homology to the polynucleotide sequence of SEQ ID NO: 46, and the second gRNA comprises the polynucleotide sequence of any one of SEQ ID NOs: 4-18 and variants thereof having at least 85% homology to the polynucleotide sequence of any one of SEQ ID NOs: 4-18.

In some embodiments, according to any of the systems described herein comprising a donor template, the donor template comprises a coding cassette, and the donor template is configured such that the coding cassette is capable of being integrated into a genomic locus targeted by a gRNA in the system by homology directed repair (HDR). In some embodiments, the coding cassette is flanked on both sides by homology arms corresponding to sequences in the targeted genomic locus. In some embodiments, the homology arms correspond to sequences in the targeted genomic locus that include a target site for a gRNA is the system. In some embodiments, one or both of the homology arms comprise a sequence corresponding to a target site for a gRNA in the system. In some embodiments, the homology arms are configured such that integration of the coding cassette into the genomic locus removes the genomic target site for the gRNA or otherwise modifies the genomic target site such that it is no longer a target for the gRNA. In some embodiments, the sequence in the homology arms corresponding to the target site comprises a change in the PAM sequence of the target site such that it is not a target for the gRNA. In some embodiments, one of the homology arms comprises a sequence corresponding to a portion of the target site, and the other homology arm comprises a sequence corresponding to the remainder of the target site, such that integration of the coding sequence into the genomic locus interrupts the target site in the genomic locus. In some embodiments, the homology arms are at least or at least about 0.2 kb (such as at least or at least about any of 0.3 kb, 0.4 kb, 0.5 kb, 0.6 kb, 0.7 kb, 0.8 kb, 0.9 kb, 1 kb, or greater) in length. Exemplary homology arms include homology arms from donor templates having the sequence of any one of SEQ ID NOs: 19-46. In some embodiments, the donor template is encoded in an Adeno Associated Virus (AAV) vector. In some embodiments, the AAV vector is an AAV6 vector.

In some embodiments, according to any of the systems described herein comprising a donor template, the donor template comprises a coding cassette, and the donor template is configured such that the coding cassette is capable of being integrated into a genomic locus targeted by a gRNA in the system by non-homologous end joining (NHEJ). In some embodiments, the coding cassette is flanked on one or both sides by a gRNA target site. In some embodiments, the coding cassette is flanked on both sides by a gRNA target site. In some embodiments, the gRNA target site is a target site for a gRNA in the system. In some embodiments, the gRNA target site of the donor template is the reverse complement of a cell genome gRNA target site for a gRNA in the system. In some embodiments, the donor template is encoded in an Adeno Associated Virus (AAV) vector. In some embodiments, the AAV vector is an AAV6 vector.

In some embodiments, the systems described herein comprise a ribonucleoprotein (RNP) complex comprising the RGEN and the first gRNA and/or the second gRNA. In some embodiments, the RGEN is precomplexed with the first gRNA and/or the second gRNA at a molar ratio of gRNA to RGEN between 1:1 to 20:1, respectively, to form the RNP.

### Engineered Cells

In some aspects, provided herein are engineered cells, such as engineered mammalian cells (e.g., T cells), comprising nucleic acid encoding i) an anti-plasma cell construct capable of conferring to the engineered cells cytotoxicity towards a plasma cell as set forth and described herein, and ii) polypeptide components of a dimerization activatable chemical-induced signaling complex (CISC) as set forth and described herein, wherein the signaling-competent CISC is capable of producing a stimulatory signal in a signaling pathway that promotes survival and/or proliferation of the engineered cells. The CISC allows for controlling the survival and/or proliferation of the engineered cells by modulating the amount of a ligand required for CISC dimerization in contact with the engineered cells. In some embodiments, the CISC comprises a first CISC component and a second CISC component, wherein the first CISC component and the second CISC component are configured such that when expressed by the engineered cell, they dimerize in the presence of the ligand to create the signaling-competent CISC. In some embodiments, the engineered cell is unable to survive and/or proliferate in the absence of the ligand. In some embodiments, the engineered cell is defective in an endogenous signaling pathway involved in survival and/or proliferation of the cell, and the signaling-competent CISC is capable of supplementing the defective endogenous signaling pathway such that the engineered cell can survive and/or proliferate. In some embodiments, the engineered cells are engineered T cells. In some embodiments, the engineered T cells comprising an anti-plasma cell CAR as described herein, such as, for example, an anti-BCMA CAR, degranulate in the presence of, or following contact with, its target antigen. In some embodiments, the engineered T cells localize to sites of plasma cell neoplasm tumors, such as, for example, multiple myeloma, in an individual. In some embodiments, the engineered T cells localize to the sites of plasma cell residency in the body, for example, to the bone marrow and intestines. In some embodiments, the engineered T cells are human.

In some embodiments, the engineered cells described herein comprise nucleic acid encoding an anti-plasma cell construct. In some embodiments, the anti-plasma cell construct is an anti-plasma cell chimeric antigen receptor (CAR). The anti-plasma cell CAR recognizes an antigen present on the surface of a plasma cell. In some embodiments, the anti-plasma cell CAR recognizes an antigen selectively expressed on the surface of a plasma cell. In some embodiments, the plasma cell is a non-malignant plasma cell. In some embodiments, the anti-plasma cell CAR recognizes CD27 (Tumor Necrosis Factor Receptor Superfamily, Member 7, TNFRSF7), CD126 (interleukin-6 receptor, IL6R), CD138 (syndecan 1), CD269 (B-cell maturation antigen, BCMA), or CD319 (SLAM family member 7, SLAMF7). In some embodiments, the anti-plasma cell CAR is an anti-BCMA CAR. In some embodiments, the anti-BCMA CAR recognizes wild-type BCMA. Antibody moieties specific for BCMA are known in the art, and the anti-BCMA CAR may comprise any of these anti-BCMA antibody moieties. For example, in some embodiments, the anti-BCMA CAR comprises an antibody moiety derived from the anti-BCMA antibody C11D5.3. In some embodiments, the anti-BCMA CAR comprises an anti-BCMA scFv comprising heavy chain and light chain CDR3s derived from the anti-BCMA antibody C11D5.3. In some embodiments, the anti-BCMA CAR comprises an anti-BCMA scFv, wherein each of the anti-BCMA scFv CDRs are derived from the anti-BCMA antibody C11D5.3. In some embodiments, the anti-BCMA scFv comprises the amino acid sequence of SEQ ID NO: 55 or a variant thereof having at least 85% homology to the amino acid sequence of SEQ ID NO: 55.

In some embodiments, the engineered cells described herein comprise nucleic acid encoding an anti-BCMA CAR. In some embodiments, the anti-BCMA CAR comprises an extracellular BCMA recognition domain, a transmembrane domain, a co-stimulatory domain, and a cytoplasmic signaling domain. In some embodiments, the extracellular BCMA recognition domain is an antibody moiety that can specifically bind to BCMA. In some embodiments, the antibody moiety is an anti-BCMA scFv. In some embodiments, the anti-BCMA scFv comprises a heavy chain variable domain (V_{H}) comprising heavy chain complementarity-determining region (HC-CDR)1, HC-CDR2, and HC-CDR3, and a light chain variable domain (V_{L}) comprising light chain complementarity-determining region (LC-CDR)1, LC-CDR2, and LC-CDR3, wherein some of the CDRs are derived from an anti-BCMA antibody. In some embodiments, the HC-CDR3 and the LC-CD3 are derived from the anti-BCMA antibody. In some embodiments, the HC-CDR1, the HC-CDR2, the HC-CDR3, the LC-CDR1, the LC-CDR2, and the LC-CDR3 are derived from the anti-BCMA antibody. In some embodiments, the anti-BCMA antibody is C11D5.3. In some embodiments, the anti-BCMA scFv comprises the amino acid sequence of SEQ ID NO: 55 or a variant thereof having at least 85% homology to the amino acid sequence of SEQ ID NO: 55. In some embodiments, the anti-BCMA CAR transmembrane domain comprises a CD8 transmembrane domain. In some embodiments, the CD8 transmembrane domain comprises the amino acid sequence of SEQ ID NO: 56 or a variant thereof having at least 85% homology to the amino acid sequence of SEQ ID NO: 56. In some embodiments, the anti-BCMA CAR co-stimulatory domain comprises a 4-1BB and/or a CD28 co-stimulatory domain. In some embodiments, the CD28 co-stimulatory domain comprises the amino acid sequence of SEQ ID NO: 57 or a variant thereof having at least 85% homology to the amino acid sequence of SEQ ID NO: 57. In some embodiments, the 4-1BB co-stimulatory transmembrane domain comprises the amino acid sequence of SEQ ID NO: 58 or a variant thereof having at least 85% homology to the amino acid sequence of SEQ ID NO: 58. In some embodiments, the anti-BCMA CAR cytoplasmic signaling domain comprises a CD3-ζ cytoplasmic signaling domain. In some embodiments, the CD3-ζ cytoplasmic signaling domain comprises the amino acid sequence of SEQ ID NO: 59 or a variant thereof having at least 85% homology to the amino acid sequence of SEQ ID NO: 59. In some embodiments, the anti-BCMA CAR comprises the amino acid sequence of SEQ ID NO: 60 or 61 or a variant thereof having at least 85% homology to the amino acid sequence of SEQ ID NO: 60 or 61.

In some embodiments, according to any of the engineered cells described herein, an exogenous nucleic acid encoding the anti-plasma cell construct is inserted into the genome of the engineered cells. In some embodiments, the exogenous nucleic acid is inserted into an endogenous TRA gene. In some embodiments, the exogenous nucleic acid is inserted into the region of the endogenous TRA gene encoding the TRAC domain. In some embodiments, insertion of the exogenous nucleic acid results in a non-functional TRAC domain. The TRAC domain is non-functional if the resulting cell is unable to express a functional native (unmodified) T cell receptor. In some embodiments, the exogenous nucleic acid is inserted into an endogenous IL2RG gene. In some embodiments, the exogenous nucleic acid is inserted into an endogenous IL2RG gene such that expression of the anti-plasma cell construct is under the control of one or more endogenous IL2RG regulatory elements. In some embodiments, the exogenous nucleic acid further comprises a promoter operably linked to the portion of the exogenous nucleic acid encoding the anti-plasma cell construct, such that expression of the anti-plasma cell construct in the engineered cells is under the control of the promoter. In some embodiments, the promoter is a myeloproliferative sarcoma virus enhancer, negative control region deleted, dl587rev primer-binding site substituted (MND) promoter. In some embodiments, the MND promoter comprises the polynucleotide sequence of SEQ ID NO: 74 or a variant thereof having at least 85% homology to the polynucleotide sequence of SEQ ID NO: 74.

In some embodiments, the engineered cells described herein comprise nucleic acid encoding a dimeric CISC comprising a first CISC component and a second CISC component. In some embodiments, the first CISC component comprises a first extracellular binding domain or portion thereof, a first transmembrane domain, and a first signaling domain or portion thereof. In some embodiments, the first CISC component further comprises a first hinge domain. In some embodiments, the second CISC component comprises a second extracellular binding domain or portion thereof, a second transmembrane domain, and a second signaling domain or portion thereof. In some embodiments, the second CISC component further comprises a second hinge domain. In some embodiments, the first and second CISC components may be configured such that when expressed, they dimerize in the presence of a ligand. In some embodiments, the first extracellular binding domain or portion thereof comprises an FK506 binding protein (FKBP) domain or a portion thereof, and the second extracellular binding domain or portion thereof comprises an FKBP rapamycin binding (FRB) domain or a portion thereof. In some embodiments, the second extracellular binding domain or portion thereof comprises an FK506 binding protein (FKBP) domain or a portion thereof, and the first extracellular binding domain or portion thereof comprises an FKBP rapamycin binding (FRB) domain or a portion thereof. In some embodiments, the ligand is rapamycin or a rapalog. In some embodiments, the first signaling domain is a signaling domain derived from IL2Rγ and/or the first transmembrane domain is a transmembrane domain derived from IL2Rγ, and the second signaling domain is a signaling domain derived from IL2Rβ and/or the second transmembrane domain is a transmembrane domain derived from IL2Rβ. In some embodiments, the second signaling domain is a signaling domain derived from IL2Rγ and/or the second transmembrane domain is a transmembrane domain derived from IL2Rγ, and the first signaling domain is a signaling domain derived from IL2Rβ and/or the first transmembrane domain is a transmembrane domain derived from IL2Rβ.

In some embodiments, the engineered cells described herein comprise nucleic acid encoding a dimeric CISC comprising a first CISC component and a second CISC component, wherein the CISC comprises IL2Rγ and IL2Rβ signaling domains. In some embodiments, the first CISC component comprises a portion of IL2Rγ including a signaling domain and the second CISC component comprises a portion of IL2Rβ including a signaling domain, or the second CISC component comprises a portion of IL2Rγ including a signaling domain and the first CISC component comprises a portion of IL2Rβ including a signaling domain. In some embodiments, the first CISC component comprises a portion of IL2Rγ comprising the amino acid sequence of SEQ ID NO: 50 or a variant thereof having at least 85% homology to the amino acid sequence of SEQ ID NO: 50 and the second CISC component comprises a portion of IL2Rβ comprising the amino acid sequence of SEQ ID NO: 51 or a variant thereof having at least 85% homology to the amino acid sequence of SEQ ID NO: 51, or the second CISC component comprises a portion of IL2Rγ comprising the amino acid sequence of SEQ ID NO: 50 or a variant thereof having at least 85% homology to the amino acid sequence of SEQ ID NO: 50 and the first CISC component comprises a portion of IL2Rβ comprising the amino acid sequence of SEQ ID NO: 51 or a variant thereof having at least 85% homology to the amino acid sequence of SEQ ID NO: 51. In some embodiments, the first extracellular binding domain or portion thereof comprises an FK506 binding protein (FKBP) domain or a portion thereof, and the second extracellular binding domain or portion thereof comprises an FKBP rapamycin binding (FRB) domain or a portion thereof. In some embodiments, the second extracellular binding domain or portion thereof comprises an FK506 binding protein (FKBP) domain or a portion thereof, and the first extracellular binding domain or portion thereof comprises an FKBP rapamycin binding (FRB) domain or a portion thereof. In some embodiments, the FKBP domain comprises the amino acid sequence of SEQ ID NO: 47 or a variant thereof having at least 85% homology to the amino acid sequence of SEQ ID NO: 47. In some embodiments, the FRB comprises the amino acid sequence of SEQ ID NO: 48 or a variant thereof having at least 85% homology to the amino acid sequence of SEQ ID NO: 48. In some embodiments, the first and second CISC components dimerize in the presence of rapamycin or a rapalog to form a signaling competent CISC. In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

In some embodiments, according to any of the engineered cells described herein, a first exogenous nucleic acid encoding the first CISC component or a portion thereof is inserted into the genome of the engineered cells and/or a second exogenous nucleic acid encoding the second CISC component or a portion thereof is inserted into the genome of the engineered cells. In some embodiments, the first exogenous nucleic acid is inserted into an endogenous TRA gene and/or the second exogenous nucleic acid is inserted into an endogenous TRA gene. In some embodiments, the first exogenous nucleic acid is inserted into the region of the endogenous TRA gene encoding the TRAC domain and/or the second exogenous nucleic acid is inserted into the region of the endogenous TRA gene encoding the TRAC domain. In some embodiments, insertion of exogenous nucleic acid results in a non-functional TRAC domain. In some embodiments, the first exogenous nucleic acid is inserted into an endogenous IL2RG gene and/or the second exogenous nucleic acid is inserted into an endogenous IL2RG gene. In some embodiments, exogenous nucleic acid encoding a CISC component comprising a portion of IL2Rγ is inserted into the endogenous IL2RG gene. In some embodiments, exogenous nucleic acid encoding a CISC component comprising a portion of IL2Rγ is inserted into the endogenous IL2RG gene such that expression of the CISC component is under the control of one or more endogenous IL2RG regulatory elements. In some embodiments, exogenous nucleic acid encoding an N-terminal fragment of a CISC component comprising a portion of IL2Rγ is inserted into the endogenous IL2RG gene such that i) expression of the CISC component is under the control of one or more endogenous IL2RG regulatory elements, and ii) the exogenous nucleic acid encoding the N-terminal fragment of the CISC component is inserted in frame with the endogenous IL2RG gene, and the remaining C-terminal portion of the CISC component is encoded by a C-terminal portion of the coding sequence of the endogenous IL2RG gene. In some embodiments, the first exogenous nucleic acid further comprises a first promoter operably linked to the portion of the exogenous nucleic acid encoding the first CISC component or portion thereof, such that expression of the first CISC component in the engineered cells is under the control of the first promoter. In some embodiments, the second exogenous nucleic acid further comprises a second promoter operably linked to the portion of the exogenous nucleic acid encoding the second CISC component or portion thereof, such that expression of the second CISC component in the engineered cells is under the control of the second promoter. In some embodiments, a single exogenous nucleic acid encoding the first CISC component or portion thereof and the second CISC component of portion thereof is inserted into the genome of the engineered cells. In some embodiments, the single exogenous nucleic acid further comprises a single promoter operably linked to the portions of the exogenous nucleic acid encoding the first and second CISC components or portions thereof, such that expression of the first and second CISC components in the engineered cells is under the control of the single promoter. In some embodiments, the first, second, and/or single promoter is a myeloproliferative sarcoma virus enhancer, negative control region deleted, dl587rev primer-binding site substituted (MND) promoter. In some embodiments, the MND promoter comprises the polynucleotide sequence of SEQ ID NO: 74 or a variant thereof having at least 85% homology to the polynucleotide sequence of SEQ ID NO: 74.

In some embodiments, the engineered cells are T cells, or precursor cells capable of differentiating into T cells. In some embodiments, the engineered cells are CD3+, CD8+, and/or CD4+T lymphocytes. In some embodiments, the engineered cells are CD8+T cytotoxic lymphocyte cells, which may include naïve CD8+ T cells, central memory CD8+ T cells, effector memory CD8+ T cells, or bulk CD8+ T cells.

The lymphocytes (T lymphocytes) can be collected in accordance with known techniques and enriched or depleted by known techniques such as affinity binding to antibodies such as flow cytometry and/or immunomagnetic selection. After enrichment and/or depletion steps, in vitro expansion of the desired T lymphocytes can be carried out in accordance with known techniques or variations thereof that will be apparent to those skilled in the art. In some embodiments, the T cells are autologous T cells obtained from a patient.

For example, the desired T cell population or subpopulation can be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g. for a time sufficient to expand the numbers of T cells). The non-dividing feeder cells can comprise gamma-irradiated PBMC feeder cells. In some embodiments, the PBMC are irradiated with gamma rays in the range of 3000 to 3600 rads to prevent cell division. In some embodiments, the PBMC are irradiated with gamma rays of 3000, 3100, 3200, 3300, 3400, 3500 or 3600 rads or any value of rads between any two endpoints of any of the listed values to prevent cell division. The order of addition of the T cells and feeder cells to the culture media can be reversed if desired. The culture can generally be incubated under conditions of temperature and the like that are suitable for the growth of T lymphocytes. For the growth of human T lymphocytes, for example, the temperature will generally be at least 25° C., at least 30° C., or at least 37° C. In some embodiments, the temperature for the growth of human T lymphocytes is 22, 24, 26, 28, 30, 32, 34, 36, 37° C., or any other temperature between any two endpoints of any of the listed values.

After isolation of T lymphocytes both cytotoxic and helper T lymphocytes can be sorted into naïve, memory, and effector T cell subpopulations either before or after expansion.

CD8+ cells can be obtained by using methods known in the art. In some embodiments, CD8+ cells are further sorted into naïve, central memory, and effector memory cells by identifying cell surface antigens that are associated with each of those types of CD8+ cells. In some embodiments, memory T cells are present in both CD62L+ and CD62L− subsets of CD8+ peripheral blood lymphocytes. PBMC are sorted into CD62L-CD8+ and CD62L+CD8+ fractions after staining with anti-CD8 and anti-CD62L antibodies. In some embodiments, the expression of phenotypic markers of central memory T_{CM} include CD45RO, CD62L, CCR7, CD28, CD3, and/or CD127 and are negative or low for granzyme B. In some embodiments, central memory T cells are CD45RO+, CD62L+, and/or CD8+ T cells. In some embodiments, effector TE are negative for CD62L, CCR7, CD28, and/or CD127, and positive for granzyme B and/or perforin. In some embodiments, naïve CD8+T lymphocytes are characterized by the expression of phenotypic markers of naïve T cells comprising CD62L, CCR7, CD28, CD3, CD127, and/or CD45RA.

Whether a cell, such as a mammalian cell, or cell population, such as a population of mammalian cells, is selected for expansion depends upon whether the cell or population of cells has undergone two distinct genetic modification events. If a cell, such as a mammalian cell, or a population of cells, such as a population of mammalian cells, has undergone one or fewer genetic modification events, then the addition of a ligand will result in no dimerization. However, if the cell, such as a mammalian cell, or the population of cells, such as a population of mammalian cells, has undergone two genetic modification events, then the addition of the ligand will result in dimerization of the CISC component, and subsequent signaling cascade. Thus, a cell, such as a mammalian cell, or a population of cells, such as a population of mammalian cells, may be selected based on its response to contact with the ligand. In some embodiments, the ligand may be added in an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values.

In some embodiments, a cell, such as a mammalian cell, or a population of cells, such as a population of mammalian cells, may be positive for the dimeric CISC based on the expression of a marker as a result of a signaling pathway. Thus, a cell population positive for the dimeric CISC may be determined by flow cytometry using staining with a specific antibody for the surface marker and an isotype matched control antibody.

In some embodiments, the engineered cells described herein further comprise nucleic acid encoding an anti-cytotoxic T cell construct. In some embodiments, the anti-cytotoxic T cell construct is capable of conferring to the engineered cells cytotoxicity towards a cytotoxic T cell that recognizes the engineered cells as foreign, wherein the edited T cell is non-cytotoxic towards cytotoxic T cells that do not recognize the engineered cells as foreign. In some embodiments, the anti-cytotoxic T cell construct is a chimeric receptor comprising an extracellular β2-microglobulin domain, a transmembrane domain, a co-stimulatory domain, and a cytoplasmic signaling domain. In some embodiments, the extracellular β2-microglobulin domain comprises the amino acid sequence of SEQ ID NO: 62 or a variant thereof having at least 85% homology to the amino acid sequence of SEQ ID NO: 62. In some embodiments, the chimeric receptor transmembrane domain comprises a CD8 transmembrane domain polypeptide. In some embodiments, the chimeric receptor CD8 transmembrane domain comprises the amino acid sequence of SEQ ID NO: 63 or a variant thereof having at least 85% homology to the amino acid sequence of SEQ ID NO: 63. In some embodiments, the chimeric receptor co-stimulatory domain comprises a 4-1BB co-stimulatory domain. In some embodiments, the chimeric receptor 4-1BB co-stimulatory domain comprises the amino acid sequence of SEQ ID NO: 64 or a variant thereof having at least 85% homology to the amino acid sequence of SEQ ID NO: 64. In some embodiments, the chimeric receptor cytoplasmic signaling domain comprises a CD3-ζ cytoplasmic signaling domain. In some embodiments, the chimeric receptor CD3-ζ cytoplasmic signaling domain comprises the amino acid sequence of SEQ ID NO: 59 or a variant thereof having at least 85% homology to the amino acid sequence of SEQ ID NO: 59. In some embodiments, the chimeric receptor comprises the amino acid sequence of SEQ ID NO: 65 or a variant thereof having at least 85% homology to the amino acid sequence of SEQ ID NO: 65.

In some embodiments, according to any of the engineered cells described herein comprising nucleic acid encoding an anti-cytotoxic T cell construct, an exogenous nucleic acid encoding the anti-cytotoxic T cell construct is inserted into the genome of the engineered cells. In some embodiments, the exogenous nucleic acid is inserted into an endogenous TRA gene. In some embodiments, the exogenous nucleic acid is inserted into the region of the endogenous TIM gene encoding the TRAC domain. In some embodiments, insertion of the exogenous nucleic acid results in a non-functional TRAC domain. In some embodiments, the exogenous nucleic acid is inserted into an endogenous IL2RG gene. In some embodiments, the exogenous nucleic acid is inserted into an endogenous IL2RG gene such that expression of the anti-cytotoxic T cell construct is under the control of one or more endogenous IL2RG regulatory elements. In some embodiments, the exogenous nucleic acid further comprises a promoter operably linked to the portion of the exogenous nucleic acid encoding the anti-cytotoxic T cell construct, such that expression of the anti-cytotoxic T cell construct in the engineered cells is under the control of the promoter. In some embodiments, the promoter is a myeloproliferative sarcoma virus enhancer, negative control region deleted, dl587rev primer-binding site substituted (MND) promoter. In some embodiments, the MND promoter comprises the polynucleotide sequence of SEQ ID NO: 74 or a variant thereof having at least 85% homology to the polynucleotide sequence of SEQ ID NO: 74.

In some embodiments, the engineered cells described herein further comprise nucleic acid encoding a selectable marker. In some embodiments, the selectable marker is capable of conferring to the engineered cells the ability to survive in a selective condition, such as in the presence of a toxin or in the absence of a nutrient. In some embodiments, the selectable marker is a surface marker that allow for selection of cells expressing the selectable marker. In some embodiments, the selectable marker is a truncated low-affinity nerve growth factor receptor (tLNGFR) polypeptide. In some embodiments, the tLNGFR polypeptide comprises the amino acid sequence of SEQ ID NO: 66 or a variant thereof having at least 85% homology to the amino acid sequence of SEQ ID NO: 66.

In some embodiments, according to any of the engineered cells described herein comprising nucleic acid encoding a selectable marker, an exogenous nucleic acid encoding the selectable marker is inserted into the genome of the engineered cells. In some embodiments, the exogenous nucleic acid is inserted into an endogenous TRA gene. In some embodiments, the exogenous nucleic acid is inserted into the region of the endogenous TRA gene encoding the TRAC domain. In some embodiments, insertion of the exogenous nucleic acid results in a non-functional TRAC domain. In some embodiments, the exogenous nucleic acid is inserted into an endogenous IL2RG gene. In some embodiments, the exogenous nucleic acid is inserted into an endogenous IL2RG gene such that expression of the selectable marker is under the control of one or more endogenous IL2RG regulatory elements. In some embodiments, the exogenous nucleic acid further comprises a promoter operably linked to the portion of the exogenous nucleic acid encoding the selectable marker, such that expression of the selectable marker in the engineered cells is under the control of the promoter. In some embodiments, the promoter is a myeloproliferative sarcoma virus enhancer, negative control region deleted, dl587rev primer-binding site substituted (MND) promoter. In some embodiments, the MND promoter comprises the polynucleotide sequence of SEQ ID NO: 74 or a variant thereof having at least 85% homology to the polynucleotide sequence of SEQ ID NO: 74.

In some embodiments, the engineered cells described herein further comprise nucleic acid encoding a polypeptide that confers resistance to one or more calcineurin inhibitors. In some embodiments, the polypeptide that confers resistance to one or more calcineurin inhibitors confers resistance to tacrolimus (FK506) and/or cyclosporin A (CsA). In some embodiments, the polypeptide that confers resistance to one or more calcineurin inhibitors is a mutant calcineurin (CN) polypeptide. In some embodiments, the mutant CN polypeptide confers resistance to tacrolimus (FK506) and cyclosporin A (CsA). In some embodiments, the mutant CN polypeptide is CNb30 (SEQ ID NO: 67).

In some embodiments, according to any of the engineered cells described herein comprising nucleic acid encoding a polypeptide that confers resistance to one or more calcineurin inhibitors, an exogenous nucleic acid encoding the polypeptide that confers resistance to one or more calcineurin inhibitors is inserted into the genome of the engineered cells. In some embodiments, the exogenous nucleic acid is inserted into an endogenous TRA gene. In some embodiments, the exogenous nucleic acid is inserted into the region of the endogenous TRA gene encoding the TRAC domain. In some embodiments, insertion of the exogenous nucleic acid results in a non-functional TRAC domain. In some embodiments, the exogenous nucleic acid is inserted into an endogenous IL2RG gene. In some embodiments, the exogenous nucleic acid is inserted into an endogenous IL2RG gene such that expression of the selectable marker is under the control of one or more endogenous IL2RG regulatory elements. In some embodiments, the exogenous nucleic acid further comprises a promoter operably linked to the portion of the exogenous nucleic acid encoding the polypeptide that confers resistance to one or more calcineurin inhibitors, such that expression of the polypeptide that confers resistance to one or more calcineurin inhibitors in the engineered cells is under the control of the promoter. In some embodiments, the promoter is a myeloproliferative sarcoma virus enhancer, negative control region deleted, dl587rev primer-binding site substituted (MND) promoter. In some embodiments, the MND promoter comprises the polynucleotide sequence of SEQ ID NO: 74 or a variant thereof having at least 85% homology to the polynucleotide sequence of SEQ ID NO: 74.

In some embodiments, the engineered cells described herein further comprise nucleic acid encoding a polypeptide that confers resistance to rapamycin. In some embodiments, the polypeptide is an FKBP-rapamycin binding (FRB) domain polypeptide of the mammalian target of rapamycin (mTOR) kinase. In some embodiments, the polypeptide that confers resistance rapamycin comprises the amino acid sequence of SEQ ID NO: 68 or 69 or a variant thereof having at least 85% homology to the amino acid sequence of SEQ ID NO: 68 or 69.

In some embodiments, according to any of the engineered cells described herein comprising nucleic acid encoding a polypeptide that confers resistance to rapamycin, an exogenous nucleic acid encoding the polypeptide that confers resistance to rapamycin is inserted into the genome of the engineered cells. In some embodiments, the exogenous nucleic acid is inserted into an endogenous TRA gene. In some embodiments, the exogenous nucleic acid is inserted into the region of the endogenous TRA gene encoding the TRAC domain. In some embodiments, insertion of the exogenous nucleic acid results in a non-functional TRAC domain. In some embodiments, the exogenous nucleic acid is inserted into an endogenous IL2RG gene. In some embodiments, the exogenous nucleic acid is inserted into an endogenous IL2RG gene such that expression of the selectable marker is under the control of one or more endogenous IL2RG regulatory elements. In some embodiments, the exogenous nucleic acid further comprises a promoter operably linked to the portion of the exogenous nucleic acid encoding the polypeptide that confers resistance to rapamycin, such that expression of the polypeptide that confers resistance to rapamycin in the engineered cells is under the control of the promoter. In some embodiments, the promoter is a myeloproliferative sarcoma virus enhancer, negative control region deleted, dl587rev primer-binding site substituted (MND) promoter. In some embodiments, the MND promoter comprises the polynucleotide sequence of SEQ ID NO: 74 or a variant thereof having at least 85% homology to the polynucleotide sequence of SEQ ID NO: 74.

In some embodiments, according to any of the engineered cells described herein, the engineered cells comprise nucleic acid encoding the following system components: i) an anti-plasma cell construct; ii) a first CISC component comprising an IL2Rβ signaling domain; iii) a polypeptide that confers resistance to rapamycin; iv) a selectable marker; v) a polypeptide that confers resistance to one or more calcineurin inhibitors; and vi) a second CISC component comprising an IL2Rγ signaling domain. In some embodiments, the engineered cells comprise nucleic acid comprising a first coding cassette and nucleic acid comprising a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the engineered cells comprise nucleic acid comprising a first polycistronic expression cassette comprising a first promoter operably linked to the first coding cassette, such that expression of the first polycistronic expression cassette is under the control of the first promoter. In some embodiments, the first promoter is an exogenous promoter, and the engineered cells comprise a first exogenous nucleic acid inserted in an endogenous gene, wherein the first exogenous nucleic acid comprises a synthetic polyA sequence upstream of the first polycistronic expression cassette. In some embodiments, the exogenous promoter is a murine stem cell virus (MSCV) promoter. In some embodiments, the first promoter is an endogenous promoter of a first endogenous gene, and the engineered cells comprise a first exogenous nucleic acid inserted in the first endogenous gene, wherein the first exogenous nucleic acid comprises nucleic acid encoding a 2A self-cleaving peptide upstream of the first coding cassette. In some embodiments, the first endogenous gene is an endogenous TRA gene. In some embodiments, the first exogenous nucleic acid is inserted into the region of the endogenous TRA gene encoding the TRAC domain. In some embodiments, insertion of the first exogenous nucleic acid results in a non-functional TRAC domain. In some embodiments, the engineered cells comprise nucleic acid comprising a second polycistronic expression cassette comprising a second promoter operably linked to the second coding cassette, such that expression of the second polycistronic expression cassette is under the control of the second promoter. In some embodiments, the second promoter is an exogenous promoter, and the engineered cells comprise a second exogenous nucleic acid inserted in a second endogenous gene, wherein the second exogenous nucleic acid comprises the second promoter operably linked to the second coding cassette. In some embodiments, the second promoter is an MND promoter. In some embodiments, the second endogenous gene is an endogenous IL2RG gene. In some embodiments, the second endogenous gene is an endogenous IL2RG gene, the second exogenous nucleic acid comprises a fragment of the nucleic acid encoding the second CISC component, and the second exogenous nucleic acid is inserted into the endogenous IL2RG gene such that the fragment of the nucleic acid encoding the second CISC component is linked to an endogenous IL2RG gene sequence, and the fragment of the nucleic acid encoding the second CISC component linked to the endogenous IL2RG gene sequence together encode the second CISC component. In some embodiments, the first polycistronic expression cassette comprises a sequence of contiguous nucleotides from any one of SEQ ID NOs: 28-39. In some embodiments, the second polycistronic expression cassette comprises a sequence of contiguous nucleotides from any one of SEQ ID NOs: 40-43.

In some embodiments, according to any of the engineered cells described herein comprising a polycistronic expression cassette, the polycistronic expression cassette comprises nucleic acid encoding a 2A self-cleaving peptide between adjacent system component-encoding nucleic acids. In some embodiments, the polycistronic expression cassette comprises nucleic acid encoding a 2A self-cleaving peptide between each of the adjacent system component-encoding nucleic acids. For example, in some embodiments, the polycistronic expression cassette comprises, in order from 5′ to 3′, nucleic acid encoding a polypeptide that confers resistance to rapamycin, nucleic acid encoding a 2A self-cleaving peptide, nucleic acid encoding a selectable marker, nucleic acid encoding a 2A self-cleaving peptide, nucleic acid encoding a polypeptide that confers resistance to one or more calcineurin inhibitors, nucleic acid encoding a 2A self-cleaving peptide, and nucleic acid encoding a second CISC component or a fragment thereof. In some embodiments, each of the 2A self-cleaving peptides is, independently, a T2A self-cleaving peptide or a P2A self-cleaving peptide. In some embodiments, the T2A self-cleaving peptide comprises the amino acid sequence of SEQ ID NO: 72 or a variant thereof having at least 85% homology to the amino acid sequence of SEQ ID NO: 72. In some embodiments, the P2A self-cleaving peptide comprises the amino acid sequence of SEQ ID NO: 73 or a variant thereof having at least 85% homology to the amino acid sequence of SEQ ID NO: 73.

In some embodiments, according to any of the engineered cells described herein, the engineered cells comprise nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a selectable marker; vi) a polypeptide that confers resistance to one or more calcineurin inhibitors; and vii) a second CISC component comprising an IL2Rγ signaling domain. In some embodiments, the engineered cells comprise nucleic acid comprising a first coding cassette and nucleic acid comprising a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the anti-cytotoxic T cell construct, the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the engineered cells comprise nucleic acid comprising a first polycistronic expression cassette comprising a first promoter operably linked to the first coding cassette, such that expression of the first polycistronic expression cassette is under the control of the first promoter. In some embodiments, the first promoter is an exogenous promoter, and the engineered cells comprise a first exogenous nucleic acid inserted in an endogenous gene, wherein the first exogenous nucleic acid comprises a synthetic polyA sequence upstream of the first polycistronic expression cassette. In some embodiments, the exogenous promoter is a murine stem cell virus (MSCV) promoter. In some embodiments, the first promoter is an endogenous promoter of a first endogenous gene, and the engineered cells comprise a first exogenous nucleic acid inserted in the first endogenous gene, wherein the first exogenous nucleic acid comprises nucleic acid encoding a 2A self-cleaving peptide upstream of the first coding cassette. In some embodiments, the first endogenous gene is an endogenous TRA gene. In some embodiments, the first exogenous nucleic acid is inserted into the region of the endogenous TRA gene encoding the TRAC domain. In some embodiments, insertion of the first exogenous nucleic acid results in a non-functional TRAC domain. In some embodiments, the engineered cells comprise nucleic acid comprising a second polycistronic expression cassette comprising a second promoter operably linked to the second coding cassette, such that expression of the second polycistronic expression cassette is under the control of the second promoter. In some embodiments, the second promoter is an exogenous promoter, and the engineered cells comprise a second exogenous nucleic acid inserted in a second endogenous gene, wherein the second exogenous nucleic acid comprises the second promoter operably linked to the second coding cassette. In some embodiments, the second promoter is an MND promoter. In some embodiments, the second endogenous gene is an endogenous IL2RG gene. In some embodiments, the second endogenous gene is an endogenous IL2RG gene, the second exogenous nucleic acid comprises a fragment of the nucleic acid encoding the second CISC component, and the second exogenous nucleic acid is inserted into the endogenous IL2RG gene such that the fragment of the nucleic acid encoding the second CISC component is linked to an endogenous IL2RG gene sequence, and the fragment of the nucleic acid encoding the second CISC component linked to the endogenous IL2RG gene sequence together encode the second CISC component. In some embodiments, the first polycistronic expression cassette comprises a sequence of contiguous nucleotides from any one of SEQ ID NOs: 28-39. In some embodiments, the second polycistronic expression cassette comprises a sequence of contiguous nucleotides from SEQ ID NO: 44.

In some embodiments, according to any of the engineered cells described herein, the engineered cells comprise nucleic acid encoding the following system components: i) an anti-plasma cell construct; ii) a first CISC component comprising an IL2Rβ signaling domain; iii) a polypeptide that confers resistance to rapamycin; iv) a polypeptide that confers resistance to one or more calcineurin inhibitors; and v) a second CISC component comprising an IL2Rγ signaling domain. In some embodiments, the engineered cells comprise nucleic acid comprising a first coding cassette and nucleic acid comprising a second coding cassette. In some embodiments, the first coding cassette comprises the nucleic acid encoding the anti-plasma cell construct. In some embodiments, the second coding cassette comprises the nucleic acid encoding the polypeptide that confers resistance to rapamycin, the nucleic acid encoding the polypeptide that confers resistance to one or more calcineurin inhibitors, the nucleic acid encoding the first CISC component, and the nucleic acid encoding the second CISC component or a fragment thereof. In some embodiments, the engineered cells comprise nucleic acid comprising a first polycistronic expression cassette comprising a first promoter operably linked to the first coding cassette, such that expression of the first polycistronic expression cassette is under the control of the first promoter. In some embodiments, the first promoter is an exogenous promoter, and the engineered cells comprise a first exogenous nucleic acid inserted in an endogenous gene, wherein the first exogenous nucleic acid comprises a synthetic polyA sequence upstream of the first polycistronic expression cassette. In some embodiments, the exogenous promoter is a murine stem cell virus (MSCV) promoter. In some embodiments, the first promoter is an endogenous promoter of a first endogenous gene, and the engineered cells comprise a first exogenous nucleic acid inserted in the first endogenous gene, wherein the first exogenous nucleic acid comprises nucleic acid encoding a 2A self-cleaving peptide upstream of the first coding cassette. In some embodiments, the first endogenous gene is an endogenous TRA gene. In some embodiments, the first exogenous nucleic acid is inserted into the region of the endogenous TRA gene encoding the TRAC domain. In some embodiments, insertion of the first exogenous nucleic acid results in a non-functional TRAC domain. In some embodiments, the engineered cells comprise nucleic acid comprising a second polycistronic expression cassette comprising a second promoter operably linked to the second coding cassette, such that expression of the second polycistronic expression cassette is under the control of the second promoter. In some embodiments, the second promoter is an exogenous promoter, and the engineered cells comprise a second exogenous nucleic acid inserted in a second endogenous gene, wherein the second exogenous nucleic acid comprises the second promoter operably linked to the second coding cassette. In some embodiments, the second promoter is an MND promoter. In some embodiments, the second endogenous gene is an endogenous IL2RG gene. In some embodiments, the second endogenous gene is an endogenous IL2RG gene, the second exogenous nucleic acid comprises a fragment of the nucleic acid encoding the second CISC component, and the second exogenous nucleic acid is inserted into the endogenous IL2RG gene such that the fragment of the nucleic acid encoding the second CISC component is linked to an endogenous IL2RG gene sequence, and the fragment of the nucleic acid encoding the second CISC component linked to the endogenous IL2RG gene sequence together encode the second CISC component. In some embodiments, the first polycistronic expression cassette comprises a sequence of contiguous nucleotides from any one of SEQ ID NOs: 19-24. In some embodiments, the second polycistronic expression cassette comprises a sequence of contiguous nucleotides from SEQ ID NO: 45.

In some embodiments, according to any of the engineered cells described herein, the engineered cells comprise nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a polypeptide that confers resistance to one or more calcineurin inhibitors; and vi) a second CISC component comprising an IL2Rγ signaling domain. In some embodiments, the engineered cells comprise nucleic acid comprising a first coding cassette and nucleic acid comprising a second coding cassette. In some embodiments, the first coding cassette comprises the nucleic acid encoding the anti-plasma cell construct and the nucleic acid encoding the polypeptide that confers resistance to one or more calcineurin inhibitors. In some embodiments, the second coding cassette comprises the nucleic acid encoding the polypeptide that confers resistance to rapamycin, the nucleic acid encoding the first CISC component, and the nucleic acid encoding the second CISC component or a fragment thereof. In some embodiments, the engineered cells comprise nucleic acid comprising a first polycistronic expression cassette comprising a first promoter operably linked to the first coding cassette, such that expression of the first polycistronic expression cassette is under the control of the first promoter. In some embodiments, the first promoter is an exogenous promoter, and the engineered cells comprise a first exogenous nucleic acid inserted in an endogenous gene, wherein the first exogenous nucleic acid comprises a synthetic polyA sequence upstream of the first polycistronic expression cassette. In some embodiments, the exogenous promoter is a murine stem cell virus (MSCV) promoter. In some embodiments, the first promoter is an endogenous promoter of a first endogenous gene, and the engineered cells comprise a first exogenous nucleic acid inserted in the first endogenous gene, wherein the first exogenous nucleic acid comprises nucleic acid encoding a 2A self-cleaving peptide upstream of the first coding cassette. In some embodiments, the first endogenous gene is an endogenous TRA gene. In some embodiments, the first exogenous nucleic acid is inserted into the region of the endogenous TRA gene encoding the TRAC domain. In some embodiments, insertion of the first exogenous nucleic acid results in a non-functional TRAC domain. In some embodiments, the engineered cells comprise nucleic acid comprising a second polycistronic expression cassette comprising a second promoter operably linked to the second coding cassette, such that expression of the second polycistronic expression cassette is under the control of the second promoter. In some embodiments, the second promoter is an exogenous promoter, and the engineered cells comprise a second exogenous nucleic acid inserted in a second endogenous gene, wherein the second exogenous nucleic acid comprises the second promoter operably linked to the second coding cassette. In some embodiments, the second promoter is an MND promoter. In some embodiments, the second endogenous gene is an endogenous IL2RG gene. In some embodiments, the second endogenous gene is an endogenous IL2RG gene, the second exogenous nucleic acid comprises a fragment of the nucleic acid encoding the second CISC component, and the second exogenous nucleic acid is inserted into the endogenous IL2RG gene such that the fragment of the nucleic acid encoding the second CISC component is linked to an endogenous IL2RG gene sequence, and the fragment of the nucleic acid encoding the second CISC component linked to the endogenous IL2RG gene sequence together encode the second CISC component. In some embodiments, the first polycistronic expression cassette comprises a sequence of contiguous nucleotides from any one of SEQ ID NOs: 25-27. In some embodiments, the second polycistronic expression cassette comprises a sequence of contiguous nucleotides from SEQ ID NO: 46.

### Method of Editing Genome

In some embodiments, provided herein is a method of editing the genome of a cell, in particular, editing the cell genome to allow for expression of i) an anti-plasma cell construct capable of conferring to the cell cytotoxicity towards a plasma cell, and ii) polypeptide components of a dimerization activatable chemical-induced signaling complex (CISC), wherein the signaling-competent CISC is capable of producing a stimulatory signal in a signaling pathway that promotes survival and/or proliferation of the cell.

In one aspect, provided herein is a method of editing the genome of a cell to produce an engineered cell, the method comprising providing to the cell a) a first gRNA and/or a second gRNA according to any of the embodiments described herein, b) an RGEN or a nucleic acid encoding the RGEN according to any of the embodiments described herein, and c) one or more donor templates according to any of the embodiments described herein comprising nucleic acid encoding i) an anti-plasma cell construct capable of conferring to the engineered cell cytotoxicity towards a plasma cell; and ii) polypeptide components of a dimerization activatable chemical-induced signaling complex (CISC), wherein the signaling-competent CISC is capable of producing a stimulatory signal in a signaling pathway that promotes survival and/or proliferation of the engineered cell. In some embodiments, the CISC comprises a first CISC component and a second CISC component, wherein the first CISC component and the second CISC component are configured such that when expressed by the engineered cell, they dimerize in the presence of a ligand to create the signaling-competent CISC. In some embodiments, the engineered cell is unable to survive and/or proliferate in the absence of the ligand. In some embodiments, the engineered cell is defective in an endogenous signaling pathway involved in survival and/or proliferation of the cell, and the signaling-competent CISC is capable of supplementing the defective endogenous signaling pathway such that the engineered cell can survive and/or proliferate. In some embodiments, the first CISC component comprises an IL2Rβ signaling domain. In some embodiments, the first extracellular binding domain of the first CISC component comprises an FRB domain. In some embodiments, the first CISC component comprises the amino acid sequence of SEQ ID NO: 54 or a variant thereof having at least 85% homology to the amino acid sequence of SEQ ID NO: 54. In some embodiments, the second CISC component comprises an IL2Rγ signaling domain. In some embodiments, the second extracellular binding domain of the second CISC component comprises an FKBP domain. In some embodiments, the second CISC component comprises the amino acid sequence of SEQ ID NO: 53 or a variant thereof having at least 85% homology to the amino acid sequence of SEQ ID NO: 53. In some embodiments, the one or more donor templates further comprise nucleic acid encoding one or more of iii) an anti-cytotoxic T cell construct; iv) a selectable marker; v) a polypeptide that confers resistance to one or more calcineurin inhibitors; or vii) a polypeptide that confers resistance to rapamycin. In some embodiments, the anti-plasma cell construct is an anti-BCMA CAR. In some embodiments, the anti-BCMA CAR comprises the amino acid sequence of SEQ ID NO: 60 or 61 or a variant thereof having at least 85% homology to the amino acid sequence of SEQ ID NO: 60 or 61. In some embodiments, the first extracellular binding domain of the first CISC component comprises an FRB domain. In some embodiments, the first CISC component comprises the amino acid sequence of SEQ ID NO: 54 or a variant thereof having at least 85% homology to the amino acid sequence of SEQ ID NO: 54. In some embodiments, the polypeptide that confers resistance to rapamycin is an FRB domain polypeptide. In some embodiments, the FRB domain polypeptide comprises the amino acid sequence of SEQ ID NO: 68 or 69 or a variant thereof having at least 85% homology to the amino acid sequence of SEQ ID NO: 68 or 69. In some embodiments, the selectable marker is a tLNGFR polypeptide. In some embodiments, the tLNGFR polypeptide comprises the amino acid sequence of SEQ ID NO: 66 or a variant thereof having at least 85% homology to the amino acid sequence of SEQ ID NO: 66. In some embodiments, the polypeptide that confers resistance to one or more calcineurin inhibitors is a mutant CN polypeptide. In some embodiments, the mutant CN polypeptide is CNb30 (SEQ ID NO: 67). In some embodiments, the second extracellular binding domain of the second CISC component comprises an FKBP domain. In some embodiments, the second CISC component comprises the amino acid sequence of SEQ ID NO: 53 or a variant thereof having at least 85% homology to the amino acid sequence of SEQ ID NO: 53. In some embodiments, the cell is a T cell, such as a cytotoxic T cell. In some embodiments, the cell is a T cell precursor, such as a cell capable of differentiating into a cytotoxic T cell.

In some embodiments, according to any of the methods of editing the genome of a cell described herein, the one or more donor templates comprise nucleic acid encoding the following system components: i) an anti-plasma cell construct; ii) a first CISC component comprising an IL2Rβ signaling domain; iii) a polypeptide that confers resistance to rapamycin; iv) a selectable marker; v) a polypeptide that confers resistance to one or more calcineurin inhibitors; and vi) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #4-#7. In some embodiments, the first donor template comprises a sequence of contiguous nucleotides from any one of SEQ ID NOs: 28-39. For example, in some embodiments, the first donor template comprises the nucleotide sequence of any one of SEQ ID NOs: 101-104. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #8. In some embodiments, the second donor template comprises a sequence of contiguous nucleotides from any one of SEQ ID NOs: 40-43. For example, in some embodiments, the second donor template comprises the nucleotide sequence of SEQ ID NO: 105. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 28-39 and variants thereof having at least 85% homology to the polynucleotide sequence of any one of SEQ ID NOs: 28-39. In some embodiments, the first AAV vector comprises the polynucleotide sequence of any one of SEQ ID NOs: 101-104 and variants thereof having at least 85% homology to the polynucleotide sequence of any one of SEQ ID NOs: 101-104. In some embodiments, the second AAV vector comprises the polynucleotide sequence of any one of SEQ ID NOs: 40-43 or a variant thereof having at least 85% homology to the polynucleotide sequence of any one of SEQ ID NOs: 40-43. In some embodiments, the second AAV vector comprises the polynucleotide sequence of SEQ ID NO: 105 or a variant thereof having at least 85% homology to the polynucleotide sequence of SEQ ID NO: 105.

In some embodiments, according to any of the methods of editing the genome of a cell described herein, the one or more donor templates comprise nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a selectable marker; vi) a polypeptide that confers resistance to one or more calcineurin inhibitors; and vii) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the anti-cytotoxic T cell construct, the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TIM gene. In some embodiments, the first donor template is inserted into the region of the endogenous TIM gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #4-#7. In some embodiments, the first donor template comprises a sequence of contiguous nucleotides from any one of SEQ ID NOs: 28-39. For example, in some embodiments, the first donor template comprises the nucleotide sequence of any one of SEQ ID NOs: 101-104. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #9. In some embodiments, the second donor template comprises a sequence of contiguous nucleotides from SEQ ID NO: 44. For example, in some embodiments, the second donor template comprises the nucleotide sequence of SEQ ID NO: 106. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 28-39 and variants thereof having at least 85% homology to the polynucleotide sequence of any one of SEQ ID NOs: 28-39. In some embodiments, the first AAV vector comprises the polynucleotide sequence of any one of SEQ ID NOs: 101-104 and variants thereof having at least 85% homology to the polynucleotide sequence of any one of SEQ ID NOs: 101-104. In some embodiments, the second AAV vector comprises the polynucleotide sequence of SEQ ID NO: 44 or a variant thereof having at least 85% homology to the polynucleotide sequence of SEQ ID NO: 44. In some embodiments, the second AAV vector comprises the polynucleotide sequence of SEQ ID NO: 106 or a variant thereof having at least 85% homology to the polynucleotide sequence of SEQ ID NO: 106.

In some embodiments, according to any of the methods of editing the genome of a cell described herein, the one or more donor templates comprise nucleic acid encoding the following system components: i) an anti-plasma cell construct; ii) a first CISC component comprising an IL2Rβ signaling domain; iii) a polypeptide that confers resistance to rapamycin; iv) a polypeptide that confers resistance to one or more calcineurin inhibitors; and v) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct. In some embodiments, the second coding cassette comprises the nucleic acid encoding the polypeptide that confers resistance to rapamycin, the nucleic acid encoding the polypeptide that confers resistance to one or more calcineurin inhibitors, the nucleic acid encoding the first CISC component, and the nucleic acid encoding the second CISC component or a fragment thereof. In some embodiments, the first donor template comprises a synthetic polyA sequence upstream of a first promoter operably linked to the first coding cassette, such that expression of the nucleic acid encoding the anti-plasma cell construct is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding sequence, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #1 and #2. In some embodiments, the first donor template comprises a sequence of contiguous nucleotides from any one of SEQ ID NOs: 19-24. For example, in some embodiments, the first donor template comprises the nucleotide sequence of any one of SEQ ID NOs: 98-99. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #10. In some embodiments, the second donor template comprises a sequence of contiguous nucleotides from SEQ ID NO: 45. For example, in some embodiments, the second donor template comprises the nucleotide sequence of SEQ ID NO: 107. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 19-24 and variants thereof having at least 85% homology to the polynucleotide sequence of any one of SEQ ID NOs: 19-24. In some embodiments, the first AAV vector comprises the polynucleotide sequence of any one of SEQ ID NOs: 98-99 and variants thereof having at least 85% homology to the polynucleotide sequence of any one of SEQ ID NOs: 98-99. In some embodiments, the second AAV vector comprises the polynucleotide sequence of SEQ ID NO: 45 or a variant thereof having at least 85% homology to the polynucleotide sequence of SEQ ID NO: 45. In some embodiments, the second AAV vector comprises the polynucleotide sequence of SEQ ID NO: 107 or a variant thereof having at least 85% homology to the polynucleotide sequence of SEQ ID NO: 107.

In some embodiments, according to any of the methods of editing the genome of a cell described herein, the one or more donor templates comprise nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a polypeptide that confers resistance to one or more calcineurin inhibitors; and vi) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the polypeptide that confers resistance to one or more calcineurin inhibitors. In some embodiments, the second coding cassette comprises the nucleic acid encoding the polypeptide that confers resistance to rapamycin, the nucleic acid encoding the first CISC component, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template construct #3. In some embodiments, the first donor template comprises a sequence of contiguous nucleotides from any one of SEQ ID NOs: 25-27. For example, in some embodiments, the first donor template comprises the nucleotide sequence of SEQ ID NO: 100. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #11. In some embodiments, the second donor template comprises a sequence of contiguous nucleotides from SEQ ID NO: 46. For example, in some embodiments, the second donor template comprises the nucleotide sequence of SEQ ID NO: 108. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 25-27 and variants thereof having at least 85% homology to the polynucleotide sequence of any one of SEQ ID NOs: 25-27. In some embodiments, the first AAV vector comprises the polynucleotide sequence of SEQ ID NO: 100 and variants thereof having at least 85% homology to the polynucleotide sequence of SEQ ID NO: 100. In some embodiments, the second AAV vector comprises the polynucleotide sequence of SEQ ID NO: 46 or a variant thereof having at least 85% homology to the polynucleotide sequence of SEQ ID NO: 46. In some embodiments, the second AAV vector comprises the polynucleotide sequence of SEQ ID NO: 108 or a variant thereof having at least 85% homology to the polynucleotide sequence of SEQ ID NO: 108.

In some embodiments, according to any of the methods of editing the genome of a cell described herein, the method comprises providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first vector comprises the polynucleotide sequence of any one of SEQ ID NOs: 28, 31, 34, and 37 and variants thereof having at least 85% homology to the polynucleotide sequence of any one of SEQ ID NOs: 28, 31, 34, and 37, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44; (B) the first gRNA comprises the polynucleotide sequence of SEQ ID NO: 2 or a variant thereof having at least 85% homology to the polynucleotide sequence of SEQ ID NO: 2, the first vector comprises the polynucleotide sequence of any one of SEQ ID NOs: 29, 32, 35, and 38 and variants thereof having at least 85% homology to the polynucleotide sequence of any one of SEQ ID NOs: 29, 32, 35, and 38, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44; or (C) the first gRNA comprises the polynucleotide sequence of SEQ ID NO: 3 or a variant thereof having at least 85% homology to the polynucleotide sequence of SEQ ID NO: 3, the first vector comprises the polynucleotide sequence of any one of SEQ ID NOs: 30, 33, 36, and 39 and variants thereof having at least 85% homology to the polynucleotide sequence of any one of SEQ ID NOs: 30, 33, 36, and 39, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44.

In some embodiments, according to any of the methods of editing the genome of a cell described herein, the method comprises providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 19 or 22 or a variant thereof having at least 85% homology to the polynucleotide sequence of SEQ ID NO: 19 or 22, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 20 or 23 or a variant thereof having at least 85% homology to the polynucleotide sequence of SEQ ID NO: 20 or 23, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 21 or 24 or a variant thereof having at least 85% homology to the polynucleotide sequence of SEQ ID NO: 21 or 24, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45.

In some embodiments, according to any of the methods of editing the genome of a cell described herein, the method comprises providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 25 or a variant thereof having at least 85% homology to the polynucleotide sequence of SEQ ID NO: 25, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 26 or a variant thereof having at least 85% homology to the polynucleotide sequence of SEQ ID NO: 26, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 27 or a variant thereof having at least 85% homology to the polynucleotide sequence of SEQ ID NO: 27, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46.

In some embodiments, according to any of the methods of editing the genome of a cell described herein, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9. In some embodiments, the nucleic acid encoding the RGEN is a ribonucleic acid (RNA) sequence. In some embodiments, the RNA sequence encoding the RGEN is linked to the first gRNA or the second gRNA via a covalent bond. In some embodiments, the RGEN is precomplexed with the first gRNA and/or the second gRNA, forming an RNP complex, prior to the provision to the cell. In some embodiments, the RGEN is precomplexed with the first gRNA and/or the second gRNA at a molar ratio of gRNA to RGEN between 1:1 to 20:1, respectively.

In some embodiments, according to any of the methods of editing the genome of a cell described herein, the cell is a T cell. In some embodiments, the T cell is a CD8+ cytotoxic T lymphocyte or a CD3+ pan T cell. In some embodiments, the T cell is a member of a pool of T cells derived from multiple donors. In some embodiments, the multiple donors are human donors. In some embodiments, the cell is cytotoxic to plasma cells.

### Method of Treatment

In some embodiments, provided herein is a method of treating a disease or condition in a subject in need thereof, wherein the disease or condition is characterized by adverse antibody production, the method comprising: 1) editing the genome of T cells according to any of the methods described herein, thereby producing engineered T cells and administering the engineered T cells to the subject; or 2) editing the genome of T cells in the subject according to any of the methods described herein, thereby producing engineered T cells in the subject. In some embodiments, the T cells of a) are autologous to the subject. In some embodiments, the T cells of a) are allogenic to the subject. In some embodiments, the T cells of a) comprise a pool of T cells derived from multiple donors. In some embodiments, the multiple donors are human donors. In some embodiments, the T cells comprise CD8+ cytotoxic T cells or CD3+ pan T cells. In some embodiments, the subject is human. In some embodiments, the disease or condition is graft-versus-host disease (GvHD), antibody-mediated autoimmunity, plasma cell neoplasm, or light-chain amyloidosis. In some embodiments, the plasma cell neoplasm is plasma cell myeloma (e.g., multiple myeloma). In some embodiments, the disease or condition is GvHD, and the subject has previously received an organ transplant.

In some embodiments, according to any of the methods of treating a disease or condition described herein, editing the genome of T cells to produce engineered T cells comprises providing to the T cells a) a first gRNA and/or a second gRNA according to any of the embodiments described herein, b) an RGEN or a nucleic acid encoding the RGEN according to any of the embodiments described herein, and c) one or more donor templates according to any of the embodiments described herein comprising nucleic acid encoding i) an anti-plasma cell construct capable of conferring to the engineered cells cytotoxicity towards a plasma cell; and ii) polypeptide components of a dimerization activatable chemical-induced signaling complex (CISC), wherein the signaling-competent CISC is capable of producing a stimulatory signal in a signaling pathway that promotes survival and/or proliferation of the engineered cells. In some embodiments, the CISC comprises a first CISC component and a second CISC component, wherein the first CISC component and the second CISC component are configured such that when expressed by the engineered cells, they dimerize in the presence of a ligand to create the signaling-competent CISC. In some embodiments, the engineered cells are unable to survive and/or proliferate in the absence of the ligand. In some embodiments, the engineered cells are defective in an endogenous signaling pathway involved in survival and/or proliferation of the cells, and the signaling-competent CISC is capable of supplementing the defective endogenous signaling pathway such that the engineered cells can survive and/or proliferate. In some embodiments, the first CISC component comprises an IL2Rβ signaling domain. In some embodiments, the first extracellular binding domain of the first CISC component comprises an FRB domain. In some embodiments, the first CISC component comprises the amino acid sequence of SEQ ID NO: 54 or a variant thereof having at least 85% homology to the amino acid sequence of SEQ ID NO: 54. In some embodiments, the second CISC component comprises an IL2Rγ signaling domain. In some embodiments, the second extracellular binding domain of the second CISC component comprises an FKBP domain. In some embodiments, the second CISC component comprises the amino acid sequence of SEQ ID NO: 53 or a variant thereof having at least 85% homology to the amino acid sequence of SEQ ID NO: 53. In some embodiments, the anti-plasma cell construct is an anti-BCMA CAR. In some embodiments, the anti-BCMA CAR comprises the amino acid sequence of SEQ ID NO: 60 or 61 or a variant thereof having at least 85% homology to the amino acid sequence of SEQ ID NO: 60 or 61. In some embodiments, the one or more donor templates further comprise nucleic acid encoding one or more of iii) an anti-cytotoxic T cell construct; iv) a selectable marker; v) a polypeptide that confers resistance to one or more calcineurin inhibitors; or vi) a polypeptide that confers resistance to rapamycin. In some embodiments, the polypeptide that confers resistance to rapamycin is an FRB domain polypeptide. In some embodiments, the FRB domain polypeptide comprises the amino acid sequence of SEQ ID NO: 68 or 69 or a variant thereof having at least 85% homology to the amino acid sequence of SEQ ID NO: 68 or 69. In some embodiments, the selectable marker is a tLNGFR polypeptide. In some embodiments, the tLNGFR polypeptide comprises the amino acid sequence of SEQ ID NO: 66 or a variant thereof having at least 85% homology to the amino acid sequence of SEQ ID NO: 66. In some embodiments, the polypeptide that confers resistance to one or more calcineurin inhibitors is a mutant CN polypeptide. In some embodiments, the mutant CN polypeptide is CNb30 (SEQ ID NO: 67).

In some embodiments, according to any of the methods of treating a disease or condition described herein, the one or more donor templates comprise nucleic acid encoding the following system components: i) an anti-plasma cell construct; ii) a first CISC component comprising an IL2Rβ signaling domain; iii) a polypeptide that confers resistance to rapamycin; iv) a selectable marker; v) a polypeptide that confers resistance to one or more calcineurin inhibitors; and vi) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #4-#7. In some embodiments, the first donor template comprises a sequence of contiguous nucleotides from any one of SEQ ID NOs: 28-39. For example, in some embodiments, the first donor template comprises the nucleotide sequence of any one of SEQ ID NOs: 101-104. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #8. In some embodiments, the second donor template comprises a sequence of contiguous nucleotides from any one of SEQ ID NOs: 40-43. For example, in some embodiments, the second donor template comprises the nucleotide sequence of SEQ ID NO: 105. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 28-39 and variants thereof having at least 85% homology to the polynucleotide sequence of any one of SEQ ID NOs: 28-39. In some embodiments, the first AAV vector comprises the polynucleotide sequence of any one of SEQ ID NOs: 101-104 and variants thereof having at least 85% homology to the polynucleotide sequence of any one of SEQ ID NOs: 101-104. In some embodiments, the second AAV vector comprises the polynucleotide sequence of any one of SEQ ID NOs: 40-43 or a variant thereof having at least 85% homology to the polynucleotide sequence of any one of SEQ ID NOs: 40-43. In some embodiments, the second AAV vector comprises the polynucleotide sequence of SEQ ID NO: 105 or a variant thereof having at least 85% homology to the polynucleotide sequence of SEQ ID NO: 105.

In some embodiments, according to any of the methods of treating a disease or condition described herein, the one or more donor templates comprise nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a selectable marker; vi) a polypeptide that confers resistance to one or more calcineurin inhibitors; and vii) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the first CISC component. In some embodiments, the second coding cassette comprises the nucleic acid encoding the anti-cytotoxic T cell construct, the nucleic acid encoding the polypeptide that confers resistance to rapamycin, the nucleic acid encoding the selectable marker, the nucleic acid encoding the polypeptide that confers resistance to one or more calcineurin inhibitors, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #4-#7. In some embodiments, the first donor template comprises a sequence of contiguous nucleotides from any one of SEQ ID NOs: 28-39. For example, in some embodiments, the first donor template comprises the nucleotide sequence of any one of SEQ ID NOs: 101-104. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #9. In some embodiments, the second donor template comprises a sequence of contiguous nucleotides from SEQ ID NO: 44. For example, in some embodiments, the second donor template comprises the nucleotide sequence of SEQ ID NO: 106. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 28-39 and variants thereof having at least 85% homology to the polynucleotide sequence of any one of SEQ ID NOs: 28-39. In some embodiments, the first AAV vector comprises the polynucleotide sequence of any one of SEQ ID NOs: 101-104 and variants thereof having at least 85% homology to the polynucleotide sequence of any one of SEQ ID NOs: 101-104. In some embodiments, the second AAV vector comprises the polynucleotide sequence of SEQ ID NO: 44 or a variant thereof having at least 85% homology to the polynucleotide sequence of SEQ ID NO: 44. In some embodiments, the second AAV vector comprises the polynucleotide sequence of SEQ ID NO: 106 or a variant thereof having at least 85% homology to the polynucleotide sequence of SEQ ID NO: 106.

In some embodiments, according to any of the methods of treating a disease or condition described herein, the one or more donor templates comprise nucleic acid encoding the following system components: i) an anti-plasma cell construct; ii) a first CISC component comprising an IL2Rβ signaling domain; iii) a polypeptide that confers resistance to rapamycin; iv) a polypeptide that confers resistance to one or more calcineurin inhibitors; and v) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct. In some embodiments, the second coding cassette comprises the nucleic acid encoding the polypeptide that confers resistance to rapamycin, the nucleic acid encoding the polypeptide that confers resistance to one or more calcineurin inhibitors, the nucleic acid encoding the first CISC component, and the nucleic acid encoding the second CISC component or a fragment thereof. In some embodiments, the first donor template comprises a synthetic polyA sequence upstream of a first promoter operably linked to the first coding cassette, such that expression of the nucleic acid encoding the anti-plasma cell construct is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding sequence, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template constructs #1 and #2. In some embodiments, the first donor template comprises a sequence of contiguous nucleotides from any one of SEQ ID NOs: 19-24. For example, in some embodiments, the first donor template comprises the nucleotide sequence of any one of SEQ ID NOs: 98-99. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #10. In some embodiments, the second donor template comprises a sequence of contiguous nucleotides from SEQ ID NO: 45. For example, in some embodiments, the second donor template comprises the nucleotide sequence of SEQ ID NO: 107. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 19-24 and variants thereof having at least 85% homology to the polynucleotide sequence of any one of SEQ ID NOs: 19-24. In some embodiments, the first AAV vector comprises the polynucleotide sequence of any one of SEQ ID NOs: 98-99 and variants thereof having at least 85% homology to the polynucleotide sequence of any one of SEQ ID NOs: 98-99. In some embodiments, the second AAV vector comprises the polynucleotide sequence of SEQ ID NO: 45 or a variant thereof having at least 85% homology to the polynucleotide sequence of SEQ ID NO: 45. In some embodiments, the second AAV vector comprises the polynucleotide sequence of SEQ ID NO: 107 or a variant thereof having at least 85% homology to the polynucleotide sequence of SEQ ID NO: 107.

In some embodiments, according to any of the methods of treating a disease or condition described herein, the one or more donor templates comprise nucleic acid encoding the following system components: i) an anti-plasma cell construct; ii) a first CISC component comprising an IL2Rβ signaling domain; iii) an anti-cytotoxic T cell construct; iv) a polypeptide that confers resistance to rapamycin; v) a polypeptide that confers resistance to one or more calcineurin inhibitors; and vi) a second CISC component comprising an IL2Rγ signaling domain or fragment thereof. In some embodiments, the one or more donor templates comprise a first donor template and a second donor template. In some embodiments, the first donor template is configured to be inserted in a first endogenous gene and the second donor template is configured to be inserted in a second endogenous gene. In some embodiments, the first donor template comprises a first coding cassette and the second donor template comprises a second coding cassette. In some embodiments, the first coding cassette comprises the nucleic acid encoding the anti-plasma cell construct and the nucleic acid encoding the polypeptide that confers resistance to one or more calcineurin inhibitors. In some embodiments, the second coding cassette comprises the nucleic acid encoding the polypeptide that confers resistance to rapamycin, the nucleic acid encoding the first CISC component, and the nucleic acid encoding the second CISC component or a fragment thereof. In some embodiments, the first donor template comprises a synthetic polyA sequence upstream of a first polycistronic expression cassette comprising a first promoter operably linked to the first coding cassette, such that expression of the first polycistronic expression cassette is under the control of the first promoter. In some embodiments, the first promoter is a murine stem cell virus (MSCV) promoter. In some embodiments, the first donor template comprises nucleic acid encoding a portion of a first polycistronic expression cassette comprising nucleic acid encoding a 2A self-cleaving peptide upstream of the first coding cassette, wherein the first donor template is configured such that when inserted into the first endogenous gene, the portion of the first polycistronic expression cassette is linked to a sequence of the first endogenous gene, and the portion of the first polycistronic expression cassette linked to the sequence of the first endogenous gene together comprise the first polycistronic expression cassette. In some embodiments, the first endogenous gene is an endogenous TRA gene. In some embodiments, the first donor template is inserted into the region of the endogenous TRA gene encoding the TRAC domain. In some embodiments, insertion of the first donor template results in a non-functional TRAC domain. In some embodiments, the second donor template comprises a second polycistronic expression cassette or portion thereof comprising a second promoter operably linked to the second coding cassette, such that expression of the second polycistronic expression cassette is under the control of the second promoter. In some embodiments, the second promoter is an MND promoter. In some embodiments, the second endogenous gene is an endogenous IL2RG gene. In some embodiments, the second endogenous gene is an endogenous IL2RG gene, the second donor template comprises a portion of the second polycistronic expression cassette comprising nucleic acid comprising a fragment of the nucleic acid encoding the second CISC component, and the second donor template is configured such that when inserted into the endogenous IL2RG gene the fragment of the nucleic acid encoding the second CISC component is linked to an endogenous IL2RG gene sequence, the fragment of the nucleic acid encoding the second CISC component linked to the endogenous IL2RG gene sequence together encode the second CISC component, and the portion of the second polycistronic expression cassette linked to the endogenous IL2RG gene sequence together comprise the second polycistronic expression cassette. Exemplary configurations for the first donor template are shown in FIG. 1, donor template construct #3. In some embodiments, the first donor template comprises a sequence of contiguous nucleotides from any one of SEQ ID NOs: 25-27. For example, in some embodiments, the first donor template comprises the nucleotide sequence of SEQ ID NO: 100. In some embodiments, the first donor template is flanked by homology arms corresponding to sequences in the TRA gene. Exemplary homology arms for the first donor template include homology arms having the polynucleotide sequences of SEQ ID NOs: 80 and 81, SEQ ID NOs: 82 and 83, or SEQ ID NOs: 84 and 85. Exemplary configurations for the second donor template are shown in FIG. 1, donor template construct #11. In some embodiments, the second donor template comprises a sequence of contiguous nucleotides from SEQ ID NO: 46. For example, in some embodiments, the second donor template comprises the nucleotide sequence of SEQ ID NO: 108. In some embodiments, the second donor template is flanked by homology arms corresponding to sequences in the IL2RG gene. Exemplary homology arms for the second donor template include homology arms having the polynucleotide sequences of SEQ ID NOs: 86 and 87, SEQ ID NOs: 88 and 89, or SEQ ID NOs: 90 and 91. In some embodiments, the first donor template is a first AAV vector and/or the second donor template is a second AAV vector. In some embodiments, the first AAV vector comprises the polynucleotide sequence of any one of SEQ ID NOs: 25-27 and variants thereof having at least 85% homology to the polynucleotide sequence of any one of SEQ ID NOs: 25-27. In some embodiments, the first AAV vector comprises the polynucleotide sequence of SEQ ID NO: 100 and variants thereof having at least 85% homology to the polynucleotide sequence of SEQ ID NO: 100. In some embodiments, the second AAV vector comprises the polynucleotide sequence of SEQ ID NO: 46 or a variant thereof having at least 85% homology to the polynucleotide sequence of SEQ ID NO: 46. In some embodiments, the second AAV vector comprises the polynucleotide sequence of SEQ ID NO: 108 or a variant thereof having at least 85% homology to the polynucleotide sequence of SEQ ID NO: 108.

In some embodiments, according to any of the methods of treating a disease or condition described herein, the method comprises providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first vector comprises the polynucleotide sequence of any one of SEQ ID NOs: 28, 31, 34, and 37 and variants thereof having at least 85% homology to the polynucleotide sequence of any one of SEQ ID NOs: 28, 31, 34, and 37, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44; (B) the first gRNA comprises the polynucleotide sequence of SEQ ID NO: 2 or a variant thereof having at least 85% homology to the polynucleotide sequence of SEQ ID NO: 2, the first vector comprises the polynucleotide sequence of any one of SEQ ID NOs: 29, 32, 35, and 38 and variants thereof having at least 85% homology to the polynucleotide sequence of any one of SEQ ID NOs: 29, 32, 35, and 38, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44; or (C) the first gRNA comprises the polynucleotide sequence of SEQ ID NO: 3 or a variant thereof having at least 85% homology to the polynucleotide sequence of SEQ ID NO: 3, the first vector comprises the polynucleotide sequence of any one of SEQ ID NOs: 30, 33, 36, and 39 and variants thereof having at least 85% homology to the polynucleotide sequence of any one of SEQ ID NOs: 30, 33, 36, and 39, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44.

In some embodiments, according to any of the methods of treating a disease or condition described herein, the method comprises providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 19 or 22 or a variant thereof having at least 85% homology to the polynucleotide sequence of SEQ ID NO: 19 or 22, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 20 or 23 or a variant thereof having at least 85% homology to the polynucleotide sequence of SEQ ID NO: 20 or 23, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 21 or 24 or a variant thereof having at least 85% homology to the polynucleotide sequence of SEQ ID NO: 21 or 24, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45.

In some embodiments, according to any of the methods of treating a disease or condition described herein, the method comprises providing to the cell a first gRNA, a second gRNA, an RGEN or a nucleic acid encoding the RGEN, a first vector, and a second vector, wherein (A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 25 or a variant thereof having at least 85% homology to the polynucleotide sequence of SEQ ID NO: 25, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; (B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 26 or a variant thereof having at least 85% homology to the polynucleotide sequence of SEQ ID NO: 26, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; or (C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 27 or a variant thereof having at least 85% homology to the polynucleotide sequence of SEQ ID NO: 27, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46.

In some embodiments, according to any of the methods of treating a disease or condition described herein, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9. In some embodiments, the nucleic acid encoding the RGEN is a ribonucleic acid (RNA) sequence. In some embodiments, the RNA sequence encoding the RGEN is linked to the first gRNA or the second gRNA via a covalent bond. In some embodiments, the RGEN is precomplexed with the first gRNA and/or the second gRNA, forming an RNP complex, prior to the provision to the cell. In some embodiments, the RGEN is precomplexed with the first gRNA and/or the second gRNA at a molar ratio of gRNA to RGEN between 1:1 to 20:1, respectively.

In some embodiments, according to any of the methods of treating a disease or condition described herein, the cell is a T cell. In some embodiments, the T cell is a CD8+ cytotoxic T lymphocyte or a CD3+ pan T cell. In some embodiments, the T cell is a member of a pool of T cells derived from multiple donors. In some embodiments, the multiple donors are human donors. In some embodiments, the cell is cytotoxic to plasma cells.

In some embodiments, the methods of treating a disease or condition described herein further comprise administering rapamycin or a rapalog to the subject. In some embodiments, the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof. In some embodiments, the rapamycin or the rapalog is administered in a concentration from 0.05 nM to 100 nM.

In another aspect, provided herein is an engineered T cell according to any of the embodiments described herein for use in the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is an engineered T cell according to any of the embodiments described herein for use in the manufacture of a medicament for the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is a system according to any of the embodiments described herein for use in the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is a system according to any of the embodiments described herein for use in the manufacture of a medicament for the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is one or more gRNAs, one or more donor templates, a kit, a syringe, and/or a catheter according to any of the embodiments described herein for use in the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

In another aspect, provided herein is one or more gRNAs, one or more donor templates, a kit, a syringe, and/or a catheter according to any of the embodiments described herein for use in the manufacture of a medicament for the treatment of graft vs host disease (GvHD) or an autoimmune disease, or a disease or condition characterized by adverse antibody production. In some embodiments, the autoimmune disease is an antibody-mediated autoimmune disease. In some embodiments, the disease or condition is light-chain amyloidosis.

### Compositions

Provided herein are compositions that comprise a genetically modified cell, such as a mammalian cell, prepared as set forth in this disclosure. In some embodiments, the cells, such as mammalian cells, include the protein sequences as described in the embodiments herein. In some embodiments, the compositions include T cells that have a CISC comprising an extracellular binding domain, a hinge domain, a transmembrane domain, and signaling domain. In some embodiments, the CISC is an IL2R-CISC. In other embodiments, the composition further comprises a cell, such as a mammalian cell, preparation comprising CD8+ T cells that have a CISC comprising an extracellular binding domain, a hinge domain, a transmembrane domain, and a signaling domain. In some embodiments, the CISC components dimerize in the presence of a ligand (for example, rapamycin or a rapalog), which may occur simultaneously or sequentially. In some embodiments, each of these populations can be combined with one another or other cell types to provide a composition.

In some embodiments, the cells of the composition are CD8+ cells. The CD8+ cell can be a T cytotoxic lymphocyte cell, a naïve CD8+ T cell, central memory CD8+ T cell, effector memory CD8+ T cell and/or bulk CD8+ T cell. In some embodiments, the CD8+ cytotoxic T lymphocyte cell is a central memory T cell, wherein the central memory T cell comprises a CD45RO+, CD62L+, and/or CD8+ T cell. In yet other embodiments, the CD8+ cytotoxic T lymphocyte cell is a central memory T cell.

In some embodiments, the compositions comprise T cell precursors. In some embodiments, the compositions comprise hematopoietic stem cells. In some embodiments, the composition comprises a host cell, wherein the host cell is a CD8+T cytotoxic lymphocyte cell selected from the group consisting of naïve CD8+ T cells, central memory CD8+ T cells, effector memory CD8+ T cells and bulk CD8+ T cells, and a second host cell, wherein the second host cell is a precursor T cell. In some embodiments, the precursor T cell is a hematopoietic stem cell.

In some compositions, the cells are NK cells.

In some embodiments, the cell is CD8+. In some embodiments, the cell is a CD8+T cytotoxic lymphocyte cell selected from the group consisting of naïve CD8+ T-cells, central memory CD8+ T-cells, effector memory CD8+ T-cells and bulk CD8+ T-cells. In some embodiments, the cell is a precursor T-cell. In some embodiments, the cell is a stem cell. In some embodiments, the cell is a hematopoietic stem cell or NK cell. In some embodiments, the cell further comprises a chimeric antigen receptor.

Also provided herein are kits and systems including the cells, expression vectors, and protein sequences provided and described herein. Thus, for example, provided herein is a kit comprising one or more of: a protein sequence as described herein; an expression vector as described herein; and/or a cell as described herein. Also provided is a system for selectively activation a signal into an interior of a cell, the system comprising a cell as described herein, wherein the cell comprises an expression vector as described herein comprising a nucleic acid encoding a protein sequence as described herein.

Method of Making a Cell that Expresses a Dimeric CISC Component

In some embodiments described herein, it may be desired to introduce a protein sequence or an expression vector into a host cell, such as a mammalian cell, e.g., a lymphocyte, to be used for drug regulated cytokine signaling and/or for the selective expansion of cells that express the dimeric CISC components. For example, the dimeric CISC can allow for cytokine signaling in cells that have the introduced CISC components for transmitting signals to the interior of a cell, such as a mammalian cell, upon contact with a ligand. In addition, the selective expansion of cells, such as mammalian cells, can be controlled to select for only those cells that have undergone two specific genetic modification events, as described herein. Preparation of these cells can be carried out in accordance with known techniques that will be apparent to those skilled in the art based upon the present disclosure.

In some embodiments, a method of making a CISC-bearing cell, such as a mammalian cell, is provided, wherein the cell expresses a dimeric CISC. The method can include delivering to a cell, such as a mammalian cell, the protein sequence of any one of the embodiments or embodiments described herein or the expression vector of the embodiments or embodiments described herein and delivering to the cell, such as a mammalian cell. In some embodiments, the protein sequence comprises a first and a second sequence. In some embodiments, the first sequence encodes for a first CISC component comprising a first extracellular binding domain, a hinge domain, a linker of a specified length, wherein the length is optionally optimized, a transmembrane domain, and a signaling domain. In some embodiments, the second sequence encodes for a second CISC component comprising a second extracellular binding domain, a hinge domain, a linker of a specified length, wherein the length is optionally optimized, a transmembrane domain, and a signaling domain. In some embodiments, the spacer is 1, 2, 3, 4, 5, 6, 7, 8, 9, 10, 11, 12, 13, 14 or 15 amino acids in length or a length within a range defined by any two of the aforementioned lengths. In some embodiments, the signaling domain comprises an interleukin-2 signaling domain, such as an IL2RB or an IL2RG domain. In some embodiments, the extracellular binding domain is a binding domain that binds to rapamycin or a rapalog, comprising FKBP or FRB or a portion thereof. In some embodiments, the cell is a CD8+ cell. In some embodiments, the cell is a CD8+T cytotoxic lymphocyte cell selected from the group consisting of naïve CD8+ T-cells, central memory CD8+ T-cells, effector memory CD8+ T-cells and bulk CD8+ T-cells. In some embodiments, the cell is a precursor T-cell. In some embodiments, the cell is a stem cell. In some embodiments, the cell is a hematopoietic stem cell. In some embodiments, the cell is an NK cell.

### Method of Activating a Signal in the Interior of a Cell

In some embodiments, a method described herein employs a step of activating a signal in the interior of a cell, such as a mammalian cell. The method can include providing a cell, such as a mammalian cell, as described herein, wherein the cell comprises a protein sequence as set forth herein or an expression vector as set forth herein. In some embodiments, the method further comprises expressing the protein sequence encoding a dimeric CISC as described herein, or expression the vector as described herein. In some embodiments, the method comprises contacting the cell, such as a mammalian cell, with a ligand, which causes the first and second CISC components to dimerize, which transduces a signal into the interior of the cell. In some embodiments, the ligand is rapamycin or rapalog. In some embodiments an effective amount of a ligand for inducing dimerization is provided an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values.

In some embodiments, the ligand used in these approaches is rapamycin or a rapalog, comprising, for example, everolimus, CCI-779, C20-methallylrapamycin, C16-(S)-3-methylindolerapamycin, C16-iRap, AP21967, sodium mycophenolic acid, benidipine hydrochloride, AP23573, or AP1903, or metabolites, derivatives, and/or combinations thereof. Additional useful rapalogs may include, for example, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and/or alternative substitution on the cyclohexyl ring or replacement of the cyclohexyl ring with a substituted cyclopentyl ring. Additional useful rapalogs may include novolimus, pimecrolimus, ridaforolimus, tacrolimus, temsirolimus, umirolimus, or zotarolimus, or metabolites, derivatives, and/or combinations thereof.

In some embodiments, detecting a signal in the interior of the cell, such as a mammalian cell, can be achieved by a method of detecting a marker that is the result of a signaling pathway. Thus, for example, a signal may be detected by determining the levels of Akt or other signaling marker in a cell, such as a mammalian cell, through a process of Western blot, flow cytometry, or other protein detection and quantification method. Markers for detection may include, for example, JAK, Akt, STAT, NF-κ, MAPK, PI3K, JNK, ERK, or Ras, or other cellular signaling markers that are indicative of a cellular signaling event.

In some embodiments, transduction of a signal affects cytokine signaling. In some embodiments, transduction of the signal affects IL2R signaling. In some embodiments, transduction of the signal affects phosphorylation of a downstream target of a cytokine receptor. In some embodiments, the method of activating a signal induces proliferation in CISC-expressing cells, such as mammalian cells, and a concomitant anti-proliferation in non-CISC expressing cells.

For cellular signaling to take place, not only must cytokine receptors dimerize or heterodimerize, but they must be in the proper configuration for a conformational change to take place (Kim, M. J. et al. (2007). J. Biol. Chem., 282(19):14253-14261). Thus, dimerization in conjunction with the correct conformational positioning of signaling domains are desired processes for appropriate signaling, because receptor dimerization or heterodimerization alone is insufficient to drive receptor activation. The chemical-induced signaling complexes described herein are generally in the correct orientation for downstream signaling events to occur.

### Method of Selective Expansion of Cell Populations

In some embodiments, a method described herein employs a step of selectively expanding a population of cells, such as mammalian cells. In some embodiments, the method comprises providing a cell, such as a mammalian cell, as described herein, wherein the cell comprises a protein sequence as set forth herein or an expression vector as set forth herein. In some embodiments, the method further comprises expressing the protein sequence encoding a dimeric CISC as described herein, or expression the vector as described herein. In some embodiments, the method comprises contacting the cell, such as a mammalian cell, with a ligand, which causes the first and second CISC components to dimerize, which transduces a signal into the interior of the cell. In some embodiments, the ligand is rapamycin or rapalog. In some embodiments an effective amount of a ligand provided for inducing dimerization is an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values. In some embodiments, where the ligand is a rapalog, an effective amount of the ligand provided for inducing dimerization is an amount of 100 nM, 200 nM, 300 nM, 400 nM, 500 nM, 600 nM, 700 nM, 800 nM, 900 nM, 1000 nM, or greater, or a concentration within a range defined by any two of the aforementioned values.

In some embodiments, the ligand used is rapamycin or a rapalog, comprising, for example, everolimus, CCI-779, C20-methallylrapamycin, C16-(S)-3-methylindolerapamycin, C16-iRap, AP21967, sodium mycophenolic acid, benidipine hydrochloride, or AP23573, AP1903, or metabolites, derivatives, and/or combinations thereof. Additional useful rapalogs may include, for example, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and/or alternative substitution on the cyclohexyl ring or replacement of the cyclohexyl ring with a substituted cyclopentyl ring. Additional useful rapalogs may include novolimus, pimecrolimus, ridaforolimus, tacrolimus, temsirolimus, umirolimus, or zotarolimus, or metabolites, derivatives, and/or combinations thereof.

In some embodiments, the selective expansion of a population of cells, such as mammalian cells, takes place only when two distinct genetic modification events have taken place. One genetic modification event is one component of the dimeric chemical-induced signaling complex, and the other genetic modification event is the other component of the dimeric chemical-induced signaling complex. When both events take place within the population of cells, such as a population of mammalian cells, the chemical-induced signaling complex components dimerize in the presence of a ligand, resulting in an active chemical-induced signaling complex and generation of a signal into the interior of the cells.

### Nucleic Acids

### Genome-Targeting Nucleic Acid or Guide RNA

The present disclosure provides a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that hybridizes to a target nucleic acid sequence of interest and a CRISPR repeat sequence. In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

Exemplary genome-targeting nucleic acids are described in WO 2018/002719.

### Donor DNA or Donor Template

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage, and can lead to disruption or alteration of gene expression. HDR, which is also known as homologous recombination (HR) can occur when a homologous repair template, or donor, is available.

The homologous donor template has sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few base pairs flanking the cleavage site to drive a favored end-joining DNA repair outcome. In some instances, it can be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide (or donor or donor sequence or polynucleotide donor template) herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

When an exogenous DNA molecule is supplied in sufficient concentration inside the nucleus of a cell in which the double-strand break occurs, the exogenous DNA can be inserted at the double-strand break during the NHEJ repair process and thus become a permanent addition to the genome. These exogenous DNA molecules are referred to as donor templates in some embodiments. If the donor template contains a coding sequence for one or more system components described herein optionally together with relevant regulatory sequences such as promoters, enhancers, polyA sequences and/or splice acceptor sequences, the one or more system components can be expressed from the integrated nucleic acid in the genome resulting in permanent expression for the life of the cell. Moreover, the integrated nucleic acid of the donor DNA template can be transmitted to the daughter cells when the cell divides.

In the presence of sufficient concentrations of a donor DNA template that contains flanking DNA sequences with homology to the DNA sequence either side of the double-strand break (referred to as homology arms), the donor DNA template can be integrated via the HDR pathway. The homology arms act as substrates for homologous recombination between the donor template and the sequences either side of the double-strand break. This can result in an error free insertion of the donor template in which the sequences either side of the double-strand break are not altered from that in the unmodified genome.

Supplied donors for editing by HDR vary markedly but generally contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors can be used. These oligonucleotides range in size from less than 100 nt to over many kb, though longer ssDNA can also be generated and used. Double-stranded donors are often used, including PCR amplicons, plasmids, and mini-circles. In general, it has been found that an AAV vector is a very effective means of delivery of a donor template, though the packaging limits for individual donors is <5 kb. Active transcription of the donor increased HDR three-fold, indicating the inclusion of promoter can increase conversion. Conversely, CpG methylation of the donor can decrease gene expression and HDR.

In some embodiments, the donor DNA can be supplied with the nuclease or independently by a variety of different methods, for example by transfection, nanoparticle, micro-injection, or viral transduction. A range of tethering options can be used to increase the availability of the donors for HDR in some embodiments. Examples include attaching the donor to the nuclease, attaching to DNA binding proteins that bind nearby, or attaching to proteins that are involved in DNA end binding or repair.

In addition to genome editing by NHEJ or HDR, site-specific gene insertions can be conducted that use both the NHEJ pathway and HR. A combination approach can be applicable in certain settings, possibly including intron/exon borders. NHEJ can prove effective for ligation in the intron, while the error-free HDR can be better suited in the coding region.

In embodiments, an exogenous sequence that is intended to be inserted into a genome comprises one or more system components described herein. In some embodiments, the exogenous sequence comprises nucleic acid encoding one or more of i) an anti-plasma cell construct; ii) a first CISC component comprising an IL2Rβ signaling domain; iii) an anti-cytotoxic T cell construct; iv) a polypeptide that confers resistance to rapamycin; v) a selectable marker; vi) a polypeptide that confers resistance to one or more calcineurin inhibitors; and vii) a second CISC component comprising an IL2Rγ signaling domain or fragment thereof. In some embodiments, the anti-plasma cell construct is an anti-BCMA CAR. In some embodiments, the anti-BCMA CAR comprises the amino acid sequence of SEQ ID NO: 60 or 61 or a variant thereof having at least 85% homology to the amino acid sequence of SEQ ID NO: 60 or 61. In some embodiments, the first extracellular binding domain of the first CISC component comprises an FRB domain. In some embodiments, the first CISC component comprises the amino acid sequence of SEQ ID NO: 54 or a variant thereof having at least 85% homology to the amino acid sequence of SEQ ID NO: 54. In some embodiments, the anti-cytotoxic T cell construct is a chimeric receptor comprising an extracellular β2-microglobulin domain, a transmembrane domain, a co-stimulatory domain, and a cytoplasmic signaling domain. In some embodiments, the chimeric receptor comprises the amino acid sequence of SEQ ID NO: 65 or a variant thereof having at least 85% homology to the amino acid sequence of SEQ ID NO: 65. In some embodiments, the polypeptide that confers resistance to rapamycin is an FRB domain polypeptide. In some embodiments, the FRB domain polypeptide comprises the amino acid sequence of SEQ ID NO: 68 or 69 or a variant thereof having at least 85% homology to the amino acid sequence of SEQ ID NO: 68 or 69. In some embodiments, the selectable marker is a tLNGFR polypeptide. In some embodiments, the tLNGFR polypeptide comprises the amino acid sequence of SEQ ID NO: 66 or a variant thereof having at least 85% homology to the amino acid sequence of SEQ ID NO: 66. In some embodiments, the polypeptide that confers resistance to one or more calcineurin inhibitors is a mutant CN polypeptide. In some embodiments, the mutant CN polypeptide is CNb30 (SEQ ID NO: 67). In some embodiments, the second extracellular binding domain of the second CISC component comprises an FKBP domain. In some embodiments, the second CISC component comprises the amino acid sequence of SEQ ID NO: 53 or a variant thereof having at least 85% homology to the amino acid sequence of SEQ ID NO: 53.

### Nucleic Acid Encoding a Site-Directed Polypeptide or DNA Endonuclease

In some embodiments, the methods of genome edition and compositions therefore can use a nucleic acid sequence encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

### Vectors

In another aspect, the present disclosure provides a nucleic acid having a nucleotide sequence encoding a genome-targeting nucleic acid of the disclosure, a site-directed polypeptide of the disclosure, and/or any nucleic acid or proteinaceous molecule necessary to carry out the embodiments of the methods of the disclosure. In some embodiments, such a nucleic acid is a vector (e.g., a recombinant expression vector).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Additional vectors contemplated for eukaryotic target cells include, but are not limited to, the vectors pCTx-1, pCTx-2, and pCTx-3. Other vectors can be used so long as they are compatible with the host cell.

In some embodiments, a vector has one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. In some embodiments, the vector is a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas endonuclease, various promoters such as RNA polymerase III promoters, including for example U6 and H1, can be advantageous. Descriptions of and parameters for enhancing the use of such promoters are known in art, and additional information and approaches are regularly being described; see, e.g., Ma, H. et al. (2014). Mol. Ther.-Nucleic Acids 3:e161, doi:10.1038/mtna.2014.12.

The expression vector can also contain a ribosome binding site for translation initiation and a transcription terminator. The expression vector can also include appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

In some embodiments, a promoter is an inducible promoter (e.g., a heat shock promoter, tetracycline-regulated promoter, steroid-regulated promoter, metal-regulated promoter, estrogen receptor-regulated promoter, etc.). In some embodiments, a promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.). In some embodiments, a vector does not have a promoter for at least one gene to be expressed in a host cell if the gene is going to be expressed, after it is inserted into a genome, under an endogenous promoter present in the genome.

### Site-Directed Polypeptide or DNA Endonuclease

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The process of integrating non-native nucleic acid into genomic DNA is an example of genome editing.

A site-directed polypeptide is a nuclease used in genome editing to cleave DNA. The site-directed polypeptide can be administered to a cell or a patient as either: one or more polypeptides, or one or more nucleic acids encoding the polypeptide.

In the context of a CRISPR/Cas or CRISPR/Cpf1 system, the site-directed polypeptide can bind to a guide RNA that, in turn, specifies the site in the target DNA to which the polypeptide is directed. In embodiments of CRISPR/Cas or CRISPR/Cpf1 systems herein, the site-directed polypeptide is an endonuclease, such as a DNA endonuclease. Such an RNA-guided site-directed polypeptide is also referred to herein as an RNA-guided endonuclease, or RGEN.

Exemplary site-directed polypeptides are described in WO 2018/002719.

### Target Sequence Selection

In some embodiments, shifts in the location of the 5′ boundary and/or the 3′ boundary relative to particular reference loci are used to facilitate or enhance particular applications of gene editing, which depend in part on the endonuclease system selected for the editing, as further described and illustrated herein.

In a first, non-limiting aspect of such target sequence selection, many endonuclease systems have rules or criteria that guide the initial selection of potential target sites for cleavage, such as the requirement of a PAM sequence motif in a particular position adjacent to the DNA cleavage sites in the case of CRISPR Type II or Type V endonucleases.

In another, non-limiting aspect of target sequence selection or optimization, the frequency of “off-target” activity for a particular combination of target sequence and gene editing endonuclease (i.e. the frequency of DSBs occurring at sites other than the selected target sequence) is assessed relative to the frequency of on-target activity. In some cases, cells that have been correctly edited at the desired locus can have a selective advantage relative to other cells. Illustrative, but non-limiting, examples of a selective advantage include the acquisition of attributes such as enhanced rates of replication, persistence, resistance to certain conditions, enhanced rates of successful engraftment or persistence in vivo following introduction into a patient, and other attributes associated with the maintenance or increased numbers or viability of such cells. In other cases, cells that have been correctly edited at the desired locus can be positively selected for by one or more screening methods used to identify, sort or otherwise select for cells that have been correctly edited. Both selective advantage and directed selection methods can take advantage of the phenotype associated with the correction. In some embodiments, cells can be edited two or more times in order to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

In embodiments, whether any selective advantage is applicable or any directed selection is to be applied in a particular case, target sequence selection is also guided by consideration of off-target frequencies in order to enhance the effectiveness of the application and/or reduce the potential for undesired alterations at sites other than the desired target. As described further and illustrated herein and in the art, the occurrence of off-target activity is influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs are regularly being induced and repaired in normal cells. During repair, the original sequence can be reconstructed with complete fidelity, however, in some cases, small insertions or deletions (referred to as “indels”) are introduced at the DSB site.

DSBs can also be specifically induced at particular locations, as in the case of the endonucleases systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can have as few as ten base pairs or less, can also be used to bring about desired deletions. For example, a single DSB is introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which can or cannot be desired given the particular circumstances.

The examples provided herein further illustrate the selection of various target regions for the creation of DSBs designed to insert one or more system components described herein, as well as the selection of specific target sequences within such regions that are designed to minimize off-target events relative to on-target events.

### Targeted Integration

In some embodiments, a method provided herein is to integrate nucleic acid encoding one or more system components described herein at a specific location in the genome of target cells (e.g., T cells), which is referred to as “targeted integration”. In some embodiments, targeted integration is enabled by using a sequence specific nuclease to generate a double-stranded break in the genomic DNA.

The CRISPR-Cas system used in some embodiments has the advantage that a large number of genomic targets can be rapidly screened to identify an optimal CRISPR-Cas design. The CRISPR-Cas system uses a RNA molecule called a single guide RNA (sgRNA) that targets an associated Cas nuclease (for example the Cas9 nuclease) to a specific sequence in DNA. This targeting occurs by Watson-Crick based pairing between the sgRNA and the sequence of the genome within the approximately 20 bp targeting sequence of the sgRNA. Once bound at a target site the Cas nuclease cleaves both strands of the genomic DNA creating a double-strand break. The only requirement for designing a sgRNA to target a specific DNA sequence is that the target sequence must contain a protospacer adjacent motif (PAM) sequence at the 3′ end of the sgRNA sequence that is complementary to the genomic sequence. In the case of the Cas9 nuclease from Streptococcus pyogenes, the PAM sequence is NRG (where R is A or G and N is any base), or the more restricted PAM sequence NGG. Therefore, sgRNA molecules that target any region of the genome can be designed in silico by locating the 20 bp sequence adjacent to all PAM motifs. PAM motifs occur on average very 15 bp in the genome of eukaryotes. However, sgRNA designed by in silico methods will generate double-strand breaks in cells with differing efficiencies and it is not possible to predict the cutting efficiencies of a series of sgRNA molecule using in silico methods. Because sgRNA can be rapidly synthesized in vitro this enables the rapid screening of all potential sgRNA sequences in a given genomic region to identify the sgRNA that results in the most efficient cutting. Generally when a series of sgRNA within a given genomic region are tested in cells a range of cleavage efficiencies between 0 and 90% is observed. In silico algorithms as well as laboratory experiments can also be used to determine the off-target potential of any given sgRNA. While a perfect match to the 20 bp recognition sequence of a sgRNA will primarily occur only once in most eukaryotic genomes there will be a number of additional sites in the genome with 1 or more base pair mismatches to the sgRNA. These sites can be cleaved at variable frequencies which are often not predictable based on the number or location of the mismatches. Cleavage at additional off-target sites that were not identified by the in silico analysis can also occur. Thus, screening a number of sgRNA in a relevant cell type to identify sgRNA that have the most favorable off-target profile is a critical component of selecting an optimal sgRNA for therapeutic use. A favorable off target profile will take into account not only the number of actual off-target sites and the frequency of cutting at these sites, but also the location in the genome of these sites. For example, off-target sites close to or within functionally important genes, particularly oncogenes or anti-oncogenes would be considered as less favorable than sites in intergenic regions with no known function. Thus, the identification of an optimal sgRNA cannot be predicted simply by in silico analysis of the genomic sequence of an organism but requires experimental testing. While in silico analysis can be helpful in narrowing down the number of guides to test it cannot predict guides that have high on target cutting or predict guides with low desirable off-target cutting. The ability of a given sgRNA to promote cleavage by a Cas enzyme can relate to the accessibility of that specific site in the genomic DNA which can be determined by the chromatin structure in that region. While the majority of the genomic DNA in a quiescent differentiated cell exists in highly condensed heterochromatin, regions that are actively transcribed exists in more open chromatin states that are known to be more accessible to large molecules such as proteins like the Cas protein. Even within actively transcribed genes some specific regions of the DNA are more accessible than others due to the presence or absence of bound transcription factors or other regulatory proteins. Predicting sites in the genome or within a specific genomic locus or region of a genomic locus is not possible and therefore would need to be determined experimentally in a relevant cell type. Once some sites are selected as potential sites for insertion, it can be possible to add some variations to such a site, e.g. by moving a few nucleotides upstream or downstream from the selected sites, with or without experimental tests.

In some embodiments, gRNAs that can be used in the methods disclosed herein comprise a spacer comprising the polynucleotide sequence of any one of SEQ ID NOs: 1-18 or any derivatives thereof having at least about 85% nucleotide sequence identity any one of SEQ ID NOs: 1-18.

### Nucleic Acid Modifications

In some embodiments, polynucleotides introduced into cells have one or more modifications that can be used independently or in combination, for example, to enhance activity, stability or specificity, alter delivery, reduce innate immune responses in host cells, or for other enhancements, as further described herein and known in the art.

In certain embodiments, modified polynucleotides are used in a CRISPR/Cas system described herein (such as a CRISPR/Cas9/Cpf1 system), in which case the guide RNAs (either single-molecule guides or double-molecule guides) and/or a DNA or an RNA encoding a Cas or Cpf1 endonuclease introduced into a cell can be modified, as described and illustrated below. Such modified polynucleotides can be used in the CRISPR/Cas system to edit any one or more genomic loci.

Using a CRISPR/Cas system for purposes of non-limiting illustrations of such uses, modifications of guide RNAs can be used to enhance the formation or stability of the CRISPR/Cas genome editing complex having guide RNAs, which can be single-molecule guides or double-molecule, and a Cas or Cpf1 endonuclease. Modifications of guide RNAs can also or alternatively be used to enhance the initiation, stability or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas or Cpf1 endonuclease is introduced into the cell to be edited via an RNA that needs to be translated in order to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (i.e. the endonuclease), and/or modifications that decrease the likelihood or degree to which the RNAs introduced into cells elicit innate immune responses.

Combinations of modifications, such as the foregoing and others, can likewise be used. In the case of CRISPR/Cas systems, for example, one or more types of modifications can be made to guide RNAs (including those exemplified above), and/or one or more types of modifications can be made to RNAs encoding Cas endonuclease (including those exemplified above).

Exemplary modified nucleic acids are described in WO 2018/002719.

### Delivery

In some embodiments, any nucleic acid molecules used in the methods provided herein, e.g. a nucleic acid encoding a genome-targeting nucleic acid of the disclosure and/or a site-directed polypeptide are packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Exemplary delivery methods and reagents are described in WO 2018/002719.

The present disclosure has been described above with reference to specific alternatives. However, other alternatives than the above described are equally possible within the scope of the disclosure. Different method steps than those described above, may be provided within the scope of the disclosure. The different features and steps described herein may be combined in other combinations than those described.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

In addition, where features or aspects of the disclosure are described in terms of Markush groups, those skilled in the art will recognize that the disclosure is also thereby described in terms of any individual member or subgroup of members of the Markush group.

Any of the features of an alternative of the first through eleventh aspects is applicable to all aspects and alternatives identified herein. Moreover, any of the features of an alternative of the first through eleventh aspects is independently combinable, partly or wholly with other alternatives described herein in any way, e.g., one, two, or three or more alternatives may be combinable in whole or in part. Further, any of the features of an alternative of the first through eleventh aspects may be made optional to other aspects or alternatives. Although described above in terms of various example alternatives and implementations, it should be understood that the various features, aspects and functionality described in one or more of the individual alternatives are not limited in their applicability to the particular alternative with which they are described, but instead may be applied, alone or in various combinations, to one or more of the other alternatives of the present application, whether or not such alternatives are described and whether or not such features are presented as being a part of a described alternative. Thus, the breadth and scope of the present application should not be limited by any of the above-described example alternatives.

All references cited herein are incorporated herein by reference in their entirety. To the extent publications and patents or patent applications incorporated by reference contradict the disclosure contained in the specification, the specification is intended to supersede and/or take precedence over any such contradictory material. To the extent publications and patents or patent applications incorporated by reference herein contradict the disclosure contained in the specification, the specification is intended to supersede and/or take precedence over any such contradictory material.

The details of one or more embodiments of the disclosure are set forth in the accompanying description below. Any materials and methods similar or equivalent to those described herein can be used in the practice or testing of the present disclosure. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Some embodiments of the disclosures provided herewith are further illustrated by the following non-limiting examples.

### EXAMPLES

### Materials and Methods

### Reagents

Adeno-associated virus (AAV) was produced from triple transfection of 293 cells and purified via iodixanol gradient centrifugation. All AAVs are of serotype 2/6. Single-guide RNAs (sgRNA) were ordered from Synthego and used as per the manufacturer's recommendations. The target-binding portion of the sgRNA sequences are as follows: TRAC 1: 5′-ACAAAACTGTGCTAGACATG-3′ (SEQ ID NO: 3); TRAC 2: 5′-AGAGCAACAGTGCTGTGGCC-3′ (SEQ ID NO: 1); TRAC 3: 5′-TCTCTCAGCTGGTACACGGC-3′ (SEQ ID NO: 2); IL2RG GC8: 5′-GGTTATCTCTGTTGGCTCCA-3′ (SEQ ID NO: 11); IL2RG GC10: 5′-AAGGCTGATAATCAATCCCA-3′ (SEQ ID NO: 13); and IL2RG GC12: 5′-CCACGGCTTCCAATGCAAAC-3′ (SEQ ID NO: 15). Cas9 enzyme (TrueCut V2) was purchased from Thermo Fisher Scientific. Cas9 and sgRNAs were complexed in phosphate-buffered saline for at least 10 minutes at room temperature prior to use.

Isogenic pairs of cell lines that express/do not express BCMA were created in three different ways. First, RPMI-8266 cells were transfected with Cas9 and an sgRNA targeting the 5′-tattaagctcagtcccaaac-3′ (SEQ ID NO: 78) sequence in the coding region of BCMA. The cell pool was stained with a PE-conjugated anti-human-BCMA antibody (Biolegend 357503) and cells without staining isolated. Second, in normally BCMA-negative K562 cells, BCMA expression was placed under the control of the low-level PPP1R12C (AAVS1) promoter by integration of an SA-2A-BCMA-BGH polyA construct into the AAVS1 locus using a sgRNA targeting 5′-ggggccactagggacaggat-3′ (SEQ ID NO: 79). Cells were cloned by limiting dilution and BCMA expression confirmed by flow cytometry. Third, in K562 cells BCMA expression was placed under control of the strong MND promoter by integration of an MND-BCMA-BGH polyA construct into the AAVS1 locus using a sgRNA targeting 5′-ggggccactagggacaggat-3′ (SEQ ID NO: 79). Cells were cloned by limiting dilution and BCMA expression confirmed by flow cytometry. Two clones were isolated: one with high BCMA expression and another with very high BCMA expression.

### T Cell Culture

Primary human CD3+ T cells were isolated from individual whole blood leukopaks. T cells were cultured in AIM-V medium plus 5% human AB serum plus 50 ng/mL IL-2. Cells were stimulated to proliferate using anti-CD3/CD28 magnetic beads (Miltenyi Biotec, 130-091-441). Cells were incubated with beads at a 1:1 ratio at a starting concentration of 0.5e6 cells/mL for three days. The beads were then removed and the cells allowed to divide for one day prior to transfection.

### T Cell Transfection and Infection

Cells were transfected with pre-complexed RNP consisting of 60 pmol sgRNA and 12 pmol Cas9 using a Lonza 4D nucleofector and program EO-115. One hour after post-transfection cells were infected with an AAV2/6 containing BCMA-CAR/CISCβ or TNP-CAR/CISCβ targeting constructs for a TRAC gene and/or FRB/tLNGFR/CNb30/CISCγ targeting constructs for an IL2RG gene at MOIs ranging from 1,000-100,000 (generally 50,000 each).

### Flow Cytometry

Five days post-gene editing the T cells were analyzed by flow cytometry for expression of TRAC, IL2RG, the CAR, and the CISC. TRAC expression was probed by staining the cells with APC-conjugated mouse anti-human α/β TCR (clone IP26; Biolegend-catalog #306702). IL2RG expression was monitored with an PE-conjugated with mouse anti-human CD132 (BV421; BD Biosciences catalog #566222). BCMA CAR expression was detected using biotinylated-BCMA (Acro Biosystems BC7-H82F0) and PE-conjugated streptavidin (BD Biosciences catalog #554061); BCMA and TNP CAR expression was detected using a PE-conjugated goat anti-mouse-Fv F(ab)2 (Jackson Immunoresearch 115-066-072). tLNGFR expression was monitored with an APC-conjugated mouse anti-human CD271 (clone REA844; Miltenyi Biotec 130-112-791). The CISC expression was visualized with a custom-made biotin:rapamycin conjugate and the PE-conjugated streptavidin. All flow cytometry was performed on an Attune NxT (ThermoFisher).

### Cytotoxicity Assay

Fifty-thousand target cells (RPMI-8226 or RPMI-8226 BCMA-KO; K562 or K562 BCMA-low or K562-BCMA high or K562-BCMA very high) were labelled with eFluor 670 (Invitrogen #50-246-095) and incubated with CART cells at effector:target ratios of 0.5:1, 1:1, 2:1, 4:1, 8:1 for 16 hours. The cell pool was stained with DAPI (Invitrogen #D3571) to detect dead cells, mixed with Countbrite counting beads (Invitrogen #C36950) for volume normalization, and the eFluor 670-positive, DAPI-negative and eFluor-positive, DAPI-positive cells quantitated. Percent viability was determined as the fraction of live cells times 100%; percent cytotoxicity calculated as 100% minus the percent viability.

### IFN-γELISA

An IFN-gamma ELISA kit was purchased from R&D Systems and used according to the manufacturer's instructions. Culture supernatant was measured after 16 hours of incubation.

### Mouse Xenograft Assay

Five million RPMI-8226 or RPMI-8226 BCMA-KO cells were injected subcutaneously into NSG mice. After 2.5 weeks of tumor growth, BCMA CAR- or TNP CAR-modified T cells were injected intravenously and tumor size monitored with calipers.

### CISC-Mediated Cell Expansion

T cell pools with the CISC integrated into a TRAC gene and an IL2RG gene were grown in AIM-V medium plus 5% AB serum plus 10 nM rapamycin without IL-2.

### Example 1: Characterization of gRNAs

gRNAs Targeting the TRAC Gene

To evaluate the ability of gRNAs specific for the TRAC gene to effect targeted cleavage, gRNAs including the spacers TRAC 1 (SEQ ID NO: 3), TRAC 2 (SEQ ID NO: 1), and TRAC 3 (SEQ ID NO: 2) were ordered from Synthego and evaluated in primary human CD8+ or CD3+ T cells transfected with Cas9/gRNA RNPs including the respective gRNA by electroporation following three days of activation with anti-CD3/CD8/CD28 beads. Forty-eight hours after transfection, the cells were analyzed for cleavage efficiency at the on-target site for each gRNA using the TIDES protocol (Brinkman, E. K. et al. (2014). Nucleic Acids Res., 42(22):e168), in which PCR primers flanking the predicted cleavage site are used to amplify the genomic DNA from treated cells, followed by Sanger sequencing of the PCR product. When a double-strand break is created in the genome of a cell, the cell attempts to repair the double-strand break. This repair process is error prone, which can result in the deletion or insertion of nucleotides at the site of the double-strand break. Because breaks that are perfectly repaired are re-cleaved by the Cas9 nuclease, whereas insertion or deletion of nucleotides will prevent Cas9 cleavage, there will be an accumulation of insertions and deletions that are representative of the cutting efficiency. The sequencing chromatogram data were then analyzed using a computer algorithm that calculates the frequency of inserted or deleted bases at the predicted cleavage site. The frequency of inserted or deleted bases (INDELs) was used to calculate the overall cleavage frequency. The cells were analyzed at day two post-editing for INDEL efficiency, cell viability, and total cell counts, which were similar for all 3 gRNAs tested (Table 1, results from 2 independent experiments). The gRNAs resulted in an INDEL efficiency of ranging from 54% to 64% for both CD8+ and CD3+ T cells, with cell viabilities of ranging from 77% to 89%, indicating that these gRNAs efficiently cleave at their target sites in T cells without inducing cytotoxicity.

TABLE 1

| | | INDEL | Cell | Cell |
|---|---|---|---|---|
| | | Frequency (%) | Viability (%) | count |
| CD8 + T cells | TRAC 1 | 62.05 | 84 | 5.66E+05 |
| | TRAC 2 | 59.5 | 88.5 | 7.84E+05 |
| | TRAC 3 | 64.05 | 85.5 | 7.39E+05 |
| CD3 + T cells | TRAC 1 | 56.3 | 76.5 | 6.16E+05 |
| | TRAC 2 | 53.85 | 80 | 7.77E+05 |
| | TRAC 3 | 56.85 | 82.5 | 9.45E+05 |

The cells were further analyzed by flow cytometry at day seven post-editing for TCR and CD3 expression (Table 2). Each of the gRNAs was able to reduce TCR expression in both CD8+ and CD3+ T cells by about 90% or more as compared to untreated controls. Surface CD3 expression, which depends on TCR expression, was also reduced in cells treated with each of the gRNAs. These results support the findings for INDEL efficiency, and indicate that editing with the gRNAs was able to repress TCR expression in T cells, silencing signaling through the endogenous TCR in the edited cells.

TABLE 2

| | | TCR + cells (%) | CD3 + cells (%) |
|---|---|---|---|
| CD8 + T cells | Control | 99.55 | 93.6 |
| | TRAC 1 | 9.63 | 23.65 |
| | TRAC 2 | 8.1 | 24.34 |
| | TRAC 3 | 2.33 | 17.39 |
| CD3 + T cells | Control | 98.53 | 96.06 |
| | TRAC 1 | 4.53 | 53.98 |
| | TRAC 2 | 8.63 | 43.17 |
| | TRAC 3 | 14.72 | 43.96 |

To evaluate targeted integration of a donor template at the TRAC gene mediated by gRNAs TRAC 1, TRAC 2 and TRAC 3, primary human CD3+ T cells were transfected with Cas9/gRNA RNPs including the respective gRNA by electroporation immediately followed by transduction with a corresponding AAV vector with homology arms specific for each gRNA and carrying a donor template encoding a CISC and an mCherry marker (SEQ ID NOs: 94-96) for integration at a multiplicity of infection (MOI) of 50,000. Forty-eight hours after transduction, the cells were analyzed for integration efficiency using flow cytometry for mCherry and TCR expression. As shown in Table 3 (results from two independent experiments with different T cell lots), targeted integration of the donor templates was achieved for each of the three gRNAs tested, and the amount of TCR−/CISC+ cells ranged from about 12% to about 18%.

TABLE 3

| | TCR + | CISC + | TCR−/CISC+ |
|---|---|---|---|
| | cells (%) | cells (%) | cells (%) |
| Untreated | 90.55 | 0 | 0 |
| TRAC 1 RNP | 44.5 | 0 | 0 |
| TRAC 2 RNP | 44.8 | 0 | 0 |
| TRAC 3 RNP | 55.45 | 0 | 0 |
| TRAC 1 RNP +AAV | 28.85 | 18.65 | 17.5 |
| TRAC 2 RNP +AAV | 41.35 | 16.4 | 15.2 |
| TRAC 3 RNP +AAV | 47.9 | 12.75 | 11.85 |

gRNAs Targeting the IL2RG Locus

To evaluate the ability of gRNAs specific for the IL2RG locus to affect targeted cleavage, 15 gRNAs including the spacers GC1 (SEQ ID NO: 4), GC2 (SEQ ID NO: 5), GC3 (SEQ ID NO: 6), GC4 (SEQ ID NO: 7), GC5 (SEQ ID NO: 8), GC6 (SEQ ID NO: 9), GC7 (SEQ ID NO: 10), GC8 (SEQ ID NO: 11), GC9 (SEQ ID NO: 12), GC10 (SEQ ID NO: 13), GC11 (SEQ ID NO: 14), GC12 (SEQ ID NO: 15), GC13 (SEQ ID NO: 16), GC14 (SEQ ID NO: 17), and GC15 (SEQ ID NO: 18) targeting exon 6 of the IL2RG gene were ordered from Synthego and evaluated in primary human CD3+ T cells transfected with Cas9/gRNA RNPs including the respective gRNA by electroporation following three days of activation with anti-CD3/CD8/CD28 beads. Forty-eight hours after transfection, the cells were analyzed for cleavage efficiency at the on-target site for each gRNA using the TIDES protocol as described above. The cells were analyzed one day post-editing for INDEL efficiency, which ranged from about 15% to about 80%, indicating that a number of the gRNAs efficiently cleave at their target sites in T cells (Table 4, results from 3 independent experiments).

TABLE 4

| | Average | |
|---|---|---|
| | INDEL | |
| gRNA | Frequency | Standard |
| Spacer | (%) | Deviation |
| GC3 | 77.53 | 3.95 |
| GC2 | 74.67 | 6.57 |
| GC10 | 71.77 | 17.24 |
| GC8 | 66.40 | 3.44 |
| GC12 | 58.43 | 12.03 |
| GC15 | 46.77 | 13.17 |
| GC1 | 46.43 | 19.90 |
| GC4 | 41.07 | 23.40 |
| GC13 | 35.60 | 4.20 |
| GC9 | 31.37 | 14.28 |
| GC7 | 31.07 | 15.37 |
| GC14 | 28.23 | 20.65 |
| GC11 | 15.60 | 10.00 |
| GC6 | 14.80 | 8.51 |
| GCS | 13.03 | 6.56 |
| No RNP | 1.63 | 1.27 |

To evaluate targeted integration of a donor template at the ILR2G locus mediated by gRNAs GC8, GC10, and GC12, primary human CD3+ T cells were transfected with Cas9/gRNA RNPs including the respective gRNA by electroporation alone, or immediately followed by transduction with a corresponding AAV vector with homology arms specific for each gRNA (homology arms of SEQ ID NOs: 86 and 87 for GC8; homology arms of SEQ ID NOs: 88 and 89 for GC10; and homology arms of SEQ ID NOs: 90 and 91 for GC12) and carrying a donor template encoding a CISC and a tLNGFR marker (SEQ ID NO: 97) for integration at a multiplicity of infection (MOI) of 50,000. Forty-eight hours after transduction, the cells were analyzed for integration efficiency using flow cytometry for tLNGFR and for INDEL efficiency. As shown in Table 5 (results from two independent experiments with different T cell lots), targeted integration of the donor templates was achieved for each of the three gRNAs tested, and the amount of CISC+ cells (as indicated by tLNGFR expression) ranged from about 11% to about 29%.

TABLE 5

| | INDEL Frequency (%) | CISC + cells (%) |
|---|---|---|
| Untreated | 4.7 | 0.1 |
| GC8 RNP | 24.3 | 0.1 |
| GC10 RNP | 53.25 | 0.05 |
| GC12 RNP | 27.75 | 0.05 |
| GC8 RNP + AAV | 3.4 | 10.85 |
| GC10 RNP + AAV | 30.85 | 28.55 |
| GC12 RNP + AAV | 24.85 | 11.9 |

### Off-Target Analysis

Off-target sites for human IL2RG-targeting gRNAs GC8, GC10, and GC12 were evaluated in primary human CD3+ cells using the GUIDE-seq method (Tsai, S. Q. et al. (2015). Nat. Biotechnol., 33(2):187-197). GUIDE-seq is an empirical method used to identify cleavage sites. GUIDE-seq relies on the spontaneous capture of an oligonucleotide at the site of a double-strand break in chromosomal DNA. In brief, following transfection of cells with a guide RNA/Cas9 RNP complex and double-stranded oligonucleotide, genomic DNA is purified from the cells, sonicated, and a series of adapter ligations are performed to create a library. The oligonucleotide-containing libraries are subjected to high-throughput DNA sequencing, and the output is processed using the default GUIDE-seq software to identify sites of oligonucleotide capture.

Samples without transfection of RNP containing SpCas9 and the sgRNA were processed in parallel. Sites (+/−1 kb) found in both RNP-containing and RNP-naive samples were excluded from further analysis.

The Y-adapter was prepared by annealing the Common Adapter to each of the sample barcode adapters (A01-A16) that contain the 8-mer molecular index. Genomic DNA extracted from the CD3+ T cells that were nucleofected with RNP and the GUIDE-seq ODN was quantified using a Qubit fluorometer (ThermoFisher Scientific) and all samples were normalized to 400 ng in 120 μl volume of TE buffer. The genomic DNA was sheared to an average length of 200 bp according to the standard operating procedure for the Covaris S220 sonicator. To confirm average fragment length, 1 μl of the sample was analyzed on a TapeStation (Agilent) according to manufacturer's protocol. Samples of sheared DNA were cleaned using AMPure XP SPRI beads according to the manufacturer's protocol and eluted in 17 μl of TE buffer. The end repair reaction was performed on the genomic DNA by mixing 1.2 μl of dNTP mix (5 mM each dNTP), 3 μl of 10×T4 DNA ligase buffer, 2.4 μl of End-Repair Mix, 2.4 μl of 10× Platinum Taq Buffer (Mg²⁺ free), and 0.6 μl of Taq Polymerase (non-hotstart) and 14 μl sheared DNA sample (from previous step) for a total volume of 22.5 μl per tube and incubated in a thermocycler (12° C., 15 minutes; 37° C., 15 minutes; 72° C., 15 minutes; 4° C. hold). To this was added 1 μl annealed Y Adapter (10 μM) and 2 μl T4 DNA ligase, and the mixture was incubated in a thermocycler (16° C., 30 minutes; 22° C., 30 minutes; 4° C. hold). The sample was cleaned using AMPure XP SPRI beads according to manufacturer's protocol and eluted in 23 μl of TE Buffer. One μl of sample was run on a TapeStation according to manufacturer's protocol to confirm ligation of adapters to fragments. To prepare the GUIDE-seq library a reaction was prepared containing 14 μl nuclease-free H₂O, 3.6 μl 10× Platinum Taq Buffer, 0.7 μl dNTP mix (10 mM each), 1.4 μl MgCl₂, 50 mM, 0.36 μl Platinum Taq Polymerase, 1.2 μl sense or antisense gene specific primer (10 μM), 1.8 μl TMAC (0.5 M), 0.6 μl P5_1 (10 μM) and 10 μl of the sample from the previous step. This mix was incubated in a thermocycler (95° C., 5 minutes, then 15 cycles of 95° C., 30 seconds; 70° C. (minus 1° C. per cycle) for 2 minutes; 72° C., 30 seconds; followed by 10 cycles of 95° C., 30 seconds; 55° C., 1 minute; 72° C., 30 seconds; followed by 72° C., 5 minutes). The PCR reaction was cleaned using AMPure XP SPRI beads according to manufacturer protocol and eluted in 15 μl of TE Buffer. 1 μl of sample was checked on TapeStation according to manufacturer's protocol to track sample progress. A second PCR was performed by mixing 6.5 μl Nuclease-free H₂O, 3.6 μl 10× Platinum Taq Buffer (Mg²⁺ free), 0.7 μl dNTP mix (10 mM each), 1.4 μl MgCl₂ (50 mM), 0.4 μl Platinum Taq Polymerase, 1.2 μl of Gene Specific Primer (GSP) 2 (sense: +, or antisense: −), 1.8 μl TMAC (0.5 M), 0.6 μl P5_2 (10 μM) and 15 μl of the PCR product from the previous step.

GUIDE-seq was completed on multiple independent cell sample replicates (from independent transfections) for each gRNA and the results are shown in Tables 6 and 7. These results demonstrate generally favorable on-target/off-target profiles for gRNA spacers GC8, GC10, and GC12.

TABLE 6
Summary of GUIDE-seq results for gRNAs with spacers
GC8, GC10, and GC12 in CD3+ T cells

| Guide | GUIDE-seq | Present in Multiple | On-Target Read |
|---|---|---|---|
| Name | Off-Targets | Replicates | Count |
| GC8 | 930 | 3 | 4348 |
| GC10 | 1227 | 14 | 5384 |
| GC12 | 1368 | 4 | 2352 |

TABLE 7
Details of the off-target sites detected by GUIDE-seq in at least 2 of the cell sample replicates

| | | | | | |
|---|---|---|---|---|---|
| | | Location | | | Off-Target/ |
| Chromosome | Position¹ | Type | Gene | Full Gene Name | On-Target |

GC8

| | | | |
|---|---|---|---|
| chr1 | 125180094 | Intergenic | 1.54% |
| chr16 | 46399022 | Intergenic | 0.46% |
| chr16 | 46390807 | Intergenic | 0.14% |

GC10

| | | | | | |
|---|---|---|---|---|---|
| chr3 | 108840645 | Intronic | TRAT1 | T cell receptor associated | 3.05% |
| | | | | transmembrane adaptor 1 | |
| chrUn_KI270438v1 | 104161 | | | | 1.60% |
| chr13 | 18212170 | Intronic | FAM230C | family with sequence similarity | 1.02% |
| | | | | 230 member C | |
| chrUn_KI270438v1 | 109477 | | | | 0.97% |
| chr21 | 17142630 | Intergenic | | | 0.71% |
| chr12 | 62289934 | Intronic | USP15 | ubiquitin specific peptidase 15 | 0.48% |
| chrUn_KI270467v1 | 2622 | | | | 0.48% |
| chrUn_KI270438v1 | 109447 | | | | 0.39% |
| chrUn_KI270438v1 | 104938 | | | | 0.28% |
| chrUn_KI270467v1 | 3365 | | | | 0.26% |
| chr5 | 159185831 | Intronic | RNF145 | ring finger protein 145 | 0.20% |
| chrUn_KI270467v1 | 2297 | | | | 0.17% |
| chrUn_KI270467v1 | 2459 | | | | 0.17% |
| chrUn_KI270467v1 | 2830 | | | | 0.13% |

GC12

| | | | | | |
|---|---|---|---|---|---|
| chr13 | 18212170 | Intronic | FAM230C | family with sequence similarity | 1.02% |
| | | | | 230 member C | |
| chrUn_KI270467v1 | 2459 | | | | 0.77% |
| chrUn_KI270590v1 | 2621 | | | | 0.38% |
| chrUn_KI270467v1 | 2660 | | | | 0.34% |

¹Position refers to the genomic location in Genome Reference Consortium Human Build 38 (hg38). The NCBI Genome Data Viewer was used to annotate each position (www.ncbi.nlm.nih.gov/genome/gdv).

While the percentage of off-target to on-target reads provides an overall representation of whether a gRNA is specific to its intended target, other factors may be involved. For example, an off-target site for a candidate gRNA in an exon of an essential gene required for survival of an organism could render the gRNA unsuitable for use in the clinic. On the other hand, an off-target site in a non-coding or intronic region may pose less concern. Considerations useful for evaluating a gRNA intended for therapeutic use include 1) the number of off-target sites, 2) the location of the off-target sites, 3) the frequency of off-target editing compared to on-target editing, and 4) the degree of homology of the off-target site to the gRNA spacer sequence.

Potential off-target sites were validated by reproducing the experiment in cell sample replicates. Accordingly, applicant conducted experiments to identify potential off-target sites in cells edited using gRNAs targeting IL2RG exon 6. Off-target sites that were detected in multiple cell sample replicates are reported in Table 7. Comparison of the read counts for each off-target site to the on-target site in GUIDE-seq provides an estimate of the off-target frequencies of the off-target sites for each sgRNA. These data are summarized in Table 7 along with information on the genomic site and whether the off-target site lies within the coding region of a gene. A spacer seed sequence consisting of the seven nucleotides of the spacer corresponding to the target sequence adjacent to the protospacer adjacent motif (PAM) has been shown by Zheng, T. et al. to be sensitive to mismatches (Zheng, T. et al. (2017). Sci. Rep., 7, 40638.). Predicted off-target sites with mismatches corresponding to the sgRNA spacer seed sequence would not be expected to be edited efficiently. Such off-target sites with mismatches in this seed region are likely to be false positives. True off-target frequencies can be confirmed by deep sequencing methods such as amplicon sequencing (see Medinger, R. et al. (2010). Mol. Ecol., 19(Suppl. 1):32-40).

The on-target site and potential off-target sites for human TRAC-targeting gRNA spacer TRAC 1 (SEQ ID NO: 3) were evaluated in primary human CD3+ cells using amplicon sequencing. A pair of PCR primers was designed to amplify ˜200 bp of the region of interest with the potential cleavage site located approximately in the middle. Barcoded amplicons were generated from RNP-treated and mock-transfected cells, multiplexed, and subjected to high-throughput DNA sequencing. Sequence reads were demultiplexed, paired-end reads aligned and merged using Pandaseq 2.11 (Masella, A. P., et al. (2012). BMC bioinformatics, 13(1), 31), and the frequency of INDELs was determined for each target site with custom software that uses the Biopython 1.69 pairwise2 aligner. For each target site, a minimum of 10,000 sequence reads and an average of 40,000 across the collection of reads was performed. As shown in Table 8, the INDEL frequency for the on-target site was about 85%. Three potential off-target sites with INDEL frequencies greater than 0.2% were identified, but these appear to have resulted from noise in the sequencing runs. These results indicate a highly favorable on-target/off-target profile for gRNA spacer TRAC 1.

TABLE 8

| Target Site Locus | INDEL Frequency (%) |
|---|---|
| on-target site | 84.89 |
| chr1_151031887 | 0.5 |
| chr10_42385299 | 0.27 |
| chr4_175681976 | 0.22 |
| chr4_64499999 | 0.17 |
| chr19_55086187 | 0.16 |
| chr1_192338993 | 0.14 |
| chr11_83606941 | 0.14 |
| chr19_54783512 | 0.13 |
| chr19_27731991 | 0.12 |
| chr11_31817474 | 0.11 |
| chr18_21359558 | 0.11 |
| chr5_16698674 | 0.1 |
| chr19_55143375 | 0.07 |
| chr1_91846342 | 0.06 |
| chr13_100290751 | 0.05 |
| chr10_37704866 | 0.04 |
| chr4_152822294 | 0.02 |
| chr8_32397899 | 0.02 |
| chr16_48670703 | 0.02 |
| chr13_100546989 | 0.02 |
| chr20_41690279 | 0.01 |
| chr5_131598919 | 0.01 |
| chr7_61970309 | 0.01 |
| chr9_120595625 | 0.01 |
| chr1_109932513 | 0.01 |
| chr8_59715325 | 0.01 |
| chr14_77738868 | 0.01 |
| chr1_100337774 | 0 |
| chr11_12874646 | 0 |
| chr20_20928859 | 0 |
| chr6_16112813 | 0 |
| chr7_157040012 | 0 |
| chr2_242214607 | −0.01 |
| chr1_104671743 | −0.01 |
| chr17_61008724 | −0.01 |
| chr11_115032260 | −0.01 |
| chr15_92478803 | −0.03 |
| chr2_173826344 | −0.03 |
| chrX_150198527 | −0.03 |
| chr15_64155080 | −0.06 |
| chr11_71948806 | −0.09 |
| chr12_2987230 | −0.16 |
| chr6_100380971 | −0.26 |
| chr4_157542466 | −1.1 |
| chr2_236746479 | −1.22 |
| chr2_179621956 | −8.34 |

Overall, the results from the GUIDE-seq and amplicon sequencing analysis in CD3+ T cells demonstrated that gRNAs with spacers GC8, GC10, GC12, and TRAC 1 are good candidates for further use, such as in adoptive cell therapy or other cell-based therapy.

Screening of additional gRNAs with target sites in human TRAC and IL2RG genes for their on-target/off-target profile in human cells using the GUIDE-seq and/or amplicon sequencing methodologies described herein is contemplated as an approach to identify additional gRNA molecules that could be used to target these genes for the purpose of creating anti-BCMA CAR T cells.

### Example 2: Generation and Characterization of Anti-BCMA CAR-Expressing T Cells by Targeted Integration at a TRAC Gene

T cells with targeted integration of an expression cassette encoding an anti-BCMA CAR into a TRAC gene were generated using TRAC-targeting Cas9/sgRNA RNPs in combination with AAV donor templates designed for integration by HDR. In general, donor templates designed for HDR-mediated integrations should be configured such that the integration site is close to the gRNA target site, for example less than 10 bp away (blog.addgene.org/crispr-101-homology-directed-repair). The AAV donor templates contained an expression cassette having its own promoter and flanked by homology arms including a target site for the sgRNA in the RNP (FIG. 1, donor template constructs #1 and #6).

Primary human CD3+ T cells were isolated from individual whole blood leukopaks. The isolated T cells were cultured in AIM-V medium plus 5% human AB serum plus 50 ng/mL IL-2. The cells were stimulated to proliferate using anti-CD3/CD28 magnetic beads (Miltenyi Biotec, 130-091-441) at a 1:1 ratio at a starting concentration of 0.5e6 cell s/mL for three days. The beads were then removed and the cells were allowed to divide for one day prior to transfection.

Cas9/sgRNA RNPs targeting the TRAC gene were prepared by combining 60 pmol TRAC 3 sgRNA (spacer sequence: TCTCTCAGCTGGTACACGGC (SEQ ID NO: 2)) and 12 pmol Cas9 (TrueCut V2, Thermo Fisher Scientific) in phosphate-buffered saline for at least 10 minutes at room temperature. The cells were transfected with the RNPs using a Lonza 4D nucleofector and program EO-115. One hour post-transfection, cells were infected with donor AAV2/6 vectors for expression of an anti-BCMA CAR with a CD28 co-stimulatory domain (#1 TRAC 3, SEQ ID NO: 20), an anti-BCMA CAR with a 4-1BB co-stimulatory domain (#6 TRAC 3, SEQ ID NO: 35), an anti-TNP CAR with a CD28 co-stimulatory domain (SEQ ID NO: 92), or an anti-TNP CAR with a 4-1BB co-stimulatory domain (SEQ ID NO: 93) at an MOI of 20,000.

Five days after editing, the cells were stained with anti-mouse Fv-biotin followed by streptavidin-PE and analyzed by flow cytometry. As shown in Table 9, between 9% and 12% of T cells treated with the anti-BCMA CAR donors showed CAR expression. These results demonstrate that targeted integration of an expression cassette into a TRAC gene in T cells allows for expression of a CAR from the integrated cassette.

TABLE 9

| Treatment | CAR+ cells (%) |
|---|---|
| #1 TRAC 3 | 11.59 |
| #6 TRAC 3 | 9.40 |
| α-TNP/CD28/CD3ζCAR | 23.09 |
| α-TNP/41BB/CD3ζCAR | 11.98 |

### Example 3: Simultaneous Analysis of TRAC and CAR Expression

To evaluate the effect of targeted integration of a heterologous sequence into a TRAC gene on TCR expression, T cells treated as in Example 2 were stained five days post-treatment simultaneously with an anti-α/β TCR antibody and biotinylated BCMA/streptavidin-PE and analyzed by flow cytometry. Approximately 90% of T cells lacked TCR expression when treated with the TRAC-targeting Cas9/sgRNA RNP, and between 18% and 22% of T cells treated with the TRAC-targeting Cas9/sgRNA RNP and an anti-BCMA CAR AAV donor were TCR-negative and expressed an anti-BCMA CAR (Table 10). These results indicate that editing T cells using a TRAC-targeting Cas9/gRNA RNP was effective for knocking out TCR expression in the edited cells.

TABLE 10

| | α-BCMA | α-BCMA | α-BCMA | α-BCMA |
|---|---|---|---|---|
| | CAR+/ | CAR+/ | CAR−/ | CAR−/ |
| | TCR− cells | TCR+ cells | TCR− cells | TCR+ cells |
| Treatment | (%) | (%) | (%) | (%) |
| AAV only | 0.00 | 0.15 | 0.85 | 98.99 |
| RNP only | 0.05 | 0.01 | 90.72 | 9.22 |
| #1 TRAC 3 | 18.05 | 1.01 | 70.71 | 10.23 |
| #6 TRAC 3 | 22.36 | 1.15 | 67.16 | 9.33 |
| α-TNP/CD28/CD3ζ | 0.12 | 0.00 | 88.63 | 11.25 |
| CAR | | | | |
| α-TNP/41BB/CD3ζ | 0.05 | 0.02 | 88.38 | 11.54 |
| CAR | | | | |

### Example 4: CAR Persistence to Day Twelve Post-Transfection

To evaluate the persistence of anti-BCMA CAR expression in anti-BCMA CAR T cells, T cells treated as in Example 2 were stained at day twelve post-transfection simultaneously with an anti-α/β TCR antibody and biotinylated BCMA/streptavidin-PE and analyzed by flow cytometry. Approximately 90% of cells lacked TCR expression when treated with a TRAC-targeting RNP, and between 22% and 30% of T cells treated with an anti-BCMA CAR AAV donor were TCR-negative and expressed an anti-BCMA CAR (Table 11). These results demonstrate that anti-BCMA CAR expression persists at least to day twelve post-transfection in edited T cells.

TABLE 11

| | α-BCMA | α-BCMA | α-BCMA | α-BCMA |
|---|---|---|---|---|
| | CAR+/ | CAR+/ | CAR−/ | CAR−/ |
| | TCR− cells | TCR+ cells | TCR− cells | TCR+ cells |
| Treatment | (%) | (%) | (%) | (%) |
| #1 TRAC 3 | 29.84 | 0.95 | 62.45 | 6.76 |
| #6 TRAC 3 | 21.81 | 0.95 | 66.51 | 10.73 |
| α-TNP/CD28/CD3ζ | 0.18 | 0.00 | 88.94 | 10.88 |
| CAR | | | | |
| α-TNP/41BB/CD3ζ | 0.00 | 0.01 | 92.48 | 7.51 |
| CAR | | | | |

In another experiment, T cells treated as in Example 2 were evaluated for CAR expression at day twelve post-transfection by staining with an anti-mouse antibody that recognizes the extracellular antibody moiety of each of the CARs followed by flow cytometry analysis. a/(3 TCR expression was not evaluated in this experiment as the anti-mouse variable chain CAR detection reagent interferes with the mouse TCR antibody. Between 18% and 30% of T cells expressed a CAR (Table 12).

TABLE 12

| Treatment | CAR+ cells (%) | CAR− cells (%) |
|---|---|---|
| #1 TRAC 3 | 21.71 | 78.29 |
| #6 TRAC 3 | 18.90 | 81.10 |
| α-TNP/CD28/CD3ζCAR | 30.21 | 69.79 |
| α-TNP/41BB/CD3ζCAR | 23.09 | 76.90 |

### Example 5: More AAV Gives More CAR-Positive Cells

To evaluate the effect of the amount of AAV donor used for transduction on anti-BCMA CAR expression, T cells were edited as in Example 2 but with MOIs of either 25,000, 50,000, or 100,000. Cells were stained simultaneously with anti-a/3 TCR antibody and biotinylated BCMA/streptavidin-PE and analyzed by flow cytometry five days after editing. Greater than 95% of cells lacked TCR expression when treated with a TRAC-targeting RNP, and between 20% and 60% of T cells expressed an anti-BCMA CAR, with the amount of anti-BCMA CAR+ cells positively correlating with AAV donor MOI (Table 13). These results demonstrate a dose-response for AAV donor MOI on donor integration efficiency.

TABLE 13

| | α-BCMA | α-BCMA | α-BCMA | α-BCMA |
|---|---|---|---|---|
| | CAR+/ | CAR+/ | CAR−/ | CAR−/ |
| | TCR− cells | TCR+ cells | TCR− cells | TCR+ cells |
| Treatment | (%) | (%) | (%) | (%) |
| #1 TRAC 3, 25k | 20.55 | 0.52 | 74.31 | 4.62 |
| MOI | | | | |
| #1 TRAC 3, 50k | 42.76 | 0.50 | 53.28 | 3.47 |
| MOI | | | | |
| #1 TRAC 3, 100k | 60.92 | 0.29 | 38.21 | 0.57 |
| MOI | | | | |
| #6 TRAC 3, 25k | 26.74 | 0.42 | 69.44 | 3.40 |
| MOI | | | | |
| #6 TRAC 3, 50k | 37.16 | 0.35 | 58.04 | 4.45 |
| MOI | | | | |
| #6 TRAC 3, 100k | 40.26 | 0.25 | 57.62 | 1.86 |
| MOI | | | | |

### Example 6: Anti-BCMA CAR T Cells are Cytotoxic to BCMA-Expressing Cells

This example demonstrates the cytotoxicity of anti-BCMA CAR T cells towards BCMA-expressing cells. T cells were transfected with RNPs containing either the TRAC 3 or TRAC 1 sgRNAs and then infected with a corresponding AAV donor (α-BCMA/CD28/CD3z TRAC 1, SEQ ID NO: 21; α-BCMA/CD28/CD3z TRAC 3, SEQ ID NO: 20; or α-BCMA/41BB/CD3z-CISCβ TRAC 1, SEQ ID NO: 36) at an MOI of 50,000. Either 14 (TRAC 1 sgRNA) or 22 (TRAC 3 sgRNA) days post-transfection, the T cells were used in a cytotoxicity assay with either wild-type K562 cells (non-BCMA-expressing) or K562 Very High-BCMA (K562 VH-BCMA) cells (BCMA-expressing) as the target cells at an effector:target ratio of 8:1. K562 VH-BCMA target cell viability (as determined by DAPI staining) dropped from 93% to 26% after co-culture with anti-BCMA CAR T cells, whereas K562 target cell viability remained at about 82% after co-culture with anti-BCMA CAR T cells (Table 14), demonstrating that the anti-BCMA CART cells are cytotoxic to BCMA-expressing cells, and the cytotoxicity depends on BCMA expression.

TABLE 14

| Effector T cell | | Target cell |
|---|---|---|
| (AAV donor treatment) | Target cell | viability (%) |
| No AAV donor | K562 VH-BCMA | 93.45 |
| α-BCMA/CD28/CD3z | K562 VH-BCMA | 25.99 |
| TRAC 3 | | |
| a-BCMA/CD28/CD3z | K562 | 82.94 |
| TRAC 1 | | |
| α-BCMA/41BB/CD3z- | K562 | 81.94 |
| CISCβ TRAC 1 | | |

### Example 7: Cytotoxicity Requires a CAR that Binds BCMA

This example demonstrates the cytotoxicity of CAR T cells towards BCMA-expressing cells depends on CAR specificity for BCMA. T cells were transfected with RNPs containing the TRAC 1 sgRNAs and then infected with corresponding AAV donors encoding an anti-TNP CAR (α-TNP/CD28/CD3ζ CAR TRAC 1, SEQ ID NO: 92; or α-TNP/41BB/CD3ζ CAR TRAC 1, SEQ ID NO: 93) or corresponding AAV donors encoding an anti-BCMA CAR (α-BCMA/CD28/CD3z TRAC 1, SEQ ID NO: 21; or α-BCMA/41BB/CD3z-CISCβ TRAC 1, SEQ ID NO: 36) at an MOI of 50,000. Fourteen days post-transfection the T cells were used in a cytotoxicity assay with K562 Very High-BCMA (K562 VH-BCMA) as the target cells at an effector:target ratio (E:T) of 8:1. Target cell viability dropped from 93% to ˜40% after exposure to anti-BCMA CAR T cells, while exposure to anti-TNP CAR T cells reduced viability by only ˜10% (Table 15). These results demonstrate the dependence of anti-BCMA CAR T cell cytotoxicity on the anti-BCMA CAR specificity.

TABLE 15

| Effector T cell | | Target cell |
|---|---|---|
| (AAV donor treatment) | Target cell | viability (%) |
| α-BCMA/CD28/CD3z | K562 VH-BCMA | 38.90 |
| TRAC 1 | | |
| α-BCMA/41BB/CD3z- | K562 VH-BCMA | 43.40 |
| CISCβ TRAC 1 | | |
| α-TNP/CD28/CD3ζCAR | K562 VH-BCMA | 82.29 |
| α-TNP/41BB/CD3ζCAR | K562 VH-BCMA | 79.95 |

Additional experiments were performed to further demonstrate CAR-specific cytotoxicity using the anti-BCMA CAR construct with the 41BB costimulatory domain. Either TNP-specific or BCMA-specific CAR T cells were co-cultured as described above with K562 VH-BCMA cells at varying CAR T:target cell ratios (2:1 to 16:1). After co-culture, the cells were stained with DAPI and the frequency of DAPI-positive and DAPI-negative cells was measured. Exposure to BCMA-specific, but not TNP-specific, CAR T cells caused the viability of the culture to decline in rough proportion to the E:T ratio, reaching a nadir of ˜13% at a 16:1 E:T ratio (Table 16). In the absence of CART exposure, the target cells were >95% viable (not shown). These results further demonstrate the requirement of BCMA specificity of the CAR T effector cells for killing of BCMA-expressing target cells.

TABLE 16

| | Target cell viability (%) | | | |
|---|---|---|---|---|
| Effector T cell | 2:1¹ | 4:1¹ | 8:1¹ | 16:1¹ |
| Anti-BCMA CAR T cell | 79.97 | 68.58 | 27.05 | 13.10 |
| Anti-TNP CAR T cell | 90.41 | 80.87 | 83.52 | 77.20 |

¹Effector-to-Target (E:T) Ratio

### Example 8: Targeted Integration into the IL2RG Locus

This examples demonstrates targeted integration into an IL2RG gene using a gRNA targeting the gene and a compatible donor template. T cells were transfected with RNPs containing the GC8, GC10, or GC12 sgRNAs targeting exon 6 of the IL2RG gene and then infected with an FRB/tLNGFR/CNb30/CISC-gamma donor AAV (SEQ ID NO: 40) at an MOI of 50,000. Conditions with RNP only and AAV only were included as controls. Cells were stained simultaneously with anti-IL2RG and anti-LNGFR antibodies and analyzed by flow cytometry one and a half days after transfection. Four, four, and six percent of cells expressed the tLNGFR transgene when using the GC8, GC10, and GC12 sgRNAs, respectively (Table 17). For all RNP-treated samples, greater than 85% of cells lost IL2RG expression.

TABLE 17

| | tLNGFR+/ | tLNGFR+/ | tLNGFR−/ | tLNGFR−/ |
|---|---|---|---|---|
| | IL2RG− | IL2RG+ | IL2RG− | IL2RG+ |
| Treatment | cells (%) | cells (%) | cells (%) | cells (%) |
| AAV donor only | 0.31 | 0.56 | 39.77 | 59.36 |
| GC8 RNP only | 0.63 | 0.21 | 91.90 | 7.25 |
| GC10 RNP only | 0.89 | 0.23 | 97.64 | 1.25 |
| GC12 RNP only | 0.67 | 0.27 | 89.54 | 9.51 |
| GC8 RNP + AAV | 3.93 | 0.36 | 89.83 | 5.88 |
| donor | | | | |
| GC10 RNP + AAV | 4.25 | 0.42 | 93.80 | 1.54 |
| donor | | | | |
| GC12 RNP + AAV | 6.24 | 0.35 | 86.49 | 6.92 |
| donor | | | | |

In another experiment, T cells are transfected with RNPs containing the GC8, GC10, or GC12 sgRNAs and then infected with a corresponding sgRNA-specific FRB/tLNGFR/CNb30/CISC-gamma donor AAV mutated to prevent recleavage of the integrated transgene and to promote correct homology-dependent DNA repair (e.g., SEQ ID NO: 41, 42, or 43 for GC8, GC10, or GC12 sgRNAs, respectively), e.g., at an MOI of 50,000. Cells are stained simultaneously with anti-IL2RG and anti-LNGFR antibodies and analyzed by flow cytometry post-transfection (e.g., one and a half days post-transfection) for tLNGFR transgene expression and IL2RG expression.

### Example 9: Simultaneous TI into a TRAC Gene and an IL2RG Gene

T cells are transfected with a TRAC-targeting RNP (e.g., TRAC 3, TRAC 2, or TRAC 1 RNP) along with an IL2RG-targeting RNP (e.g., GC8, GC10, or GC12 RNP). Following transfection (e.g., thirty minutes post-transfection), cells are infected with a donor AAV encoding anti-BCMA CAR/CISC-b targeted to a TRAC gene (e.g., SEQ ID NOs: 28-39) and a donor AAV encoding FRB/tLNGFR/CNb30/CISC-gamma targeted to an IL2RG gene (e.g., SEQ ID NOs: 40-44) (e.g., both at MOIs of 50,000). Cells are recovered into medium containing rapamycin or a rapalog (e.g., 1 nM rapamycin) and maintained in rapamycin/rapalog-containing medium. Cells are assayed by flow cytometry post-transfection (e.g., five days post-transfection) for TRAC expression, CAR expression, IL2RG expression, and/or tLNGFR expression.

Flow cytometry was performed to illustrate the efficiency of dual targeted integration. CD8+ T cells were stimulated with CD3/CD28 beads for three days, the beads removed, and then one day later the cells were treated with TRAC 1 RNP+BCMA CAR-CISCβ AAV and IL2RG GC12 RNP+FRB-tLNGFR-CNb30-CISCγ AAV. Donor AAV was used at a multiplicity of infection of 25,000; TRAC 1 RNP contained 30 pmol guide RNA and 6 pmol Cas9; and IL2RG GC12 RNP contained 60 pmol guide RNA and 12 pmol Cas9. Cells were recovered into medium containing 1 nM rapamycin and maintained in rapamycin-containing medium. One, three, and seven days post-treatment cells were analyzed by flow cytometry for the presence of tLNGFR and for the presence of an anti-BCMA CAR. In these experiments, the efficiencies of single locus targeting ranged from about 20%, whereas the double-targeting frequency (e.g., simultaneous targeted integration at both loci) was approximately 8% (Table 18).

TABLE 18

| | Day post-treatment | | |
|---|---|---|---|
| | 1 | 3 | 7 |
| tLNGFR+/ | 4.61 | 8.24 | 6.35 |
| CAR+ cells (%) | | | |
| tLNGFR+/ | 6.99 | 10.62 | 13.54 |
| CAR− cells (%) | | | |
| tLNGFR+/ | 11.63 | 14.14 | 10.34 |
| CAR+ cells (%) | | | |
| tLNGFR−/ | 76.77 | 67.01 | 69.77 |
| CAR− cells (%) | | | |
| Viability (%) | 96 | 97 | 95 |

### Example 10: Simultaneous TI into TRAC and IL2RG Gives CISC-Regulatable T Cells

Modified cells from Example 9 or corresponding unmodified cells are expanded in the presence of rapamycin, e.g., for two weeks or to at least 100-fold expansion. After this expansion, cells are transferred into rapamycin-free medium optionally supplemented with IL-2 (e.g., 100 ng/mL IL-2), and the viability of the cells is monitored (e.g., monitored every day for seven days).

### Example 11: Simultaneous TI into TRAC and IL2RG Gives Cyclosporin-Resistant Cells

Modified cells from Example 9 or corresponding unmodified cells are grown in the presence of cyclosporinA and rapamycin (or a rapalog), and the proliferation and/or viability of the cells is monitored.

### Example 12: Simultaneous TI into TRAC and IL2RG Gives BCMA- and B2M-CAR-Expressing Cells

T cells are transfected with a TRAC-targeting RNP (e.g., TRAC 3, TRAC 2, or TRAC 1 RNP) along with an IL2RG-targeting RNP (e.g., GC8, GC10, or GC12 RNP). Following transfection (e.g., thirty minutes post-transfection), cells are infected with a donor AAV encoding anti-BCMA CAR/CISC-b targeted to TRAC (e.g., SEQ ID NOs: 28-39) and a donor AAV encoding B2M-CAR/FRB/CNb30/CISC-gamma targeted to IL2RG (e.g., SEQ ID NO: 44) (e.g., both at MOIs of 50,000). Cells are recovered into medium containing rapamycin (e.g., 1 nM rapamycin) and maintained in rapamycin-containing medium. Cells are assayed by flow cytometry post-transfection (e.g., five days post-transfection) for TRAC expression, anti-BCMA CAR expression, IL2RG expression, and/or B2M CAR expression.

### Example 13: BCMA/B2M CAR T Cells Kill Two Different Target Cell Types

Modified cells from Example 12 and corresponding unmodified cells are used in a cytotoxicity assay as described in Examples 6 and 7 with BCMA-expressing target cells or T lymphocyte target cells derived from an unrelated T cell donor from which the modified cells are derived.

### Example 14: BCMA CAR T Cells Kill Multiple Myeloma Cells In Vivo

Modified cells from Example 5 and Example 9 are injected intravenously into NSG mice bearing established xenograft multiple myeloma tumors (e.g., derived from the RPMI-8226 cell line or a BCMA-negative pool of RPMI-8226 cells). Tumor size is monitored (e.g., monitored every day for two weeks post-injection).

Five million RPMI-8226 cells were implanted into NSG mice and allowed to form tumors. After nineteen days of tumor growth, mice were injected with PBS, eight million TNP CAR T cells, or eight million BCMA CAR T cells. An untreated mouse, a mouse treated with anti-TNP CAR T cells, and a mouse with regression of the tumor in response to treatment with anti-BCMA CAR T cells were sacrificed, tumors were dissociated, and the resulting cell suspensions were analyzed by flow cytometry for human CD45 as a marker for CAR T cells that infiltrated the respective tumors (CD45 is a leukocyte marker, and is not expressed in RPMI-8226 cells). Only the mouse treated with the anti-BCMA CAR T cells showed tumor infiltration of the administered human T cells, with 12.00% of the cells from the tumor being hCD45+, as compared to 0.05% and 0.19% for the control mouse and the mouse treated with anti-TNP CAR T cells, respectively. This population of hCD45+ cells was further analyzed by flow cytometry for human CD8 and CAR expression. As shown in Table 19, about 96% of the hCD45+ tumor infiltrating lymphocytes (TILs) were CD8+, and 14.66% were CD8+ and CAR+. These results demonstrate that tumor infiltration of administered lymphocytes was anti-BCMA CAR T cell treatment-specific. Exhaustion markers such as LAGS, TIM3, and PD1, were not detectable (not shown).

TABLE 19

| hCD8+/ | hCD8+/ | hCD8−/ | hCD8−/ |
|---|---|---|---|
| CAR+ cells (%) | CAR− cells (%) | CAR+ cells (%) | CAR− cells (%) |
| 14.66 | 81.08 | 0.33 | 3.94 |

### Example 15: CAR-Specific and Antigen-Specific T Cell Degranulation

To evaluate degranulation in T cells edited to express a CAR, anti-TNP CAR T cells or anti-BCMA CAR T cells were incubated for 18 hours with BCMA protein, K562 cells, or K562 VH-BCMA cells in the presence of an anti-CD107a antibody (CD107a is a marker for degranulation in cytotoxic T cells) and monensin (to avoid internalization of CD107a). After incubation, cells were analyzed for CD107a expression by flow cytometry, and results are shown in Table 20. The percentage of anti-BCMA CAR+ T cells in the anti-BCMA CAR+ T cell+BCMA protein condition was 25% (data not shown), and the percentage of degranulated cells in this condition was 22%, suggesting that nearly all of the anti-BCMA CAR+ T cells treated with BCMA protein were activated for degranulation. By contrast, only 0.24% of cells in the anti-TNP CAR T cell+BCMA protein condition were degranulated. These results demonstrate CAR-specific, antigen-specific T cell degranulation for the anti-BCMA CAR T cells. Weaker stimulation was observed with K562 VH-BCMA cells, and the degranulation was still target-specific and CAR-specific.

TABLE 20

| | CD107a+ cells (%) | |
|---|---|---|
| | Anti-BCMA CAR | Anti-TNP CAR |
| Treatment | T cells | T cells |
| No stimulus | 0.06 | 0.02 |
| BCMA protein | 22.20 | 0.24 |
| K562 cells | 1.53 | 0.85 |
| K562 VH-BCMA cells | 3.82 | 0.53 |

SEQUENCE LISTING

| SEQ | | |
|---|---|---|
| ID | | |
| NO | Sequence | Description |
| 1 | AGAGCAACAGTGCTGTGGCC | TRAC |
| | | gRNA |
| | | spacer |
| | | TRAC 2 |
| 2 | TCTCTCAGCTGGTACACGGC | TRAC |
| | | gRNA |
| | | spacer |
| | | TRAC 3 |
| 3 | ACAAAACTGTGCTAGACATG | TRAC |
| | | gRNA |
| | | spacer |
| | | TRAC 1 |
| 4 | ACCAGTGCCTGGCATGTAGT | IL2RG |
| | | gRNA |
| | | spacer GC1 |
| 5 | CCAGTGCCTGGCATGTAGTA | IL2RG |
| | | gRNA |
| | | spacer GC2 |
| 6 | CAGTGCCTGGCATGTAGTAG | IL2RG |
| | | gRNA |
| | | spacer GC3 |
| 7 | GTAGGGGCACAACAAATATA | IL2RG |
| | | gRNA |
| | | spacer GC4 |
| 8 | GAATCCTTTCCTGTTTGCAT | IL2RG |
| | | gRNA |
| | | spacer GC5 |
| 9 | CCTGTTTGCATTGGAAGCCG | IL2RG |
| | | gRNA |
| | | spacer GC6 |
| 10 | GAAGCCGTGGTTATCTCTGT | IL2RG |
| | | gRNA |
| | | spacer GC7 |
| 11 | GGTTATCTCTGTTGGCTCCA | IL2RG |
| | | gRNA |
| | | spacer GC8 |
| 12 | GTTATCTCTGTTGGCTCCAT | IL2RG |
| | | gRNA |
| | | spacer GC9 |
| 13 | AAGGCTGATAATCAATCCCA | IL2RG |
| | | gRNA |
| | | spacer |
| | | GC10 |
| 14 | GGAGCCAACAGAGATAACCA | IL2RG |
| | | gRNA |
| | | spacer |
| | | GC11 |
| 15 | CCACGGCTTCCAATGCAAAC | IL2RG |
| | | gRNA |
| | | spacer |
| | | GC12 |
| 16 | GCTTCCAATGCAAACAGGAA | IL2RG |
| | | gRNA |
| | | spacer |
| | | GC13 |
| 17 | TAGAAAAAAGAAAAGCAAAG | IL2RG |
| | | gRNA |
| | | spacer |
| | | GC14 |
| 18 | TTGTGCCCCTACTACATGCC | IL2RG |
| | | gRNA |
| | | spacer |
| | | GC15 |
| 19 | cctgcaggcagctgcgcgctcgctcgctcactgaggccgcccgggcaaagcccgggcgtcgggcgacctttggtcgcccggcct | TRAC AAV |
| | cagtgagcgagcgagcgcgcagagagggagtggccaactccatcactaggggttcctgcggccgcccaagattgatagcttgtg | #1 TRAC 2: |
| | cctgtccctgagtcccagtccatcacgagcagctggtttctaagatgctatttcccgtataaagcatgagaccgtgacttgccagcccc | HA TRAC |
| | acagagccccgcccttgtccatcactggcatctggactccagcctgggttggggcaaagagggaaatgagatcatgtcctaaccct | 2-C11D5.3- |
| | gatcctcttgtcccacagatatccagaaccctgaccctgccgtgtaccagctgagagactctaaatccagtgacaagtctgtctgccta | CD8-CD28- |
| | ttcaccgattttgattctcaaacaaatgtgtcacaaagtaaggattctgatgtgtatatcacagacaaaactgtgctagacatgaggtcta | CD3z-HA |
| | tggacttcaagagcaacagtgctgtgtgaatgaatgattaattaataaaagatctttattttcattagatctgtgtgttggttttttgtgtgatc | TRAC 2 |
| | ctcgagggaatgaaagaccccacctgtaggtttggcaagctagcttaagtaacgccattttgcaaggcatggaaaatacataactga | |
| | gaatagagaagttcagatcaaggttaggaacagagagacagcagaatatgggccaaacaggatatctgtggtaagcagttcctgcc | |
| | ccggctcagggccaagaacagatggtccccagatgcggtcccgccctcagcagtttctagagaaccatcagatgtttccagggtgc | |
| | cccaaggacctgaaaatgaccctgtgccttatttgaactaaccaatcagttcgcttctcgcttctgttcgcgcgcttctgctccccgagct | |
| | caataaaagagcccacaacccctcactcggcgcgcgccagtccggtaccagtcgccaccatggccctgcctgtgacagctctgct | |
| | cctccctctggccctgctgctccatgccgccagacccgacatcgtgctgacccagagcccccccagcctggccatgtctctgggca | |
| | agagagccaccatcagctgccgggccagcgagagcgtgaccatcctgggcagccacctgatccactggtatcagcagaagcccg | |
| | gccagccccccaccctgctgatccagctcgccagcaatgtgcagaccggcgtgcccgccagattcagcggcagcggcagcaga | |
| | accgacttcaccctgaccatcgaccccgtggaagaggacgacgtggccgtgtactactgcctgcagagccggaccatcccccgga | |
| | cctttggcggaggcaccaaactggaaatcaagggcagcaccagcggctccggcaagcctggctctggcgagggcagcacaaag | |
| | ggacagattcagctggtgcagagcggccctgagctgaagaaacccggcgagacagtgaagatcagctgcaaggcctccggctac | |
| | accttcaccgactacagcatcaactgggtgaaaagagcccctggcaagggcctgaagtggatgggctggatcaacaccgagaca | |
| | agagagcccgcctacgcctacgacttccggggcagattcgccttcagcctggaaaccagcgccagcaccgcctacctgcagatca | |
| | acaacctgaagtacgaggacaccgccacctacttttgcgccctggactacagctacgccatggactactggggccagggcaccag | |
| | cgtgaccgtgtccagcttcgtgcccgtgttcctgcccgccaaacctaccaccacccctgcccctagacctcccaccccagccccaa | |
| | caatcgccagccagcctctgtctctgcggcccgaagcctgtagacctgctgccggcggagccgtgcacaccagaggcctggactt | |
| | cgcctgcgacatctacatctgggcccctctggccggcacctgtggcgtgctgctgctgagcctggtgatcaccctgtactgcaacca | |
| | ccggaacagaagcaagcggagccggctgctgcacagcgactacatgaacatgaccccaagacggcctggccccacccggaag | |
| | cactaccagccttacgcccctcccagagacttcgccgcctaccggtccagagtgaagttcagcagatccgccgacgcccctgccta | |
| | ccagcagggacagaaccagctgtacaacgagctgaacctgggcagacgggaagagtacgacgtgctggacaagcggagaggc | |
| | cgggaccccgagatgggcggaaagcccagacggaagaacccccaggaaggcctgtataacgaactgcagaaagacaagatgg | |
| | ccgaggcctacagcgagatcggcatgaagggcgagcggaggcgcggcaagggccacgatggcctgtaccagggcctgagca | |
| | ccgccaccaaggacacctacgacgccctgcacatgcaggccctgccccccagatgaaagcttgataatcaacctctggattacaaa | |
| | atttgtgaaagattgactggtattcttaactatgttgctccttttacgctatgtggatacgctgctttaatgcctttgtatcatgctattgcttcc | |
| | cgtatggctttcattttctcctccttgtataaatcctggttagttcttgccacggcggaactcatcgccgcctgccttgcccgctgctggac | |
| | aggggctcggctgttgggcactgacaattccgtggaacttgtttattgcagcttataatggttacaaataaagcaatagcatcacaaatt | |
| | tcacaaataaagcatttttttcactgcattctagttgtggtttgtccaaactcatcaatgtatcttacgccggcgtgagcctggagcaacaa | |
| | atctgactttgcatgtgcaaacgccttcaacaacagcattattccagaagacaccttcttccccagcccaggtaagggcagctttggtg | |
| | ccttcgcaggctgtttccttgcttcaggaatggccaggttctgcccagagctctggtcaatgatgtctaaaactcctctgattggtggtct | |
| | cggccttatccattgccaccaaaaccctattttactaagaaacagtgagccttgttctggcagtccagagaatgacacgggaaaaaa | |
| | gcagatgaagagaaggtggcaggagagggcacgtggcccagcctcagtctctccaactgagttcctgcctgcctgcctttgctcag | |
| | actgtttgccccttactgctcttctaggcctccctaggaacccctagtgatggagttggccactccctctctgcgcgctcgctcgctcac | |
| | tgaggccgggcgaccaaaggtcgcccgacgcccgggctttgcccgggcggcctcagtgagcgagcgagcgcgcagctgcctg | |
| | cagg | |
| 20 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggttcctgcggccgccccatgcctgcctttactct | #1 TRAC 3: |
| | gccagagttatattgctggggttttgaagaagatcctattaaataaaagaataagcagtattattaagtagccctgcatttcaggtacctt | HA TRAC |
| | gagtggcaggccaggcctggccgtgaacgttcactgaaatcatggcctcttggccaagattgatagcttgtgcctgtccctgagtccc | 3-C11D5.3- |
| | agtccatcacgagcagctggtttctaagatgctatttcccgtataaagcatgagaccgtgacttgccagccccacagagccccgccct | CD8-CD28- |
| | tgtccatcactggcatctggactccagcctgggttggggcaaagagggaaatgagatcatgtcctaaccctgatcctcttgtcccaca | CD3z-HA |
| | gatatccagaaccctgaccctgcctgaatgaatgattaattaataaaagatctttattttcattagatctgtgtgttggttttttgtgtgatcct | TRAC 3 |
| | cgagggaatgaaagaccccacctgtaggtttggcaagctagcttaagtaacgccattttgcaaggcatggaaaatacataactgaga | |
| | atagagaagttcagatcaaggttaggaacagagagacagcagaatatgggccaaacaggatatctgtggtaagcagttcctgcccc | |
| | ggctcagggccaagaacagatggtccccagatgcggtcccgccctcagcagtttctagagaaccatcagatgtttccagggtgccc | |
| | caaggacctgaaaatgaccctgtgccttatttgaactaaccaatcagttcgcttctcgcttctgttcgcgcgcttctgctccccgagctca | |
| | ataaaagagcccacaacccctcactcggcgcgcgccagtccggtaccagtcgccaccatggccctgcctgtgacagctctgctcct | |
| | ccctctggccctgctgctccatgccgccagacccgacatcgtgctgacccagagcccccccagcctggccatgtctctgggcaaga | |
| | gagccaccatcagctgccgggccagcgagagcgtgaccatcctgggcagccacctgatccactggtatcagcagaagcccggcc | |
| | agccccccaccctgctgatccagctcgccagcaatgtgcagaccggcgtgcccgccagattcagcggcagcggcagcagaacc | |
| | gacttcaccctgaccatcgaccccgtggaagaggacgacgtggccgtgtactactgcctgcagagccggaccatcccccggacct | |
| | ttggcggaggcaccaaactggaaatcaagggcagcaccagcggctccggcaagcctggctctggcgagggcagcacaaaggg | |
| | acagattcagctggtgcagagcggccctgagctgaagaaacccggcgagacagtgaagatcagctgcaaggcctccggctacac | |
| | cttcaccgactacagcatcaactgggtgaaaagagcccctggcaagggcctgaagtggatgggctggatcaacaccgagacaag | |
| | agagcccgcctacgcctacgacttccggggcagattcgccttcagcctggaaaccagcgccagcaccgcctacctgcagatcaac | |
| | aacctgaagtacgaggacaccgccacctacttttgcgccctggactacagctacgccatggactactggggccagggcaccagcg | |
| | tgaccgtgtccagcttcgtgcccgtgttcctgcccgccaaacctaccaccacccctgcccctagacctcccaccccagccccaacaa | |
| | tcgccagccagcctctgtctctgcggcccgaagcctgtagacctgctgccggcggagccgtgcacaccagaggcctggacttcgc | |
| | ctgcgacatctacatctgggcccctctggccggcacctgtggcgtgctgctgctgagcctggtgatcaccctgtactgcaaccaccg | |
| | gaacagaagcaagcggagccggctgctgcacagcgactacatgaacatgaccccaagacggcctggccccacccggaagcact | |
| | accagccttacgcccctcccagagacttcgccgcctaccggtccagagtgaagttcagcagatccgccgacgcccctgcctacca | |
| | gcagggacagaaccagctgtacaacgagctgaacctgggcagacgggaagagtacgacgtgctggacaagcggagaggccgg | |
| | gaccccgagatgggcggaaagcccagacggaagaacccccaggaaggcctgtataacgaactgcagaaagacaagatggccg | |
| | aggcctacagcgagatcggcatgaagggcgagcggaggcgcggcaagggccacgatggcctgtaccagggcctgagcaccg | |
| | ccaccaaggacacctacgacgccctgcacatgcaggccctgccccccagatgaaagcttgataatcaacctctggattacaaaattt | |
| | gtgaaagattgactggtattcttaactatgttgctcatttacgctatgtggatacgctgctttaatgcattgtatcatgctattgcttcccgt | |
| | atggctttcattttctcctccttgtataaatcctggttagttcttgccacggcggaactcatcgccgcctgccttgcccgctgctggacag | |
| | gggctcggctgttgggcactgacaattccgtggaacttgtttattgcagcttataatggttacaaataaagcaatagcatcacaaatttca | |
| | caaataaagcatttttttcactgcattctagttgtggtttgtccaaactcatcaatgtatcttacgccggcgtgagtgtaccagctgagaga | |
| | ctctaaatccagtgacaagtctgtctgcctattcaccgattttgattctcaaacaaatgtgtcacaaagtaaggattctgatgtgtatatca | |
| | cagacaaaactgtgctagacatgaggtctatggacttcaagagcaacagtgctgtggcctggagcaacaaatctgactttgcatgtgc | |
| | aaacgccttcaacaacagcattattccagaagacaccttcttccccagcccaggtaagggcagctttggtgccttcgcaggctgtttcc | |
| | ttgcttcaggaatggccaggttctgcccagagctctggtcaatgatgtctaaaactcctctgattggtggtctcggccttatccattgcca | |
| | ccaaaaccctctttttactaagacctaggaacccctagtgatggagttggccactccctctctgcgcgctcgctcgctcactgaggcc | |
| | gggcgaccaaaggtcgcccgacgcccgggctttgcccgggcggcctcagtgagcgagcgagcgcgcagctgcctgcagg | |
| 21 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgctggccgtgaacgttcactg | #1 TRAC 1: |
| | aaatcatggcctcttggccaagattgatagcttgtgcctgtccctgagtcccagtccatcacgagcagctggtttctaagatgctatttcc | HA TRAC |
| | cgtataaagcatgagaccgtgacttgccagccccacagagccccgcccttgtccatcactggcatctggactccagcctgggagg | 1-C11D5.3- |
| | ggcaaagagggaaatgagatcatgtcctaaccctgatcctcttgtcccacagatatccagaaccctgaccctgccgtgtaccagctg | CD8-CD28- |
| | agagactctaaatccagtgacaagtctgtctgcctattcaccgattttgattctcaaacaaatgtgtcacaaagtaaggattctgatgtgt | CD3z-HA |
| | atatcacagacaaaactgtgctagactgaatgaatgattaattaataaaagatctttattttcattagatctgtgtgttggttttttgtgtgatc | TRAC 1 |
| | ctcgagggaatgaaagaccccacctgtaggtttggcaagctagcttaagtaacgccattttgcaaggcatggaaaatacataactga | |
| | gaatagagaagttcagatcaaggttaggaacagagagacagcagaatatgggccaaacaggatatctgtggtaagcagttcctgcc | |
| | ccggctcagggccaagaacagatggtccccagatgcggtcccgccctcagcagtttctagagaaccatcagatgtttccagggtgc | |
| | cccaaggacctgaaaatgaccctgtgccttatttgaactaaccaatcagttcgcttctcgcttctgttcgcgcgcttctgctccccgagct | |
| | caataaaagagcccacaacccctcactcggcgcgcgccagtccggtaccagtcgccaccatggccctgcctgtgacagctctgct | |
| | cctccctctggccctgctgctccatgccgccagacccgacatcgtgctgacccagagcccccccagcctggccatgtctctgggca | |
| | agagagccaccatcagctgccgggccagcgagagcgtgaccatcctgggcagccacctgatccactggtatcagcagaagcccg | |
| | gccagccccccaccctgctgatccagctcgccagcaatgtgcagaccggcgtgcccgccagattcagcggcagcggcagcaga | |
| | accgacttcaccctgaccatcgaccccgtggaagaggacgacgtggccgtgtactactgcctgcagagccggaccatcccccgga | |
| | cctttggcggaggcaccaaactggaaatcaagggcagcaccagcggctccggcaagcctggctctggcgagggcagcacaaag | |
| | ggacagattcagctggtgcagagcggccctgagctgaagaaacccggcgagacagtgaagatcagctgcaaggcctccggctac | |
| | accttcaccgactacagcatcaactgggtgaaaagagcccctggcaagggcctgaagtggatgggctggatcaacaccgagaca | |
| | agagagcccgcctacgcctacgacttccggggcagattcgccttcagcctggaaaccagcgccagcaccgcctacctgcagatca | |
| | acaacctgaagtacgaggacaccgccacctacttttgcgccctggactacagctacgccatggactactggggccagggcaccag | |
| | cgtgaccgtgtccagcttcgtgcccgtgttcctgcccgccaaacctaccaccacccctgcccctagacctcccaccccagccccaa | |
| | caatcgccagccagcctctgtctctgcggcccgaagcctgtagacctgctgccggcggagccgtgcacaccagaggcctggactt | |
| | cgcctgcgacatctacatctgggcccctctggccggcacctgtggcgtgctgctgctgagcctggtgatcaccctgtactgcaacca | |
| | ccggaacagaagcaagcggagccggctgctgcacagcgactacatgaacatgaccccaagacggcctggccccacccggaag | |
| | cactaccagccttacgcccctcccagagacttcgccgcctaccggtccagagtgaagttcagcagatccgccgacgcccctgccta | |
| | ccagcagggacagaaccagctgtacaacgagctgaacctgggcagacgggaagagtacgacgtgctggacaagcggagaggc | |
| | cgggaccccgagatgggcggaaagcccagacggaagaacccccaggaaggcctgtataacgaactgcagaaagacaagatgg | |
| | ccgaggcctacagcgagatcggcatgaagggcgagcggaggcgcggcaagggccacgatggcctgtaccagggcctgagca | |
| | ccgccaccaaggacacctacgacgccctgcacatgcaggccctgccccccagatgaaagcttgataatcaacctctggattacaaa | |
| | atttgtgaaagattgactggtattcttaactatgttgctccttttacgctatgtggatacgctgctttaatgcctttgtatcatgctattgcttcc | |
| | cgtatggctttcattttctcctccttgtataaatcctggttagttcttgccacggcggaactcatcgccgcctgccttgcccgctgctggac | |
| | aggggctcggctgttgggcactgacaattccgtggaacttgtttattgcagcttataatggttacaaataaagcaatagcatcacaaatt | |
| | tcacaaataaagcatttttttcactgcattctagttgtggtttgtccaaactcatcaatgtatcttacgccggcgtgaatgaggtctatggac | |
| | ttcaagagcaacagtgctgtggcctggagcaacaaatctgactttgcatgtgcaaacgccttcaacaacagcattattccagaagaca | |
| | ccttcttccccagcccaggtaagggcagctttggtgccttcgcaggctgtttccttgcttcaggaatggccaggttctgcccagagctc | |
| | tggtcaatgatgtctaaaactcctctgattggtggtctcggccttatccattgccaccaaaaccctctttttactaagaaacagtgagcctt | |
| | gttctggcagtccagagaatgacacgggaaaaaagcagatgaagagaaggtggcaggagagggcacgtggcccagcctcagtc | |
| | tctccaactgagttcctgcctgcctgcctttgccctaggaacccctagtgatggagttggccactccctctctgcgcgctcgctcgctc | |
| | actgaggccgggcgaccaaaggtcgcccgacgcccgggctttgcccgggcggcctcagtgagcgagcgagcgcgcagctgcc | |
| | tgcagg | |
| 22 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgcccaagattgatagcttgtg | #2 TRAC 2: |
| | cctgtccctgagtcccagtccatcacgagcagctggtttctaagatgctatttcccgtataaagcatgagaccgtgacttgccagcccc | HA TRAC |
| | acagagccccgcccttgtccatcactggcatctggactccagcctgggttggggcaaagagggaaatgagatcatgtcctaaccct | 2-2A- |
| | gatcctcttgtcccacagatatccagaaccctgaccctgccgtgtaccagctgagagactctaaatccagtgacaagtctgtctgccta | C11D5.3- |
| | ttcaccgattttgattctcaaacaaatgtgtcacaaagtaaggattctgatgtgtatatcacagacaaaactgtgctagacatgaggtcta | CD8-CD28- |
| | tggacttcaagagcaacagtgctgtgggttccggggagggccgagggtcattgctgacgtgtggagacgtggaggagaatcctgg | CD3z-HA |
| | ccccatggccctgcctgtgacagctctgctcctccctctggccctgctgctccatgccgccagacccgacatcgtgctgacccagag | TRAC 2 |
| | cccccccagcctggccatgtctctgggcaagagagccaccatcagctgccgggccagcgagagcgtgaccatcctgggcagcca | |
| | cctgatccactggtatcagcagaagcccggccagccccccaccctgctgatccagctcgccagcaatgtgcagaccggcgtgccc | |
| | gccagattcagcggcagcggcagcagaaccgacttcaccctgaccatcgaccccgtggaagaggacgacgtggccgtgtactac | |
| | tgcctgcagagccggaccatcccccggacctttggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcttcgtgcccgtgttcctgcccgccaaacctaccaccacccct | |
| | gcccctagacctcccaccccagccccaacaatcgccagccagcctctgtctctgcggcccgaagcctgtagacctgctgccggcg | |
| | gagccgtgcacaccagaggcctggacttcgcctgcgacatctacatctgggcccctctggccggcacctgtggcgtgctgctgctg | |
| | agcctggtgatcaccctgtactgcaaccaccggaacagaagcaagcggagccggctgctgcacagcgactacatgaacatgacc | |
| | ccaagacggcctggccccacccggaagcactaccagccttacgcccctcccagagacttcgccgcctaccggtccagagtgaagt | |
| | tcagcagatccgccgacgcccctgcctaccagcagggacagaaccagctgtacaacgagctgaacctgggcagacgggaagag | |
| | tacgacgtgctggacaagcggagaggccgggaccccgagatgggcggaaagcccagacggaagaacccccaggaaggcctg | |
| | tataacgaactgcagaaagacaagatggccgaggcctacagcgagatcggcatgaagggcgagcggaggcgcggcaagggcc | |
| | acgatggcctgtaccagggcctgagcaccgccaccaaggacacctacgacgccctgcacatgcaggccctgccccccagatgaa | |
| | agcttgataatcaacctctggattacaaaatttgtgaaagattgactggtattcttaactatgttgctccttttacgctatgtggatacgctg | |
| | ctttaatgcattgtatcatgctattgcttcccgtatggctttcattttctcctccttgtataaatcctggttagttcttgccacggcggaactca | |
| | tcgccgcctgccttgcccgctgctggacaggggctcggctgttgggcactgacaattccgtggaacttgtttattgcagcttataatgg | |
| | ttacaaataaagcaatagcatcacaaatttcacaaataaagcatttttttcactgcattctagttgtggtttgtccaaactcatcaatgtatct | |
| | tacgccggcgtgagcctggagcaacaaatctgactttgcatgtgcaaacgccttcaacaacagcattattccagaagacaccttcttc | |
| | cccagcccaggtaagggcagctttggtgccttcgcaggctgtttccttgcttcaggaatggccaggttctgcccagagctctggtcaa | |
| | tgatgtctaaaactcctctgattggtggtctcggccttatccattgccaccaaaaccctctttttactaagaaacagtgagccttgttctgg | |
| | cagtccagagaatgacacgggaaaaaagcagatgaagagaaggtggcaggagagggcacgtggcccagcctcagtctctccaa | |
| | ctgagttcctgcctgcctgcctttgctcagactgtttgccccttactgctcttctaggcctccctaggaacccctagtgatggagttggcc | |
| | actccctctctgcgcgctcgctcgctcactgaggccgggcgaccaaaggtcgcccgacgcccgggctttgcccgggcggcctca | |
| | gtgagcgagcgagcgcgcagctgcctgcagg | |
| 23 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggttcctgcggccgccccatgcctgcctttactct | #2 TRAC 3: |
| | gccagagttatattgctggggttttgaagaagatcctattaaataaaagaataagcagtattattaagtagccctgcatttcaggtacctt | HA TRAC |
| | gagtggcaggccaggcctggccgtgaacgttcactgaaatcatggcctcttggccaagattgatagcttgtgcctgtccctgagtccc | 3-2A- |
| | agtccatcacgagcagctggtttctaagatgctatttcccgtataaagcatgagaccgtgacttgccagccccacagagccccgccct | C11D5.3- |
| | tgtccatcactggcatctggactccagcctgggttggggcaaagagggaaatgagatcatgtcctaaccctgatcctcttgtcccaca | CD8-CD28- |
| | gatatccagaaccctgaccctgccggttccggggagggccgagggtcattgctgacgtgtggagacgtggaggagaatcctggcc | CD3z-HA |
| | ccatggccctgcctgtgacagctctgctcctccctctggccctgctgctccatgccgccagacccgacatcgtgctgacccagagcc | TRAC 3 |
| | cccccagcctggccatgtctctgggcaagagagccaccatcagctgccgggccagcgagagcgtgaccatcctgggcagccacc | |
| | tgatccactggtatcagcagaagcccggccagccccccaccctgctgatccagctcgccagcaatgtgcagaccggcgtgcccgc | |
| | cagattcagcggcagcggcagcagaaccgacttcaccctgaccatcgaccccgtggaagaggacgacgtggccgtgtactactg | |
| | cctgcagagccggaccatcccccggacctttggcggaggcaccaaactggaaatcaagggcagcaccagcggctccggcaagc | |
| | ctggctctggcgagggcagcacaaagggacagattcagctggtgcagagcggccctgagctgaagaaacccggcgagacagtg | |
| | aagatcagctgcaaggcctccggctacaccttcaccgactacagcatcaactgggtgaaaagagcccctggcaagggcctgaagt | |
| | ggatgggctggatcaacaccgagacaagagagcccgcctacgcctacgacttccggggcagattcgccttcagcctggaaacca | |
| | gcgccagcaccgcctacctgcagatcaacaacctgaagtacgaggacaccgccacctacttttgcgccctggactacagctacgcc | |
| | atggactactggggccagggcaccagcgtgaccgtgtccagcttcgtgcccgtgttcctgcccgccaaacctaccaccacccctgc | |
| | ccctagacctcccaccccagccccaacaatcgccagccagcctctgtctctgcggcccgaagcctgtagacctgctgccggcgga | |
| | gccgtgcacaccagaggcctggacttcgcctgcgacatctacatctgggcccctctggccggcacctgtggcgtgctgctgctgag | |
| | cctggtgatcaccctgtactgcaaccaccggaacagaagcaagcggagccggctgctgcacagcgactacatgaacatgacccc | |
| | aagacggcctggccccacccggaagcactaccagccttacgcccctcccagagacttcgccgcctaccggtccagagtgaagttc | |
| | agcagatccgccgacgcccctgcctaccagcagggacagaaccagctgtacaacgagctgaacctgggcagacgggaagagta | |
| | cgacgtgctggacaagcggagaggccgggaccccgagatgggcggaaagcccagacggaagaacccccaggaaggcctgta | |
| | taacgaactgcagaaagacaagatggccgaggcctacagcgagatcggcatgaagggcgagcggaggcgcggcaagggcca | |
| | cgatggcctgtaccagggcctgagcaccgccaccaaggacacctacgacgccctgcacatgcaggccctgccccccagatgaaa | |
| | gcttgataatcaacctctggattacaaaatttgtgaaagattgactggtattcttaactatgttgctccttttacgctatgtggatacgctgct | |
| | ttaatgcctagtatcatgctattgcttcccgtatggctttcattttctcctccttgtataaatcctggttagttcttgccacggcggaactcatc | |
| | gccgcctgccttgcccgctgctggacaggggctcggctgttgggcactgacaattccgtggaacttgtttattgcagcttataatggtt | |
| | acaaataaagcaatagcatcacaaatttcacaaataaagcatttttttcactgcattctagttgtggtttgtccaaactcatcaatgtatctta | |
| | cgccggcgtgagtgtaccagctgagagactctaaatccagtgacaagtctgtctgcctattcaccgattttgattctcaaacaaatgtgt | |
| | cacaaagtaaggattctgatgtgtatatcacagacaaaactgtgctagacatgaggtctatggacttcaagagcaacagtgctgtggc | |
| | ctggagcaacaaatctgactttgcatgtgcaaacgccttcaacaacagcattattccagaagacaccttcttccccagcccaggtaag | |
| | ggcagattggtgccttcgcaggctgtttccttgcttcaggaatggccaggttctgcccagagctctggtcaatgatgtctaaaactcct | |
| | ctgattggtggtctcggccttatccattgccaccaaaaccctctttttactaagacctaggaacccctagtgatggagttggccactccc | |
| | tctctgcgcgctcgctcgctcactgaggccgggcgaccaaaggtcgcccgacgcccgggctttgcccgggcggcctcagtgagc | |
| | gagcgagcgcgcagctgcctgcagg | |
| 24 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgctggccgtgaacgttcactg | #2 TRAC 1: |
| | aaatcatggcctcttggccaagattgatagcttgtgcctgtccctgagtcccagtccatcacgagcagctggtttctaagatgctatttcc | HA TRAC |
| | cgtataaagcatgagaccgtgacttgccagccccacagagccccgcccttgtccatcactggcatctggactccagcctgggagg | 1-2A- |
| | ggcaaagagggaaatgagatcatgtcctaaccctgatcctcttgtcccacagatatccagaaccctgaccctgccgtgtaccagctg | C11D5.3- |
| | agagactctaaatccagtgacaagtctgtctgcctattcaccgattttgattctcaaacaaatgtgtcacaaagtaaggattctgatgtgt | CD8-CD28- |
| | atatcacagacaaaactgtgctagacggttccggggagggccgagggtcattgctgacgtgtggagacgtggaggagaatcctgg | CD3z-HA |
| | ccccatggccctgcctgtgacagctctgctcctccctctggccctgctgctccatgccgccagacccgacatcgtgctgacccagag | TRAC 1 |
| | cccccccagcctggccatgtctctgggcaagagagccaccatcagctgccgggccagcgagagcgtgaccatcctgggcagcca | |
| | cctgatccactggtatcagcagaagcccggccagccccccaccctgctgatccagctcgccagcaatgtgcagaccggcgtgccc | |
| | gccagattcagcggcagcggcagcagaaccgacttcaccctgaccatcgaccccgtggaagaggacgacgtggccgtgtactac | |
| | tgcctgcagagccggaccatcccccggacctttggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcttcgtgcccgtgttcctgcccgccaaacctaccaccacccct | |
| | gcccctagacctcccaccccagccccaacaatcgccagccagcctctgtctctgcggcccgaagcctgtagacctgctgccggcg | |
| | gagccgtgcacaccagaggcctggacttcgcctgcgacatctacatctgggcccctctggccggcacctgtggcgtgctgctgctg | |
| | agcctggtgatcaccctgtactgcaaccaccggaacagaagcaagcggagccggctgctgcacagcgactacatgaacatgacc | |
| | ccaagacggcctggccccacccggaagcactaccagccttacgcccctcccagagacttcgccgcctaccggtccagagtgaagt | |
| | tcagcagatccgccgacgcccctgcctaccagcagggacagaaccagctgtacaacgagctgaacctgggcagacgggaagag | |
| | tacgacgtgctggacaagcggagaggccgggaccccgagatgggcggaaagcccagacggaagaacccccaggaaggcctg | |
| | tataacgaactgcagaaagacaagatggccgaggcctacagcgagatcggcatgaagggcgagcggaggcgcggcaagggcc | |
| | acgatggcctgtaccagggcctgagcaccgccaccaaggacacctacgacgccctgcacatgcaggccctgccccccagatgaa | |
| | agcttgataatcaacctctggattacaaaatttgtgaaagattgactggtattcttaactatgttgctccttttacgctatgtggatacgctg | |
| | attaatgcctttgtatcatgctattgcttcccgtatggctttcattttctcctccttgtataaatcctggttagttcttgccacggcggaactca | |
| | tcgccgcctgccttgcccgctgctggacaggggctcggctgttgggcactgacaattccgtggaacttgtttattgcagcttataatgg | |
| | ttacaaataaagcaatagcatcacaaatttcacaaataaagcatttttttcactgcattctagttgtggtttgtccaaactcatcaatgtatct | |
| | tacgccggcgtgaatgaggtctatggacttcaagagcaacagtgctgtggcctggagcaacaaatctgactttgcatgtgcaaacgc | |
| | cttcaacaacagcattattccagaagacaccttcttccccagcccaggtaagggcagctttggtgccttcgcaggctgtttccttgcttc | |
| | aggaatggccaggttctgcccagagctctggtcaatgatgtctaaaactcctctgattggtggtctcggccttatccattgccaccaaa | |
| | accctctttttactaagaaacagtgagccttgttctggcagtccagagaatgacacgggaaaaaagcagatgaagagaaggtggca | |
| | ggagagggcacgtggcccagcctcagtctctccaactgagttcctgcctgcctgcattgccctaggaacccctagtgatggagttg | |
| | gccactccctctctgcgcgctcgctcgctcactgaggccgggcgaccaaaggtcgcccgacgcccgggctttgcccgggcggcc | |
| | tcagtgagcgagcgagcgcgcagctgcctgcagg | |
| 25 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgcccaagattgatagcttgtg | #3 TRAC 2: |
| | cctgtccctgagtcccagtccatcacgagcagctggtttctaagatgctatttcccgtataaagcatgagaccgtgacttgccagcccc | HA TRAC |
| | acagagccccgcccttgtccatcactggcatctggactccagcctgggttggggcaaagagggaaatgagatcatgtcctaaccct | 2-2A- |
| | gatcctcttgtcccacagatatccagaaccctgaccctgccgtgtaccagctgagagactctaaatccagtgacaagtctgtctgccta | C11D5.3- |
| | ttcaccgattttgattctcaaacaaatgtgtcacaaagtaaggattctgatgtgtatatcacagacaaaactgtgctagacatgaggtcta | CD8-CD28- |
| | tggacttcaagagcaacagtgctgtgggttccggggagggccgagggtcattgctgacgtgtggagacgtggaggagaatcctgg | CD3z- |
| | ccccatggccctgcctgtgacagctctgctcctccctctggccctgctgctccatgccgccagacccgacatcgtgctgacccagag | CNb30-HA |
| | cccccccagcctggccatgtctctgggcaagagagccaccatcagctgccgggccagcgagagcgtgaccatcctgggcagcca | TRAC 2 |
| | cctgatccactggtatcagcagaagcccggccagccccccaccctgctgatccagctcgccagcaatgtgcagaccggcgtgccc | |
| | gccagattcagcggcagcggcagcagaaccgacttcaccctgaccatcgaccccgtggaagaggacgacgtggccgtgtactac | |
| | tgcctgcagagccggaccatcccccggacctttggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcttcgtgcccgtgttcctgcccgccaaacctaccaccacccct | |
| | gcccctagacctcccaccccagccccaacaatcgccagccagcctctgtctctgcggcccgaagcctgtagacctgctgccggcg | |
| | gagccgtgcacaccagaggcctggacttcgcctgcgacatctacatctgggcccctctggccggcacctgtggcgtgctgctgctg | |
| | agcctggtgatcaccctgtactgcaaccaccggaacagaagcaagcggagccggctgctgcacagcgactacatgaacatgacc | |
| | ccaagacggcctggccccacccggaagcactaccagccttacgcccctcccagagacttcgccgcctaccggtccagagtgaagt | |
| | tcagcagatccgccgacgcccctgcctaccagcagggacagaaccagctgtacaacgagctgaacctgggcagacgggaagag | |
| | tacgacgtgctggacaagcggagaggccgggaccccgagatgggcggaaagcccagacggaagaacccccaggaaggcctg | |
| | tataacgaactgcagaaagacaagatggccgaggcctacagcgagatcggcatgaagggcgagcggaggcgcggcaagggcc | |
| | acgatggcctgtaccagggcctgagcaccgccaccaaggacacctacgacgccctgcacatgcaggccctgccccccagaggc | |
| | agcggcgaaggcagaggatccctgcttacatgtggcgacgtggaagagaaccctggccccatgggcaacgaggccagctaccct | |
| | ctggagatgtgctcccacttcgacgccgacgagatcaagcggctgggcaagcgcttcaagaagctggacctggacaacagcggc | |
| | agcctgagcgtggaggagtttatgtctctgcccgagctgcagcagaaccccctggtgcagcgcgtgatcgacatcttcgacaccga | |
| | cggcaacggcgaggtggacttcaaggagttcatcgagggcgtgagccagttcagcgtgaagggcgacaaggagcagaagctgc | |
| | ggttcgccttccggatctacgatatggataaagatggctatatttctaatggcgagctgttccaggtgctgaagatgatggtgggcaac | |
| | aataccaagctggccgatacccagctgcagcagatcgtggacaagaccatcatcaacgccgacaaggacggcgacggcagaatc | |
| | agcttcgaggagttctgtgccgtggtgggaggcctggatattcacaaaaaaatggtggtggacgtgtgaaagcttgataatcaacctc | |
| | tggattacaaaatttgtgaaagattgactggtattcttaactatgttgctccttttacgctatgtggatacgctgctttaatgcctttgtatcat | |
| | gctattgcttcccgtatggctttcattttctcctccttgtataaatcctggttagttcttgccacggcggaactcatcgccgcctgccttgcc | |
| | cgctgctggacaggggctcggctgttgggcactgacaattccgtggaacttgtttattgcagcttataatggttacaaataaagcaata | |
| | gcatcacaaatttcacaaataaagcatttttttcactgcattctagttgtggtttgtccaaactcatcaatgtatcttacgccggcgtgagcc | |
| | tggagcaacaaatctgactttgcatgtgcaaacgccttcaacaacagcattattccagaagacaccttcttccccagcccaggtaagg | |
| | gcagctttggtgccttcgcaggctgtttccttgcttcaggaatggccaggttctgcccagagctctggtcaatgatgtctaaaactcctct | |
| | gattggtggtctcggccttatccattgccaccaaaaccctctttttactaagaaacagtgagccttgttctggcagtccagagaatgaca | |
| | cgggaaaaaagcagatgaagagaaggtggcaggagagggcacgtggcccagcctcagtctctccaactgagttcctgcctgcct | |
| | gcctttgctcagactgtttgccccttactgctcttctaggcctccctaggaacccctagtgatggagttggccactccctctctgcgcgct | |
| | cgctcgctcactgaggccgggcgaccaaaggtcgcccgacgcccgggctttgcccgggcggcctcagtgagcgagcgagcgc | |
| | gcagctgcctgcagg | |
| 26 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggttcctgcggccgccccatgcctgcctttactct | #3 TRAC 3: |
| | gccagagttatattgctggggttttgaagaagatcctattaaataaaagaataagcagtattattaagtagccctgcatttcaggtacctt | HA TRAC |
| | gagtggcaggccaggcctggccgtgaacgttcactgaaatcatggcctcttggccaagattgatagcttgtgcctgtccctgagtccc | 3-2A- |
| | agtccatcacgagcagctggtttctaagatgctatttcccgtataaagcatgagaccgtgacttgccagccccacagagccccgccct | C11D5.3- |
| | tgtccatcactggcatctggactccagcctgggttggggcaaagagggaaatgagatcatgtcctaaccctgatcctcttgtcccaca | CD8-CD28- |
| | gatatccagaaccctgaccctgccggttccggggagggccgagggtcattgctgacgtgtggagacgtggaggagaatcctggcc | CD3z- |
| | ccatggccctgcctgtgacagctctgctcctccctctggccctgctgctccatgccgccagacccgacatcgtgctgacccagagcc | CNb30-HA |
| | cccccagcctggccatgtctctgggcaagagagccaccatcagctgccgggccagcgagagcgtgaccatcctgggcagccacc | TRAC 3 |
| | tgatccactggtatcagcagaagcccggccagccccccaccctgctgatccagctcgccagcaatgtgcagaccggcgtgcccgc | |
| | cagattcagcggcagcggcagcagaaccgacttcaccctgaccatcgaccccgtggaagaggacgacgtggccgtgtactactg | |
| | cctgcagagccggaccatcccccggacctttggcggaggcaccaaactggaaatcaagggcagcaccagcggctccggcaagc | |
| | ctggctctggcgagggcagcacaaagggacagattcagctggtgcagagcggccctgagctgaagaaacccggcgagacagtg | |
| | aagatcagctgcaaggcctccggctacaccttcaccgactacagcatcaactgggtgaaaagagcccctggcaagggcctgaagt | |
| | ggatgggctggatcaacaccgagacaagagagcccgcctacgcctacgacttccggggcagattcgccttcagcctggaaacca | |
| | gcgccagcaccgcctacctgcagatcaacaacctgaagtacgaggacaccgccacctacttttgcgccctggactacagctacgcc | |
| | atggactactggggccagggcaccagcgtgaccgtgtccagcttcgtgcccgtgttcctgcccgccaaacctaccaccacccctgc | |
| | ccctagacctcccaccccagccccaacaatcgccagccagcctctgtctctgcggcccgaagcctgtagacctgctgccggcgga | |
| | gccgtgcacaccagaggcctggacttcgcctgcgacatctacatctgggcccctctggccggcacctgtggcgtgctgctgctgag | |
| | cctggtgatcaccctgtactgcaaccaccggaacagaagcaagcggagccggctgctgcacagcgactacatgaacatgacccc | |
| | aagacggcctggccccacccggaagcactaccagccttacgcccctcccagagacttcgccgcctaccggtccagagtgaagttc | |
| | agcagatccgccgacgcccctgcctaccagcagggacagaaccagctgtacaacgagctgaacctgggcagacgggaagagta | |
| | cgacgtgctggacaagcggagaggccgggaccccgagatgggcggaaagcccagacggaagaacccccaggaaggcctgta | |
| | taacgaactgcagaaagacaagatggccgaggcctacagcgagatcggcatgaagggcgagcggaggcgcggcaagggcca | |
| | cgatggcctgtaccagggcctgagcaccgccaccaaggacacctacgacgccctgcacatgcaggccctgccccccagaggca | |
| | gcggcgaaggcagaggatccctgcttacatgtggcgacgtggaagagaaccctggccccatgggcaacgaggccagctaccctc | |
| | tggagatgtgctcccacttcgacgccgacgagatcaagcggctgggcaagcgcttcaagaagctggacctggacaacagcggca | |
| | gcctgagcgtggaggagtttatgtctctgcccgagctgcagcagaaccccctggtgcagcgcgtgatcgacatcttcgacaccgac | |
| | ggcaacggcgaggtggacttcaaggagttcatcgagggcgtgagccagttcagcgtgaagggcgacaaggagcagaagctgcg | |
| | gttcgccttccggatctacgatatggataaagatggctatatttctaatggcgagctgttccaggtgctgaagatgatggtgggcaaca | |
| | ataccaagctggccgatacccagctgcagcagatcgtggacaagaccatcatcaacgccgacaaggacggcgacggcagaatca | |
| | gcttcgaggagttctgtgccgtggtgggaggcctggatattcacaaaaaaatggtggtggacgtgtgaaagcttgataatcaacctct | |
| | ggattacaaaatttgtgaaagattgactggtattcttaactatgttgctcatttacgctatgtggatacgctgctttaatgcctttgtatcatg | |
| | ctattgcttcccgtatggctttcattttctcctccttgtataaatcctggttagttcttgccacggcggaactcatcgccgcctgccttgccc | |
| | gctgctggacaggggctcggctgttgggcactgacaattccgtggaacttgtttattgcagcttataatggttacaaataaagcaatag | |
| | catcacaaatttcacaaataaagcatttttttcactgcattctagttgtggtttgtccaaactcatcaatgtatcttacgccggcgtgagtgt | |
| | accagctgagagactctaaatccagtgacaagtctgtctgcctattcaccgattttgattctcaaacaaatgtgtcacaaagtaaggatt | |
| | ctgatgtgtatatcacagacaaaactgtgctagacatgaggtctatggacttcaagagcaacagtgctgtggcctggagcaacaaatc | |
| | tgactttgcatgtgcaaacgccttcaacaacagcattattccagaagacaccttcttccccagcccaggtaagggcagctttggtgcct | |
| | tcgcaggctgtttccttgcttcaggaatggccaggttctgcccagagctctggtcaatgatgtctaaaactcctctgattggtggtctcg | |
| | gccttatccattgccaccaaaaccctctttttactaagacctaggaacccctagtgatggagttggccactccctctctgcgcgctcgct | |
| | cgctcactgaggccgggcgaccaaaggtcgcccgacgcccgggctttgcccgggcggcctcagtgagcgagcgagcgcgcag | |
| | ctgcctgcagg | |
| 27 | cctgcaggcagctgcgcgctcgctcgctcactgaggccgcccgggcaaagcccgggcgtcgggcgacctttggtcgcccggcct | TRAC AAV |
| | cagtgagcgagcgagcgcgcagagagggagtggccaactccatcactaggggttcctgcggccgctggccgtgaacgttcactg | #3 TRAC 1: |
| | aaatcatggcctcttggccaagattgatagcttgtgcctgtccctgagtcccagtccatcacgagcagctggtttctaagatgctatttcc | HA TRAC |
| | cgtataaagcatgagaccgtgacttgccagccccacagagccccgcccttgtccatcactggcatctggactccagcctgggttgg | 1-2A- |
| | ggcaaagagggaaatgagatcatgtcctaaccctgatcctcttgtcccacagatatccagaaccctgaccctgccgtgtaccagctg | C11D5.3- |
| | agagactctaaatccagtgacaagtctgtctgcctattcaccgattttgattctcaaacaaatgtgtcacaaagtaaggattctgatgtgt | CD8-CD28- |
| | atatcacagacaaaactgtgctagacggttccggggagggccgagggtcattgctgacgtgtggagacgtggaggagaatcctgg | CD3z- |
| | ccccatggccctgcctgtgacagctctgctcctccctctggccctgctgctccatgccgccagacccgacatcgtgctgacccagag | CNb30-HA |
| | cccccccagcctggccatgtctctgggcaagagagccaccatcagctgccgggccagcgagagcgtgaccatcctgggcagcca | TRAC 1 |
| | cctgatccactggtatcagcagaagcccggccagccccccaccctgctgatccagctcgccagcaatgtgcagaccggcgtgccc | |
| | gccagattcagcggcagcggcagcagaaccgacttcaccctgaccatcgaccccgtggaagaggacgacgtggccgtgtactac | |
| | tgcctgcagagccggaccatcccccggacctttggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcttcgtgcccgtgttcctgcccgccaaacctaccaccacccct | |
| | gcccctagacctcccaccccagccccaacaatcgccagccagcctctgtctctgcggcccgaagcctgtagacctgctgccggcg | |
| | gagccgtgcacaccagaggcctggacttcgcctgcgacatctacatctgggcccctctggccggcacctgtggcgtgctgctgctg | |
| | agcctggtgatcaccctgtactgcaaccaccggaacagaagcaagcggagccggctgctgcacagcgactacatgaacatgacc | |
| | ccaagacggcctggccccacccggaagcactaccagccttacgcccctcccagagacttcgccgcctaccggtccagagtgaagt | |
| | tcagcagatccgccgacgcccctgcctaccagcagggacagaaccagctgtacaacgagctgaacctgggcagacgggaagag | |
| | tacgacgtgctggacaagcggagaggccgggaccccgagatgggcggaaagcccagacggaagaacccccaggaaggcctg | |
| | tataacgaactgcagaaagacaagatggccgaggcctacagcgagatcggcatgaagggcgagcggaggcgcggcaagggcc | |
| | acgatggcctgtaccagggcctgagcaccgccaccaaggacacctacgacgccctgcacatgcaggccctgccccccagaggc | |
| | agcggcgaaggcagaggatccctgcttacatgtggcgacgtggaagagaaccctggccccatgggcaacgaggccagctaccct | |
| | ctggagatgtgctcccacttcgacgccgacgagatcaagcggctgggcaagcgcttcaagaagctggacctggacaacagcggc | |
| | agcctgagcgtggaggagtttatgtctctgcccgagctgcagcagaaccccctggtgcagcgcgtgatcgacatcttcgacaccga | |
| | cggcaacggcgaggtggacttcaaggagttcatcgagggcgtgagccagttcagcgtgaagggcgacaaggagcagaagctgc | |
| | ggttcgccttccggatctacgatatggataaagatggctatatttctaatggcgagctgttccaggtgctgaagatgatggtgggcaac | |
| | aataccaagctggccgatacccagctgcagcagatcgtggacaagaccatcatcaacgccgacaaggacggcgacggcagaatc | |
| | agcttcgaggagttctgtgccgtggtgggaggcctggatattcacaaaaaaatggtggtggacgtgtgaaagcttgataatcaacctc | |
| | tggattacaaaatttgtgaaagattgactggtattcttaactatgttgctccttttacgctatgtggatacgctgctttaatgcctttgtatcat | |
| | gctattgcttcccgtatggctttcattttctcctccttgtataaatcctggttagttcttgccacggcggaactcatcgccgcctgccttgcc | |
| | cgctgctggacaggggctcggctgttgggcactgacaattccgtggaacttgtttattgcagcttataatggttacaaataaagcaata | |
| | gcatcacaaatttcacaaataaagcatttttttcactgcattctagttgtggtttgtccaaactcatcaatgtatcttacgccggcgtgaatg | |
| | aggtctatggacttcaagagcaacagtgctgtggcctggagcaacaaatctgactttgcatgtgcaaacgccttcaacaacagcatta | |
| | ttccagaagacaccttcttccccagcccaggtaagggcagctttggtgccttcgcaggctgtttccttgcttcaggaatggccaggttc | |
| | tgcccagagctctggtcaatgatgtctaaaactcctctgattggtggtctcggccttatccattgccaccaaaaccctctttttactaaga | |
| | aacagtgagccttgactggcagtccagagaatgacacgggaaaaaagcagatgaagagaaggtggcaggagagggcacgtgg | |
| | cccagcctcagtctctccaactgagacctgcctgcctgcctttgccctaggaacccctagtgatggagttggccactccctctctgcg | |
| | cgctcgctcgctcactgaggccgggcgaccaaaggtcgcccgacgcccgggctttgcccgggcggcctcagtgagcgagcgag | |
| | cgcgcagctgcctgcagg | |
| 28 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgcccaagattgatagcttgtg | #4 TRAC 2: |
| | cctgtccctgagtcccagtccatcacgagcagctggatctaagatgctatttcccgtataaagcatgagaccgtgacttgccagcccc | HA TRAC |
| | acagagccccgcccttgtccatcactggcatctggactccagcctgggttggggcaaagagggaaatgagatcatgtcctaaccct | 2-C11D5.3- |
| | gatcctcttgtcccacagatatccagaaccctgaccctgccgtgtaccagctgagagactctaaatccagtgacaagtctgtctgccta | CD8-CD28- |
| | ttcaccgattttgattctcaaacaaatgtgtcacaaagtaaggattctgatgtgtatatcacagacaaaactgtgctagacatgaggtcta | CD3z-P2A- |
| | tggacttcaagagcaacagtgctgtgtgaatgaatgattaattaataaaagatctttattttcattagatctgtgtgttggttttttgtgtgatc | CISCβ-HA |
| | ctcgagggaatgaaagaccccacctgtaggtttggcaagctagcttaagtaacgccattagcaaggcatggaaaatacataactga | TRAC 2 |
| | gaatagagaagttcagatcaaggttaggaacagagagacagcagaatatgggccaaacaggatatctgtggtaagcagacctgcc | |
| | ccggctcagggccaagaacagatggtccccagatgcggtcccgccctcagcagtttctagagaaccatcagatgtttccagggtgc | |
| | cccaaggacctgaaaatgaccctgtgccttatttgaactaaccaatcagttcgcttctcgcttctgttcgcgcgcttctgctccccgagct | |
| | caataaaagagcccacaacccctcactcggcgcgcgccagtccggtaccagtcgccaccatggccctgcctgtgacagctctgct | |
| | cctccctctggccctgctgctccatgccgccagacccgacatcgtgctgacccagagcccccccagcctggccatgtctctgggca | |
| | agagagccaccatcagctgccgggccagcgagagcgtgaccatcctgggcagccacctgatccactggtatcagcagaagcccg | |
| | gccagccccccaccctgctgatccagctcgccagcaatgtgcagaccggcgtgcccgccagattcagcggcagcggcagcaga | |
| | accgacttcaccctgaccatcgaccccgtggaagaggacgacgtggccgtgtactactgcctgcagagccggaccatcccccgga | |
| | cctttggcggaggcaccaaactggaaatcaagggcagcaccagcggctccggcaagcctggctctggcgagggcagcacaaag | |
| | ggacagattcagctggtgcagagcggccctgagctgaagaaacccggcgagacagtgaagatcagctgcaaggcctccggctac | |
| | accttcaccgactacagcatcaactgggtgaaaagagcccctggcaagggcctgaagtggatgggctggatcaacaccgagaca | |
| | agagagcccgcctacgcctacgacttccggggcagattcgccttcagcctggaaaccagcgccagcaccgcctacctgcagatca | |
| | acaacctgaagtacgaggacaccgccacctacttagcgccctggactacagctacgccatggactactggggccagggcaccag | |
| | cgtgaccgtgtccagcttcgtgcccgtgacctgcccgccaaacctaccaccacccctgcccctagacctcccaccccagccccaa | |
| | caatcgccagccagcctctgtctctgcggcccgaagcctgtagacctgctgccggcggagccgtgcacaccagaggcctggactt | |
| | cgcctgcgacatctacatctgggcccctctggccggcacctgtggcgtgctgctgctgagcctggtgatcaccctgtactgcaacca | |
| | ccggaacagaagcaagcggagccggctgctgcacagcgactacatgaacatgaccccaagacggcctggccccacccggaag | |
| | cactaccagccttacgcccctcccagagacttcgccgcctaccggtccagagtgaagttcagcagatccgccgacgcccctgccta | |
| | ccagcagggacagaaccagctgtacaacgagctgaacctgggcagacgggaagagtacgacgtgctggacaagcggagaggc | |
| | cgggaccccgagatgggcggaaagcccagacggaagaacccccaggaaggcctgtataacgaactgcagaaagacaagatgg | |
| | ccgaggcctacagcgagatcggcatgaagggcgagcggaggcgcggcaagggccacgatggcctgtaccagggcctgagca | |
| | ccgccaccaaggacacctacgacgccctgcacatgcaggccctgccccccagaggatccggcgctacaaattatcactgctgaaa | |
| | caggcgggtgatgtggaggagaaccctggacccatgccacttggcctgctctggctgggcttggcattgctcggcgcgctccacg | |
| | cccaggctgaactgatccgcgtggccatattgtggcatgagatgtggcatgagggattggaggaggcgagtaggctgtactttggg | |
| | gaaaggaatgttaaagggatgtttgaggtccttgaacccctccacgctatgatggaaagaggacctcaaacgcttaaagagacgtca | |
| | ttcaatcaagcctatggacgggatcttatggaagctcaagaatggtgtcgaaaatacatgaaaagcgggaatgttaaggacctcacg | |
| | caagcctgggatctgtattaccatgttttccgacgcatttctaaacaaggaaaagatactatcccatggttggggcacttgctcgttggg | |
| | ctcagtggggcgtttggattcatcatcctcgtatatctgttgattaattgtcggaacacaggtccctggcttaaaaaagttttgaagtgtaa | |
| | caccccggatccttctaaattttttagtcaacttagttcagaacacgggggcgatgttcaaaagtggctgagttccccgtttcccagttca | |
| | agtttctcccctgggggtctcgcccccgagatatcacctcttgaagtgctcgagcgggacaaagttacacagcttcttttgcaacagg | |
| | ataaggttccggagccggcgtctctcagctctaaccattcactcacttcttgtttcaccaaccaagggtattttttcttccatctgcctgatg | |
| | ccttggagattgaggcttgtcaggtgtactttacctatgacccctatagtgaggaagaccctgacgaaggcgtagctggcgccccca | |
| | ctggctccagtccacagcctcttcagcctctgtcaggggaggacgacgcatattgtacgttcccctcacgggacgaccttctgctgttt | |
| | tcaccctcactgctcggcggaccctccccgccaagcacggcacctggggggagtggggcaggagaagaaaggatgcctcctagt | |
| | ttgcaggagcgggacctcgcgactgggatccgcaacccctcggaccacccacccctggcgtacctgatctggtcgacttccaacc | |
| | acctccggagcttgtcctcagagaggccggagaggaagtcccagacgcggggccaagagagggtgtgtcatttccctggtcccg | |
| | ccctccgggacagggtgagtttcgggcgctgaatgcgaggctcccccttaataccgatgcgtacctgtcattgcaggaacttcaggg | |
| | ccaggatcctacccacctggtgtgaaagcttgataatcaacctctggattacaaaatttgtgaaagattgactggtattcttaactatgttg | |
| | ctccttttacgctatgtggatacgctgctttaatgcctttgtatcatgctattgcttcccgtatggctttcattttctcctccttgtataaatcctg | |
| | gttagttcttgccacggcggaactcatcgccgcctgccttgcccgctgctggacaggggctcggctgttgggcactgacaattccgt | |
| | ggaacttgtttattgcagcttataatggttacaaataaagcaatagcatcacaaatttcacaaataaagcatttttttcactgcattctagttg | |
| | tggtttgtccaaactcatcaatgtatcttacgccggcgtgagcctggagcaacaaatctgactttgcatgtgcaaacgccttcaacaac | |
| | agcattattccagaagacaccttcttccccagcccaggtaagggcagctttggtgccttcgcaggctgtttccttgcttcaggaatggc | |
| | caggttctgcccagagctctggtcaatgatgtctaaaactcctctgattggtggtctcggccttatccattgccaccaaaaccctcttttta | |
| | ctaagaaacagtgagccttgttctggcagtccagagaatgacacgggaaaaaagcagatgaagagaaggtggcaggagagggc | |
| | acgtggcccagcctcagtctctccaactgagttcctgcctgcctgcctttgctcagactgtttgccccttactgctcttctaggcctccct | |
| | aggaacccctagtgatggagttggccactccctctctgcgcgctcgctcgctcactgaggccgggcgaccaaaggtcgcccgacg | |
| | cccgggctttgcccgggcggcctcagtgagcgagcgagcgcgcagctgcctgcagg | |
| 29 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgccccatgcctgcctttactct | #4 TRAC 3: |
| | gccagagttatattgctggggttttgaagaagatcctattaaataaaagaataagcagtattattaagtagccctgcatttcaggtttcctt | HA TRAC |
| | gagtggcaggccaggcctggccgtgaacgttcactgaaatcatggcctcttggccaagattgatagcttgtgcctgtccctgagtccc | 3-C11D5.3- |
| | agtccatcacgagcagctggtttctaagatgctatttcccgtataaagcatgagaccgtgacttgccagccccacagagccccgccct | CD8-CD28- |
| | tgtccatcactggcatctggactccagcctgggttggggcaaagagggaaatgagatcatgtcctaaccctgatcctcttgtcccaca | CD3z-P2A- |
| | gatatccagaaccctgaccctgcctgaatgaatgattaattaataaaagatctttattttcattagatctgtgtgttggttttttgtgtgatcct | CISCβ-HA |
| | cgagggaatgaaagaccccacctgtaggtttggcaagctagcttaagtaacgccattttgcaaggcatggaaaatacataactgaga | TRAC 3 |
| | atagagaagttcagatcaaggttaggaacagagagacagcagaatatgggccaaacaggatatctgtggtaagcagttcctgcccc | |
| | ggctcagggccaagaacagatggtccccagatgcggtcccgccctcagcagtttctagagaaccatcagatgtttccagggtgccc | |
| | caaggacctgaaaatgaccctgtgccttatttgaactaaccaatcagttcgcttctcgcttctgttcgcgcgcttctgctccccgagctca | |
| | ataaaagagcccacaacccctcactcggcgcgcgccagtccggtaccagtcgccaccatggccctgcctgtgacagctctgctcct | |
| | ccctctggccctgctgctccatgccgccagacccgacatcgtgctgacccagagcccccccagcctggccatgtctctgggcaaga | |
| | gagccaccatcagctgccgggccagcgagagcgtgaccatcctgggcagccacctgatccactggtatcagcagaagcccggcc | |
| | agccccccaccctgctgatccagctcgccagcaatgtgcagaccggcgtgcccgccagattcagcggcagcggcagcagaacc | |
| | gacttcaccctgaccatcgaccccgtggaagaggacgacgtggccgtgtactactgcctgcagagccggaccatcccccggacct | |
| | ttggcggaggcaccaaactggaaatcaagggcagcaccagcggctccggcaagcctggctctggcgagggcagcacaaaggg | |
| | acagattcagctggtgcagagcggccctgagctgaagaaacccggcgagacagtgaagatcagctgcaaggcctccggctacac | |
| | cttcaccgactacagcatcaactgggtgaaaagagcccctggcaagggcctgaagtggatgggctggatcaacaccgagacaag | |
| | agagcccgcctacgcctacgacttccggggcagattcgccttcagcctggaaaccagcgccagcaccgcctacctgcagatcaac | |
| | aacctgaagtacgaggacaccgccacctacttttgcgccctggactacagctacgccatggactactggggccagggcaccagcg | |
| | tgaccgtgtccagcttcgtgcccgtgttcctgcccgccaaacctaccaccacccctgcccctagacctcccaccccagccccaacaa | |
| | tcgccagccagcctctgtctctgcggcccgaagcctgtagacctgctgccggcggagccgtgcacaccagaggcctggacttcgc | |
| | ctgcgacatctacatctgggcccctctggccggcacctgtggcgtgctgctgctgagcctggtgatcaccctgtactgcaaccaccg | |
| | gaacagaagcaagcggagccggctgctgcacagcgactacatgaacatgaccccaagacggcctggccccacccggaagcact | |
| | accagccttacgcccctcccagagacttcgccgcctaccggtccagagtgaagttcagcagatccgccgacgcccctgcctacca | |
| | gcagggacagaaccagctgtacaacgagctgaacctgggcagacgggaagagtacgacgtgctggacaagcggagaggccgg | |
| | gaccccgagatgggcggaaagcccagacggaagaacccccaggaaggcctgtataacgaactgcagaaagacaagatggccg | |
| | aggcctacagcgagatcggcatgaagggcgagcggaggcgcggcaagggccacgatggcctgtaccagggcctgagcaccg | |
| | ccaccaaggacacctacgacgccctgcacatgcaggccctgccccccagaggatccggcgctacaaatttttcactgctgaaacag | |
| | gcgggtgatgtggaggagaaccctggacccatgccacttggcctgctctggctgggcttggcattgctcggcgcgctccacgccca | |
| | ggctgaactgatccgcgtggccatattgtggcatgagatgtggcatgagggattggaggaggcgagtaggctgtactttggggaaa | |
| | ggaatgttaaagggatgtttgaggtccttgaacccctccacgctatgatggaaagaggacctcaaacgcttaaagagacgtcattcaa | |
| | tcaagcctatggacgggatcttatggaagctcaagaatggtgtcgaaaatacatgaaaagcgggaatgttaaggacctcacgcaag | |
| | cctgggatctgtattaccatgttttccgacgcatttctaaacaaggaaaagatactatcccatggttggggcacttgctcgttgggctca | |
| | gtggggcgtttggattcatcatcctcgtatatctgttgattaattgtcggaacacaggtccctggcttaaaaaagttttgaagtgtaacac | |
| | cccggatccttctaaattttttagtcaacttagttcagaacacgggggcgatgttcaaaagtggctgagttccccgtttcccagttcaagt | |
| | ttctcccctgggggtctcgcccccgagatatcacctcttgaagtgctcgagcgggacaaagttacacagcttcttttgcaacaggataa | |
| | ggttccggagccggcgtctctcagctctaaccattcactcacttcttgtttcaccaaccaagggtattttttcttccatctgcctgatgcctt | |
| | ggagattgaggcttgtcaggtgtactttacctatgacccctatagtgaggaagaccctgacgaaggcgtagctggcgcccccactgg | |
| | ctccagtccacagcctcttcagcctctgtcaggggaggacgacgcatattgtacgttcccctcacgggacgaccttctgctgttttcac | |
| | cctcactgctcggcggaccctccccgccaagcacggcacctggggggagtggggcaggagaagaaaggatgcctcctagtttgc | |
| | aggagcgggttcctcgcgactgggatccgcaacccctcggaccacccacccctggcgtacctgatctggtcgacttccaaccacct | |
| | ccggagcttgtcctcagagaggccggagaggaagtcccagacgcggggccaagagagggtgtgtcatttccctggtcccgccct | |
| | ccgggacagggtgagtttcgggcgctgaatgcgaggctcccccttaataccgatgcgtacctgtcattgcaggaacttcagggcca | |
| | ggatcctacccacctggtgtgaaagcttgataatcaacctctggattacaaaatttgtgaaagattgactggtattcttaactatgttgctc | |
| | atttacgctatgtggatacgctgctttaatgcctttgtatcatgctattgcttcccgtatggctttcattttctcctccttgtataaatcctggtt | |
| | agttcttgccacggcggaactcatcgccgcctgccttgcccgctgctggacaggggctcggctgttgggcactgacaattccgtgga | |
| | acttgtttattgcagcttataatggttacaaataaagcaatagcatcacaaatttcacaaataaagcatttttttcactgcattctagttgtgg | |
| | tttgtccaaactcatcaatgtatcttacgccggcgtgagtgtaccagctgagagactctaaatccagtgacaagtctgtctgcctattca | |
| | ccgattttgattctcaaacaaatgtgtcacaaagtaaggattctgatgtgtatatcacagacaaaactgtgctagacatgaggtctatgg | |
| | acttcaagagcaacagtgctgtggcctggagcaacaaatctgactttgcatgtgcaaacgccttcaacaacagcattattccagaaga | |
| | caccttcttccccagcccaggtaagggcagctttggtgccttcgcaggctgtttccttgcttcaggaatggccaggttctgcccagagc | |
| | tctggtcaatgatgtctaaaactcctctgattggtggtctcggccttatccattgccaccaaaaccctctttttactaagacctaggaaccc | |
| | ctagtgatggagaggccactccctctctgcgcgctcgctcgctcactgaggccgggcgaccaaaggtcgcccgacgcccgggctt | |
| | tgcccgggcggcctcagtgagcgagcgagcgcgcagctgcctgcagg | |
| 30 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgctggccgtgaacgttcactg | #4 TRAC 1: |
| | aaatcatggcctcttggccaagattgatagcttgtgcctgtccctgagtcccagtccatcacgagcagctggtttctaagatgctatttcc | HA TRAC |
| | cgtataaagcatgagaccgtgacttgccagccccacagagccccgcccttgtccatcactggcatctggactccagcctgggttgg | 1-C11D5.3- |
| | ggcaaagagggaaatgagatcatgtcctaaccctgatcctcttgtcccacagatatccagaaccctgaccctgccgtgtaccagctg | CD8-CD28- |
| | agagactctaaatccagtgacaagtctgtctgcctattcaccgattagattctcaaacaaatgtgtcacaaagtaaggattctgatgtgt | CD3z-P2A- |
| | atatcacagacaaaactgtgctagactgaatgaatgattaattaataaaagatctttattttcattagatctgtgtgttggttttttgtgtgatc | CISCβ-HA |
| | ctcgagggaatgaaagaccccacctgtaggtttggcaagctagcttaagtaacgccattagcaaggcatggaaaatacataactga | TRAC 1 |
| | gaatagagaagttcagatcaaggttaggaacagagagacagcagaatatgggccaaacaggatatctgtggtaagcagacctgcc | |
| | ccggctcagggccaagaacagatggtccccagatgcggtcccgccctcagcagtttctagagaaccatcagatgtttccagggtgc | |
| | cccaaggacctgaaaatgaccctgtgccttatttgaactaaccaatcagttcgcttctcgcttctgttcgcgcgcttctgctccccgagct | |
| | caataaaagagcccacaacccctcactcggcgcgcgccagtccggtaccagtcgccaccatggccctgcctgtgacagctctgct | |
| | cctccctctggccctgctgctccatgccgccagacccgacatcgtgctgacccagagcccccccagcctggccatgtctctgggca | |
| | agagagccaccatcagctgccgggccagcgagagcgtgaccatcctgggcagccacctgatccactggtatcagcagaagcccg | |
| | gccagccccccaccctgctgatccagctcgccagcaatgtgcagaccggcgtgcccgccagattcagcggcagcggcagcaga | |
| | accgacttcaccctgaccatcgaccccgtggaagaggacgacgtggccgtgtactactgcctgcagagccggaccatcccccgga | |
| | cctttggcggaggcaccaaactggaaatcaagggcagcaccagcggctccggcaagcctggctctggcgagggcagcacaaag | |
| | ggacagattcagctggtgcagagcggccctgagctgaagaaacccggcgagacagtgaagatcagctgcaaggcctccggctac | |
| | accttcaccgactacagcatcaactgggtgaaaagagcccctggcaagggcctgaagtggatgggctggatcaacaccgagaca | |
| | agagagcccgcctacgcctacgacttccggggcagattcgccttcagcctggaaaccagcgccagcaccgcctacctgcagatca | |
| | acaacctgaagtacgaggacaccgccacctacttagcgccctggactacagctacgccatggactactggggccagggcaccag | |
| | cgtgaccgtgtccagcttcgtgcccgtgacctgcccgccaaacctaccaccacccctgcccctagacctcccaccccagccccaa | |
| | caatcgccagccagcctctgtctctgcggcccgaagcctgtagacctgctgccggcggagccgtgcacaccagaggcctggactt | |
| | cgcctgcgacatctacatctgggcccctctggccggcacctgtggcgtgctgctgctgagcctggtgatcaccctgtactgcaacca | |
| | ccggaacagaagcaagcggagccggctgctgcacagcgactacatgaacatgaccccaagacggcctggccccacccggaag | |
| | cactaccagccttacgcccctcccagagacttcgccgcctaccggtccagagtgaagttcagcagatccgccgacgcccctgccta | |
| | ccagcagggacagaaccagctgtacaacgagctgaacctgggcagacgggaagagtacgacgtgctggacaagcggagaggc | |
| | cgggaccccgagatgggcggaaagcccagacggaagaacccccaggaaggcctgtataacgaactgcagaaagacaagatgg | |
| | ccgaggcctacagcgagatcggcatgaagggcgagcggaggcgcggcaagggccacgatggcctgtaccagggcctgagca | |
| | ccgccaccaaggacacctacgacgccctgcacatgcaggccctgccccccagaggatccggcgctacaaattatcactgctgaaa | |
| | caggcgggtgatgtggaggagaaccctggacccatgccacttggcctgctctggctgggcttggcattgctcggcgcgctccacg | |
| | cccaggctgaactgatccgcgtggccatattgtggcatgagatgtggcatgagggattggaggaggcgagtaggctgtactttggg | |
| | gaaaggaatgttaaagggatgtttgaggtccttgaacccctccacgctatgatggaaagaggacctcaaacgcttaaagagacgtca | |
| | ttcaatcaagcctatggacgggatcttatggaagctcaagaatggtgtcgaaaatacatgaaaagcgggaatgttaaggacctcacg | |
| | caagcctgggatctgtattaccatgttttccgacgcatttctaaacaaggaaaagatactatcccatggttggggcacttgctcgttggg | |
| | ctcagtggggcgtttggattcatcatcctcgtatatctgttgattaattgtcggaacacaggtccctggcttaaaaaagttttgaagtgtaa | |
| | caccccggatccttctaaattttttagtcaacttagttcagaacacgggggcgatgttcaaaagtggctgagttccccgtttcccagttca | |
| | agtttctcccctgggggtctcgcccccgagatatcacctcttgaagtgctcgagcgggacaaagttacacagcttcttttgcaacagg | |
| | ataaggttccggagccggcgtctctcagctctaaccattcactcacttcttgtttcaccaaccaagggtattttttcttccatctgcctgatg | |
| | ccttggagattgaggcttgtcaggtgtactttacctatgacccctatagtgaggaagaccctgacgaaggcgtagctggcgccccca | |
| | ctggctccagtccacagcctcttcagcctctgtcaggggaggacgacgcatattgtacgttcccctcacgggacgaccttctgctgttt | |
| | tcaccctcactgctcggcggaccctccccgccaagcacggcacctggggggagtggggcaggagaagaaaggatgcctcctagt | |
| | ttgcaggagcgggacctcgcgactgggatccgcaacccctcggaccacccacccctggcgtacctgatctggtcgacttccaacc | |
| | acctccggagcttgtcctcagagaggccggagaggaagtcccagacgcggggccaagagagggtgtgtcatttccctggtcccg | |
| | ccctccgggacagggtgagtttcgggcgctgaatgcgaggctcccccttaataccgatgcgtacctgtcattgcaggaacttcaggg | |
| | ccaggatcctacccacctggtgtgaaagcttgataatcaacctctggattacaaaatttgtgaaagattgactggtattcttaactatgttg | |
| | ctccttttacgctatgtggatacgctgctttaatgcctttgtatcatgctattgcttcccgtatggctttcattttctcctccttgtataaatcctg | |
| | gttagttcttgccacggcggaactcatcgccgcctgccttgcccgctgctggacaggggctcggctgttgggcactgacaattccgt | |
| | ggaacttgtttattgcagcttataatggttacaaataaagcaatagcatcacaaatttcacaaataaagcatttttttcactgcattctagttg | |
| | tggtagtccaaactcatcaatgtatcttacgccggcgtgaatgaggtctatggacttcaagagcaacagtgctgtggcctggagcaac | |
| | aaatctgactttgcatgtgcaaacgccttcaacaacagcattattccagaagacaccttcttccccagcccaggtaagggcagattgg | |
| | tgccttcgcaggctgtttccttgcttcaggaatggccaggttctgcccagagctctggtcaatgatgtctaaaactcctctgattggtggt | |
| | ctcggccttatccattgccaccaaaaccctctttttactaagaaacagtgagccttgttctggcagtccagagaatgacacgggaaaaa | |
| | agcagatgaagagaaggtggcaggagagggcacgtggcccagcctcagtctctccaactgagttcctgcctgcctgcctttgccct | |
| | aggaacccctagtgatggagttggccactccctctctgcgcgctcgctcgctcactgaggccgggcgaccaaaggtcgcccgacg | |
| | cccgggctttgcccgggcggcctcagtgagcgagcgagcgcgcagctgcctgcagg | |
| 31 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgcccaagattgatagcttgtg | #5 TRAC 2: |
| | cctgtccctgagtcccagtccatcacgagcagctggtttctaagatgctatttcccgtataaagcatgagaccgtgacttgccagcccc | HA TRAC |
| | acagagccccgcccttgtccatcactggcatctggactccagcctgggttggggcaaagagggaaatgagatcatgtcctaaccct | 2-2A- |
| | gatcctcttgtcccacagatatccagaaccctgaccctgccgtgtaccagctgagagactctaaatccagtgacaagtctgtctgccta | C11D5.3- |
| | ttcaccgattttgattctcaaacaaatgtgtcacaaagtaaggattctgatgtgtatatcacagacaaaactgtgctagacatgaggtcta | CD8-CD28- |
| | tggacttcaagagcaacagtgctgtgggttccggggagggccgagggtcattgctgacgtgtggagacgtggaggagaatcctgg | CD3z- |
| | ccccatggccctgcctgtgacagctctgctcctccctctggccctgctgctccatgccgccagacccgacatcgtgctgacccagag | CISCβ-HA |
| | cccccccagcctggccatgtctctgggcaagagagccaccatcagctgccgggccagcgagagcgtgaccatcctgggcagcca | TRAC 2 |
| | cctgatccactggtatcagcagaagcccggccagccccccaccctgctgatccagctcgccagcaatgtgcagaccggcgtgccc | |
| | gccagattcagcggcagcggcagcagaaccgacttcaccctgaccatcgaccccgtggaagaggacgacgtggccgtgtactac | |
| | tgcctgcagagccggaccatcccccggacctttggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcttcgtgcccgtgttcctgcccgccaaacctaccaccacccct | |
| | gcccctagacctcccaccccagccccaacaatcgccagccagcctctgtctctgcggcccgaagcctgtagacctgctgccggcg | |
| | gagccgtgcacaccagaggcctggacttcgcctgcgacatctacatctgggcccctctggccggcacctgtggcgtgctgctgctg | |
| | agcctggtgatcaccctgtactgcaaccaccggaacagaagcaagcggagccggctgctgcacagcgactacatgaacatgacc | |
| | ccaagacggcctggccccacccggaagcactaccagccttacgcccctcccagagacttcgccgcctaccggtccagagtgaagt | |
| | tcagcagatccgccgacgcccctgcctaccagcagggacagaaccagctgtacaacgagctgaacctgggcagacgggaagag | |
| | tacgacgtgctggacaagcggagaggccgggaccccgagatgggcggaaagcccagacggaagaacccccaggaaggcctg | |
| | tataacgaactgcagaaagacaagatggccgaggcctacagcgagatcggcatgaagggcgagcggaggcgcggcaagggcc | |
| | acgatggcctgtaccagggcctgagcaccgccaccaaggacacctacgacgccctgcacatgcaggccctgccccccagaggat | |
| | ccggcgctacaaatttttcactgctgaaacaggcgggtgatgtggaggagaaccctggacccatgccacttggcctgctctggctgg | |
| | gcttggcattgctcggcgcgctccacgcccaggctgaactgatccgcgtggccatattgtggcatgagatgtggcatgagggattgg | |
| | aggaggcgagtaggctgtactttggggaaaggaatgttaaagggatgtttgaggtccttgaacccctccacgctatgatggaaagag | |
| | gacctcaaacgcttaaagagacgtcattcaatcaagcctatggacgggatcttatggaagctcaagaatggtgtcgaaaatacatgaa | |
| | aagcgggaatgttaaggacctcacgcaagcctgggatctgtattaccatgttttccgacgcatttctaaacaaggaaaagatactatcc | |
| | catggttggggcacttgctcgttgggctcagtggggcgtttggattcatcatcctcgtatatctgttgattaattgtcggaacacaggtcc | |
| | ctggcttaaaaaagttttgaagtgtaacaccccggatccttctaaattttttagtcaacttagttcagaacacgggggcgatgttcaaaag | |
| | tggctgagttccccgtttcccagttcaagtttctcccctgggggtctcgcccccgagatatcacctcttgaagtgctcgagcgggacaa | |
| | agttacacagcttcttttgcaacaggataaggttccggagccggcgtctctcagctctaaccattcactcacttcttgtttcaccaaccaa | |
| | gggtattttttcttccatctgcctgatgccttggagattgaggcttgtcaggtgtactttacctatgacccctatagtgaggaagaccctga | |
| | cgaaggcgtagctggcgcccccactggctccagtccacagcctcttcagcctctgtcaggggaggacgacgcatattgtacgttcc | |
| | cctcacgggacgaccttctgctgttttcaccctcactgctcggcggaccctccccgccaagcacggcacctggggggagtggggc | |
| | aggagaagaaaggatgcctcctagtttgcaggagcgggttcctcgcgactgggatccgcaacccctcggaccacccacccctggc | |
| | gtacctgatctggtcgacttccaaccacctccggagcttgtcctcagagaggccggagaggaagtcccagacgcggggccaaga | |
| | gagggtgtgtcatttccctggtcccgccctccgggacagggtgagtttcgggcgctgaatgcgaggctcccccttaataccgatgcg | |
| | tacctgtcattgcaggaacttcagggccaggatcctacccacctggtgtgaaagcttgataatcaacctctggattacaaaatttgtgaa | |
| | agattgactggtattcttaactatgttgctcatttacgctatgtggatacgctgctttaatgcctttgtatcatgctattgcttcccgtatggct | |
| | ttcattttctcctccttgtataaatcctggttagttcttgccacggcggaactcatcgccgcctgccttgcccgctgctggacaggggctc | |
| | ggctgttgggcactgacaattccgtggaacttgtttattgcagcttataatggttacaaataaagcaatagcatcacaaatttcacaaata | |
| | aagcatttttttcactgcattctagttgtggtttgtccaaactcatcaatgtatcttacgccggcgtgagcctggagcaacaaatctgacttt | |
| | gcatgtgcaaacgccttcaacaacagcattattccagaagacaccttcttccccagcccaggtaagggcagctttggtgccttcgcag | |
| | gctgtttccttgcttcaggaatggccaggttctgcccagagctctggtcaatgatgtctaaaactcctctgattggtggtctcggccttat | |
| | ccattgccaccaaaaccctattttactaagaaacagtgagccttgttctggcagtccagagaatgacacgggaaaaaagcagatga | |
| | agagaaggtggcaggagagggcacgtggcccagcctcagtctctccaactgagttcctgcctgcctgcctttgctcagactgtttgc | |
| | cccttactgctcttctaggcctccctaggaacccctagtgatggagttggccactccctctctgcgcgctcgctcgctcactgaggccg | |
| | ggcgaccaaaggtcgcccgacgcccgggctttgcccgggcggcctcagtgagcgagcgagcgcgcagctgcctgcagg | |
| 32 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgccccatgcctgcctttactct | #5 TRAC 3: |
| | gccagagttatattgctggggttttgaagaagatcctattaaataaaagaataagcagtattattaagtagccctgcatttcaggtttcctt | HA TRAC |
| | gagtggcaggccaggcctggccgtgaacgttcactgaaatcatggcctcttggccaagattgatagcttgtgcctgtccctgagtccc | 3-2A- |
| | agtccatcacgagcagctggtttctaagatgctatttcccgtataaagcatgagaccgtgacttgccagccccacagagccccgccct | C11D5.3- |
| | tgtccatcactggcatctggactccagcctgggttggggcaaagagggaaatgagatcatgtcctaaccctgatcctcttgtcccaca | CD8-CD28- |
| | gatatccagaaccctgaccctgccggttccggggagggccgagggtcattgctgacgtgtggagacgtggaggagaatcctggcc | CD3z- |
| | ccatggccctgcctgtgacagctctgctcctccctctggccctgctgctccatgccgccagacccgacatcgtgctgacccagagcc | CISCβ-HA |
| | cccccagcctggccatgtctctgggcaagagagccaccatcagctgccgggccagcgagagcgtgaccatcctgggcagccacc | TRAC 3 |
| | tgatccactggtatcagcagaagcccggccagccccccaccctgctgatccagctcgccagcaatgtgcagaccggcgtgcccgc | |
| | cagattcagcggcagcggcagcagaaccgacttcaccctgaccatcgaccccgtggaagaggacgacgtggccgtgtactactg | |
| | cctgcagagccggaccatcccccggacctttggcggaggcaccaaactggaaatcaagggcagcaccagcggctccggcaagc | |
| | ctggctctggcgagggcagcacaaagggacagattcagctggtgcagagcggccctgagctgaagaaacccggcgagacagtg | |
| | aagatcagctgcaaggcctccggctacaccttcaccgactacagcatcaactgggtgaaaagagcccctggcaagggcctgaagt | |
| | ggatgggctggatcaacaccgagacaagagagcccgcctacgcctacgacttccggggcagattcgccttcagcctggaaacca | |
| | gcgccagcaccgcctacctgcagatcaacaacctgaagtacgaggacaccgccacctacttttgcgccctggactacagctacgcc | |
| | atggactactggggccagggcaccagcgtgaccgtgtccagcttcgtgcccgtgttcctgcccgccaaacctaccaccacccctgc | |
| | ccctagacctcccaccccagccccaacaatcgccagccagcctctgtctctgcggcccgaagcctgtagacctgctgccggcgga | |
| | gccgtgcacaccagaggcctggacttcgcctgcgacatctacatctgggcccctctggccggcacctgtggcgtgctgctgctgag | |
| | cctggtgatcaccctgtactgcaaccaccggaacagaagcaagcggagccggctgctgcacagcgactacatgaacatgacccc | |
| | aagacggcctggccccacccggaagcactaccagccttacgcccctcccagagacttcgccgcctaccggtccagagtgaagttc | |
| | agcagatccgccgacgcccctgcctaccagcagggacagaaccagctgtacaacgagctgaacctgggcagacgggaagagta | |
| | cgacgtgctggacaagcggagaggccgggaccccgagatgggcggaaagcccagacggaagaacccccaggaaggcctgta | |
| | taacgaactgcagaaagacaagatggccgaggcctacagcgagatcggcatgaagggcgagcggaggcgcggcaagggcca | |
| | cgatggcctgtaccagggcctgagcaccgccaccaaggacacctacgacgccctgcacatgcaggccctgccccccagaggatc | |
| | cggcgctacaaatttttcactgctgaaacaggcgggtgatgtggaggagaaccctggacccatgccacttggcctgctctggctggg | |
| | cttggcattgctcggcgcgctccacgcccaggctgaactgatccgcgtggccatattgtggcatgagatgtggcatgagggattgga | |
| | ggaggcgagtaggctgtactttggggaaaggaatgttaaagggatgtttgaggtccttgaacccctccacgctatgatggaaagagg | |
| | acctcaaacgcttaaagagacgtcattcaatcaagcctatggacgggatcttatggaagctcaagaatggtgtcgaaaatacatgaaa | |
| | agcgggaatgttaaggacctcacgcaagcctgggatctgtattaccatgttttccgacgcatttctaaacaaggaaaagatactatccc | |
| | atggttggggcacttgctcgttgggctcagtggggcgtttggattcatcatcctcgtatatctgttgattaattgtcggaacacaggtccc | |
| | tggcttaaaaaagttttgaagtgtaacaccccggatccttctaaattttttagtcaacttagttcagaacacgggggcgatgttcaaaagt | |
| | ggctgagttccccgtttcccagttcaagtttctcccctgggggtctcgcccccgagatatcacctcttgaagtgctcgagcgggacaa | |
| | agttacacagcttcttttgcaacaggataaggttccggagccggcgtctctcagctctaaccattcactcacttcttgtttcaccaaccaa | |
| | gggtattttttcttccatctgcctgatgccttggagattgaggcttgtcaggtgtactttacctatgacccctatagtgaggaagaccctga | |
| | cgaaggcgtagctggcgcccccactggctccagtccacagcctcttcagcctctgtcaggggaggacgacgcatattgtacgttcc | |
| | cctcacgggacgaccttctgctgttttcaccctcactgctcggcggaccctccccgccaagcacggcacctggggggagtggggc | |
| | aggagaagaaaggatgcctcctagtttgcaggagcgggttcctcgcgactgggatccgcaacccctcggaccacccacccctggc | |
| | gtacctgatctggtcgacttccaaccacctccggagcttgtcctcagagaggccggagaggaagtcccagacgcggggccaaga | |
| | gagggtgtgtcatttccctggtcccgccctccgggacagggtgagtttcgggcgctgaatgcgaggctcccccttaataccgatgcg | |
| | tacctgtcattgcaggaacttcagggccaggatcctacccacctggtgtgaaagcttgataatcaacctctggattacaaaatttgtgaa | |
| | agattgactggtattcttaactatgttgctcatttacgctatgtggatacgctgctttaatgcctttgtatcatgctattgcttcccgtatggct | |
| | ttcattttctcctccttgtataaatcctggttagttcttgccacggcggaactcatcgccgcctgccttgcccgctgctggacaggggctc | |
| | ggctgttgggcactgacaattccgtggaacttgtttattgcagcttataatggttacaaataaagcaatagcatcacaaatttcacaaata | |
| | aagcatttttttcactgcattctagttgtggtttgtccaaactcatcaatgtatcttacgccggcgtgagtgtaccagctgagagactctaa | |
| | atccagtgacaagtctgtctgcctattcaccgattttgattctcaaacaaatgtgtcacaaagtaaggattctgatgtgtatatcacagac | |
| | aaaactgtgctagacatgaggtctatggacttcaagagcaacagtgctgtggcctggagcaacaaatctgactttgcatgtgcaaacg | |
| | ccttcaacaacagcattattccagaagacaccttcttccccagcccaggtaagggcagctttggtgccttcgcaggctgtttccttgctt | |
| | caggaatggccaggttctgcccagagctctggtcaatgatgtctaaaactcctctgattggtggtctcggccttatccattgccaccaa | |
| | aaccctctttttactaagacctaggaacccctagtgatggagttggccactccctctctgcgcgctcgctcgctcactgaggccgggc | |
| | gaccaaaggtcgcccgacgcccgggctttgcccgggcggcctcagtgagcgagcgagcgcgcagctgcctgcagg | |
| 33 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgctggccgtgaacgttcactg | #5 TRAC 1: |
| | aaatcatggcctcttggccaagattgatagcttgtgcctgtccctgagtcccagtccatcacgagcagctggtttctaagatgctatttcc | HA TRAC |
| | cgtataaagcatgagaccgtgacttgccagccccacagagccccgcccttgtccatcactggcatctggactccagcctgggagg | 1-2A- |
| | ggcaaagagggaaatgagatcatgtcctaaccctgatcctcttgtcccacagatatccagaaccctgaccctgccgtgtaccagctg | C11D5.3- |
| | agagactctaaatccagtgacaagtctgtctgcctattcaccgattagattctcaaacaaatgtgtcacaaagtaaggattctgatgtgt | CD8-CD28- |
| | atatcacagacaaaactgtgctagacggttccggggagggccgagggtcattgctgacgtgtggagacgtggaggagaatcctgg | CD3z- |
| | ccccatggccctgcctgtgacagctctgctcctccctctggccctgctgctccatgccgccagacccgacatcgtgctgacccagag | CISCβ-HA |
| | cccccccagcctggccatgtctctgggcaagagagccaccatcagctgccgggccagcgagagcgtgaccatcctgggcagcca | TRAC 1 |
| | cctgatccactggtatcagcagaagcccggccagccccccaccctgctgatccagctcgccagcaatgtgcagaccggcgtgccc | |
| | gccagattcagcggcagcggcagcagaaccgacttcaccctgaccatcgaccccgtggaagaggacgacgtggccgtgtactac | |
| | tgcctgcagagccggaccatcccccggacattggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcttcgtgcccgtgttcctgcccgccaaacctaccaccacccct | |
| | gcccctagacctcccaccccagccccaacaatcgccagccagcctctgtctctgcggcccgaagcctgtagacctgctgccggcg | |
| | gagccgtgcacaccagaggcctggacttcgcctgcgacatctacatctgggcccctctggccggcacctgtggcgtgctgctgctg | |
| | agcctggtgatcaccctgtactgcaaccaccggaacagaagcaagcggagccggctgctgcacagcgactacatgaacatgacc | |
| | ccaagacggcctggccccacccggaagcactaccagccttacgcccctcccagagacttcgccgcctaccggtccagagtgaagt | |
| | tcagcagatccgccgacgcccctgcctaccagcagggacagaaccagctgtacaacgagctgaacctgggcagacgggaagag | |
| | tacgacgtgctggacaagcggagaggccgggaccccgagatgggcggaaagcccagacggaagaacccccaggaaggcctg | |
| | tataacgaactgcagaaagacaagatggccgaggcctacagcgagatcggcatgaagggcgagcggaggcgcggcaagggcc | |
| | acgatggcctgtaccagggcctgagcaccgccaccaaggacacctacgacgccctgcacatgcaggccctgccccccagaggat | |
| | ccggcgctacaaatttttcactgctgaaacaggcgggtgatgtggaggagaaccctggacccatgccacttggcctgctctggctgg | |
| | gcttggcattgctcggcgcgctccacgcccaggctgaactgatccgcgtggccatattgtggcatgagatgtggcatgagggattgg | |
| | aggaggcgagtaggctgtactttggggaaaggaatgttaaagggatgtttgaggtccttgaacccctccacgctatgatggaaagag | |
| | gacctcaaacgcttaaagagacgtcattcaatcaagcctatggacgggatcttatggaagctcaagaatggtgtcgaaaatacatgaa | |
| | aagcgggaatgttaaggacctcacgcaagcctgggatctgtattaccatgttttccgacgcatttctaaacaaggaaaagatactatcc | |
| | catggttggggcacttgctcgttgggctcagtggggcgtttggattcatcatcctcgtatatctgttgattaattgtcggaacacaggtcc | |
| | ctggcttaaaaaagttttgaagtgtaacaccccggatccttctaaattttttagtcaacttagttcagaacacgggggcgatgttcaaaag | |
| | tggctgagttccccgtttcccagttcaagtttctcccctgggggtctcgcccccgagatatcacctcttgaagtgctcgagcgggacaa | |
| | agttacacagcttcttttgcaacaggataaggttccggagccggcgtctctcagctctaaccattcactcacttcttgtttcaccaaccaa | |
| | gggtattttttcttccatctgcctgatgccttggagattgaggcttgtcaggtgtactttacctatgacccctatagtgaggaagaccctga | |
| | cgaaggcgtagctggcgcccccactggctccagtccacagcctcttcagcctctgtcaggggaggacgacgcatattgtacgttcc | |
| | cctcacgggacgaccttctgctgttttcaccctcactgctcggcggaccctccccgccaagcacggcacctggggggagtggggc | |
| | aggagaagaaaggatgcctcctagtttgcaggagcgggttcctcgcgactgggatccgcaacccctcggaccacccacccctggc | |
| | gtacctgatctggtcgacttccaaccacctccggagcttgtcctcagagaggccggagaggaagtcccagacgcggggccaaga | |
| | gagggtgtgtcatttccctggtcccgccctccgggacagggtgagtttcgggcgctgaatgcgaggctcccccttaataccgatgcg | |
| | tacctgtcattgcaggaacttcagggccaggatcctacccacctggtgtgaaagcttgataatcaacctctggattacaaaatttgtgaa | |
| | agattgactggtattcttaactatgttgctcatttacgctatgtggatacgctgctttaatgcctttgtatcatgctattgcttcccgtatggct | |
| | ttcattttctcctccttgtataaatcctggttagttcttgccacggcggaactcatcgccgcctgccttgcccgctgctggacaggggctc | |
| | ggctgttgggcactgacaattccgtggaacttgtttattgcagcttataatggttacaaataaagcaatagcatcacaaatttcacaaata | |
| | aagcatttttttcactgcattctagttgtggtttgtccaaactcatcaatgtatcttacgccggcgtgaatgaggtctatggacttcaagag | |
| | caacagtgctgtggcctggagcaacaaatctgactttgcatgtgcaaacgccttcaacaacagcattattccagaagacaccttcttcc | |
| | ccagcccaggtaagggcagctttggtgccttcgcaggctgtttccttgcttcaggaatggccaggttctgcccagagctctggtcaat | |
| | gatgtctaaaactcctctgattggtggtctcggccttatccattgccaccaaaaccctctttttactaagaaacagtgagccttgttctggc | |
| | agtccagagaatgacacgggaaaaaagcagatgaagagaaggtggcaggagagggcacgtggcccagcctcagtctctccaac | |
| | tgagttcctgcctgcctgcctttgccctaggaacccctagtgatggagttggccactccctctctgcgcgctcgctcgctcactgaggc | |
| | cgggcgaccaaaggtcgcccgacgcccgggctttgcccgggcggcctcagtgagcgagcgagcgcgcagctgcctgcagg | |
| 34 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgcccaagattgatagcttgtg | #6 TRAC 2: |
| | cctgtccctgagtcccagtccatcacgagcagctggtttctaagatgctatttcccgtataaagcatgagaccgtgacttgccagcccc | HA TRAC |
| | acagagccccgcccttgtccatcactggcatctggactccagcctgggttggggcaaagagggaaatgagatcatgtcctaaccct | 2-C11D5.3- |
| | gatcctcttgtcccacagatatccagaaccctgaccctgccgtgtaccagctgagagactctaaatccagtgacaagtctgtctgccta | CD8-41BB- |
| | ttcaccgattttgattctcaaacaaatgtgtcacaaagtaaggattctgatgtgtatatcacagacaaaactgtgctagacatgaggtcta | CD3z-P2A- |
| | tggacttcaagagcaacagtgctgtgtgaatgaatgattaattaataaaagatctttattttcattagatctgtgtgttggttttttgtgtgatc | CISCβ-HA |
| | ctcgagggaatgaaagaccccacctgtaggtttggcaagctagcttaagtaacgccattagcaaggcatggaaaatacataactga | TRAC 2 |
| | gaatagagaagttcagatcaaggttaggaacagagagacagcagaatatgggccaaacaggatatctgtggtaagcagttcctgcc | |
| | ccggctcagggccaagaacagatggtccccagatgcggtcccgccctcagcagtttctagagaaccatcagatgtttccagggtgc | |
| | cccaaggacctgaaaatgaccctgtgccttatttgaactaaccaatcagttcgcttctcgcttctgttcgcgcgcttctgctccccgagct | |
| | caataaaagagcccacaacccctcactcggcgcgcgccagtccggtaccagtcgccaccatggccctgcctgtgacagctctgct | |
| | cctccctctggccctgctgctccatgccgccagacccgacatcgtgctgacccagagcccccccagcctggccatgtctctgggca | |
| | agagagccaccatcagctgccgggccagcgagagcgtgaccatcctgggcagccacctgatccactggtatcagcagaagcccg | |
| | gccagccccccaccctgctgatccagctcgccagcaatgtgcagaccggcgtgcccgccagattcagcggcagcggcagcaga | |
| | accgacttcaccctgaccatcgaccccgtggaagaggacgacgtggccgtgtactactgcctgcagagccggaccatcccccgga | |
| | cattggcggaggcaccaaactggaaatcaagggcagcaccagcggctccggcaagcctggctctggcgagggcagcacaaag | |
| | ggacagattcagctggtgcagagcggccctgagctgaagaaacccggcgagacagtgaagatcagctgcaaggcctccggctac | |
| | accttcaccgactacagcatcaactgggtgaaaagagcccctggcaagggcctgaagtggatgggctggatcaacaccgagaca | |
| | agagagcccgcctacgcctacgacttccggggcagattcgccttcagcctggaaaccagcgccagcaccgcctacctgcagatca | |
| | acaacctgaagtacgaggacaccgccacctacttttgcgccctggactacagctacgccatggactactggggccagggcaccag | |
| | cgtgaccgtgtccagcgccgccgccttcgtgcccgtgttcctgcccgccaaacctaccaccacccctgcccctagacctcccaccc | |
| | cagccccaacaatcgccagccagcctctgtctctgcggcccgaagcctgtagacctgctgccggcggagccgtgcacaccagag | |
| | gcctggacttcgcctgcgacatctacatctgggcccctctggccggcacctgtggcgtgctgctgctgagcctggtgatcaccctgta | |
| | ctgcaaccaccggaacagattcagcgtcgtgaagcggggcagaaagaagctgctgtacatcttcaagcagcccttcatgcggccc | |
| | gtgcagaccacacaagaggaagatggctgctcctgcagattccctgaggaagaagaaggcggctgcgagctgagagtgaagttc | |
| | agcagatccgccgacgcccctgcctaccagcagggacagaaccagctgtacaacgagctgaacctgggcagacgggaagagta | |
| | cgacgtgctggacaagcggagaggccgggaccccgagatgggcggaaagcccagacggaagaacccccaggaaggcctgta | |
| | taacgaactgcagaaagacaagatggccgaggcctacagcgagatcggcatgaagggcgagcggaggcgcggcaagggcca | |
| | cgatggcctgtaccagggcctgagcaccgccaccaaggacacctacgacgccctgcacatgcaggccctgccccccagaggatc | |
| | cggcgctacaaatttttcactgctgaaacaggcgggtgatgtggaggagaaccctggacccatgccacttggcctgctctggctggg | |
| | cttggcattgctcggcgcgctccacgcccaggctgaactgatccgcgtggccatattgtggcatgagatgtggcatgagggattgga | |
| | ggaggcgagtaggctgtactttggggaaaggaatgttaaagggatgtttgaggtccttgaacccctccacgctatgatggaaagagg | |
| | acctcaaacgcttaaagagacgtcattcaatcaagcctatggacgggatcttatggaagctcaagaatggtgtcgaaaatacatgaaa | |
| | agcgggaatgttaaggacctcacgcaagcctgggatctgtattaccatgttttccgacgcatttctaaacaaggaaaagatactatccc | |
| | atggttggggcacttgctcgttgggctcagtggggcgtttggattcatcatcctcgtatatctgttgattaattgtcggaacacaggtccc | |
| | tggcttaaaaaagttttgaagtgtaacaccccggatccttctaaattttttagtcaacttagttcagaacacgggggcgatgttcaaaagt | |
| | ggctgagttccccgtttcccagttcaagtttctcccctgggggtctcgcccccgagatatcacctcttgaagtgctcgagcgggacaa | |
| | agttacacagcttcttttgcaacaggataaggttccggagccggcgtctctcagctctaaccattcactcacttcttgtttcaccaaccaa | |
| | gggtattttttcttccatctgcctgatgccttggagattgaggcttgtcaggtgtactttacctatgacccctatagtgaggaagaccctga | |
| | cgaaggcgtagctggcgcccccactggctccagtccacagcctcttcagcctctgtcaggggaggacgacgcatattgtacgttcc | |
| | cctcacgggacgaccttctgctgttttcaccctcactgctcggcggaccctccccgccaagcacggcacctggggggagtggggc | |
| | aggagaagaaaggatgcctcctagtttgcaggagcgggttcctcgcgactgggatccgcaacccctcggaccacccacccctggc | |
| | gtacctgatctggtcgacttccaaccacctccggagcttgtcctcagagaggccggagaggaagtcccagacgcggggccaaga | |
| | gagggtgtgtcatttccctggtcccgccctccgggacagggtgagtttcgggcgctgaatgcgaggctcccccttaataccgatgcg | |
| | tacctgtcattgcaggaacttcagggccaggatcctacccacctggtgtgaaagcttgataatcaacctctggattacaaaatttgtgaa | |
| | agattgactggtattcttaactatgttgctcatttacgctatgtggatacgctgctttaatgcctttgtatcatgctattgcttcccgtatggct | |
| | ttcattttctcctccttgtataaatcctggttagttcttgccacggcggaactcatcgccgcctgccttgcccgctgctggacaggggctc | |
| | ggctgttgggcactgacaattccgtggaacttgtttattgcagcttataatggttacaaataaagcaatagcatcacaaatttcacaaata | |
| | aagcatttttttcactgcattctagttgtggtttgtccaaactcatcaatgtatcttacgccggcgtgagcctggagcaacaaatctgacttt | |
| | gcatgtgcaaacgccttcaacaacagcattattccagaagacaccttcttccccagcccaggtaagggcagctttggtgccttcgcag | |
| | gctgtttccttgcttcaggaatggccaggttctgcccagagctctggtcaatgatgtctaaaactcctctgattggtggtctcggccttat | |
| | ccattgccaccaaaaccctattttactaagaaacagtgagccttgttctggcagtccagagaatgacacgggaaaaaagcagatga | |
| | agagaaggtggcaggagagggcacgtggcccagcctcagtctctccaactgagttcctgcctgcctgcctttgctcagactgtttgc | |
| | cccttactgctcttctaggcctccctaggaacccctagtgatggagttggccactccctctctgcgcgctcgctcgctcactgaggccg | |
| | ggcgaccaaaggtcgcccgacgcccgggctttgcccgggcggcctcagtgagcgagcgagcgcgcagctgcctgcagg | |
| 35 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgccccatgcctgcctttactct | #6 TRAC 3: |
| | gccagagttatattgctggggttttgaagaagatcctattaaataaaagaataagcagtattattaagtagccctgcatttcaggtttcctt | HA TRAC |
| | gagtggcaggccaggcctggccgtgaacgttcactgaaatcatggcctcttggccaagattgatagcttgtgcctgtccctgagtccc | 3-C11D5.3- |
| | agtccatcacgagcagctggtttctaagatgctatttcccgtataaagcatgagaccgtgacttgccagccccacagagccccgccct | CD8-41BB- |
| | tgtccatcactggcatctggactccagcctgggttggggcaaagagggaaatgagatcatgtcctaaccctgatcctcttgtcccaca | CD3z-P2A- |
| | gatatccagaaccctgaccctgcctgaatgaatgattaattaataaaagatctttattttcattagatctgtgtgttggttttttgtgtgatcct | CISCβ-HA |
| | cgagggaatgaaagaccccacctgtaggtttggcaagctagcttaagtaacgccattttgcaaggcatggaaaatacataactgaga | TRAC 3 |
| | atagagaagttcagatcaaggttaggaacagagagacagcagaatatgggccaaacaggatatctgtggtaagcagttcctgcccc | |
| | ggctcagggccaagaacagatggtccccagatgcggtcccgccctcagcagtttctagagaaccatcagatgtttccagggtgccc | |
| | caaggacctgaaaatgaccctgtgccttatttgaactaaccaatcagttcgcttctcgcttctgttcgcgcgcttctgctccccgagctca | |
| | ataaaagagcccacaacccctcactcggcgcgcgccagtccggtaccagtcgccaccatggccctgcctgtgacagctctgctcct | |
| | ccctctggccctgctgctccatgccgccagacccgacatcgtgctgacccagagcccccccagcctggccatgtctctgggcaaga | |
| | gagccaccatcagctgccgggccagcgagagcgtgaccatcctgggcagccacctgatccactggtatcagcagaagcccggcc | |
| | agccccccaccctgctgatccagctcgccagcaatgtgcagaccggcgtgcccgccagattcagcggcagcggcagcagaacc | |
| | gacttcaccctgaccatcgaccccgtggaagaggacgacgtggccgtgtactactgcctgcagagccggaccatcccccggacct | |
| | ttggcggaggcaccaaactggaaatcaagggcagcaccagcggctccggcaagcctggctctggcgagggcagcacaaaggg | |
| | acagattcagctggtgcagagcggccctgagctgaagaaacccggcgagacagtgaagatcagctgcaaggcctccggctacac | |
| | cttcaccgactacagcatcaactgggtgaaaagagcccctggcaagggcctgaagtggatgggctggatcaacaccgagacaag | |
| | agagcccgcctacgcctacgacttccggggcagattcgccttcagcctggaaaccagcgccagcaccgcctacctgcagatcaac | |
| | aacctgaagtacgaggacaccgccacctacttagcgccctggactacagctacgccatggactactggggccagggcaccagcg | |
| | tgaccgtgtccagcgccgccgccttcgtgcccgtgacctgcccgccaaacctaccaccacccctgcccctagacctcccacccca | |
| | gccccaacaatcgccagccagcctctgtctctgcggcccgaagcctgtagacctgctgccggcggagccgtgcacaccagaggc | |
| | ctggacttcgcctgcgacatctacatctgggcccctctggccggcacctgtggcgtgctgctgctgagcctggtgatcaccctgtact | |
| | gcaaccaccggaacagattcagcgtcgtgaagcggggcagaaagaagctgctgtacatcttcaagcagcccttcatgcggcccgt | |
| | gcagaccacacaagaggaagatggctgctcctgcagattccctgaggaagaagaaggcggctgcgagctgagagtgaagttcag | |
| | cagatccgccgacgcccctgcctaccagcagggacagaaccagctgtacaacgagctgaacctgggcagacgggaagagtacg | |
| | acgtgctggacaagcggagaggccgggaccccgagatgggcggaaagcccagacggaagaacccccaggaaggcctgtata | |
| | acgaactgcagaaagacaagatggccgaggcctacagcgagatcggcatgaagggcgagcggaggcgcggcaagggccacg | |
| | atggcctgtaccagggcctgagcaccgccaccaaggacacctacgacgccctgcacatgcaggccctgccccccagaggatccg | |
| | gcgctacaaatttacactgctgaaacaggcgggtgatgtggaggagaaccctggacccatgccacttggcctgctctggctgggct | |
| | tggcattgctcggcgcgctccacgcccaggctgaactgatccgcgtggccatattgtggcatgagatgtggcatgagggattggag | |
| | gaggcgagtaggctgtactttggggaaaggaatgttaaagggatgtttgaggtccttgaacccctccacgctatgatggaaagagga | |
| | cctcaaacgcttaaagagacgtcattcaatcaagcctatggacgggatcttatggaagctcaagaatggtgtcgaaaatacatgaaaa | |
| | gcgggaatgttaaggacctcacgcaagcctgggatctgtattaccatgttttccgacgcatttctaaacaaggaaaagatactatccca | |
| | tggttggggcacttgctcgttgggctcagtggggcgtttggattcatcatcctcgtatatctgttgattaattgtcggaacacaggtccct | |
| | ggcttaaaaaagttttgaagtgtaacaccccggatccttctaaattttttagtcaacttagttcagaacacgggggcgatgttcaaaagtg | |
| | gctgagttccccgtttcccagttcaagtttctcccctgggggtctcgcccccgagatatcacctcttgaagtgctcgagcgggacaaa | |
| | gttacacagcttcttttgcaacaggataaggttccggagccggcgtctctcagctctaaccattcactcacttcttgtttcaccaaccaag | |
| | ggtattttacttccatctgcctgatgccttggagattgaggcttgtcaggtgtactttacctatgacccctatagtgaggaagaccctgac | |
| | gaaggcgtagctggcgcccccactggctccagtccacagcctcttcagcctctgtcaggggaggacgacgcatattgtacgaccc | |
| | ctcacgggacgaccttctgctgttacaccctcactgctcggcggaccctccccgccaagcacggcacctggggggagtggggca | |
| | ggagaagaaaggatgcctcctagtttgcaggagcgggttcctcgcgactgggatccgcaacccctcggaccacccacccctggcg | |
| | tacctgatctggtcgacttccaaccacctccggagcttgtcctcagagaggccggagaggaagtcccagacgcggggccaagaga | |
| | gggtgtgtcatttccctggtcccgccctccgggacagggtgagtttcgggcgctgaatgcgaggctcccccttaataccgatgcgta | |
| | cctgtcattgcaggaacttcagggccaggatcctacccacctggtgtgaaagcttgataatcaacctctggattacaaaatagtgaaa | |
| | gattgactggtattcttaactatgagctcatttacgctatgtggatacgctgctttaatgcctttgtatcatgctattgcttcccgtatggctt | |
| | tcattttctcctccttgtataaatcctggttagttcttgccacggcggaactcatcgccgcctgccttgcccgctgctggacaggggctc | |
| | ggctgttgggcactgacaattccgtggaacttgtttattgcagcttataatggttacaaataaagcaatagcatcacaaatttcacaaata | |
| | aagcatttttttcactgcattctagttgtggtttgtccaaactcatcaatgtatcttacgccggcgtgagtgtaccagctgagagactctaa | |
| | atccagtgacaagtctgtctgcctattcaccgattagattctcaaacaaatgtgtcacaaagtaaggattctgatgtgtatatcacagac | |
| | aaaactgtgctagacatgaggtctatggacttcaagagcaacagtgctgtggcctggagcaacaaatctgactttgcatgtgcaaacg | |
| | ccttcaacaacagcattattccagaagacaccttcttccccagcccaggtaagggcagctaggtgccttcgcaggctgtttccttgctt | |
| | caggaatggccaggttctgcccagagctctggtcaatgatgtctaaaactcctctgattggtggtctcggccttatccattgccaccaa | |
| | aaccctctttttactaagacctaggaacccctagtgatggagttggccactccctctctgcgcgctcgctcgctcactgaggccgggc | |
| | gaccaaaggtcgcccgacgcccgggctttgcccgggcggcctcagtgagcgagcgagcgcgcagctgcctgcagg | |
| 36 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgctggccgtgaacgttcactg | #6 TRAC 1: |
| | aaatcatggcctcttggccaagattgatagcttgtgcctgtccctgagtcccagtccatcacgagcagctggtttctaagatgctatttcc | HA TRAC |
| | cgtataaagcatgagaccgtgacttgccagccccacagagccccgcccttgtccatcactggcatctggactccagcctgggttgg | 1-C11D5.3- |
| | ggcaaagagggaaatgagatcatgtcctaaccctgatcctcttgtcccacagatatccagaaccctgaccctgccgtgtaccagctg | CD8-41BB- |
| | agagactctaaatccagtgacaagtctgtctgcctattcaccgattagattctcaaacaaatgtgtcacaaagtaaggattctgatgtgt | CD3z-P2A- |
| | atatcacagacaaaactgtgctagactgaatgaatgattaattaataaaagatctttattttcattagatctgtgtgttggttttttgtgtgatc | CISCβ-HA |
| | ctcgagggaatgaaagaccccacctgtaggtttggcaagctagcttaagtaacgccattagcaaggcatggaaaatacataactga | TRAC 1 |
| | gaatagagaagttcagatcaaggttaggaacagagagacagcagaatatgggccaaacaggatatctgtggtaagcagacctgcc | |
| | ccggctcagggccaagaacagatggtccccagatgcggtcccgccctcagcagtttctagagaaccatcagatgtttccagggtgc | |
| | cccaaggacctgaaaatgaccctgtgccttatttgaactaaccaatcagttcgcttctcgcttctgttcgcgcgcttctgctccccgagct | |
| | caataaaagagcccacaacccctcactcggcgcgcgccagtccggtaccagtcgccaccatggccctgcctgtgacagctctgct | |
| | cctccctctggccctgctgctccatgccgccagacccgacatcgtgctgacccagagcccccccagcctggccatgtctctgggca | |
| | agagagccaccatcagctgccgggccagcgagagcgtgaccatcctgggcagccacctgatccactggtatcagcagaagcccg | |
| | gccagccccccaccctgctgatccagctcgccagcaatgtgcagaccggcgtgcccgccagattcagcggcagcggcagcaga | |
| | accgacttcaccctgaccatcgaccccgtggaagaggacgacgtggccgtgtactactgcctgcagagccggaccatcccccgga | |
| | cctttggcggaggcaccaaactggaaatcaagggcagcaccagcggctccggcaagcctggctctggcgagggcagcacaaag | |
| | ggacagattcagctggtgcagagcggccctgagctgaagaaacccggcgagacagtgaagatcagctgcaaggcctccggctac | |
| | accttcaccgactacagcatcaactgggtgaaaagagcccctggcaagggcctgaagtggatgggctggatcaacaccgagaca | |
| | agagagcccgcctacgcctacgacttccggggcagattcgccttcagcctggaaaccagcgccagcaccgcctacctgcagatca | |
| | acaacctgaagtacgaggacaccgccacctacttttgcgccctggactacagctacgccatggactactggggccagggcaccag | |
| | cgtgaccgtgtccagcgccgccgccttcgtgcccgtgttcctgcccgccaaacctaccaccacccctgcccctagacctcccaccc | |
| | cagccccaacaatcgccagccagcctctgtctctgcggcccgaagcctgtagacctgctgccggcggagccgtgcacaccagag | |
| | gcctggacttcgcctgcgacatctacatctgggcccctctggccggcacctgtggcgtgctgctgctgagcctggtgatcaccctgta | |
| | ctgcaaccaccggaacagattcagcgtcgtgaagcggggcagaaagaagctgctgtacatcttcaagcagcccttcatgcggccc | |
| | gtgcagaccacacaagaggaagatggctgctcctgcagattccctgaggaagaagaaggcggctgcgagctgagagtgaagttc | |
| | agcagatccgccgacgcccctgcctaccagcagggacagaaccagctgtacaacgagctgaacctgggcagacgggaagagta | |
| | cgacgtgctggacaagcggagaggccgggaccccgagatgggcggaaagcccagacggaagaacccccaggaaggcctgta | |
| | taacgaactgcagaaagacaagatggccgaggcctacagcgagatcggcatgaagggcgagcggaggcgcggcaagggcca | |
| | cgatggcctgtaccagggcctgagcaccgccaccaaggacacctacgacgccctgcacatgcaggccctgccccccagaggatc | |
| | cggcgctacaaatttttcactgctgaaacaggcgggtgatgtggaggagaaccctggacccatgccacttggcctgctctggctggg | |
| | cttggcattgctcggcgcgctccacgcccaggctgaactgatccgcgtggccatattgtggcatgagatgtggcatgagggattgga | |
| | ggaggcgagtaggctgtactttggggaaaggaatgttaaagggatgtttgaggtccttgaacccctccacgctatgatggaaagagg | |
| | acctcaaacgcttaaagagacgtcattcaatcaagcctatggacgggatcttatggaagctcaagaatggtgtcgaaaatacatgaaa | |
| | agcgggaatgttaaggacctcacgcaagcctgggatctgtattaccatgttttccgacgcatttctaaacaaggaaaagatactatccc | |
| | atggttggggcacttgctcgttgggctcagtggggcgtttggattcatcatcctcgtatatctgttgattaattgtcggaacacaggtccc | |
| | tggcttaaaaaagttttgaagtgtaacaccccggatccttctaaattttttagtcaacttagttcagaacacgggggcgatgttcaaaagt | |
| | ggctgagttccccgtttcccagttcaagtttctcccctgggggtctcgcccccgagatatcacctcttgaagtgctcgagcgggacaa | |
| | agttacacagcttcttttgcaacaggataaggttccggagccggcgtctctcagctctaaccattcactcacttcttgtttcaccaaccaa | |
| | gggtattttttcttccatctgcctgatgccttggagattgaggcttgtcaggtgtactttacctatgacccctatagtgaggaagaccctga | |
| | cgaaggcgtagctggcgcccccactggctccagtccacagcctcttcagcctctgtcaggggaggacgacgcatattgtacgttcc | |
| | cctcacgggacgaccttctgctgttttcaccctcactgctcggcggaccctccccgccaagcacggcacctggggggagtggggc | |
| | aggagaagaaaggatgcctcctagtttgcaggagcgggttcctcgcgactgggatccgcaacccctcggaccacccacccctggc | |
| | gtacctgatctggtcgacttccaaccacctccggagcttgtcctcagagaggccggagaggaagtcccagacgcggggccaaga | |
| | gagggtgtgtcatttccctggtcccgccctccgggacagggtgagtttcgggcgctgaatgcgaggctcccccttaataccgatgcg | |
| | tacctgtcattgcaggaacttcagggccaggatcctacccacctggtgtgaaagcttgataatcaacctctggattacaaaatttgtgaa | |
| | agattgactggtattcttaactatgttgctcatttacgctatgtggatacgctgctttaatgcctttgtatcatgctattgcttcccgtatggct | |
| | ttcattttctcctccttgtataaatcctggttagttcttgccacggcggaactcatcgccgcctgccttgcccgctgctggacaggggctc | |
| | ggctgttgggcactgacaattccgtggaacttgtttattgcagcttataatggttacaaataaagcaatagcatcacaaatttcacaaata | |
| | aagcatttttttcactgcattctagttgtggtttgtccaaactcatcaatgtatcttacgccggcgtgaatgaggtctatggacttcaagag | |
| | caacagtgctgtggcctggagcaacaaatctgactttgcatgtgcaaacgccttcaacaacagcattattccagaagacaccttcttcc | |
| | ccagcccaggtaagggcagctttggtgccttcgcaggctgtttccttgcttcaggaatggccaggttctgcccagagctctggtcaat | |
| | gatgtctaaaactcctctgattggtggtctcggccttatccattgccaccaaaaccctctttttactaagaaacagtgagccttgttctggc | |
| | agtccagagaatgacacgggaaaaaagcagatgaagagaaggtggcaggagagggcacgtggcccagcctcagtctctccaac | |
| | tgagttcctgcctgcctgcctttgccctaggaacccctagtgatggagttggccactccctctctgcgcgctcgctcgctcactgaggc | |
| | cgggcgaccaaaggtcgcccgacgcccgggctttgcccgggcggcctcagtgagcgagcgagcgcgcagctgcctgcagg | |
| 37 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgcccaagattgatagcttgtg | #7 TRAC 2: |
| | cctgtccctgagtcccagtccatcacgagcagctggtttctaagatgctatttcccgtataaagcatgagaccgtgacttgccagcccc | HA TRAC |
| | acagagccccgcccttgtccatcactggcatctggactccagcctgggttggggcaaagagggaaatgagatcatgtcctaaccct | 2-2A- |
| | gatcctcttgtcccacagatatccagaaccctgaccctgccgtgtaccagctgagagactctaaatccagtgacaagtctgtctgccta | C11D5.3- |
| | ttcaccgattttgattctcaaacaaatgtgtcacaaagtaaggattctgatgtgtatatcacagacaaaactgtgctagacatgaggtcta | AAA-CD8- |
| | tggacttcaagagcaacagtgctgtgggttccggggagggccgagggtcattgctgacgtgtggagacgtggaggagaatcctgg | 41BB- |
| | ccccatggccctgcctgtgacagctctgctcctccctctggccctgctgctccatgccgccagacccgacatcgtgctgacccagag | CD3z-P2A- |
| | cccccccagcctggccatgtctctgggcaagagagccaccatcagctgccgggccagcgagagcgtgaccatcctgggcagcca | CISCβ-HA |
| | cctgatccactggtatcagcagaagcccggccagccccccaccctgctgatccagctcgccagcaatgtgcagaccggcgtgccc | TRAC 2 |
| | gccagattcagcggcagcggcagcagaaccgacttcaccctgaccatcgaccccgtggaagaggacgacgtggccgtgtactac | |
| | tgcctgcagagccggaccatcccccggacctttggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcgccgccgccttcgtgcccgtgttcctgcccgccaaacctac | |
| | caccacccctgcccctagacctcccaccccagccccaacaatcgccagccagcctctgtctctgcggcccgaagcctgtagacctg | |
| | ctgccggcggagccgtgcacaccagaggcctggacttcgcctgcgacatctacatctgggcccctctggccggcacctgtggcgt | |
| | gctgctgctgagcctggtgatcaccctgtactgcaaccaccggaacagattcagcgtcgtgaagcggggcagaaagaagctgctgt | |
| | acatcttcaagcagcccttcatgcggcccgtgcagaccacacaagaggaagatggctgctcctgcagattccctgaggaagaaga | |
| | aggcggctgcgagctgagagtgaagttcagcagatccgccgacgcccctgcctaccagcagggacagaaccagctgtacaacg | |
| | agctgaacctgggcagacgggaagagtacgacgtgctggacaagcggagaggccgggaccccgagatgggcggaaagccca | |
| | gacggaagaacccccaggaaggcctgtataacgaactgcagaaagacaagatggccgaggcctacagcgagatcggcatgaag | |
| | ggcgagcggaggcgcggcaagggccacgatggcctgtaccagggcctgagcaccgccaccaaggacacctacgacgccctgc | |
| | acatgcaggccctgccccccagaggatccggcgctacaaatttttcactgctgaaacaggcgggtgatgtggaggagaaccctgg | |
| | acccatgccacttggcctgctctggctgggcttggcattgctcggcgcgctccacgcccaggctgaactgatccgcgtggccatatt | |
| | gtggcatgagatgtggcatgagggattggaggaggcgagtaggctgtactttggggaaaggaatgttaaagggatgtttgaggtcct | |
| | tgaacccctccacgctatgatggaaagaggacctcaaacgcttaaagagacgtcattcaatcaagcctatggacgggatcttatgga | |
| | agctcaagaatggtgtcgaaaatacatgaaaagcgggaatgttaaggacctcacgcaagcctgggatctgtattaccatgttttccga | |
| | cgcatttctaaacaaggaaaagatactatcccatggttggggcacttgctcgttgggctcagtggggcgtttggattcatcatcctcgta | |
| | tatctgttgattaattgtcggaacacaggtccctggcttaaaaaagttttgaagtgtaacaccccggatccttctaaattttttagtcaactt | |
| | agttcagaacacgggggcgatgttcaaaagtggctgagttccccgtttcccagttcaagtttctcccctgggggtctcgcccccgaga | |
| | tatcacctcttgaagtgctcgagcgggacaaagttacacagcttcttttgcaacaggataaggttccggagccggcgtctctcagctct | |
| | aaccattcactcacttcttgtttcaccaaccaagggtattttttcttccatctgcctgatgccttggagattgaggcttgtcaggtgtacttta | |
| | cctatgacccctatagtgaggaagaccctgacgaaggcgtagctggcgcccccactggctccagtccacagcctcttcagcctctgt | |
| | caggggaggacgacgcatattgtacgttcccctcacgggacgaccttctgctgttttcaccctcactgctcggcggaccctccccgc | |
| | caagcacggcacctggggggagtggggcaggagaagaaaggatgcctcctagtttgcaggagcgggttcctcgcgactgggatc | |
| | cgcaacccctcggaccacccacccctggcgtacctgatctggtcgacttccaaccacctccggagcttgtcctcagagaggccgga | |
| | gaggaagtcccagacgcggggccaagagagggtgtgtcatttccctggtcccgccctccgggacagggtgagtttcgggcgctg | |
| | aatgcgaggctcccccttaataccgatgcgtacctgtcattgcaggaacttcagggccaggatcctacccacctggtgtgaaagcttg | |
| | ataatcaacctctggattacaaaatttgtgaaagattgactggtattcttaactatgttgctccttttacgctatgtggatacgctgctttaat | |
| | gcctttgtatcatgctattgcttcccgtatggctttcattttctcctccttgtataaatcctggttagttcttgccacggcggaactcatcgcc | |
| | gcctgccttgcccgctgctggacaggggctcggctgttgggcactgacaattccgtggaacttgtttattgcagcttataatggttaca | |
| | aataaagcaatagcatcacaaatttcacaaataaagcatttttttcactgcattctagttgtggtttgtccaaactcatcaatgtatcttacgc | |
| | cggcgtgagcctggagcaacaaatctgactttgcatgtgcaaacgccttcaacaacagcattattccagaagacaccttcttccccag | |
| | cccaggtaagggcagctttggtgccttcgcaggctgtttccttgcttcaggaatggccaggttctgcccagagctctggtcaatgatgt | |
| | ctaaaactcctctgattggtggtctcggccttatccattgccaccaaaaccctctttttactaagaaacagtgagccttgttctggcagtc | |
| | cagagaatgacacgggaaaaaagcagatgaagagaaggtggcaggagagggcacgtggcccagcctcagtctctccaactgag | |
| | ttcctgcctgcctgcctttgctcagactgtttgccccttactgctcttctaggcctccctaggaacccctagtgatggagttggccactcc | |
| | ctctctgcgcgctcgctcgctcactgaggccgggcgaccaaaggtcgcccgacgcccgggctttgcccgggcggcctcagtgag | |
| | cgagcgagcgcgcagctgcctgcagg | |
| 38 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgccccatgcctgcctttactct | #7 TRAC 3: |
| | gccagagttatattgctggggttttgaagaagatcctattaaataaaagaataagcagtattattaagtagccctgcatttcaggtacctt | HA TRAC |
| | gagtggcaggccaggcctggccgtgaacgttcactgaaatcatggcctcttggccaagattgatagcttgtgcctgtccctgagtccc | 3-2A- |
| | agtccatcacgagcagctggtttctaagatgctatttcccgtataaagcatgagaccgtgacttgccagccccacagagccccgccct | C11D5.3- |
| | tgtccatcactggcatctggactccagcctgggttggggcaaagagggaaatgagatcatgtcctaaccctgatcctcttgtcccaca | AAA-CD8- |
| | gatatccagaaccctgaccctgccggttccggggagggccgagggtcattgctgacgtgtggagacgtggaggagaatcctggcc | 41BB- |
| | ccatggccctgcctgtgacagctctgctcctccctctggccctgctgctccatgccgccagacccgacatcgtgctgacccagagcc | CD3z-P2A- |
| | cccccagcctggccatgtctctgggcaagagagccaccatcagctgccgggccagcgagagcgtgaccatcctgggcagccacc | CISCβ-HA |
| | tgatccactggtatcagcagaagcccggccagccccccaccctgctgatccagctcgccagcaatgtgcagaccggcgtgcccgc | TRAC 3 |
| | cagattcagcggcagcggcagcagaaccgacttcaccctgaccatcgaccccgtggaagaggacgacgtggccgtgtactactg | |
| | cctgcagagccggaccatcccccggacctttggcggaggcaccaaactggaaatcaagggcagcaccagcggctccggcaagc | |
| | ctggctctggcgagggcagcacaaagggacagattcagctggtgcagagcggccctgagctgaagaaacccggcgagacagtg | |
| | aagatcagctgcaaggcctccggctacaccttcaccgactacagcatcaactgggtgaaaagagcccctggcaagggcctgaagt | |
| | ggatgggctggatcaacaccgagacaagagagcccgcctacgcctacgacttccggggcagattcgccttcagcctggaaacca | |
| | gcgccagcaccgcctacctgcagatcaacaacctgaagtacgaggacaccgccacctacttttgcgccctggactacagctacgcc | |
| | atggactactggggccagggcaccagcgtgaccgtgtccagcgccgccgccttcgtgcccgtgttcctgcccgccaaacctacca | |
| | ccacccctgcccctagacctcccaccccagccccaacaatcgccagccagcctctgtctctgcggcccgaagcctgtagacctgct | |
| | gccggcggagccgtgcacaccagaggcctggacttcgcctgcgacatctacatctgggcccctctggccggcacctgtggcgtgc | |
| | tgctgctgagcctggtgatcaccctgtactgcaaccaccggaacagattcagcgtcgtgaagcggggcagaaagaagctgctgtac | |
| | atcttcaagcagcccttcatgcggcccgtgcagaccacacaagaggaagatggctgctcctgcagattccctgaggaagaagaag | |
| | gcggctgcgagctgagagtgaagttcagcagatccgccgacgcccctgcctaccagcagggacagaaccagctgtacaacgag | |
| | ctgaacctgggcagacgggaagagtacgacgtgctggacaagcggagaggccgggaccccgagatgggcggaaagcccaga | |
| | cggaagaacccccaggaaggcctgtataacgaactgcagaaagacaagatggccgaggcctacagcgagatcggcatgaaggg | |
| | cgagcggaggcgcggcaagggccacgatggcctgtaccagggcctgagcaccgccaccaaggacacctacgacgccctgcac | |
| | atgcaggccctgccccccagaggatccggcgctacaaatttttcactgctgaaacaggcgggtgatgtggaggagaaccctggac | |
| | ccatgccacttggcctgctctggctgggcttggcattgctcggcgcgctccacgcccaggctgaactgatccgcgtggccatattgt | |
| | ggcatgagatgtggcatgagggattggaggaggcgagtaggctgtactttggggaaaggaatgttaaagggatgtttgaggtccttg | |
| | aacccctccacgctatgatggaaagaggacctcaaacgcttaaagagacgtcattcaatcaagcctatggacgggatcttatggaag | |
| | ctcaagaatggtgtcgaaaatacatgaaaagcgggaatgttaaggacctcacgcaagcctgggatctgtattaccatgttttccgacg | |
| | catttctaaacaaggaaaagatactatcccatggttggggcacttgctcgttgggctcagtggggcgtttggattcatcatcctcgtatat | |
| | ctgttgattaattgtcggaacacaggtccctggcttaaaaaagttttgaagtgtaacaccccggatccttctaaattttttagtcaacttagt | |
| | tcagaacacgggggcgatgttcaaaagtggctgagttccccgtttcccagttcaagtttctcccctgggggtctcgcccccgagatat | |
| | cacctcttgaagtgctcgagcgggacaaagttacacagcttcttttgcaacaggataaggttccggagccggcgtctctcagctctaa | |
| | ccattcactcacttcttgtttcaccaaccaagggtattttttcttccatctgcctgatgccttggagattgaggcttgtcaggtgtactttacc | |
| | tatgacccctatagtgaggaagaccctgacgaaggcgtagctggcgcccccactggctccagtccacagcctcttcagcctctgtca | |
| | ggggaggacgacgcatattgtacgttcccctcacgggacgaccttctgctgttttcaccctcactgctcggcggaccctccccgcca | |
| | agcacggcacctggggggagtggggcaggagaagaaaggatgcctcctagtttgcaggagcgggttcctcgcgactgggatcc | |
| | gcaacccctcggaccacccacccctggcgtacctgatctggtcgacttccaaccacctccggagcttgtcctcagagaggccggag | |
| | aggaagtcccagacgcggggccaagagagggtgtgtcatttccctggtcccgccctccgggacagggtgagtttcgggcgctgaa | |
| | tgcgaggctcccccttaataccgatgcgtacctgtcattgcaggaacttcagggccaggatcctacccacctggtgtgaaagcttgat | |
| | aatcaacctctggattacaaaatttgtgaaagattgactggtattcttaactatgttgctcatttacgctatgtggatacgctgctttaatgc | |
| | ctttgtatcatgctattgcttcccgtatggctttcattttctcctccttgtataaatcctggttagttcttgccacggcggaactcatcgccgc | |
| | ctgccttgcccgctgctggacaggggctcggctgttgggcactgacaattccgtggaacttgtttattgcagcttataatggttacaaat | |
| | aaagcaatagcatcacaaatttcacaaataaagcatttttttcactgcattctagttgtggtttgtccaaactcatcaatgtatcttacgccg | |
| | gcgtgagtgtaccagctgagagactctaaatccagtgacaagtctgtctgcctattcaccgattttgattctcaaacaaatgtgtcacaa | |
| | agtaaggattctgatgtgtatatcacagacaaaactgtgctagacatgaggtctatggacttcaagagcaacagtgctgtggcctgga | |
| | gcaacaaatctgactttgcatgtgcaaacgccttcaacaacagcattattccagaagacaccttcttccccagcccaggtaagggcag | |
| | ctttggtgccttcgcaggctgtttccttgcttcaggaatggccaggttctgcccagagctctggtcaatgatgtctaaaactcctctgatt | |
| | ggtggtctcggccttatccattgccaccaaaaccctctttttactaagacctaggaacccctagtgatggagttggccactccctctctg | |
| | cgcgctcgctcgctcactgaggccgggcgaccaaaggtcgcccgacgcccgggctttgcccgggcggcctcagtgagcgagcg | |
| | agcgcgcagctgcctgcagg | |
| 39 | cctgcaggcagctgcgcgctcgctcgctcactgaggccgcccgggcaaagcccgggcgtcgggcgacctaggtcgcccggcct | TRAC AAV |
| | cagtgagcgagcgagcgcgcagagagggagtggccaactccatcactaggggacctgcggccgctggccgtgaacgttcactg | #7 TRAC 1: |
| | aaatcatggcctcttggccaagattgatagcttgtgcctgtccctgagtcccagtccatcacgagcagctggtttctaagatgctatttcc | HA TRAC |
| | cgtataaagcatgagaccgtgacttgccagccccacagagccccgcccttgtccatcactggcatctggactccagcctgggagg | 1-2A- |
| | ggcaaagagggaaatgagatcatgtcctaaccctgatcctcttgtcccacagatatccagaaccctgaccctgccgtgtaccagctg | C11D5.3- |
| | agagactctaaatccagtgacaagtctgtctgcctattcaccgattagattctcaaacaaatgtgtcacaaagtaaggattctgatgtgt | AAA-CD8- |
| | atatcacagacaaaactgtgctagacggttccggggagggccgagggtcattgctgacgtgtggagacgtggaggagaatcctgg | 41BB- |
| | ccccatggccctgcctgtgacagctctgctcctccctctggccctgctgctccatgccgccagacccgacatcgtgctgacccagag | CD3z-P2A- |
| | cccccccagcctggccatgtctctgggcaagagagccaccatcagctgccgggccagcgagagcgtgaccatcctgggcagcca | CISCβ-HA |
| | cctgatccactggtatcagcagaagcccggccagccccccaccctgctgatccagctcgccagcaatgtgcagaccggcgtgccc | TRAC 1 |
| | gccagattcagcggcagcggcagcagaaccgacttcaccctgaccatcgaccccgtggaagaggacgacgtggccgtgtactac | |
| | tgcctgcagagccggaccatcccccggacctttggcggaggcaccaaactggaaatcaagggcagcaccagcggctccggcaa | |
| | gcctggctctggcgagggcagcacaaagggacagattcagctggtgcagagcggccctgagctgaagaaacccggcgagaca | |
| | gtgaagatcagctgcaaggcctccggctacaccttcaccgactacagcatcaactgggtgaaaagagcccctggcaagggcctga | |
| | agtggatgggctggatcaacaccgagacaagagagcccgcctacgcctacgacttccggggcagattcgccttcagcctggaaac | |
| | cagcgccagcaccgcctacctgcagatcaacaacctgaagtacgaggacaccgccacctacttttgcgccctggactacagctacg | |
| | ccatggactactggggccagggcaccagcgtgaccgtgtccagcgccgccgccttcgtgcccgtgttcctgcccgccaaacctac | |
| | caccacccctgcccctagacctcccaccccagccccaacaatcgccagccagcctctgtctctgcggcccgaagcctgtagacctg | |
| | ctgccggcggagccgtgcacaccagaggcctggacttcgcctgcgacatctacatctgggcccctctggccggcacctgtggcgt | |
| | gctgctgctgagcctggtgatcaccctgtactgcaaccaccggaacagattcagcgtcgtgaagcggggcagaaagaagctgctgt | |
| | acatcttcaagcagcccttcatgcggcccgtgcagaccacacaagaggaagatggctgctcctgcagattccctgaggaagaaga | |
| | aggcggctgcgagctgagagtgaagttcagcagatccgccgacgcccctgcctaccagcagggacagaaccagctgtacaacg | |
| | agctgaacctgggcagacgggaagagtacgacgtgctggacaagcggagaggccgggaccccgagatgggcggaaagccca | |
| | gacggaagaacccccaggaaggcctgtataacgaactgcagaaagacaagatggccgaggcctacagcgagatcggcatgaag | |
| | ggcgagcggaggcgcggcaagggccacgatggcctgtaccagggcctgagcaccgccaccaaggacacctacgacgccctgc | |
| | acatgcaggccctgccccccagaggatccggcgctacaaatttttcactgctgaaacaggcgggtgatgtggaggagaaccctgg | |
| | acccatgccacttggcctgctctggctgggcttggcattgctcggcgcgctccacgcccaggctgaactgatccgcgtggccatatt | |
| | gtggcatgagatgtggcatgagggattggaggaggcgagtaggctgtactttggggaaaggaatgttaaagggatgtttgaggtcct | |
| | tgaacccctccacgctatgatggaaagaggacctcaaacgcttaaagagacgtcattcaatcaagcctatggacgggatcttatgga | |
| | agctcaagaatggtgtcgaaaatacatgaaaagcgggaatgttaaggacctcacgcaagcctgggatctgtattaccatgttttccga | |
| | cgcatttctaaacaaggaaaagatactatcccatggttggggcacttgctcgttgggctcagtggggcgtttggattcatcatcctcgta | |
| | tatctgttgattaattgtcggaacacaggtccctggcttaaaaaagttttgaagtgtaacaccccggatccttctaaattttttagtcaactt | |
| | agttcagaacacgggggcgatgttcaaaagtggctgagttccccgtttcccagttcaagtttctcccctgggggtctcgcccccgaga | |
| | tatcacctcttgaagtgctcgagcgggacaaagttacacagcttcttttgcaacaggataaggttccggagccggcgtctctcagctct | |
| | aaccattcactcacttcttgtttcaccaaccaagggtattttttcttccatctgcctgatgccttggagattgaggcttgtcaggtgtacttta | |
| | cctatgacccctatagtgaggaagaccctgacgaaggcgtagctggcgcccccactggctccagtccacagcctcttcagcctctgt | |
| | caggggaggacgacgcatattgtacgttcccctcacgggacgaccttctgctgttttcaccctcactgctcggcggaccctccccgc | |
| | caagcacggcacctggggggagtggggcaggagaagaaaggatgcctcctagtttgcaggagcgggttcctcgcgactgggatc | |
| | cgcaacccctcggaccacccacccctggcgtacctgatctggtcgacttccaaccacctccggagcttgtcctcagagaggccgga | |
| | gaggaagtcccagacgcggggccaagagagggtgtgtcatttccctggtcccgccctccgggacagggtgagtttcgggcgctg | |
| | aatgcgaggctcccccttaataccgatgcgtacctgtcattgcaggaacttcagggccaggatcctacccacctggtgtgaaagcttg | |
| | ataatcaacctctggattacaaaatttgtgaaagattgactggtattcttaactatgttgctcatttacgctatgtggatacgctgctttaat | |
| | gcctttgtatcatgctattgcttcccgtatggctttcattttctcctccttgtataaatcctggttagttcttgccacggcggaactcatcgcc | |
| | gcctgccttgcccgctgctggacaggggctcggctgttgggcactgacaattccgtggaacttgtttattgcagcttataatggttaca | |
| | aataaagcaatagcatcacaaatttcacaaataaagcatttattcactgcattctagttgtggtttgtccaaactcatcaatgtatcttacgc | |
| | cggcgtgaatgaggtctatggacttcaagagcaacagtgctgtggcctggagcaacaaatctgactttgcatgtgcaaacgccttca | |
| | acaacagcattattccagaagacaccttcttccccagcccaggtaagggcagctttggtgccttcgcaggctgtttccttgcttcagga | |
| | atggccaggttctgcccagagctctggtcaatgatgtctaaaactcctctgattggtggtctcggccttatccattgccaccaaaaccct | |
| | attttactaagaaacagtgagccttgttctggcagtccagagaatgacacgggaaaaaagcagatgaagagaaggtggcaggaga | |
| | gggcacgtggcccagcctcagtctctccaactgagttcctgcctgcctgcattgccctaggaacccctagtgatggagttggccact | |
| | ccctctctgcgcgctcgctcgctcactgaggccgggcgaccaaaggtcgcccgacgcccgggctttgcccgggcggcctcagtg | |
| | agcgagcgagcgcgcagctgcctgcagg | |
| 40 | cctgcaggcagctgcgcgctcgctcgctcactgaggccgcccgggcaaagcccgggcgtcgggcgacctttggtcgcccggcct | IL2RG |
| | cagtgagcgagcgagcgcgcagagagggagtggccaactccatcactaggggttcctgcggccgccaacctctagaaatcaagg | AAV #8: |
| | tttttctgtgtagggttgggttagcgtgttgttagagtaggggagtggattgagaaggaggctgaggggtactcaagggggctataga | HA-MND- |
| | atgtataggatttccctgaagcattcctagagagcctgcaaggtgaagatggctttggaaccagctggatctaggctgtgccacatact | nakedFRB- |
| | acctctttggccttggccacatccctaaactcttggattctgtttcctaagatgtaagatggaggtaattgttcctgcctcacaggagctgt | tLNGFR- |
| | tgtgaggattaaacagagagtatgtctttagcgcggtgcctggcaccagtgcctggcatgtagtaggggcacaacaaatataaggtc | CNb30- |
| | cactttgcttttcttttttctatagttaattaagtgtgaacagagaaacaggagaatatgggccaaacaggatatctgtggtaagcagttcc | CISCy HA |
| | tgccccggctcagggccaagaacagttggaacagcagaatatgggccaaacaggatatctgtggtaagcagttcctgccccggct | |
| | cagggccaagaacagatggtccccagatgcggtcccgccctcagcagtttctagagaaccatcagatgtttccagggtgccccaag | |
| | gacctgaaatgaccctgtgccttatttgaactaaccaatcagttcgcttctcgcttctgttcgcgcgcttctgctccccgagctctatataa | |
| | gcagagctcgtttagtgaaccgtcagatcggtaccgccgccaccatggagatgtggcatgagggtctggaagaagcgtctcgactg | |
| | tactttggtgagcgcaatgtgaagggcatgtttgaagtcctcgaaccccttcatgccatgatggaacgcggaccccagaccttgaag | |
| | gagacaagttttaaccaagcttacggaagagacctgatggaagcccaggaatggtgcaggaaatacatgaaaagcgggaatgtga | |
| | aggacttgctccaagcgtgggacctgtactatcacgtctttaggcgcattagtaagggcagcggcgccacaaatttcagcctgctga | |
| | aacaggccggcgacgtggaagagaaccctggacccatgggtgctggcgcaactggacgcgctatggatggacctcgcttgctgc | |
| | ttcttctgcttctcggggtctcattgggtggtgctaaggaagcatgcccaacgggactttatacgcatagcggagagtgttgcaaagct | |
| | tgtaacctgggcgaaggcgtcgcgcaaccttgtggtgcaaatcaaaccgtctgcgagccatgtttggactctgttacgtttagtgacgt | |
| | agtatctgcgacagagccatgcaagccttgtacggaatgtgtaggattgcagagcatgtctgccccttgtgtagaagccgacgatgc | |
| | agtttgcaggtgcgcgtatggctattaccaagacgaaacaaccggacgatgtgaagcttgccgagtttgtgaagcgggttccgggct | |
| | tgtattctcatgtcaggataagcagaacaccgtctgcgaagagtgccccgatggcacctacagcgatgaagcgaaccatgtagacc | |
| | cctgcctgccttgcaccgtttgtgaagacacggaacgacagttgcgggagtgtacccggtgggcagacgccgagtgcgaagagat | |
| | tccaggccgctggatcacgcgaagtaccccgccagaaggttccgacagtactgcaccaagcacccaagaaccagaggcgcccc | |
| | ccgagcaggacctgattgcctccaccgtggcgggtgttgttactacggttatgggctcatcccagcccgttgttacccgaggaactac | |
| | agacaacctgattccggtatattgttctatcttggcggctgtagtagttggcttggtcgcctacatcgctttcaaaagaggttccgggga | |
| | gggccgagggtcattgctgacgtgtggagacgtggaggagaatcctggccccatgggcaacgaggccagctaccctctggagat | |
| | gtgctcccacttcgacgccgacgagatcaagggctgggcaagcgcttcaagaagctggacctggacaacagcggcagcctgag | |
| | cgtggaggagtttatgtctctgcccgagctgcagcagaaccccctggtgcagcgcgtgatcgacatcttcgacaccgacggcaacg | |
| | gcgaggtggacttcaaggagttcatcgagggcgtgagccagttcagcgtgaagggcgacaaggagcagaagctgcggttcgcct | |
| | tccggatctacgatatggataaagatggctatatttctaatggcgagctgttccaggtgctgaagatgatggtgggcaacaataccaa | |
| | gctggccgatacccagctgcagcagatcgtggacaagaccatcatcaacgccgacaaggacggcgacggcagaatcagatcga | |
| | ggagttctgtgccgtggtgggaggcctggatattcacaaaaaaatggtggtggacgtgggatccggcgctacaaatttttcactgctg | |
| | aaacaggcgggtgacgtggaggagaaccctggacccatgcctctgggcctgctgtggctgggcctggccctgctgggcgccctg | |
| | cacgcccaggccggcgtgcaggtggagacaatctccccaggcgacggacgcacattccctaagggggccagacctgcgtggt | |
| | gcactatacaggcatgctggaggatggcaagaagtttgacagctcccgggatagaaacaagccattcaagtttatgctgggcaagc | |
| | aggaagtgatcagaggctgggaggagggcgtggcccagatgtctgtgggccagagggccaagctgaccatcagcccagactac | |
| | gcctatggagcaacaggccacccaggaatcatcccacctcacgccaccctggtgttcgatgtggagctgctgaagctgggcgagg | |
| | gcagcaacaccagcaaagagaatcctttcctgtttgcattggaagccgtggttatctctgttggctccatgggattgattatcagccttct | |
| | ctgtgtgtatttctggctggaacggtgagatttggagaagcccagaaaaatgaggggaacggtagctgacaatagcagaggagggt | |
| | tttgcagggtattaggagtaaaggatgagacagtaagtaatgagagattacccaagagggtttggtgatggaaggaagccacagg | |
| | cacagagaacacagaatcactttatttcatatgggacaactgggagaagggtgataaaaaagctttaacctatgtgctcctgctccctc | |
| | tttctcccctgtcaggacgatgccccgaattcccaccctgaagaacctagaggatcttgttactgaataccacgggaacttttcggtga | |
| | gaacgctgtcatcaattgtctacctaggaacccctagtgatggagttggccactccctctctgcgcgctcgctcgctcactgaggccg | |
| | ggcgaccaaaggtcgcccgacgcccgggctttgcccgggcggcctcagtgagcgagcgagcgcgcagctgcctgcagg | |
| 41 | cctgcaggcagctgcgcgctcgctcgctcactgaggccgcccgggcaaagcccgggcgtcgggcgacctaggtcgcccggcct | IL2RG |
| | cagtgagcgagcgagcgcgcagagagggagtggccaactccatcactaggggacctgcggccgccaacctctagaaatcaagg | AAV #8 |
| | tttttctgtgtagggttgggttagcgtgttgttagagtaggggagtggattgagaaggaggctgaggggtactcaagggggctataga | GC8: HA- |
| | atgtataggatttccctgaagcattcctagagagcctgcaaggtgaagatggctttggaaccagctggatctaggctgtgccacatact | MND- |
| | acctctttggccttggccacatccctaaactcttggattctgtttcctaagatgtaagatggaggtaattgttcctgcctcacaggagctgt | nakedFRB- |
| | tgtgaggattaaacagagagtatgtctttagcgcggtgcctggcaccagtgcctggcatgtagtaggggcacaacaaatataaggtc | tLNGFR- |
| | cactttgcttttcttttttctatagttaattaagtgtgaacagagaaacaggagaatatgggccaaacaggatatctgtggtaagcagttcc | CNb30- |
| | tgccccggctcagggccaagaacagttggaacagcagaatatgggccaaacaggatatctgtggtaagcagttcctgccccggct | CISCy HA |
| | cagggccaagaacagatggtccccagatgcggtcccgccctcagcagtttctagagaaccatcagatgtaccagggtgccccaag | for GC8 |
| | gacctgaaatgaccctgtgccttatttgaactaaccaatcagttcgcttctcgcttctgttcgcgcgcttctgctccccgagctctatataa | |
| | gcagagctcgtttagtgaaccgtcagatcggtaccgccgccaccatggagatgtggcatgagggtctggaagaagcgtctcgactg | |
| | tactttggtgagcgcaatgtgaagggcatgtttgaagtcctcgaaccccttcatgccatgatggaacgcggaccccagaccttgaag | |
| | gagacaagttttaaccaagatacggaagagacctgatggaagcccaggaatggtgcaggaaatacatgaaaaggggaatgtga | |
| | aggacttgctccaagcgtgggacctgtactatcacgtattaggcgcattagtaagggcagcggcgccacaaatttcagcctgctga | |
| | aacaggccggcgacgtggaagagaaccctggacccatgggtgctggcgcaactggacgcgctatggatggacctcgcttgctgc | |
| | ttatctgatctcggggtctcattgggtggtgctaaggaagcatgcccaacgggactttatacgcatagggagagtgttgcaaagct | |
| | tgtaacctgggcgaaggcgtcgcgcaaccttgtggtgcaaatcaaaccgtctgcgagccatgtttggactctgttacgtttagtgacgt | |
| | agtatctgcgacagagccatgcaagccttgtacggaatgtgtaggattgcagagcatgtctgccccttgtgtagaagccgacgatgc | |
| | agtttgcaggtgcgcgtatggctattaccaagacgaaacaaccggacgatgtgaagcttgccgagtttgtgaagcgggttccgggct | |
| | tgtattctcatgtcaggataagcagaacaccgtctgcgaagagtgccccgatggcacctacagcgatgaagcgaaccatgtagacc | |
| | cctgcctgccttgcaccgtttgtgaagacacggaacgacagttgcgggagtgtacccggtgggcagacgccgagtgcgaagagat | |
| | tccaggccgctggatcacgcgaagtaccccgccagaaggttccgacagtactgcaccaagcacccaagaaccagaggcgcccc | |
| | ccgagcaggacctgattgcctccaccgtggcgggtgttgttactacggttatgggctcatcccagcccgttgttacccgaggaactac | |
| | agacaacctgattccggtatattgttctatcttggcggctgtagtagttggcttggtcgcctacatcgctttcaaaagaggttccgggga | |
| | gggccgagggtcattgctgacgtgtggagacgtggaggagaatcctggccccatgggcaacgaggccagctaccctctggagat | |
| | gtgctcccacttcgacgccgacgagatcaagggctgggcaagcgcttcaagaagctggacctggacaacagcggcagcctgag | |
| | cgtggaggagtttatgtctctgcccgagctgcagcagaaccccctggtgcagcgcgtgatcgacatcttcgacaccgacggcaacg | |
| | gcgaggtggacttcaaggagttcatcgagggcgtgagccagttcagcgtgaagggcgacaaggagcagaagctgcggttcgcct | |
| | tccggatctacgatatggataaagatggctatatttctaatggcgagctgttccaggtgctgaagatgatggtgggcaacaataccaa | |
| | gctggccgatacccagctgcagcagatcgtggacaagaccatcatcaacgccgacaaggacggcgacggcagaatcagatcga | |
| | ggagttctgtgccgtggtgggaggcctggatattcacaaaaaaatggtggtggacgtgggatccggcgctacaaatttttcactgctg | |
| | aaacaggcgggtgacgtggaggagaaccctggacccatgcctctgggcctgctgtggctgggcctggccctgctgggcgccctg | |
| | cacgcccaggccggcgtgcaggtggagacaatctccccaggcgacggacgcacattccctaagggggccagacctgcgtggt | |
| | gcactatacaggcatgctggaggatggcaagaagtttgacagctcccgggatagaaacaagccattcaagtttatgctgggcaagc | |
| | aggaagtgatcagaggctgggaggagggcgtggcccagatgtctgtgggccagagggccaagctgaccatcagcccagactac | |
| | gcctatggagcaacaggccacccaggaatcatcccacctcacgccaccctggtgttcgatgtggagctgctgaagctgggcgagg | |
| | gcagcaacaccagcaaagaaaacccctattgttcgctcttgaggctgtcgtgattagcgtcggatccatgggattgattatcagccttc | |
| | tctgtgtgtatttctggctggaacggtgagatttggagaagcccagaaaaatgaggggaacggtagctgacaatagcagaggaggg | |
| | ttttgcagggtattaggagtaaaggatgagacagtaagtaatgagagattacccaagagggtttggtgatggaaggaagccacagg | |
| | cacagagaacacagaatcactttatttcatatgggacaactgggagaagggtgataaaaaagattaacctatgtgacctgaccac | |
| | tttacccctgtcaggacgatgccccgaattcccaccctgaagaacctagaggatcttgttactgaataccacgggaacttttcggtga | |
| | gaacgctgtcatcaattgtctacctaggaacccctagtgatggagttggccactccctactgcgcgctcgctcgctcactgaggccg | |
| | ggcgaccaaaggtcgcccgacgcccgggattgcccgggcggcctcagtgagcgagcgagcgcgcagctgcctgcagg | |
| 42 | cctgcaggcagctgcgcgctcgctcgctcactgaggccgcccgggcaaagcccgggcgtcgggcgacctaggtcgcccggcct | IL2RG |
| | cagtgagcgagcgagcgcgcagagagggagtggccaactccatcactaggggttcctgcggccgccaacctctagaaatcaagg | AAV #8 |
| | tttttctgtgtagggttgggttagcgtgttgttagagtaggggagtggattgagaaggaggctgaggggtactcaagggggctataga | GC10: HA- |
| | atgtataggatttccctgaagcattcctagagagcctgcaaggtgaagatggattggaaccagctggatctaggctgtgccacatact | MND- |
| | acctattggccttggccacatccctaaactatggattctgtttcctaagatgtaagatggaggtaattgttcctgcctcacaggagctgt | nakedFRB- |
| | tgtgaggattaaacagagagtatgtattagcgcggtgcctggcaccagtgcctggcatgtagtaggggcacaacaaatataaggtc | tLNGFR- |
| | cactttgatttatttttctatagttaattaagtgtgaacagagaaacaggagaatatgggccaaacaggatatctgtggtaagcagttcc | CNb30- |
| | tgccccggctcagggccaagaacagttggaacagcagaatatgggccaaacaggatatctgtggtaagcagttcctgccccggct | CISCy HA |
| | cagggccaagaacagatggtccccagatgcggtcccgccctcagcagtttctagagaaccatcagatgtttccagggtgccccaag | for GC10 |
| | gacctgaaatgaccctgtgccttatttgaactaaccaatcagttcgcttctcgcttctgttcgcgcgcttctgctccccgagctctatataa | |
| | gcagagacgtttagtgaaccgtcagatcggtaccgccgccaccatggagatgtggcatgagggtctggaagaagcgtacgactg | |
| | tactttggtgagcgcaatgtgaagggcatgtttgaagtcctcgaaccccttcatgccatgatggaacgcggaccccagaccttgaag | |
| | gagacaagttttaaccaagatacggaagagacctgatggaagcccaggaatggtgcaggaaatacatgaaaaggggaatgtga | |
| | aggacttgctccaagcgtgggacctgtactatcacgtattaggcgcattagtaagggcagcggcgccacaaatttcagcctgctga | |
| | aacaggccggcgacgtggaagagaaccctggacccatgggtgctggcgcaactggacgcgctatggatggacctcgcttgctgc | |
| | ttatctgatctcggggtacattgggtggtgctaaggaagcatgcccaacgggactttatacgcatagggagagtgttgcaaagct | |
| | tgtaacctgggcgaaggcgtcgcgcaaccttgtggtgcaaatcaaaccgtctgcgagccatgtttggactctgttacgtttagtgacgt | |
| | agtatctgcgacagagccatgcaagccttgtacggaatgtgtaggattgcagagcatgtctgccccttgtgtagaagccgacgatgc | |
| | agtttgcaggtgcgcgtatggctattaccaagacgaaacaaccggacgatgtgaagcttgccgagtttgtgaagcgggttccgggct | |
| | tgtattacatgtcaggataagcagaacaccgtctgcgaagagtgccccgatggcacctacagcgatgaagcgaaccatgtagacc | |
| | cctgcctgccttgcaccgtttgtgaagacacggaacgacagttgcgggagtgtacccggtgggcagacgccgagtgcgaagagat | |
| | tccaggccgctggatcacgcgaagtaccccgccagaaggttccgacagtactgcaccaagcacccaagaaccagaggcgcccc | |
| | ccgagcaggacctgattgcctccaccgtggcgggtgttgttactacggttatgggctcatcccagcccgttgttacccgaggaactac | |
| | agacaacctgattccggtatattgttctatcttggcggctgtagtagttggcttggtcgcctacatcgctttcaaaagaggttccgggga | |
| | gggccgagggtcattgctgacgtgtggagacgtggaggagaatcctggccccatgggcaacgaggccagctaccactggagat | |
| | gtgacccacttcgacgccgacgagatcaagggctgggcaagcgcttcaagaagctggacctggacaacagcggcagcctgag | |
| | cgtggaggagtttatgtactgcccgagctgcagcagaaccccctggtgcagcgcgtgatcgacatcttcgacaccgacggcaacg | |
| | gcgaggtggacttcaaggagttcatcgagggcgtgagccagttcagcgtgaagggcgacaaggagcagaagctgcggttcgcct | |
| | tccggatctacgatatggataaagatggctatatttctaatggcgagctgttccaggtgctgaagatgatggtgggcaacaataccaa | |
| | gctggccgatacccagctgcagcagatcgtggacaagaccatcatcaacgccgacaaggacggcgacggcagaatcagatcga | |
| | ggagttctgtgccgtggtgggaggcctggatattcacaaaaaaatggtggtggacgtgggatccggcgctacaaatttttcactgctg | |
| | aaacaggcgggtgacgtggaggagaaccctggacccatgcctctgggcctgctgtggctgggcctggccctgctgggcgccctg | |
| | cacgcccaggccggcgtgcaggtggagacaataccccaggcgacggacgcacattccctaagggggccagacctgcgtggt | |
| | gcactatacaggcatgctggaggatggcaagaagtttgacagctcccgggatagaaacaagccattcaagtttatgctgggcaagc | |
| | aggaagtgatcagaggctgggaggagggcgtggcccagatgtctgtgggccagagggccaagctgaccatcagcccagactac | |
| | gcctatggagcaacaggccacccaggaatcatcccacctcacgccaccctggtgttcgatgtggagctgctgaagctgggcgagg | |
| | gcagcaacaccagcaaagaaaacccattttgttcgctatgaggctgtcgtgattagcgtcggaagtatgggattgattatcagccttc | |
| | tctgtgtgtatttctggctggaacggtgagatttggagaagcccagaaaaatgaggggaacggtagctgacaatagcagaggaggg | |
| | ttttgcagggtattaggagtaaaggatgagacagtaagtaatgagagattacccaagagggtttggtgatggaaggaagccacagg | |
| | cacagagaacacagaatcactttatttcatatgggacaactgggagaagggtgataaaaaagattaacctatgtgacctgaccac | |
| | tttacccctgtcaggacgatgccccgaattcccaccctgaagaacctagaggatcttgttactgaataccacgggaacttttcggtga | |
| | gaacgctgtcatcaattgtctacctaggaacccctagtgatggagttggccactccctactgcgcgctcgctcgctcactgaggccg | |
| | ggcgaccaaaggtcgcccgacgcccgggattgcccgggcggcctcagtgagcgagcgagcgcgcagctgcctgcagg | |
| 43 | cctgcaggcagctgcgcgctcgctcgctcactgaggccgcccgggcaaagcccgggcgtcgggcgacctaggtcgcccggcct | IL2RG |
| | cagtgagcgagcgagcgcgcagagagggagtggccaactccatcactaggggttcctgcggccgccaacctctagaaatcaagg | AAV #8 |
| | tttttctgtgtagggttgggttagcgtgttgttagagtaggggagtggattgagaaggaggctgaggggtactcaagggggctataga | GC12: HA- |
| | atgtataggatttccctgaagcattcctagagagcctgcaaggtgaagatggattggaaccagctggatctaggctgtgccacatact | MND- |
| | acctattggccttggccacatccctaaactatggattctgtttcctaagatgtaagatggaggtaattgttcctgcctcacaggagctgt | nakedFRB- |
| | tgtgaggattaaacagagagtatgtattagcgcggtgcctggcaccagtgcctggcatgtagtaggggcacaacaaatataaggtc | tLNGFR- |
| | cactttgcttttcttttttctatagttaattaagtgtgaacagagaaacaggagaatatgggccaaacaggatatctgtggtaagcagttcc | CNb30- |
| | tgccccggctcagggccaagaacagttggaacagcagaatatgggccaaacaggatatctgtggtaagcagttcctgccccggct | CISCy HA |
| | cagggccaagaacagatggtccccagatgcggtcccgccctcagcagtttctagagaaccatcagatgtaccagggtgccccaag | for GC12 |
| | gacctgaaatgaccctgtgccttatttgaactaaccaatcagttcgcttctcgcttctgttcgcgcgcttctgctccccgagctctatataa | |
| | gcagagctcgtttagtgaaccgtcagatcggtaccgccgccaccatggagatgtggcatgagggtctggaagaagcgtctcgactg | |
| | tactttggtgagcgcaatgtgaagggcatgtttgaagtcctcgaaccccttcatgccatgatggaacgcggaccccagaccttgaag | |
| | gagacaagttttaaccaagcttacggaagagacctgatggaagcccaggaatggtgcaggaaatacatgaaaagcgggaatgtga | |
| | aggacttgctccaagcgtgggacctgtactatcacgtctttaggcgcattagtaagggcagcggcgccacaaatttcagcctgctga | |
| | aacaggccggcgacgtggaagagaaccctggacccatgggtgctggcgcaactggacgcgctatggatggacctcgcttgctgc | |
| | ttcttctgcttctcggggtctcattgggtggtgctaaggaagcatgcccaacgggactttatacgcatagcggagagtgttgcaaagct | |
| | tgtaacctgggcgaaggcgtcgcgcaaccttgtggtgcaaatcaaaccgtctgcgagccatgtttggactctgttacgtttagtgacgt | |
| | agtatctgcgacagagccatgcaagccttgtacggaatgtgtaggattgcagagcatgtctgccccttgtgtagaagccgacgatgc | |
| | agtttgcaggtgcgcgtatggctattaccaagacgaaacaaccggacgatgtgaagcttgccgagtttgtgaagcgggttccgggct | |
| | tgtattctcatgtcaggataagcagaacaccgtctgcgaagagtgccccgatggcacctacagcgatgaagcgaaccatgtagacc | |
| | cctgcctgccttgcaccgtttgtgaagacacggaacgacagttgcgggagtgtacccggtgggcagacgccgagtgcgaagagat | |
| | tccaggccgctggatcacgcgaagtaccccgccagaaggttccgacagtactgcaccaagcacccaagaaccagaggcgcccc | |
| | ccgagcaggacctgattgcctccaccgtggcgggtgttgttactacggttatgggctcatcccagcccgttgttacccgaggaactac | |
| | agacaacctgattccggtatattgttctatcttggcggctgtagtagttggcttggtcgcctacatcgctttcaaaagaggttccgggga | |
| | gggccgagggtcattgctgacgtgtggagacgtggaggagaatcctggccccatgggcaacgaggccagctaccctctggagat | |
| | gtgctcccacttcgacgccgacgagatcaagcggctgggcaagcgcttcaagaagctggacctggacaacagcggcagcctgag | |
| | cgtggaggagtttatgtctctgcccgagctgcagcagaaccccctggtgcagcgcgtgatcgacatcttcgacaccgacggcaacg | |
| | gcgaggtggacttcaaggagttcatcgagggcgtgagccagttcagcgtgaagggcgacaaggagcagaagctgcggttcgcct | |
| | tccggatctacgatatggataaagatggctatatttctaatggcgagctgttccaggtgctgaagatgatggtgggcaacaataccaa | |
| | gctggccgatacccagctgcagcagatcgtggacaagaccatcatcaacgccgacaaggacggcgacggcagaatcagcttcga | |
| | ggagttctgtgccgtggtgggaggcctggatattcacaaaaaaatggtggtggacgtgggatccggcgctacaaatttttcactgctg | |
| | aaacaggcgggtgacgtggaggagaaccctggacccatgcctctgggcctgctgtggctgggcctggccctgctgggcgccctg | |
| | cacgcccaggccggcgtgcaggtggagacaatctccccaggcgacggacgcacattccctaagcggggccagacctgcgtggt | |
| | gcactatacaggcatgctggaggatggcaagaagtttgacagctcccgggatagaaacaagccattcaagtttatgctgggcaagc | |
| | aggaagtgatcagaggctgggaggagggcgtggcccagatgtctgtgggccagagggccaagctgaccatcagcccagactac | |
| | gcctatggagcaacaggccacccaggaatcatcccacctcacgccaccctggtgttcgatgtggagctgctgaagctgggcgagg | |
| | gcagcaacaccagcaaagaaaacccctattgtttgcattggaagccgtggttatctctgttggctccatgggattgattatcagccttct | |
| | ctgtgtgtatttctggctggaacggtgagatttggagaagcccagaaaaatgaggggaacggtagctgacaatagcagaggagggt | |
| | tttgcagggtattaggagtaaaggatgagacagtaagtaatgagagattacccaagagggtttggtgatggaaggaagccacagg | |
| | cacagagaacacagaatcactttatttcatatgggacaactgggagaagggtgataaaaaagctttaacctatgtgctcctgctccctc | |
| | tttctcccctgtcaggacgatgccccgaattcccaccctgaagaacctagaggatcttgttactgaataccacgggaacttttcggtga | |
| | gaacgctgtcatcaattgtctacctaggaacccctagtgatggagttggccactccctctctgcgcgctcgctcgctcactgaggccg | |
| | ggcgaccaaaggtcgcccgacgcccgggctttgcccgggcggcctcagtgagcgagcgagcgcgcagctgcctgcagg | |
| 44 | cctgcaggcagctgcgcgctcgctcgctcactgaggccgcccgggcaaagcccgggcgtcgggcgacctaggtcgcccggcct | IL2RG |
| | cagtgagcgagcgagcgcgcagagagggagtggccaactccatcactaggggttcctgcggccgccaacctctagaaatcaagg | AAV #9: |
| | tttttctgtgtagggttgggttagcgtgttgttagagtaggggagtggattgagaaggaggctgaggggtactcaagggggctataga | HA-MND- |
| | atgtataggatttccctgaagcattcctagagagcctgcaaggtgaagatggctttggaaccagctggatctaggctgtgccacatact | B2MCAR- |
| | acctctttggccttggccacatccctaaactcttggattctgtttcctaagatgtaagatggaggtaattgttcctgcctcacaggagctgt | nakedFRB- |
| | tgtgaggattaaacagagagtatgtctttagcgcggtgcctggcaccagtgcctggcatgtagtaggggcacaacaaatataaggtc | tLNGFR- |
| | cactttgcttttcttttttctatagttaattaagtgtgaacagagaaacaggagaatatgggccaaacaggatatctgtggtaagcagttcc | CNb30- |
| | tgccccggctcagggccaagaacagttggaacagcagaatatgggccaaacaggatatctgtggtaagcagttcctgccccggct | CISCy HA |
| | cagggccaagaacagatggtccccagatgcggtcccgccctcagcagtttctagagaaccatcagatgtttccagggtgccccaag | |
| | gacctgaaatgaccctgtgccttatttgaactaaccaatcagttcgcttctcgcttctgttcgcgcgcttctgctccccgagctctatataa | |
| | gcagagctcgtttagtgaaccgtcagatcggtaccgccgccaccatgagcaggtcagtggcgttggcggttctggcgatttgagttt | |
| | gagcggactggaagccatccaacgaacgcctaagatccaggtatattcacgccacccggcggaaaacggcaaaagtaacttcctt | |
| | aattgttatgtgtctggatccacccgtctgatattgaggtggacctccttaaaaacggtgaacggatcgagaaagtggagcattccga | |
| | tatagtttcagtaaggattggagatttaccttctctattacactgagttcactccgactgaaaaggatgagtacgcctgtcgggtcaac | |
| | cacgtcaccctgtctcaaccaaaaatagtcaaatgggacagagatatgtcagatatttacatatgggcaccacttgcgggcacgtgtg | |
| | gcgtcctgcttctgagtctcgtcattacgctttattgtaaacggggtagaaaaaaactcattatatatttaaacagccatttatgcggcca | |
| | gttcaaacgacgcaggaagaagacggctgtagttgcagatttccagaggaagaggaaggtggatgcgagatcgggtcaagttta | |
| | gtaggtctgcagacgctcccgcctatcaacagggtcagaatcagattataacgaactcaacctcggtcgccgagaagagtacgac | |
| | gtactcgataaaagaaggggtagagacccggaaatggggggcaaaccgcgccgcaaaaatccacaagaggggctttataatgag | |
| | cttcaaaaagacaaaatggccgaagcatacagtgagattgggatgaaaggtgaacgcagaagaggtaagggtcacgacgggctg | |
| | taccagggtttgtcaactgccacaaaggatacttatgacgctctgcatatgcaagctatcccccacgcggcagggcgaaggcaga | |
| | ggatccctgatacatgtggcgacgtggaagagaaccctggccccatggagatgtggcatgagggtctggaagaagcgtctcgact | |
| | gtactttggtgagcgcaatgtgaagggcatgtttgaagtcctcgaaccccttcatgccatgatggaacgcggaccccagaccttgaa | |
| | ggagacaagttttaaccaagcttacggaagagacctgatggaagcccaggaatggtgcaggaaatacatgaaaagcgggaatgtg | |
| | aaggacttgctccaagcgtgggacctgtactatcacgtctttaggcgcattagtaagggcagcggcgccacaaatttcagcctgctg | |
| | aaacaggccggcgacgtggaagagaaccctggacccatgggtgctggcgcaactggacgcgctatggatggacctcgcttgctg | |
| | cttatctgcttctcggggtctcattgggtggtgctaaggaagcatgcccaacgggactttatacgcatagcggagagtgttgcaaagc | |
| | ttgtaacctgggcgaaggcgtcgcgcaaccttgtggtgcaaatcaaaccgtctgcgagccatgtttggactctgttacgtttagtgacg | |
| | tagtatctgcgacagagccatgcaagccttgtacggaatgtgtaggattgcagagcatgtctgccccttgtgtagaagccgacgatgc | |
| | agtttgcaggtgcgcgtatggctattaccaagacgaaacaaccggacgatgtgaagcttgccgagtttgtgaagcgggttccgggct | |
| | tgtattctcatgtcaggataagcagaacaccgtctgcgaagagtgccccgatggcacctacagcgatgaagcgaaccatgtagacc | |
| | cctgcctgccttgcaccgtttgtgaagacacggaacgacagttgcgggagtgtacccggtgggcagacgccgagtgcgaagagat | |
| | tccaggccgctggatcacgcgaagtaccccgccagaaggttccgacagtactgcaccaagcacccaagaaccagaggcgcccc | |
| | ccgagcaggacctgattgcctccaccgtggcgggtgttgttactacggttatgggctcatcccagcccgttgttacccgaggaactac | |
| | agacaacctgattccggtatattgttctatcttggcggctgtagtagttggcttggtcgcctacatcgctttcaaaagaggttccgggga | |
| | gggccgagggtcattgctgacgtgtggagacgtggaggagaatcctggccccatgggcaacgaggccagctaccctctggagat | |
| | gtgctcccacttcgacgccgacgagatcaagcggctgggcaagcgcttcaagaagctggacctggacaacagcggcagcctgag | |
| | cgtggaggagtttatgtctctgcccgagctgcagcagaaccccctggtgcagcgcgtgatcgacatcttcgacaccgacggcaacg | |
| | gcgaggtggacttcaaggagttcatcgagggcgtgagccagttcagcgtgaagggcgacaaggagcagaagctgcggttcgcct | |
| | tccggatctacgatatggataaagatggctatatttctaatggcgagctgttccaggtgctgaagatgatggtgggcaacaataccaa | |
| | gctggccgatacccagctgcagcagatcgtggacaagaccatcatcaacgccgacaaggacggcgacggcagaatcagcttcga | |
| | ggagttctgtgccgtggtgggaggcctggatattcacaaaaaaatggtggtggacgtgggatccggcgctacaaatttttcactgctg | |
| | aaacaggcgggtgacgtggaggagaaccctggacccatgcctctgggcctgctgtggctgggcctggccctgctgggcgccctg | |
| | cacgcccaggccggcgtgcaggtggagacaatctccccaggcgacggacgcacattccctaagcggggccagacctgcgtggt | |
| | gcactatacaggcatgctggaggatggcaagaagtttgacagctcccgggatagaaacaagccattcaagtttatgctgggcaagc | |
| | aggaagtgatcagaggctgggaggagggcgtggcccagatgtctgtgggccagagggccaagctgaccatcagcccagactac | |
| | gcctatggagcaacaggccacccaggaatcatcccacctcacgccaccctggtgttcgatgtggagctgctgaagctgggcgagg | |
| | gcagcaacaccagcaaagagaatcctttcctgtttgcattggaagccgtggttatctctgttggctccatgggattgattatcagccttct | |
| | ctgtgtgtatttctggctggaacggtgagatttggagaagcccagaaaaatgaggggaacggtagctgacaatagcagaggagggt | |
| | tttgcagggtattaggagtaaaggatgagacagtaagtaatgagagattacccaagagggtttggtgatggaaggaagccacagg | |
| | cacagagaacacagaatcactttatttcatatgggacaactgggagaagggtgataaaaaagctttaacctatgtgctcctgctccctc | |
| | tttctcccctgtcaggacgatgccccgaattcccaccctgaagaacctagaggatcttgttactgaataccacgggaacttttcggtga | |
| | gaacgctgtcatcaattgtctacctaggaacccctagtgatggagttggccactccctctctgcgcgctcgctcgctcactgaggccg | |
| | ggcgaccaaaggtcgcccgacgcccgggctttgcccgggcggcctcagtgagcgagcgagcgcgcagctgcctgcagg | |
| 45 | cctgcaggcagctgcgcgctcgctcgctcactgaggccgcccgggcaaagcccgggcgtcgggcgacctaggtcgcccggcct | IL2RG |
| | cagtgagcgagcgagcgcgcagagagggagtggccaactccatcactaggggacctgcggccgccaacctctagaaatcaagg | AAV #10: |
| | tttttctgtgtagggttgggttagcgtgttgttagagtaggggagtggattgagaaggaggctgaggggtactcaagggggctataga | HA-MND- |
| | atgtataggatttccctgaagcattcctagagagcctgcaaggtgaagatggctttggaaccagctggatctaggctgtgccacatact | nakedFRB- |
| | acctctttggccttggccacatccctaaactcttggattctgtttcctaagatgtaagatggaggtaattgacctgcctcacaggagctgtCNb30- | |
| | tgtgaggattaaacagagagtatgtattagcgcggtgcctggcaccagtgcctggcatgtagtaggggcacaacaaatataaggtc | CISCβ- |
| | cactttgcttttcttttttctatagttaattaagtgtgaacagagaaacaggagaatatgggccaaacaggatatctgtggtaagcagttcc | CISCy HA |
| | tgccccggctcagggccaagaacagttggaacagcagaatatgggccaaacaggatatctgtggtaagcagttcctgccccggct | |
| | cagggccaagaacagatggtccccagatgcggtcccgccctcagcagtttctagagaaccatcagatgtttccagggtgccccaag | |
| | gacctgaaatgaccctgtgccttatttgaactaaccaatcagttcgcttctcgcttctgttcgcgcgcttctgctccccgagctctatataa | |
| | gcagagctcgtttagtgaaccgtcagatcggtaccgccgccaccatggagatgtggcatgagggtctggaagaagcgtctcgactg | |
| | tactttggtgagcgcaatgtgaagggcatgtttgaagtcctcgaaccccttcatgccatgatggaacgcggaccccagaccttgaag | |
| | gagacaagttttaaccaagatacggaagagacctgatggaagcccaggaatggtgcaggaaatacatgaaaaggggaatgtga | |
| | aggacttgctccaagcgtgggacctgtactatcacgtattaggcgcattagtaagggcagcggcgccacaaatttcagcctgctga | |
| | aacaggccggcgacgtggaagagaaccctggacccatgggcaacgaggccagctaccctctggagatgtgctcccacttcgacg | |
| | ccgacgagatcaagggctgggcaagcgcttcaagaagctggacctggacaacagcggcagcctgagcgtggaggagtttatgt | |
| | ctctgcccgagctgcagcagaaccccctggtgcagcgcgtgatcgacatcttcgacaccgacggcaacggcgaggtggacttcaa | |
| | ggagttcatcgagggcgtgagccagttcagcgtgaagggcgacaaggagcagaagctgcggttcgccttccggatctacgatatg | |
| | gataaagatggctatatttctaatggcgagctgttccaggtgctgaagatgatggtgggcaacaataccaagctggccgatacccag | |
| | ctgcagcagatcgtggacaagaccatcatcaacgccgacaaggacggcgacggcagaatcagatcgaggagttctgtgccgtg | |
| | gtgggaggcctggatattcacaaaaaaatggtggtggacgtgggcagcggcgaaggcagaggatccctgcttacatgtggcgac | |
| | gtggaagagaaccctggccccatgccacttggcctgctctggctgggcttggcattgctcggcgcgctccacgcccaggctgaact | |
| | gatccgcgtggccatattgtggcacgagatgtggcacgagggattggaggaggcgagtaggctgtactaggggaaaggaatgtta | |
| | aagggatgtttgaggtccttgaacccctccacgctatgatggaaagaggacctcaaacgcttaaagagacgtcattcaatcaagccta | |
| | tggacgggatcttatggaagctcaagaatggtgtcgaaaatacatgaaaagcgggaatgttaaggacctcacgcaagcctgggatc | |
| | tgtattaccatgttttccgacgcatttctaaacaaggaaaagatactatcccatggttggggcacttgctcgttgggctcagtggggcgt | |
| | ttggattcatcatcctcgtatatctgttgattaattgtcggaacacaggtccctggcttaaaaaagttttgaagtgtaacaccccggatcct | |
| | tctaaattttttagtcaacttagttcagaacacgggggcgatgttcaaaagtggctgagttccccgtttcccagttcaagtttctcccctgg | |
| | gggtctcgcccccgagatatcacctcttgaagtgctcgagcgggacaaagttacacagcttcttttgcaacaggataaggttccgga | |
| | gccggcgtctctcagctctaaccattcactcacttcttgtttcaccaaccaagggtattattcttccatctgcctgatgccttggagattga | |
| | ggcttgtcaggtgtactttacctatgacccctatagtgaggaagaccctgacgaaggcgtagctggcgcccccactggctccagtcc | |
| | acagcctcttcagcctctgtcaggggaggacgacgcatattgtacgttcccctcacgggacgaccttctgctgtatcaccctcactgc | |
| | tcggcggaccctccccgccaagcacggcacctggggggagtggggcaggagaagaaaggatgcctcctagtagcaggagcgg | |
| | gttcctcgcgactgggatccgcaacccctcggaccacccacccctggcgtacctgatctggtcgacttccaaccacctccggagctt | |
| | gtcctcagagaggccggagaggaagtcccagacgcggggccaagagagggtgtgtcataccctggtcccgccctccgggacag | |
| | ggtgagtacgggcgctgaatgcgaggctcccccttaataccgatgcgtacctgtcattgcaggaacttcagggccaggatcctacc | |
| | cacctggtgggatccggcgctacaaatattcactgctgaaacaggcgggtgacgtggaggagaaccctggacccatgcctctggg | |
| | cctgctgtggctgggcctggccctgctgggcgccctgcacgcccaggccggcgtgcaggtggagacaatctccccaggcgacg | |
| | gacgcacattccctaagcggggccagacctgcgtggtgcactatacaggcatgctggaggatggcaagaagtttgacagctcccg | |
| | ggatagaaacaagccattcaagtttatgctgggcaagcaggaagtgatcagaggctgggaggagggcgtggcccagatgtctgtg | |
| | ggccagagggccaagctgaccatcagcccagactacgcctatggagcaacaggccacccaggaatcatcccacctcacgccacc | |
| | ctggtgttcgatgtggagctgctgaagctgggcgagggcagcaacaccagcaaagagaatcctttcctgtttgcattggaagccgtg | |
| | gttatctctgttggctccatgggattgattatcagccttctctgtgtgtatttctggctggaacggtgagatttggagaagcccagaaaaa | |
| | tgaggggaacggtagctgacaatagcagaggagggtatgcagggtattaggagtaaaggatgagacagtaagtaatgagagatt | |
| | acccaagagggtttggtgatggaaggaagccacaggcacagagaacacagaatcactttatttcatatgggacaactgggagaag | |
| | ggtgataaaaaagctttaacctatgtgctcctgctccctctactcccctgtcaggacgatgccccgaattcccaccctgaagaacctag | |
| | aggatcttgttactgaataccacgggaacttttcggtgagaacgctgtcatcaattgtctacctaggaacccctagtgatggagaggc | |
| | cactccctctctgcgcgctcgctcgctcactgaggccgggcgaccaaaggtcgcccgacgcccgggctttgcccgggcggcctca | |
| | gtgagcgagcgagcgcgcagctgcctgcagg | |
| 46 | cctgcaggcagctgcgcgctcgctcgctcactgaggccgcccgggcaaagcccgggcgtcgggcgacctaggtcgcccggcct | IL2RG |
| | cagtgagcgagcgagcgcgcagagagggagtggccaactccatcactaggggacctgcggccgccaacctctagaaatcaagg | AAV #11: |
| | tttttctgtgtagggttgggttagcgtgttgttagagtaggggagtggattgagaaggaggctgaggggtactcaagggggctataga | HA-MND- |
| | atgtataggataccctgaagcattcctagagagcctgcaaggtgaagatggctttggaaccagctggatctaggctgtgccacatact | B2MCAR- |
| | acctctttggccttggccacatccctaaactcttggattctgtttcctaagatgtaagatggaggtaattgacctgcctcacaggagctgt | nakedFRB- |
| | tgtgaggattaaacagagagtatgtctttagcgcggtgcctggcaccagtgcctggcatgtagtaggggcacaacaaatataaggtc | CISCβ- |
| | cactttgcttttcttttttctatagttaattaagtgtgaacagagaaacaggagaatatgggccaaacaggatatctgtggtaagcagttcc | CISCy HA |
| | tgccccggctcagggccaagaacagaggaacagcagaatatgggccaaacaggatatctgtggtaagcagttcctgccccggct | |
| | cagggccaagaacagatggtccccagatgcggtcccgccctcagcagtttctagagaaccatcagatgtttccagggtgccccaag | |
| | gacctgaaatgaccctgtgccttatttgaactaaccaatcagttcgcttctcgcttctgttcgcgcgcttctgctccccgagctctatataa | |
| | gcagagctcgtttagtgaaccgtcagatcggtaccgccgccaccatgagcaggtcagtggcgttggcggttctggcgcttttgagttt | |
| | gagcggactggaagccatccaacgaacgcctaagatccaggtatattcacgccacccggcggaaaacggcaaaagtaacttcctt | |
| | aattgttatgtgtctggcttccacccgtctgatattgaggtggacctccttaaaaacggtgaacggatcgagaaagtggagcattccga | |
| | tcttagtttcagtaaggattggagcttttaccttctctattacactgagttcactccgactgaaaaggatgagtacgcctgtcgggtcaac | |
| | cacgtcaccctgtctcaaccaaaaatagtcaaatgggacagagatatgtcagatatttacatatgggcaccacttgcgggcacgtgtg | |
| | gcgtcctgcttctgagtctcgtcattacgctttattgtaaacggggtagaaaaaaactcattatatatttaaacagccatttatgcggcca | |
| | gttcaaacgacgcaggaagaagacggctgtagttgcagatttccagaggaagaggaaggtggatgcgagcttcgggtcaagata | |
| | gtaggtctgcagacgctcccgcctatcaacagggtcagaatcagctttataacgaactcaacctcggtcgccgagaagagtacgac | |
| | gtactcgataaaagaaggggtagagacccggaaatggggggcaaaccgcgccgcaaaaatccacaagaggggctttataatgag | |
| | cttcaaaaagacaaaatggccgaagcatacagtgagattgggatgaaaggtgaacgcagaagaggtaagggtcacgacgggctg | |
| | taccagggatgtcaactgccacaaaggatacttatgacgctctgcatatgcaagctcttcccccacgcggcagcggcgaaggcaga | |
| | ggatccctgcttacatgtggcgacgtggaagagaaccctggccccatggagatgtggcatgagggtctggaagaagcgtctcgact | |
| | gtactttggtgagcgcaatgtgaagggcatgtagaagtcctcgaaccccttcatgccatgatggaacgcggaccccagaccttgaa | |
| | ggagacaagttttaaccaagcttacggaagagacctgatggaagcccaggaatggtgcaggaaatacatgaaaagcgggaatgtg | |
| | aaggacttgctccaagcgtgggacctgtactatcacgtctttaggcgcattagtaagggcagcggcgccacaaatttcagcctgctg | |
| | aaacaggccggcgacgtggaagagaaccctggacccatgccacttggcctgctctggctgggcttggcattgctcggcgcgctcc | |
| | acgcccaggctgaactgatccgcgtggccatattgtggcacgagatgtggcacgagggattggaggaggcgagtaggctgtacttt | |
| | ggggaaaggaatgttaaagggatgtagaggtccttgaacccctccacgctatgatggaaagaggacctcaaacgcttaaagagac | |
| | gtcattcaatcaagcctatggacgggatcttatggaagctcaagaatggtgtcgaaaatacatgaaaagcgggaatgttaaggacctc | |
| | acgcaagcctgggatctgtattaccatgttttccgacgcatttctaaacaaggaaaagatactatcccatggttggggcacttgctcgtt | |
| | gggctcagtggggcgtttggattcatcatcctcgtatatctgttgattaattgtcggaacacaggtccctggcttaaaaaagttttgaagt | |
| | gtaacaccccggatccttctaaattttttagtcaacttagttcagaacacgggggcgatgttcaaaagtggctgagttccccgtttccca | |
| | gttcaagtactcccctgggggtctcgcccccgagatatcacctcttgaagtgctcgagcgggacaaagttacacagcttcttttgcaa | |
| | caggataaggttccggagccggcgtctctcagctctaaccattcactcacttcttgtttcaccaaccaagggtattttttcttccatctgcc | |
| | tgatgccttggagattgaggcttgtcaggtgtactttacctatgacccctatagtgaggaagaccctgacgaaggcgtagctggcgcc | |
| | cccactggctccagtccacagcctcttcagcctctgtcaggggaggacgacgcatattgtacgacccctcacgggacgaccttctg | |
| | ctgttacaccctcactgctcggcggaccctccccgccaagcacggcacctggggggagtggggcaggagaagaaaggatgcct | |
| | cctagtttgcaggagcgggacctcgcgactgggatccgcaacccctcggaccacccacccctggcgtacctgatctggtcgacttc | |
| | caaccacctccggagcttgtcctcagagaggccggagaggaagtcccagacgcggggccaagagagggtgtgtcataccctggt | |
| | cccgccctccgggacagggtgagatcgggcgctgaatgcgaggctcccccttaataccgatgcgtacctgtcattgcaggaacttc | |
| | agggccaggatcctacccacctggtgggatccggcgctacaaattatcactgctgaaacaggcgggtgacgtggaggagaaccct | |
| | ggacccatgcctctgggcctgctgtggctgggcctggccctgctgggcgccctgcacgcccaggccggcgtgcaggtggagaca | |
| | atctccccaggcgacggacgcacattccctaagcggggccagacctgcgtggtgcactatacaggcatgctggaggatggcaag | |
| | aagtagacagctcccgggatagaaacaagccattcaagtttatgctgggcaagcaggaagtgatcagaggctgggaggagggcg | |
| | tggcccagatgtctgtgggccagagggccaagctgaccatcagcccagactacgcctatggagcaacaggccacccaggaatca | |
| | tcccacctcacgccaccctggtgttcgatgtggagctgctgaagctgggcgagggcagcaacaccagcaaagagaatcctacctg | |
| | tttgcattggaagccgtggttatctctgaggctccatgggattgattatcagccttctctgtgtgtatttctggctggaacggtgagatttg | |
| | gagaagcccagaaaaatgaggggaacggtagctgacaatagcagaggagggttagcagggtctttaggagtaaaggatgagac | |
| | agtaagtaatgagagattacccaagagggtaggtgatggaaggaagccacaggcacagagaacacagaatcactttatttcatatg | |
| | ggacaactgggagaagggtgataaaaaagctttaacctatgtgctcctgctccctctttctcccctgtcaggacgatgccccgaattcc | |
| | caccctgaagaacctagaggatcflgttactgaataccacgggaacttttcggtgagaacgctgtcatcaattgtctacctaggaaccc | |
| | ctagtgatggagaggccactccctctctgcgcgctcgctcgctcactgaggccgggcgaccaaaggtcgcccgacgcccgggctt | |
| | tgcccgggcggcctcagtgagcgagcgagcgcgcagctgcctgcagg | |
| 47 | gvqvetispgdgrtfpkrgqtcvvhytgmledgkkfdssrdrnkpfkfmlgkqevirgweegvaqmsvgqrakltispdyay | FKBP CISC |
| | gatghpgiipphativfdvellklge | domain |
| 48 | elirvailwhemwhegleeasrlyfgernvkgmfevleplhammergpqtlketsfnqaygrdlmeagewcrkymksgnv | FRB CISC |
| | kdltqawdlyyhvfrriskq | domain |
| 49 | gsntskenpflfaleavvisvgsmgliisllcvyfwler | ILR2g CISC |
| | | fragment |
| 50 | gsntskenpflfaleavvisvgsmgliisllcvyfwlertmpriptlknledlvteyhgnfsawsgvskglaeslqpdyserlclvs | ILR2g CISC |
| | eippkggalgegpgaspcnqhspywappcytlkpet | domain |
| 51 | gkdtipwlghllvglsgafgfiilvyllincrntgpwlkkylkcntpdpskffsqlssehggdvqkwlsspfpsssfspgglapeis | ILR2b CISC |
| | plevlerdkvtqlllqqdkvpepaslssnhsltscftnqgyfffhlpdaleieacqvyftydpyseedpdegvagaptgsspqplq | domain |
| | plsgeddayctfpsrddlllfspsllggpsppstapggsgageermppslqervprdwdpqplgpptpgvpdlvdfqpppelvl | |
| | reageevpdagpregvsfpwsrppgqgefralnarlpintdaylslqelqgqdpthlv | |
| 52 | gvqvetispgdgrtfpkrgqtcvvhytgmledgkkfdssrdrnkpfkfmlgkqevirgweegvaqmsvgqrakltispdyay | CISCγ |
| | gatghpgiipphativfdvellklgegsntskenpflfaleavvisvgsmgliisllcvyfwler | fragment |
| 53 | gvqvetispgdgrtfpkrgqtcvvhytgmledgkkfdssrdrnkpfkfmlgkqevirgweegvaqmsvgqrakltispdyay | CISCγ |
| | gatghpgiipphativfdvellklgegsntskenpflfaleavvisvgsmgliisllcvyfwlertmpriptlknledlvteyhgnfs | component |
| | awsgvskglaeslqpdyserlclvseippkggalgegpgaspcnqhspywappcytlkpet | |
| 54 | elirvailwhemwhegleeasrlyfgernvkgmfevleplhammergpqtlketsfnqaygrdlmeagewcrkymksgnv | CISCβ |
| | kdltqawdlyyhvfaiskqgkdtipwlghllvglsgafgfiilvyllincrntgpwlkkvlkcntpdpskffsqlssehggdvqk | component |
| | wlsspfpsssfspgglapeisplevlerdkvtqlllqqdkvpepaslssnhsltscftnqgyfffhlpdaleieacqvyftydpysee | |
| | dpdegvagaptgsspqplqplsgeddayctfpsrddlllfspsllggpsppstapggsgageermppslqervprdwdpqplg | |
| | pptpgvpdlvdfqpppelvlreageevpdagpregvsfpwsrppgqgefralnarlpintdaylslqelqgqdpthlv | |
| 55 | divliqsppslamslgkratiscrasesvtilgshlihwyqqkpgqpptlliqlasnvqtgvparfsgsgsrtdftltidpveeddva | anti-BCMA |
| | vyyclqsrtiprtfgggtkleikgstsgsgkpgsgegstkgqiqlvqsgpelkkpgetvkisckasgytftdysinwykrapgkgl | scFv |
| | kwmgwintetrepayaydfrgrfafsletsastaylqinnlkyedtatyfcaldysyamdywgqgtsvtvss | |
| 56 | fvpvflpakptttpaprpptpaptiasqplslrpeacrpaaggavhtrgldfacdiyiwaplagtcgvlllslvitlycnhrn | CD8 |
| | | transmembrane |
| | | domain |
| 57 | rskrsrllhsdymnmtprrpgptrkhyqpyapprdfaayrs | CD28 co- |
| | | stimulatory |
| | | domain |
| 58 | rfsvvkrgrkkllyifkqpfmrpvqttqeedgcscrfpeeeeggcel | 4-1BB co- |
| | | stimulatory |
| | | domain |
| 59 | rvkfsrsadapayqqgqnqlynelnlgrreeydvldkagrdpemggkprrknpqeglynelqkdkmaeayseigmkgerrr | CD3 zeta |
| | gkghdglyqglstatkdtydalhmqalppr | activation |
| | | domain |
| 60 | divliqsppslamslgkratiscrasesvtilgshlihwyqqkpgqpptlliqlasnvqtgvparfsgsgsrtdftltidpveeddva | anti-BCMA |
| | vyyclqsrtiprtfgggtkleikgstsgsgkpgsgegstkgqiqlvqsgpelkkpgetvkisckasgytftdysinwykrapgkgl | CAR, CD28 |
| | kwmgwintetrepayaydfrgrfafsletsastaylqinnlkyedtatyfcaldysyamdywgqgtsvtvssfvpvflpakpttt | |
| | paprpptpaptiasqplslrpeacrpaaggavhtrgldfacdiyiwaplagtcgvlllslvitlycnhrnrskrsrllhsdymnmtpr | |
| | rpgptrkhyqpyapprdfaayrsrvkfsrsadapayqqgqnqlynelnlgrreeydvldkagrdpemggkprrknpqeglyn | |
| | elqkdkmaeayseigmkgeragkghdglyqglstatkdtydalhmqalppr | |
| 61 | divliqsppslamslgkratiscrasesvtilgshlihwyqqkpgqpptlliqlasnvqtgvparfsgsgsrtdftltidpveeddva | anti-BCMA |
| | vyyclqsrtiprtfgggtkleikgstsgsgkpgsgegstkgqiqlvqsgpelkkpgetvkisckasgytftdysinwykrapgkgl | CAR, 4- |
| | kwmgwintetrepayaydfrgrfafsletsastaylqinnlkyedtatyfcaldysyamdywgqgtsvtvssaaafvpvflpak | 1BB |
| | ptttpaprpptpaptiasqplslrpeacrpaaggavhtrgldfacdiyiwaplagtcgvlllslvitlycnhrnrfsvvkrgrkkllyif | |
| | kqpfmrpvqttqeedgcscrfpeeeeggcelrvkfsrsadapayqqgqnqlynelnlgrreeydvldkagrdpemggkprrk | |
| | npqeglynelqkdkmaeayseigmkgeragkghdglyqglstatkdtydalhmqalppr | |
| 62 | msrsvalavlallslsgleaiqrtpkiqvysrhpaengksnflncyvsgfhpsdievdllkngeriekvehsdlsfskdwsfyllyy | beta-2- |
| | teftptekdeyacrvnhvtlsqpkivkwdrdm | microglobulin |
| | | domain |
| 63 | sdiyiwaplagtcgvlllslvitlyc | CD8 |
| | | transmembrane |
| | | domain |
| 64 | krgrkkllyifkqpfmrpvqttqeedgcscrfpeeeeggcel | 4-1BB co- |
| | | stimulatory |
| | | domain |
| 65 | msrsvalavlallslsgleaiqrtpkiqvysrhpaengksnflncyvsgfhpsdievdllkngeriekvehsdlsfskdwsfyllyy | beta-2- |
| | teftptekdeyacrvnhvtlsqpkivkwdrdmsdiyiwaplagtcgvlllslvitlyckrgrkkllyifkqpfmrpvqttqeedgc | microglobulin |
| | scrfpeeeeggcelrvkfsrsadapayqqgqnqlynelnlgrreeydvldkagrdpemggkprrknpqeglynelqkdkma | chimeric |
| | eayseigmkgerrrgkghdglyqglstatkdtydalhmqalppr | receptor |
| 66 | mgagatgramdgprlllllllgvslggakeacptglythsgecckacnlgegvaqpcganqtvcepcldsvtfsdvvsatepckp | tLNGFR |
| | ctecvglqsmsapcveaddavcrcaygyyqdettgrceacrvceagsglvfscqdkqntvceecpdgtysdeanhvdpclpct | polypeptide |
| | vcedterqlrectrwadaeceeipgrwitrstppegsdstapstqepeappeqdliastvagvvttvmgssqpvvtrgttdnlipv | |
| | ycsilaavvvglvayiafkr | |
| 67 | mgneasyplemcshfdadeikrlgkrfkkldldnsgslsveefmslpelqqnplvqrvidifdtdgngevdfkefiegvsqfsv | CNb30 |
| | kgdkeqklrfafriydmdkdgyisngelfqvlkmmvgnntkladtqlqqivdktiinadkdgdgrisfeefcavvggldihkk | polypeptide |
| | mvvdv | |
| 68 | memwhegleeasrlyfgernvkgmfevleplhammergpqtlketsfnqaygrdlmeagewcrkymksgnvkdltqaw | naked FRB |
| | dlyyhvfrrisk | wild-type |
| | | polypeptide |
| 69 | memwhegleeasrlyfgernvkgmfevleplhammergpqtlketsfnqaygrdlmeagewcrkymksgnvkdllqaw | naked FRB |
| | dlyyhvfrrisk | mutant |
| | | polypeptide |
| 70 | malpvtalllplalllhaarp | CD8 signal |
| 71 | mplgllwlglallgalhaqa | ER signal |
| 72 | gsgegrgslltcgdveenpgp | T2A |
| 73 | gsgatnfsllkqagdveenpgp | P2A |
| 74 | acgtAAGCTTgtgtgaacagagaaacaggagaatatgggccaaacaggatatctgtggtaagcagacctgccccggctcag | MND |
| | ggccaagaacagttggaacagcagaatatgggccaaacaggatatctgtggtaagcagttcctgccccggctcagggccaagaac | promoter |
| | agatggtccccagatgcggtcccgccctcagcagtttctagagaaccatcagatgtttccagggtgccccaaggacctgaaatgacc | |
| | ctgtgccttatttgaactaaccaatcagttcgcttctcgcttctgttcgcgcgcttctgctccccgagctctatataagcagagctcgttta | |
| | gtgaaccgtcaAAGCTTacgt | |
| 75 | aatgaaagaccccacctgtaggtttggcaagctagcttaagtaacgccattttgcaaggcatggaaaatacataactgagaatagaga | MSCV |
| | agttcagatcaaggttaggaacagagagacagcagaatatgggccaaacaggatatctgtggtaagcagttcctgccccggctcag | promoter |
| | ggccaagaacagatggtccccagatgcggtcccgccctcagcagtttctagagaaccatcagatgtttccagggtgccccaaggac | |
| | ctgaaaatgaccctgtgccttatttgaactaaccaatcagttcgcttctcgcttctgttcgcgcgcttctgctccccgagctcaataaaag | |
| | agcccacaacccctcactcggc | |
| 76 | paalgkdtipwlghllvglsgafgfiilvyllincrntgpwllckylkcntpdpskffsqlssehggdvqkwlsspfpsssfspggla | truncated |
| | peisplevlerdlcvtqlllqqdkvpepasls1ntdaylslqelq | IL2120 |
| | | domain |
| 77 | malpvtalllplalllhaarpilwhemwhegleeasrlyfgernvkgmfevleplhammergpqtlketsfnqaygrdlmeaq | CISCβ |
| | ewcrlcymksgmldllqawdlyylwfrriskpaalgkdtipwlghllvglsgafgfiilvyllincrntgpwllckvllccntpdps | component, |
| | kffsqlssehggdvqkwlsspfpsssfspgglapeisplevlerdlcvtqlllqqdkvpepaslslntdaylslqelq | truncated |
| 78 | tattaagctcagtcccaaac | BCMA |
| | | target |
| | | sequence |
| 79 | ggggccactagggacaggat | AAVS1 |
| | | target |
| | | sequence |
| 80 | tggccgtgaacgttcactgaaatcatggcctcttggccaagattgatagcttgtgcctgtccctgagtcccagtccatc | TRAC 1 5′ |
| | acgagcagctggtttctaagatgctatttcccgtataaagcatgagaccgtgacttgccagccccacagagccccgcc | homology |
| | cttgtccatcactggcatctggactccagcctgggttggggcaaagagggaaatgagatcatgtcctaaccctgatcc | arm |
| | tcttgtcccacagatatccagaaccctgaccctgccgtgtaccagctgagagactctaaatccagtgacaagtctgtct | |
| | gcctattcaccgattttgattctcaaacaaatgtgtcacaaagtaaggattctgatgtgtatatcacagacaaaactgt | |
| | gctagac | |
| 81 | atgaggtctatggacttcaagagcaacagtgctgtggcctggagcaacaaatctgactttgcatgtgcaaacgccttc | TRAC 1 3′ |
| | aacaacagcattattccagaagacaccttcttccccagcccaggtaagggcagctttggtgccttcgcaggctgtttcc | homology |
| | ttgcttcaggaatggccaggttctgcccagagctctggtcaatgatgtctaaaactcctctgattggtggtctcggcctt | arm |
| | atccattgccaccaaaaccctctttttactaagaaacagtgagccttgttctggcagtccagagaatgacacgggaaa | |
| | aaagcagatgaagagaaggtggcaggagagggcacgtggcccagcctcagtctctccaactgagttcctgcctgcct | |
| | gcctttgc | |
| 82 | ccaagattgatagcttgtgcctgtccctgagtcccagtccatcacgagcagctggtttctaagatgctatttcccgtata | TRAC 2 5′ |
| | aagcatgagaccgtgacttgccagccccacagagccccgcccttgtccatcactggcatctggactccagcctgggtt | homology |
| | ggggcaaagagggaaatgagatcatgtcctaaccctgatcctcttgtcccacagatatccagaaccctgaccctgcc | arm |
| | gtgtaccagctgagagactctaaatccagtgacaagtctgtctgcctattcaccgattttgattctcaaacaaatgtgtc | |
| | acaaagtaaggattctgatgtgtatatcacagacaaaactgtgctagacatgaggtctatggacttcaagagcaaca | |
| | gtgctgtg | |
| 83 | gcctggagcaacaaatctgactttgcatgtgcaaacgccttcaacaacagcattattccagaagacaccttcttcccc | TRAC 2 3′ |
| | agcccaggtaagggcagctttggtgccttcgcaggctgtttccttgcttcaggaatggccaggttctgcccagagctct | homology |
| | ggtcaatgatgtctaaaactcctctgattggtggtctcggccttatccattgccaccaaaaccctctttttactaagaaa | arm |
| | cagtgagccttgttctggcagtccagagaatgacacgggaaaaaagcagatgaagagaaggtggcaggagagggc | |
| | acgtggcccagcctcagtctctccaactgagttcctgcctgcctgcctttgctcagactgtttgccccttactgctcttcta | |
| | ggcctc | |
| 84 | cccatgcctgcctttactctgccagagttatattgctggggttttgaagaagatcctattaaataaaagaataagcagt | TRAC 3 5′ |
| | attattaagtagccctgcatttcaggtttccttgagtggcaggccaggcctggccgtgaacgttcactgaaatcatggc | homology |
| | ctcttggccaagattgatagcttgtgcctgtccctgagtcccagtccatcacgagcagctggtttctaagatgctatttc | arm |
| | ccgtataaagcatgagaccgtgacttgccagccccacagagccccgcccttgtccatcactggcatctggactccagc | |
| | ctgggttggggcaaagagggaaatgagatcatgtcctaaccctgatcctcttgtcccacagatatccagaaccctgac | |
| | cctgcc | |
| 85 | gtgtaccagctgagagactctaaatccagtgacaagtctgtctgcctattcaccgattttgattctcaaacaaatgtgtc | TRAC 3 3′ |
| | acaaagtaaggattctgatgtgtatatcacagacaaaactgtgctagacatgaggtctatggacttcaagagcaaca | homology |
| | gtgctgtggcctggagcaacaaatctgactttgcatgtgcaaacgccttcaacaacagcattattccagaagacacct | arm |
| | tcttccccagcccaggtaagggcagctttggtgccttcgcaggctgtttccttgcttcaggaatggccaggttctgccca | |
| | gagctctggtcaatgatgtctaaaactcctctgattggtggtctcggccttatccattgccaccaaaaccctctttttact | |
| | aaga | |
| 86 | caacctctagaaatcaaggtttttctgtgtagggttgggttagcgtgttgttagagtaggggagtggattgagaagga | GC8 5′ |
| | ggctgaggggtactcaagggggctatagaatgtataggatttccctgaagcattcctagagagcctgcaaggtgaag | homology |
| | atggctttggaaccagctggatctaggctgtgccacatactacctctttggccttggccacatccctaaactcttggatt | arm |
| | ctgtttcctaagatgtaagatggaggtaattgttcctgcctcacaggagctgttgtgaggattaaacagagagtatgtc | |
| | tttagcgcggtgcctggcaccagtgcctggcatgtagtaggggcacaacaaatataaggtccactttgcttttctttttt | |
| | ctatag | |
| 87 | gaaaacccctttttgttcgctcttgaggctgtcgtgattagcgtcggatccatgggattgattatcagccttctctgtgtgGC83′ | |
| | tatttctggctggaacggtgagatttggagaagcccagaaaaatgaggggaacggtagctgacaatagcagaggag | homology |
| | ggttttgcagggtctttaggagtaaaggatgagacagtaagtaatgagagattacccaagagggtttggtgatggaa | arm |
| | ggaagccacaggcacagagaacacagaatcactttatttcatatgggacaactgggagaagggtgataaaaaagct | |
| | ttaacctatgtgctcctgctccctctttctcccctgtcaggacgatgccccgaattcccaccctgaagaacctagaggat | |
| | cttgttactgaataccacgggaacttttcggtgagaacgctgtcat | |
| 88 | caacctctagaaatcaaggtttttctgtgtagggttgggttagcgtgttgttagagtaggggagtggattgagaagga | GC10 5′ |
| | ggctgaggggtactcaagggggctatagaatgtataggatttccctgaagcattcctagagagcctgcaaggtgaag | homology |
| | atggctttggaaccagctggatctaggctgtgccacatactacctctttggccttggccacatccctaaactcttggatt | arm |
| | ctgtttcctaagatgtaagatggaggtaattgttcctgcctcacaggagctgttgtgaggattaaacagagagtatgtc | |
| | tttagcgcggtgcctggcaccagtgcctggcatgtagtaggggcacaacaaatataaggtccactttgcttttctttttt | |
| | ctatag | |
| 89 | gaaaacccctttttgttcgctcttgaggctgtcgtgattagcgtcggaagtatgggattgattatcagccttctctgtgtgGC103′ | |
| | tatttctggctggaacggtgagatttggagaagcccagaaaaatgaggggaacggtagctgacaatagcagaggag | homology |
| | ggttttgcagggtctttaggagtaaaggatgagacagtaagtaatgagagattacccaagagggtttggtgatggaa | arm |
| | ggaagccacaggcacagagaacacagaatcactttatttcatatgggacaactgggagaagggtgataaaaaagct | |
| | ttaacctatgtgctcctgctccctctttctcccctgtcaggacgatgccccgaattcccaccctgaagaacctagaggat | |
| | cttgttactgaataccacgggaacttttcggtgagaacgctgtcat | |
| 90 | caacctctagaaatcaaggtttttctgtgtagggttgggttagcgtgttgttagagtaggggagtggattgagaagga | GC12 5′ |
| | ggctgaggggtactcaagggggctatagaatgtataggatttccctgaagcattcctagagagcctgcaaggtgaag | homology |
| | atggctttggaaccagctggatctaggctgtgccacatactacctctttggccttggccacatccctaaactcttggatt | arm |
| | ctgtttcctaagatgtaagatggaggtaattgttcctgcctcacaggagctgttgtgaggattaaacagagagtatgtc | |
| | tttagcgcggtgcctggcaccagtgcctggcatgtagtaggggcacaacaaatataaggtccactttgcttttctttttt | |
| | ctatag | |
| 91 | gaaaacccctttttgtttgcattggaagccgtggttatctctgttggctccatgggattgattatcagccttctctgtgtgt | GC12 3′ |
| | atttctggctggaacggtgagatttggagaagcccagaaaaatgaggggaacggtagctgacaatagcagaggag | homology |
| | ggttttgcagggtctttaggagtaaaggatgagacagtaagtaatgagagattacccaagagggtttggtgatggaa | arm |
| | ggaagccacaggcacagagaacacagaatcactttatttcatatgggacaactgggagaagggtgataaaaaagct | |
| | ttaacctatgtgctcctgctccctctttctcccctgtcaggacgatgccccgaattcccaccctgaagaacctagaggat | |
| | cttgttactgaataccacgggaacttttcggtgagaacgctgtcat | |
| 92 | tggccgtgaacgttcactgaaatcatggcctcttggccaagattgatagcttgtgcctgtccctgagtcccagtccatc | AAV HA |
| | acgagcagctggtttctaagatgctatttcccgtataaagcatgagaccgtgacttgccagccccacagagccccgcc | TRAC 1- |
| | cttgtccatcactggcatctggactccagcctgggttggggcaaagagggaaatgagatcatgtcctaaccctgatcc | TNP-AAA- |
| | tcttgtcccacagatatccagaaccctgaccctgccgtgtaccagctgagagactctaaatccagtgacaagtctgtct | CD8-CD28- |
| | gcctattcaccgattttgattctcaaacaaatgtgtcacaaagtaaggattctgatgtgtatatcacagacaaaactgt | CD3z-P2A- |
| | gctagactgaatgaatgattaattaataaaagatctttattttcattagatctgtgtgttggttttttgtgtgatcctcgag | CISCb-HA |
| | ggaatgaaagaccccacctgtaggtttggcaagctagcttaagtaacgccattttgcaaggcatggaaaatacataa | TRAC 1 |
| | ctgagaatagagaagttcagatcaaggttaggaacagagagacagcagaatatgggccaaacaggatatctgtggt | |
| | aagcagttcctgccccggctcagggccaagaacagatggtccccagatgcggtcccgccctcagcagtttctagaga | |
| | accatcagatgtttccagggtgccccaaggacctgaaaatgaccctgtgccttatttgaactaaccaatcagttcgctt | |
| | ctcgcttctgttcgcgcgcttctgctccccgagctcaataaaagagcccacaacccctcactcggcgcgcgccagtccg | |
| | gtaccagtcgccaccatggccctgcctgtgacagctctgctcctccctctggccctgctgctccatgccgccagacccg | |
| | acattgtgatgacccagtctcaaaaattcatgtccacatcagtaggagacagggtcagcatcacctgcaaggccagt | |
| | cagaatgtgggtactgctgtagcctggtatcaacagaaaccaggacaatctcctaaactactgatttactcggcatcc | |
| | aatcggtacactggagtccctgatcgcttcacaggcagtggatctgggacagatttcactctcaccatcagcaatatg | |
| | cagtctgaagacctggcagattatttctgccagcaatatagcagctatcctctcacgttcggtgctgggaccaagctgg | |
| | agctgaaaggcagcaccagcggctccggcaagcctggctctggcgagggcagcacaaagggacaggtccagctgc | |
| | agcagtctggacctgagctggtgaagcctggggcttcagtgaggatatcctgcaaggcttctggctacaccttcacaa | |
| | gctactatatacactgggtgaagcagaggcctggacagggacttgagtggattggatggatttatcctggaaatgtta | |
| | atactaagtacaatgagaagttcaagggcaaggccacactgactgcagacaaatcctccagcacagcctacatgca | |
| | gctcagcagcctgacctctgaggactctgcggtctatttctgtgcaagaaactacggtagtagctacgggcttgcttac | |
| | tggggccaagggactctggtcactgtctctgcattcgtgcccgtgttcctgcccgccaaacctaccaccacccctgccc | |
| | ctagacctcccaccccagccccaacaatcgccagccagcctctgtctctgcggcccgaagcctgtagacctgctgccg | |
| | gcggagccgtgcacaccagaggcctggacttcgcctgcgacatctacatctgggcccctctggccggcacctgtggcg | |
| | tgctgctgctgagcctggtgatcaccctgtactgcaaccaccggaacagaagcaagcggagccggctgctgcacagc | |
| | gactacatgaacatgaccccaagacggcctggccccacccggaagcactaccagccttacgcccctcccagagactt | |
| | cgccgcctaccggtccagagtgaagttcagcagatccgccgacgcccctgcctaccagcagggacagaaccagctgt | |
| | acaacgagctgaacctgggcagacgggaagagtacgacgtgctggacaagcggagaggccgggaccccgagatg | |
| | ggcggaaagcccagacggaagaacccccaggaaggcctgtataacgaactgcagaaagacaagatggccgaggc | |
| | ctacagcgagatcggcatgaagggcgagcggaggcgcggcaagggccacgatggcctgtaccagggcctgagcac | |
| | cgccaccaaggacacctacgacgccctgcacatgcaggccctgccccccagaggatccggcgctacaaatttttcac | |
| | tgctgaaacaggcgggtgatgtggaggagaaccctggacccatgccacttggcctgctctggctgggcttggcattgc | |
| | tcggcgcgctccacgcccaggctgaactgatccgcgtggccatattgtggcatgagatgtggcatgagggattggag | |
| | gaggcgagtaggctgtactttggggaaaggaatgttaaagggatgtttgaggtccttgaacccctccacgctatgatg | |
| | gaaagaggacctcaaacgcttaaagagacgtcattcaatcaagcctatggacgggatcttatggaagctcaagaat | |
| | ggtgtcgaaaatacatgaaaagcgggaatgttaaggacctcacgcaagcctgggatctgtattaccatgttttccgac | |
| | gcatttctaaacaaggaaaagatactatcccatggttggggcacttgctcgttgggctcagtggggcgtttggattcat | |
| | catcctcgtatatctgttgattaattgtcggaacacaggtccctggcttaaaaaagttttgaagtgtaacaccccggat | |
| | ccttctaaattttttagtcaacttagttcagaacacgggggcgatgttcaaaagtggctgagttccccgtttcccagttc | |
| | aagtttctcccctgggggtctcgcccccgagatatcacctcttgaagtgctcgagcgggacaaagttacacagcttctt | |
| | ttgcaacaggataaggttccggagccggcgtctctcagctctaaccattcactcacttcttgtttcaccaaccaagggt | |
| | attttttcttccatctgcctgatgccttggagattgaggcttgtcaggtgtactttacctatgacccctatagtgaggaag | |
| | accctgacgaaggcgtagctggcgcccccactggctccagtccacagcctcttcagcctctgtcaggggaggacgac | |
| | gcatattgtacgttcccctcacgggacgaccttctgctgttttcaccctcactgctcggcggaccctccccgccaagcac | |
| | ggcacctggggggagtggggcaggagaagaaaggatgcctcctagtttgcaggagcgggttcctcgcgactgggat | |
| | ccgcaacccctcggaccacccacccctggcgtacctgatctggtcgacttccaaccacctccggagcttgtcctcaga | |
| | gaggccggagaggaagtcccagacgcggggccaagagagggtgtgtcatttccctggtcccgccctccgggacagg | |
| | gtgagtttcgggcgctgaatgcgaggctcccccttaataccgatgcgtacctgtcattgcaggaacttcagggccagg | |
| | atcctacccacctggtgtgaaagcttgataatcaacctctggattacaaaatttgtgaaagattgactggtattcttaa | |
| | ctatgttgctccttttacgctatgtggatacgctgctttaatgcctttgtatcatgctattgcttcccgtatggctttcatttt | |
| | ctcctccttgtataaatcctggttagttcttgccacggcggaactcatcgccgcctgccttgcccgctgctggacagggg | |
| | ctcggctgttgggcactgacaattccgtggaacttgtttattgcagcttataatggttacaaataaagcaatagcatca | |
| | caaatttcacaaataaagcatttttttcactgcattctagttgtggtttgtccaaactcatcaatgtatcttacgccggcg | |
| | tgaatgaggtctatggacttcaagagcaacagtgctgtggcctggagcaacaaatctgactttgcatgtgcaaacgcc | |
| | ttcaacaacagcattattccagaagacaccttcttccccagcccaggtaagggcagctttggtgccttcgcaggctgtt | |
| | tccttgcttcaggaatggccaggttctgcccagagctctggtcaatgatgtctaaaactcctctgattggtggtctcggc | |
| | cttatccattgccaccaaaaccctctttttactaagaaacagtgagccttgttctggcagtccagagaatgacacggga | |
| | aaaaagcagatgaagagaaggtggcaggagagggcacgtggcccagcctcagtctctccaactgagttcctgcctg | |
| | cctgcctttgc | |
| 93 | tggccgtgaacgttcactgaaatcatggcctcttggccaagattgatagcttgtgcctgtccctgagtcccagtccatc | AAV HA |
| | acgagcagctggtttctaagatgctatttcccgtataaagcatgagaccgtgacttgccagccccacagagccccgcc | TRAC 1- |
| | cttgtccatcactggcatctggactccagcctgggttggggcaaagagggaaatgagatcatgtcctaaccctgatcc | TNP-AAA- |
| | tcttgtcccacagatatccagaaccctgaccctgccgtgtaccagctgagagactctaaatccagtgacaagtctgtct | CD8-41BB- |
| | gcctattcaccgattttgattctcaaacaaatgtgtcacaaagtaaggattctgatgtgtatatcacagacaaaactgt | CD3z-P2A- |
| | gctagactgaatgaatgattaattaataaaagatctttattttcattagatctgtgtgttggttttttgtgtgatcctcgag | CISCb-HA |
| | ggaatgaaagaccccacctgtaggtttggcaagctagcttaagtaacgccattttgcaaggcatggaaaatacataa | TRAC 1 |
| | ctgagaatagagaagttcagatcaaggttaggaacagagagacagcagaatatgggccaaacaggatatctgtggt | |
| | aagcagttcctgccccggctcagggccaagaacagatggtccccagatgcggtcccgccctcagcagtttctagaga | |
| | accatcagatgtttccagggtgccccaaggacctgaaaatgaccctgtgccttatttgaactaaccaatcagttcgctt | |
| | ctcgcttctgttcgcgcgcttctgctccccgagctcaataaaagagcccacaacccctcactcggcgcgcgccagtccg | |
| | gtaccagtcgccaccatggccctgcctgtgacagctctgctcctccctctggccctgctgctccatgccgccagacccg | |
| | acattgtgatgacccagtctcaaaaattcatgtccacatcagtaggagacagggtcagcatcacctgcaaggccagt | |
| | cagaatgtgggtactgctgtagcctggtatcaacagaaaccaggacaatctcctaaactactgatttactcggcatcc | |
| | aatcggtacactggagtccctgatcgcttcacaggcagtggatctgggacagatttcactctcaccatcagcaatatg | |
| | cagtctgaagacctggcagattatttctgccagcaatatagcagctatcctctcacgttcggtgctgggaccaagctgg | |
| | agctgaaaggcagcaccagcggctccggcaagcctggctctggcgagggcagcacaaagggacaggtccagctgc | |
| | agcagtctggacctgagctggtgaagcctggggcttcagtgaggatatcctgcaaggcttctggctacaccttcacaa | |
| | gctactatatacactgggtgaagcagaggcctggacagggacttgagtggattggatggatttatcctggaaatgtta | |
| | atactaagtacaatgagaagttcaagggcaaggccacactgactgcagacaaatcctccagcacagcctacatgca | |
| | gctcagcagcctgacctctgaggactctgcggtctatttctgtgcaagaaactacggtagtagctacgggcttgcttac | |
| | tggggccaagggactctggtcactgtctctgcagccgccgccttcgtgcccgtgttcctgcccgccaaacctaccacca | |
| | cccctgcccctagacctcccaccccagccccaacaatcgccagccagcctctgtctctgcggcccgaagcctgtagac | |
| | ctgctgccggcggagccgtgcacaccagaggcctggacttcgcctgcgacatctacatctgggcccctctggccggca | |
| | cctgtggcgtgctgctgctgagcctggtgatcaccctgtactgcaaccaccggaacagattcagcgtcgtgaagcggg | |
| | gcagaaagaagctgctgtacatcttcaagcagcccttcatgcggcccgtgcagaccacacaagaggaagatggctg | |
| | ctcctgcagattccctgaggaagaagaaggcggctgcgagctgagagtgaagttcagcagatccgccgacgcccct | |
| | gcctaccagcagggacagaaccagctgtacaacgagctgaacctgggcagacgggaagagtacgacgtgctggac | |
| | aagcggagaggccgggaccccgagatgggcggaaagcccagacggaagaacccccaggaaggcctgtataacga | |
| | actgcagaaagacaagatggccgaggcctacagcgagatcggcatgaagggcgagcggaggcgcggcaagggcc | |
| | acgatggcctgtaccagggcctgagcaccgccaccaaggacacctacgacgccctgcacatgcaggccctgcccccc | |
| | agaggatccggcgctacaaatttttcactgctgaaacaggcgggtgatgtggaggagaaccctggacccatgccact | |
| | tggcctgctctggctgggcttggcattgctcggcgcgctccacgcccaggctgaactgatccgcgtggccatattgtgg | |
| | catgagatgtggcatgagggattggaggaggcgagtaggctgtactttggggaaaggaatgttaaagggatgtttga | |
| | ggtccttgaacccctccacgctatgatggaaagaggacctcaaacgcttaaagagacgtcattcaatcaagcctatg | |
| | gacgggatcttatggaagctcaagaatggtgtcgaaaatacatgaaaagcgggaatgttaaggacctcacgcaagc | |
| | ctgggatctgtattaccatgttttccgacgcatttctaaacaaggaaaagatactatcccatggttggggcacttgctc | |
| | gttgggctcagtggggcgtttggattcatcatcctcgtatatctgttgattaattgtcggaacacaggtccctggcttaa | |
| | aaaagttttgaagtgtaacaccccggatccttctaaattttttagtcaacttagttcagaacacgggggcgatgttcaa | |
| | aagtggctgagttccccgtttcccagttcaagtttctcccctgggggtctcgcccccgagatatcacctcttgaagtgct | |
| | cgagcgggacaaagttacacagcttcttttgcaacaggataaggttccggagccggcgtctctcagctctaaccattc | |
| | actcacttcttgtttcaccaaccaagggtattttttcttccatctgcctgatgccttggagattgaggcttgtcaggtgta | |
| | ctttacctatgacccctatagtgaggaagaccctgacgaaggcgtagctggcgcccccactggctccagtccacagcc | |
| | tcttcagcctctgtcaggggaggacgacgcatattgtacgttcccctcacgggacgaccttctgctgttttcaccctcac | |
| | tgctcggcggaccctccccgccaagcacggcacctggggggagtggggcaggagaagaaaggatgcctcctagttt | |
| | gcaggagcgggttcctcgcgactgggatccgcaacccctcggaccacccacccctggcgtacctgatctggtcgactt | |
| | ccaaccacctccggagcttgtcctcagagaggccggagaggaagtcccagacgcggggccaagagagggtgtgtca | |
| | tttccctggtcccgccctccgggacagggtgagtttcgggcgctgaatgcgaggctcccccttaataccgatgcgtacc | |
| | tgtcattgcaggaacttcagggccaggatcctacccacctggtgtgaaagcttgataatcaacctctggattacaaaa | |
| | tttgtgaaagattgactggtattcttaactatgttgctccttttacgctatgtggatacgctgctttaatgcctttgtatcat | |
| | gctattgcttcccgtatggctttcattttctcctccttgtataaatcctggttagttcttgccacggcggaactcatcgccg | |
| | cctgccttgcccgctgctggacaggggctcggctgttgggcactgacaattccgtggaacttgtttattgcagcttataa | |
| | tggttacaaataaagcaatagcatcacaaatttcacaaataaagcatttttttcactgcattctagttgtggtttgtcca | |
| | aactcatcaatgtatcttacgccggcgtgaatgaggtctatggacttcaagagcaacagtgctgtggcctggagcaac | |
| | aaatctgactttgcatgtgcaaacgccttcaacaacagcattattccagaagacaccttcttccccagcccaggtaag | |
| | ggcagctttggtgccttcgcaggctgtttccttgcttcaggaatggccaggttctgcccagagctctggtcaatgatgtc | |
| | taaaactcctctgattggtggtctcggccttatccattgccaccaaaaccctctttttactaagaaacagtgagccttgt | |
| | tctggcagtccagagaatgacacgggaaaaaagcagatgaagagaaggtggcaggagagggcacgtggcccagc | |
| | ctcagtctctccaactgagttcctgcctgcctgcctttgc | |
| 94 | cctgcaggcagctgcgcgctcgctcgctcactgaggccgcccgggcaaagcccgggcgtcgggcgacctttggtcgc | TRAC AAV |
| | ccggcctcagtgagcgagcgagcgcgcagagagggagtggccaactccatcactaggggttcctgcggcccgcggc | TRAC 2: HA |
| | ggcttgtgcctgtccctgagtcccagtccatcacgagcagctggtttctaagatgctatttcccgtataaagcatgaga | TRAC 2- |
| | ccgtgacttgccagccccacagagccccgcccttgtccatcactggcatctggactccagcctgggttggggcaaaga | syn pA- |
| | gggaaatgagatcatgtcctaaccctgatcctcttgtcccacagatatccagaaccctgaccctgccgtgtaccagctg | MND- |
| | agagactctaaatccagtgacaagtctgtctgcctattcaccgattttgattctcaaacaaatgtgtcacaaagtaagg | Kozak-ER- |
| | attctgatgtgtatatcacagacaaaactgtgctagacatgaggtctatggacttcaagagcaacagtgctgtttaatt | FKBP- |
| | aaatgaaataaaagatctttattttcattagatctgtgtgttggttttttgtgtgaacagagaaacaggagaatatggg | IL2RG-P2A- |
| | ccaaacaggatatctgtggtaagcagttcctgccccggctcagggccaagaacagttggaacagcagaatatgggc | ER-FRB- |
| | caaacaggatatctgtggtaagcagttcctgccccggctcagggccaagaacagatggtccccagatgcggtcccgc | IL2RB-P2A- |
| | cctcagcagtttctagagaaccatcagatgtttccagggtgccccaaggacctgaaatgaccctgtgccttatttgaac | mCherry- |
| | taaccaatcagttcgcttctcgcttctgttcgcgcgcttctgctccccgagctctatataagcagagctcgtttagtgaac | WPRE3- |
| | cgtcagatcgccgccaccatgccacttggcctgctctggctgggcttggcattgctcggcgcgctccacgcccaggctg | BGHpA-HA |
| | gcgttcaagttgaaaccattagtcccggagacggtcgaacatttcccaaacggggccagacgtgcgtggtacactac | TRAC 2 |
| | accggaatgctggaggatggaaaaaaatttgacagcagccgggacagaaacaaaccattcaagttcatgcttggta | |
| | aacaagaggtaatacggggttgggaagagggtgtggcccagatgtcagtagggcaacgcgcgaagttgaccataa | |
| | gccccgactatgcctatggggcgacaggccatcccggtataattcctccgcacgctacactggtgtttgatgttgagtt | |
| | gctgaagctggagcaaaatcttgttattccgtgggctcccgagaacctcacattgcacaaattgtccgaatcacaatt | |
| | ggagcttaattggaacaatagattcctgaatcactgccttgagcacctcgtacaataccggacagactgggatcactc | |
| | ttggacggagcagtccgtggactaccgacataaattctcactcccctcagtggatggccagaaacgctatacctttag | |
| | agtccggtcccgcttcaacccgttgtgcggcagcgcacagcactggagtgaatggagtcatccgatacactggggaa | |
| | gcaatacgtcaaaagagaacccgttcctttttgcgctggaagcagtcgtgatcagcgttggatctatggggctgatcat | |
| | ctcccttctctgcgtctatttctggctcgaaagaactatgccacgcatccctacgctgaaaaatctggaggatcttgtga | |
| | cggaatatcatggaaatttttccgcctggagtggagtttccaaaggtctcgctgaatctctgcagccagactatagtga | |
| | gcggctctgcttggtctctgagattccacctaaggggggggcgctcggggaaggcccgggcgcaagtccgtgtaatc | |
| | aacacagtccgtactgggctccaccatgctataccctcaagccggaaactggatccggcgctacaaatttttcactgct | |
| | gaaacaggcgggtgatgtggaggagaaccctggacccatgccacttggcctgctctggctgggcttggcattgctcg | |
| | gcgcgctccacgcccaggctgaactgatccgcgtggccatattgtggcatgagatgtggcatgagggattggaggag | |
| | gcgagtaggctgtactttggggaaaggaatgttaaagggatgtttgaggtccttgaacccctccacgctatgatggaa | |
| | agaggacctcaaacgcttaaagagacgtcattcaatcaagcctatggacgggatcttatggaagctcaagaatggtg | |
| | tcgaaaatacatgaaaagcgggaatgttaaggacctcacgcaagcctgggatctgtattaccatgttttccgacgcat | |
| | ttctaaacaaggaaaagatactatcccatggttggggcacttgctcgttgggctcagtggggcgtttggattcatcatc | |
| | ctcgtatatctgttgattaattgtcggaacacaggtccctggcttaaaaaagttttgaagtgtaacaccccggatccttc | |
| | taaattttttagtcaacttagttcagaacacgggggcgatgttcaaaagtggctgagttccccgtttcccagttcaagtt | |
| | tctcccctgggggtctcgcccccgagatatcacctcttgaagtgctcgagcgggacaaagttacacagcttcttttgca | |
| | acaggataaggttccggagccggcgtctctcagctctaaccattcactcacttcttgtttcaccaaccaagggtattttt | |
| | tcttccatctgcctgatgccttggagattgaggcttgtcaggtgtactttacctatgacccctatagtgaggaagaccct | |
| | gacgaaggcgtagctggcgcccccactggctccagtccacagcctcttcagcctctgtcaggggaggacgacgcata | |
| | ttgtacgttcccctcacgggacgaccttctgctgttttcaccctcactgctcggcggaccctccccgccaagcacggcac | |
| | ctggggggagtggggcaggagaagaaaggatgcctcctagtttgcaggagcgggttcctcgcgactgggatccgca | |
| | acccctcggaccacccacccctggcgtacctgatctggtcgacttccaaccacctccggagcttgtcctcagagaggc | |
| | cggagaggaagtcccagacgcggggccaagagagggtgtgtcatttccctggtcccgccctccgggacagggtgag | |
| | tttcgggcgctgaatgcgaggctcccccttaataccgatgcgtacctgtcattgcaggaacttcagggccaggatccta | |
| | cccacctggtgggaagcggagctactaacttcagcctgctgaagcaggctggagacgtggaggagaaccctggacc | |
| | tatggtgagcaagggcgaggaggataacatggccatcatcaaggagttcatgcgcttcaaggtgcacatggagggc | |
| | tccgtgaacggccacgagttcgagatcgagggcgagggcgagggccgcccctacgagggcacccagaccgccaag | |
| | ctgaaggtgaccaagggtggccccctgcccttcgcctgggacatcctgtcccctcagttcatgtacggctccaaggcct | |
| | acgtgaagcaccccgccgacatccccgactacttgaagctgtccttccccgagggcttcaagtgggagcgcgtgatg | |
| | aacttcgaggacggcggcgtggtgaccgtgacccaggactcctccctgcaggacggcgagttcatctacaaggtgaa | |
| | gctgcgcggcaccaacttcccctccgacggccccgtaatgcagaagaagaccatgggctgggaggcctcctccgagc | |
| | ggatgtaccccgaggacggcgccctgaagggcgagatcaagcagaggctgaagctgaaggacggcggccactacg | |
| | acgctgaggtcaagaccacctacaaggccaagaagcccgtgcagctgcccggcgcctacaacgtcaacatcaagtt | |
| | ggacatcacctcccacaacgaggactacaccatcgtggaacagtacgaacgcgccgagggccgccactccaccggc | |
| | ggcatggacgagctgtacaagtaggtaagataatcaacctctggattacaaaatttgtgaaagattgactggtattct | |
| | taactatgttgctccttttacgctatgtggatacgctgctttaatgcctttgtatcatgctattgcttcccgtatggctttca | |
| | ttttctcctccttgtataaatcctggttagttcttgccacggcggaactcatcgccgcctgccttgcccgctgctggacag | |
| | gggctcggctgttgggcactgacaattccgtggtgtgccttctagttgccagccatctgttgtttgcccctcccccgtgcc | |
| | ttccttgaccctggaaggtgccactcccactgtcctttcctaataaaatgaggaaattgcatcgcattgtctgagtaggt | |
| | gtcattctattctggggggtggggtggggcaggacagcaagggggaggattgggaagacaatagcaggcatgctgg | |
| | ggatgcggtgggctctacgccggcgagcaacaaatctgactttgcatgtgcaaacgccttcaacaacagcattattcc | |
| | agaagacaccttcttccccagcccaggtaagggcagctttggtgccttcgcaggctgtttccttgcttcaggaatggcc | |
| | aggttctgcccagagctctggtcaatgatgtctaaaactcctctgattggtggtctcggccttatccattgccaccaaaa | |
| | ccctctttttactaagaaacagtgagccttgttctggcagtccagagaatgacacgggaaaaaagcagatgaagaga | |
| | aggtggcaggagagggcacgtggcccagcctcagtctctccaactgagttcctgcctgcctgcctttgctcagactgtt | |
| | tgccccttactgctcttctaggcctcattctaagccccttctccaagttgcctcctagggaattgccttaggccgcagga | |
| | acccctagtgatggagttggccactccctctctgcgcgctcgctcgctcactgaggccgggcgaccaaaggtcgcccg | |
| | acgcccgggcggcctcagtgagcgagcgagcgcgcagctgcctgcagg | |
| 95 | cctgcaggcagctgcgcgctcgctcgctcactgaggccgcccgggcaaagcccgggcgtcgggcgacctttggtcgc | TRAC AAV |
| | ccggcctcagtgagcgagcgagcgcgcagagagggagtggccaactccatcactaggggttcctgcggcccgcggc | TRAC 3: HA |
| | ggtgcctttactctgccagagttatattgctggggttttgaagaagatcctattaaataaaagaataagcagtattatt | TRAC 3- |
| | aagtagccctgcatttcaggtttccttgagtggcaggccaggcctggccgtgaacgttcactgaaatcatggcctcttg | syn pA- |
| | gccaagattgatagcttgtgcctgtccctgagtcccagtccatcacgagcagctggtttctaagatgctatttcccgtat | MND- |
| | aaagcatgagaccgtgacttgccagccccacagagccccgcccttgtccatcactggcatctggactccagcctgggt | Kozak-ER- |
| | tggggcaaagagggaaatgagatcatgtcctaaccctgatcctcttgtcccacagatatccagaaccctgacttaatt | FKBP- |
| | aaatgaaataaaagatctttattttcattagatctgtgtgttggttttttgtgtgaacagagaaacaggagaatatggg | IL2RG-P2A- |
| | ccaaacaggatatctgtggtaagcagttcctgccccggctcagggccaagaacagttggaacagcagaatatgggc | ER-FRB- |
| | caaacaggatatctgtggtaagcagttcctgccccggctcagggccaagaacagatggtccccagatgcggtcccgc | IL2RB-P2A- |
| | cctcagcagtttctagagaaccatcagatgtttccagggtgccccaaggacctgaaatgaccctgtgccttatttgaac | mCherry- |
| | taaccaatcagttcgcttctcgcttctgttcgcgcgcttctgctccccgagctctatataagcagagctcgtttagtgaac | WPRE3- |
| | cgtcagatcgccgccaccatgccacttggcctgctctggctgggcttggcattgctcggcgcgctccacgcccaggctg | BGHpA-HA |
| | gcgttcaagttgaaaccattagtcccggagacggtcgaacatttcccaaacggggccagacgtgcgtggtacactac | TRAC 3 |
| | accggaatgctggaggatggaaaaaaatttgacagcagccgggacagaaacaaaccattcaagttcatgcttggta | |
| | aacaagaggtaatacggggttgggaagagggtgtggcccagatgtcagtagggcaacgcgcgaagttgaccataa | |
| | gccccgactatgcctatggggcgacaggccatcccggtataattcctccgcacgctacactggtgtttgatgttgagtt | |
| | gctgaagctggagcaaaatcttgttattccgtgggctcccgagaacctcacattgcacaaattgtccgaatcacaatt | |
| | ggagcttaattggaacaatagattcctgaatcactgccttgagcacctcgtacaataccggacagactgggatcactc | |
| | ttggacggagcagtccgtggactaccgacataaattctcactcccctcagtggatggccagaaacgctatacctttag | |
| | agtccggtcccgcttcaacccgttgtgcggcagcgcacagcactggagtgaatggagtcatccgatacactggggaa | |
| | gcaatacgtcaaaagagaacccgttcctttttgcgctggaagcagtcgtgatcagcgttggatctatggggctgatcat | |
| | ctcccttctctgcgtctatttctggctcgaaagaactatgccacgcatccctacgctgaaaaatctggaggatcttgtga | |
| | cggaatatcatggaaatttttccgcctggagtggagtttccaaaggtctcgctgaatctctgcagccagactatagtga | |
| | gcggctctgcttggtctctgagattccacctaaggggggggcgctcggggaaggcccgggcgcaagtccgtgtaatc | |
| | aacacagtccgtactgggctccaccatgctataccctcaagccggaaactggatccggcgctacaaatttttcactgct | |
| | gaaacaggcgggtgatgtggaggagaaccctggacccatgccacttggcctgctctggctgggcttggcattgctcg | |
| | gcgcgctccacgcccaggctgaactgatccgcgtggccatattgtggcatgagatgtggcatgagggattggaggag | |
| | gcgagtaggctgtactttggggaaaggaatgttaaagggatgtttgaggtccttgaacccctccacgctatgatggaa | |
| | agaggacctcaaacgcttaaagagacgtcattcaatcaagcctatggacgggatcttatggaagctcaagaatggtg | |
| | tcgaaaatacatgaaaagcgggaatgttaaggacctcacgcaagcctgggatctgtattaccatgttttccgacgcat | |
| | ttctaaacaaggaaaagatactatcccatggttggggcacttgctcgttgggctcagtggggcgtttggattcatcatc | |
| | ctcgtatatctgttgattaattgtcggaacacaggtccctggcttaaaaaagttttgaagtgtaacaccccggatccttc | |
| | taaattttttagtcaacttagttcagaacacgggggcgatgttcaaaagtggctgagttccccgtttcccagttcaagtt | |
| | tctcccctgggggtctcgcccccgagatatcacctcttgaagtgctcgagcgggacaaagttacacagcttcttttgca | |
| | acaggataaggttccggagccggcgtctctcagctctaaccattcactcacttcttgtttcaccaaccaagggtattttt | |
| | tcttccatctgcctgatgccttggagattgaggcttgtcaggtgtactttacctatgacccctatagtgaggaagaccct | |
| | gacgaaggcgtagctggcgcccccactggctccagtccacagcctcttcagcctctgtcaggggaggacgacgcata | |
| | ttgtacgttcccctcacgggacgaccttctgctgttttcaccctcactgctcggcggaccctccccgccaagcacggcac | |
| | ctggggggagtggggcaggagaagaaaggatgcctcctagtttgcaggagcgggttcctcgcgactgggatccgca | |
| | acccctcggaccacccacccctggcgtacctgatctggtcgacttccaaccacctccggagcttgtcctcagagaggc | |
| | cggagaggaagtcccagacgcggggccaagagagggtgtgtcatttccctggtcccgccctccgggacagggtgag | |
| | tttcgggcgctgaatgcgaggctcccccttaataccgatgcgtacctgtcattgcaggaacttcagggccaggatccta | |
| | cccacctggtgggaagcggagctactaacttcagcctgctgaagcaggctggagacgtggaggagaaccctggacc | |
| | tatggtgagcaagggcgaggaggataacatggccatcatcaaggagttcatgcgcttcaaggtgcacatggagggc | |
| | tccgtgaacggccacgagttcgagatcgagggcgagggcgagggccgcccctacgagggcacccagaccgccaag | |
| | ctgaaggtgaccaagggtggccccctgcccttcgcctgggacatcctgtcccctcagttcatgtacggctccaaggcct | |
| | acgtgaagcaccccgccgacatccccgactacttgaagctgtccttccccgagggcttcaagtgggagcgcgtgatg | |
| | aacttcgaggacggcggcgtggtgaccgtgacccaggactcctccctgcaggacggcgagttcatctacaaggtgaa | |
| | gctgcgcggcaccaacttcccctccgacggccccgtaatgcagaagaagaccatgggctgggaggcctcctccgagc | |
| | ggatgtaccccgaggacggcgccctgaagggcgagatcaagcagaggctgaagctgaaggacggcggccactacg | |
| | acgctgaggtcaagaccacctacaaggccaagaagcccgtgcagctgcccggcgcctacaacgtcaacatcaagtt | |
| | ggacatcacctcccacaacgaggactacaccatcgtggaacagtacgaacgcgccgagggccgccactccaccggc | |
| | ggcatggacgagctgtacaagtaggtaagataatcaacctctggattacaaaatttgtgaaagattgactggtattct | |
| | taactatgttgctccttttacgctatgtggatacgctgctttaatgcctttgtatcatgctattgcttcccgtatggctttca | |
| | ttttctcctccttgtataaatcctggttagttcttgccacggcggaactcatcgccgcctgccttgcccgctgctggacag | |
| | gggctcggctgttgggcactgacaattccgtggtgtgccttctagttgccagccatctgttgtttgcccctcccccgtgcc | |
| | ttccttgaccctggaaggtgccactcccactgtcctttcctaataaaatgaggaaattgcatcgcattgtctgagtaggt | |
| | gtcattctattctggggggtggggtggggcaggacagcaagggggaggattgggaagacaatagcaggcatgctgg | |
| | ggatgcggtgggctctacgccggcgtaccagctgagagactctaaatccagtgacaagtctgtctgcctattcaccga | |
| | ttttgattctcaaacaaatgtgtcacaaagtaaggattctgatgtgtatatcacagacaaaactgtgctagacatgag | |
| | gtctatggacttcaagagcaacagtgctgtggcctggagcaacaaatctgactttgcatgtgcaaacgccttcaacaa | |
| | cagcattattccagaagacaccttcttccccagcccaggtaagggcagctttggtgccttcgcaggctgtttccttgctt | |
| | caggaatggccaggttctgcccagagctctggtcaatgatgtctaaaactcctctgattggtggtctcggccttatccat | |
| | tgccaccaaaaccctctttttactaagaaacagtgagccttgttctggcagtcctagggaattgccttaggccgcagga | |
| | acccctagtgatggagttggccactccctctctgcgcgctcgctcgctcactgaggccgggcgaccaaaggtcgcccg | |
| | acgcccgggcggcctcagtgagcgagcgagcgcgcagctgcctgcagg | |
| 96 | cctgcaggcagctgcgcgctcgctcgctcactgaggccgcccgggcaaagcccgggcgtcgggcgacctttggtcgc | TRAC AAV |
| | ccggcctcagtgagcgagcgagcgcgcagagagggagtggccaactccatcactaggggttcctgcggcccgcggc | TRAC 1: HA |
| | ggccgcgccaggcctggccgtgaacgttcactgaaatcatggcctcttggccaagattgatagcttgtgcctgtccctg | AS-synpA- |
| | agtcccagtccatcacgagcagctggtttctaagatgctatttcccgtataaagcatgagaccgtgacttgccagcccc | MND- |
| | acagagccccgcccttgtccatcactggcatctggactccagcctgggttggggcaaagagggaaatgagatcatgt | Kozak-ER- |
| | cctaaccctgatcctcttgtcccacagatatccagaaccctgaccctgccgtgtaccagctgagagactctaaatccag | FKBP- |
| | tgacaagtctgtctgcctattcaccgattttgattctcaaacaaatgtgtcacaaagtaaggattctgatgtgtatatca | IL2RG-P2A- |
| | catgttaattaaatgaaataaaagatctttattttcattagatctgtgtgttggttttttgtgtgaacagagaaacagga | ER-FRB- |
| | gaatatgggccaaacaggatatctgtggtaagcagttcctgccccggctcagggccaagaacagttggaacagcag | IL2RB-P2A- |
| | aatatgggccaaacaggatatctgtggtaagcagttcctgccccggctcagggccaagaacagatggtccccagatg | mCherry- |
| | cggtcccgccctcagcagtttctagagaaccatcagatgtttccagggtgccccaaggacctgaaatgaccctgtgcc | WPRE3- |
| | ttatttgaactaaccaatcagttcgcttctcgcttctgttcgcgcgcttctgctccccgagctctatataagcagagctcg | BGHpA-HA |
| | tttagtgaaccgtcagatcgccgccaccatgccacttggcctgctctggctgggcttggcattgctcggcgcgctccac | TRAC 1 |
| | gcccaggctggcgttcaagttgaaaccattagtcccggagacggtcgaacatttcccaaacggggccagacgtgcgt | |
| | ggtacactacaccggaatgctggaggatggaaaaaaatttgacagcagccgggacagaaacaaaccattcaagttc | |
| | atgcttggtaaacaagaggtaatacggggttgggaagagggtgtggcccagatgtcagtagggcaacgcgcgaagt | |
| | tgaccataagccccgactatgcctatggggcgacaggccatcccggtataattcctccgcacgctacactggtgtttga | |
| | tgttgagttgctgaagctggagcaaaatcttgttattccgtgggctcccgagaacctcacattgcacaaattgtccgaa | |
| | tcacaattggagcttaattggaacaatagattcctgaatcactgccttgagcacctcgtacaataccggacagactgg | |
| | gatcactcttggacggagcagtccgtggactaccgacataaattctcactcccctcagtggatggccagaaacgctat | |
| | acctttagagtccggtcccgcttcaacccgttgtgcggcagcgcacagcactggagtgaatggagtcatccgatacac | |
| | tggggaagcaatacgtcaaaagagaacccgttcctttttgcgctggaagcagtcgtgatcagcgttggatctatgggg | |
| | ctgatcatctcccttctctgcgtctatttctggctcgaaagaactatgccacgcatccctacgctgaaaaatctggagga | |
| | tcttgtgacggaatatcatggaaatttttccgcctggagtggagtttccaaaggtctcgctgaatctctgcagccagact | |
| | atagtgagcggctctgcttggtctctgagattccacctaaggggggggcgctcggggaaggcccgggcgcaagtccg | |
| | tgtaatcaacacagtccgtactgggctccaccatgctataccctcaagccggaaactggatccggcgctacaaattttt | |
| | cactgctgaaacaggcgggtgatgtggaggagaaccctggacccatgccacttggcctgctctggctgggcttggcat | |
| | tgctcggcgcgctccacgcccaggctgaactgatccgcgtggccatattgtggcatgagatgtggcatgagggattgg | |
| | aggaggcgagtaggctgtactttggggaaaggaatgttaaagggatgtttgaggtccttgaacccctccacgctatga | |
| | tggaaagaggacctcaaacgcttaaagagacgtcattcaatcaagcctatggacgggatcttatggaagctcaaga | |
| | atggtgtcgaaaatacatgaaaagcgggaatgttaaggacctcacgcaagcctgggatctgtattaccatgttttccg | |
| | acgcatttctaaacaaggaaaagatactatcccatggttggggcacttgctcgttgggctcagtggggcgtttggattc | |
| | atcatcctcgtatatctgttgattaattgtcggaacacaggtccctggcttaaaaaagttttgaagtgtaacaccccgg | |
| | atccttctaaattttttagtcaacttagttcagaacacgggggcgatgttcaaaagtggctgagttccccgtttcccagt | |
| | tcaagtttctcccctgggggtctcgcccccgagatatcacctcttgaagtgctcgagcgggacaaagttacacagcttc | |
| | ttttgcaacaggataaggttccggagccggcgtctctcagctctaaccattcactcacttcttgtttcaccaaccaaggg | |
| | tattttttcttccatctgcctgatgccttggagattgaggcttgtcaggtgtactttacctatgacccctatagtgaggaa | |
| | gaccctgacgaaggcgtagctggcgcccccactggctccagtccacagcctcttcagcctctgtcaggggaggacga | |
| | cgcatattgtacgttcccctcacgggacgaccttctgctgttttcaccctcactgctcggcggaccctccccgccaagca | |
| | cggcacctggggggagtggggcaggagaagaaaggatgcctcctagtttgcaggagcgggttcctcgcgactggga | |
| | tccgcaacccctcggaccacccacccctggcgtacctgatctggtcgacttccaaccacctccggagcttgtcctcaga | |
| | gaggccggagaggaagtcccagacgcggggccaagagagggtgtgtcatttccctggtcccgccctccgggacagg | |
| | gtgagtttcgggcgctgaatgcgaggctcccccttaataccgatgcgtacctgtcattgcaggaacttcagggccagg | |
| | atcctacccacctggtgggaagcggagctactaacttcagcctgctgaagcaggctggagacgtggaggagaaccct | |
| | ggacctatggtgagcaagggcgaggaggataacatggccatcatcaaggagttcatgcgcttcaaggtgcacatgg | |
| | agggctccgtgaacggccacgagttcgagatcgagggcgagggcgagggccgcccctacgagggcacccagaccg | |
| | ccaagctgaaggtgaccaagggtggccccctgcccttcgcctgggacatcctgtcccctcagttcatgtacggctcca | |
| | aggcctacgtgaagcaccccgccgacatccccgactacttgaagctgtccttccccgagggcttcaagtgggagcgc | |
| | gtgatgaacttcgaggacggcggcgtggtgaccgtgacccaggactcctccctgcaggacggcgagttcatctacaa | |
| | ggtgaagctgcgcggcaccaacttcccctccgacggccccgtaatgcagaagaagaccatgggctgggaggcctcct | |
| | ccgagcggatgtaccccgaggacggcgccctgaagggcgagatcaagcagaggctgaagctgaaggacggcggcc | |
| | actacgacgctgaggtcaagaccacctacaaggccaagaagcccgtgcagctgcccggcgcctacaacgtcaacat | |
| | caagttggacatcacctcccacaacgaggactacaccatcgtggaacagtacgaacgcgccgagggccgccactcc | |
| | accggcggcatggacgagctgtacaagtaggtaagataatcaacctctggattacaaaatttgtgaaagattgactg | |
| | gtattcttaactatgttgctccttttacgctatgtggatacgctgctttaatgcctttgtatcatgctattgcttcccgtatg | |
| | gctttcattttctcctccttgtataaatcctggttagttcttgccacggcggaactcatcgccgcctgccttgcccgctgct | |
| | ggacaggggctcggctgttgggcactgacaattccgtggtgtgccttctagttgccagccatctgttgtttgcccctccc | |
| | ccgtgccttccttgaccctggaaggtgccactcccactgtcctttcctaataaaatgaggaaattgcatcgcattgtctg | |
| | agtaggtgtcattctattctggggggtggggtggggcaggacagcaagggggaggattgggaagacaatagcaggc | |
| | atgctggggatgcggtgggctctacgccggcgtggcggtctatggacttcaagagcaacagtgctgtggcctggagc | |
| | aacaaatctgactttgcatgtgcaaacgccttcaacaacagcattattccagaagacaccttcttccccagcccaggt | |
| | aagggcagctttggtgccttcgcaggctgtttccttgcttcaggaatggccaggttctgcccagagctctggtcaatgat | |
| | gtctaaaactcctctgattggtggtctcggccttatccattgccaccaaaaccctctttttactaagaaacagtgagcct | |
| | tgttctggcagtccagagaatgacacgggaaaaaagcagatgaagagaaggtggcaggagagggcacgtggccca | |
| | gcctcagtctctccaactgagttcctgcctgcctgcctttgctcagactgtttgccccttactgctccctagggaattgcc | |
| | ttaggccgcaggaacccctagtgatggagttggccactccctctctgcgcgctcgctcgctcactgaggccgggcgac | |
| | caaaggtcgcccgacgcccgggcggcctcagtgagcgagcgagcgcgcagctgcctgcagg | |
| 97 | atgaaataaaagatctttattttcattagatctgtgtgttggttttttgtgtgaacagagaaacaggagaatatgggcc | IL2RG AAV |
| | aaacaggatatctgtggtaagcagttcctgccccggctcagggccaagaacagttggaacagcagaatatgggcca | cassette: |
| | aacaggatatctgtggtaagcagttcctgccccggctcagggccaagaacagatggtccccagatgcggtcccgccc | CISC- |
| | tcagcagtttctagagaaccatcagatgtttccagggtgccccaaggacctgaaatgaccctgtgccttatttgaacta | tLNGFR |
| | accaatcagttcgcttctcgcttctgttcgcgcgcttctgctccccgagctctatataagcagagctcgtttagtgaacc | |
| | gtcagatcgccgccaccATGGGTGCTGGCGCAACTGGACGCGCTATGGATGGACCTCGCTTGC | |
| | TGCTTCTTCTGCTTCTCGGGGTCTCTTTGGGTGGTGCTAAGGAAGCATGCCCAACGGGAC | |
| | TTTATACGCATAGCGGAGAGTGTTGCAAAGCTTGTAACCTGGGCGAAGGCGTCGCGCAA | |
| | CCTTGTGGTGCAAATCAAACCGTCTGCGAGCCATGTTTGGACTCTGTTACGTTTAGTGAC | |
| | GTAGTATCTGCGACAGAGCCATGCAAGCCTTGTACGGAATGTGTAGGATTGCAGAGCAT | |
| | GTCTGCCCCTTGTGTAGAAGCCGACGATGCAGTTTGCAGGTGCGCGTATGGCTATTACCA | |
| | AGACGAAACAACCGGACGATGTGAAGCTTGCCGAGTTTGTGAAGCGGGTTCCGGGCTTG | |
| | TATTCTCCTGTCAGGATAAGCAGAACACCGTCTGCGAAGAGTGCCCCGATGGTACCTACA | |
| | GCGATGAAGCGAACCATGTAGACCCATGCCTGCCTTGCACCGTTTGTGAAGACACGGAA | |
| | CGACAGTTGCGGGAATGTACCCGGTGGGCAGACGCCGAGTGCGAAGAGATTCCAGGCC | |
| | GCTGGATCACGCGAAGTACCCCGCCAGAAGGTTCCGACAGTACTGCACCAAGCACCCAA | |
| | GAACCAGAGGCGCCCCCCGAGCAGGACCTGATTGCCTCCACCGTGGCGGGTGTTGTTAC | |
| | TACGGTTATGGGCTCATCCCAGCCCGTTGTTACCCGAGGAACTACAGACAACCTGATTCC | |
| | GGTATATTGTTCTATCTTGGCGGCTGTAGTAGTTGGCTTGGTCGCGTACATCGCTTTCAAA | |
| | AGAGGATCCGGCGCTACAAATTTTTCACTGCTGAAACAGGCGGGTGATGTGGAGGAGAA | |
| | CCCTGGACCCATGCCACTTGGCCTGCTCTGGCTGGGCTTGGCATTGCTCGGCGCGCTCCA | |
| | CGCCCAGGCTGAACTGATCCGCGTGGCCATATTGTGGCATGAGATGTGGCATGAGGGAT | |
| | TGGAGGAGGCGAGTAGGCTGTACTTTGGGGAAAGGAATGTTAAAGGGATGTTTGAGGT | |
| | CCTTGAACCCCTCCACGCTATGATGGAAAGAGGACCTCAAACGCTTAAAGAGACGTCATT | |
| | CAATCAAGCCTATGGACGGGATCTTATGGAAGCTCAAGAATGGTGTCGAAAATACATGA | |
| | AAAGCGGGAATGTTAAGGACCTCACGCAAGCCTGGGATCTGTATTACCATGTTTTCCGAC | |
| | GCATTTCTAAACAAGGAAAAGATACTATCCCATGGTTGGGGCACTTGCTCGTTGGGCTCA | |
| | GTGGGGCGTTTGGATTCATCATCCTCGTATATCTGTTGATTAATTGTCGGAACACAGGTCC | |
| | CTGGCTTAAAAAAGTTTTGAAGTGTAACACCCCGGATCCTTCTAAATTTTTTAGTCAACTT | |
| | AGTTCAGAACACGGGGGCGATGTTCAAAAGTGGCTGAGTTCCCCGTTTCCCAGTTCAAGT | |
| | TTCTCCCCTGGGGGTCTCGCCCCCGAGATATCACCTCTTGAAGTGCTCGAGCGGGACAAA | |
| | GTTACACAGCTTCTTTTGCAACAGGATAAGGTTCCGGAGCCGGCGTCTCTCAGCTCTAAC | |
| | CATTCACTCACTTCTTGTTTCACCAACCAAGGGTATTTTTTCTTCCATCTGCCTGATGCCTT | |
| | GGAGATTGAGGCTTGTCAGGTGTACTTTACCTATGACCCCTATAGTGAGGAAGACCCTGA | |
| | CGAAGGCGTAGCTGGCGCCCCCACTGGCTCCAGTCCACAGCCTCTTCAGCCTCTGTCAGG | |
| | GGAGGACGACGCATATTGTACGTTCCCCTCACGGGACGACCTTCTGCTGTTTTCACCCTCA | |
| | CTGCTCGGCGGACCCTCCCCGCCAAGCACGGCACCTGGGGGGAGTGGGGCAGGAGAAG | |
| | AAAGGATGCCTCCTAGTTTGCAGGAGCGGGTTCCTCGCGACTGGGATCCGCAACCCCTC | |
| | GGACCACCCACCCCTGGCGTACCTGATCTGGTCGACTTCCAACCACCTCCGGAGCTTGTC | |
| | CTCAGAGAGGCCGGAGAGGAAGTCCCAGACGCGGGGCCAAGAGAGGGTGTGTCATTTC | |
| | CCTGGTCCCGCCCTCCGGGACAGGGTGAGTTTCGGGCGCTGAATGCGAGGCTCCCCCTT | |
| | AATACCGATGCGTACCTGTCATTGCAGGAACTTCAGGGCCAGGATCCTACCCACCTGGTG | |
| | GGATCCGGCGCTACAAATTTTTCACTGCTGAAACAGGCGGGTGATGTGGAGGAGAACCC | |
| | TGGACCCatgcctctgggcctgctgtggctgggcctggccctgctgggcgccctgcacgcccaggccggcgtgcag | |
| | gtggagacaatctccccaggcgacggacgcacattccctaagcggggccagacctgcgtggtgcactatacaggcat | |
| | gctggaggatggcaagaagtttgacagctcccgggatagaaacaagccattcaagtttatgctgggcaagcaggaa | |
| | gtgatcagaggctgggaggagggcgtggcccagatgtctgtgggccagagggccaagctgaccatcagcccagact | |
| | acgcctatggagcaacaggccacccaggaatcatcccacctcacgccaccctggtgttcgatgtggagctgctgaag | |
| | ctgggcgagggcagcaacaccagcaaa | |
| 98 | atggccctgcctgtgacagctctgctcctccctctggccctgctgctccatgccgccagacccgacatcgtgctgaccc | TRAC AAV |
| | agagcccccccagcctggccatgtctctgggcaagagagccaccatcagctgccgggccagcgagagcgtgaccat | #1 |
| | cctgggcagccacctgatccactggtatcagcagaagcccggccagccccccaccctgctgatccagctcgccagca | cassette: |
| | atgtgcagaccggcgtgcccgccagattcagcggcagcggcagcagaaccgacttcaccctgaccatcgaccccgtg | C11D5.3- |
| | gaagaggacgacgtggccgtgtactactgcctgcagagccggaccatcccccggacctttggcggaggcaccaaac | CD8-CD28- |
| | tggaaatcaagggcagcaccagcggctccggcaagcctggctctggcgagggcagcacaaagggacagattcagc | CD3z |
| | tggtgcagagcggccctgagctgaagaaacccggcgagacagtgaagatcagctgcaaggcctccggctacacctt | |
| | caccgactacagcatcaactgggtgaaaagagcccctggcaagggcctgaagtggatgggctggatcaacaccgag | |
| | acaagagagcccgcctacgcctacgacttccggggcagattcgccttcagcctggaaaccagcgccagcaccgccta | |
| | cctgcagatcaacaacctgaagtacgaggacaccgccacctacttttgcgccctggactacagctacgccatggact | |
| | actggggccagggcaccagcgtgaccgtgtccagcttcgtgcccgtgttcctgcccgccaaacctaccaccacccctg | |
| | cccctagacctcccaccccagccccaacaatcgccagccagcctctgtctctgcggcccgaagcctgtagacctgctg | |
| | ccggcggagccgtgcacaccagaggcctggacttcgcctgcgacatctacatctgggcccctctggccggcacctgtg | |
| | gcgtgctgctgctgagcctggtgatcaccctgtactgcaaccaccggaacagaagcaagcggagccggctgctgcac | |
| | agcgactacatgaacatgaccccaagacggcctggccccacccggaagcactaccagccttacgcccctcccagag | |
| | acttcgccgcctaccggtccagagtgaagttcagcagatccgccgacgcccctgcctaccagcagggacagaaccag | |
| | ctgtacaacgagctgaacctgggcagacgggaagagtacgacgtgctggacaagcggagaggccgggaccccgag | |
| | atgggcggaaagcccagacggaagaacccccaggaaggcctgtataacgaactgcagaaagacaagatggccga | |
| | ggcctacagcgagatcggcatgaagggcgagcggaggcgcggcaagggccacgatggcctgtaccagggcctgag | |
| | caccgccaccaaggacacctacgacgccctgcacatgcaggccctgccccccaga | |
| 99 | ggttccggggagggccgagggtcattgctgacgtgtggagacgtggaggagaatcctggccccatggccctgcctgt | TRAC AAV |
| | gacagctctgctcctccctctggccctgctgctccatgccgccagacccgacatcgtgctgacccagagcccccccag | #2 |
| | cctggccatgtctctgggcaagagagccaccatcagctgccgggccagcgagagcgtgaccatcctgggcagccacc | cassette: |
| | tgatccactggtatcagcagaagcccggccagccccccaccctgctgatccagctcgccagcaatgtgcagaccggc | 2A- |
| | gtgcccgccagattcagcggcagcggcagcagaaccgacttcaccctgaccatcgaccccgtggaagaggacgacg | C11D5.3- |
| | tggccgtgtactactgcctgcagagccggaccatcccccggacctttggcggaggcaccaaactggaaatcaagggc | CD8-CD28- |
| | agcaccagcggctccggcaagcctggctctggcgagggcagcacaaagggacagattcagctggtgcagagcggc | CD3z |
| | cctgagctgaagaaacccggcgagacagtgaagatcagctgcaaggcctccggctacaccttcaccgactacagca | |
| | tcaactgggtgaaaagagcccctggcaagggcctgaagtggatgggctggatcaacaccgagacaagagagcccg | |
| | cctacgcctacgacttccggggcagattcgccttcagcctggaaaccagcgccagcaccgcctacctgcagatcaac | |
| | aacctgaagtacgaggacaccgccacctacttttgcgccctggactacagctacgccatggactactggggccaggg | |
| | caccagcgtgaccgtgtccagcttcgtgcccgtgttcctgcccgccaaacctaccaccacccctgcccctagacctccc | |
| | accccagccccaacaatcgccagccagcctctgtctctgcggcccgaagcctgtagacctgctgccggcggagccgt | |
| | gcacaccagaggcctggacttcgcctgcgacatctacatctgggcccctctggccggcacctgtggcgtgctgctgct | |
| | gagcctggtgatcaccctgtactgcaaccaccggaacagaagcaagcggagccggctgctgcacagcgactacatg | |
| | aacatgaccccaagacggcctggccccacccggaagcactaccagccttacgcccctcccagagacttcgccgccta | |
| | ccggtccagagtgaagttcagcagatccgccgacgcccctgcctaccagcagggacagaaccagctgtacaacgag | |
| | ctgaacctgggcagacgggaagagtacgacgtgctggacaagcggagaggccgggaccccgagatgggcggaaa | |
| | gcccagacggaagaacccccaggaaggcctgtataacgaactgcagaaagacaagatggccgaggcctacagcg | |
| | agatcggcatgaagggcgagcggaggcgcggcaagggccacgatggcctgtaccagggcctgagcaccgccacca | |
| | aggacacctacgacgccctgcacatgcaggccctgccccccaga | |
| 100 | ggttccggggagggccgagggtcattgctgacgtgtggagacgtggaggagaatcctggccccatggccctgcctgt | TRAC AAV |
| | gacagctctgctcctccctctggccctgctgctccatgccgccagacccgacatcgtgctgacccagagcccccccag | #3 |
| | cctggccatgtctctgggcaagagagccaccatcagctgccgggccagcgagagcgtgaccatcctgggcagccacc | cassette: |
| | tgatccactggtatcagcagaagcccggccagccccccaccctgctgatccagctcgccagcaatgtgcagaccggc | 2A- |
| | gtgcccgccagattcagcggcagcggcagcagaaccgacttcaccctgaccatcgaccccgtggaagaggacgacg | C11D5.3- |
| | tggccgtgtactactgcctgcagagccggaccatcccccggacctttggcggaggcaccaaactggaaatcaagggc | CD8-CD28- |
| | agcaccagcggctccggcaagcctggctctggcgagggcagcacaaagggacagattcagctggtgcagagcggc | CD3z- |
| | cctgagctgaagaaacccggcgagacagtgaagatcagctgcaaggcctccggctacaccttcaccgactacagca | CNb30 |
| | tcaactgggtgaaaagagcccctggcaagggcctgaagtggatgggctggatcaacaccgagacaagagagcccg | |
| | cctacgcctacgacttccggggcagattcgccttcagcctggaaaccagcgccagcaccgcctacctgcagatcaac | |
| | aacctgaagtacgaggacaccgccacctacttttgcgccctggactacagctacgccatggactactggggccaggg | |
| | caccagcgtgaccgtgtccagcttcgtgcccgtgttcctgcccgccaaacctaccaccacccctgcccctagacctccc | |
| | accccagccccaacaatcgccagccagcctctgtctctgcggcccgaagcctgtagacctgctgccggcggagccgt | |
| | gcacaccagaggcctggacttcgcctgcgacatctacatctgggcccctctggccggcacctgtggcgtgctgctgct | |
| | gagcctggtgatcaccctgtactgcaaccaccggaacagaagcaagcggagccggctgctgcacagcgactacatg | |
| | aacatgaccccaagacggcctggccccacccggaagcactaccagccttacgcccctcccagagacttcgccgccta | |
| | ccggtccagagtgaagttcagcagatccgccgacgcccctgcctaccagcagggacagaaccagctgtacaacgag | |
| | ctgaacctgggcagacgggaagagtacgacgtgctggacaagcggagaggccgggaccccgagatgggcggaaa | |
| | gcccagacggaagaacccccaggaaggcctgtataacgaactgcagaaagacaagatggccgaggcctacagcg | |
| | agatcggcatgaagggcgagcggaggcgcggcaagggccacgatggcctgtaccagggcctgagcaccgccacca | |
| | aggacacctacgacgccctgcacatgcaggccctgccccccagaggcagcggcgaaggcagaggatccctgcttac | |
| | atgtggcgacgtggaagagaaccctggccccatgggcaacgaggccagctaccctctggagatgtgctcccacttcg | |
| | acgccgacgagatcaagcggctgggcaagcgcttcaagaagctggacctggacaacagcggcagcctgagcgtgg | |
| | aggagtttatgtctctgcccgagctgcagcagaaccccctggtgcagcgcgtgatcgacatcttcgacaccgacggca | |
| | acggcgaggtggacttcaaggagttcatcgagggcgtgagccagttcagcgtgaagggcgacaaggagcagaagc | |
| | tgcggttcgccttccggatctacgatatggataaagatggctatatttctaatggcgagctgttccaggtgctgaagat | |
| | gatggtgggcaacaataccaagctggccgatacccagctgcagcagatcgtggacaagaccatcatcaacgccgac | |
| | aaggacggcgacggcagaatcagcttcgaggagttctgtgccgtggtgggaggcctggatattcacaaaaaaatgg | |
| | tggtggacgtg | |
| 101 | atggccctgcctgtgacagctctgctcctccctctggccctgctgctccatgccgccagacccgacatcgtgctgaccc | TRAC AAV |
| | agagcccccccagcctggccatgtctctgggcaagagagccaccatcagctgccgggccagcgagagcgtgaccat | #4 |
| | cctgggcagccacctgatccactggtatcagcagaagcccggccagccccccaccctgctgatccagctcgccagca | cassette: |
| | atgtgcagaccggcgtgcccgccagattcagcggcagcggcagcagaaccgacttcaccctgaccatcgaccccgtg | C11D5.3- |
| | gaagaggacgacgtggccgtgtactactgcctgcagagccggaccatcccccggacctttggcggaggcaccaaac | CD8-CD28- |
| | tggaaatcaagggcagcaccagcggctccggcaagcctggctctggcgagggcagcacaaagggacagattcagc | CD3z-P2A- |
| | tggtgcagagcggccctgagctgaagaaacccggcgagacagtgaagatcagctgcaaggcctccggctacacctt | CISCb |
| | caccgactacagcatcaactgggtgaaaagagcccctggcaagggcctgaagtggatgggctggatcaacaccgag | |
| | acaagagagcccgcctacgcctacgacttccggggcagattcgccttcagcctggaaaccagcgccagcaccgccta | |
| | cctgcagatcaacaacctgaagtacgaggacaccgccacctacttttgcgccctggactacagctacgccatggact | |
| | actggggccagggcaccagcgtgaccgtgtccagcttcgtgcccgtgttcctgcccgccaaacctaccaccacccctg | |
| | cccctagacctcccaccccagccccaacaatcgccagccagcctctgtctctgcggcccgaagcctgtagacctgctg | |
| | ccggcggagccgtgcacaccagaggcctggacttcgcctgcgacatctacatctgggcccctctggccggcacctgtg | |
| | gcgtgctgctgctgagcctggtgatcaccctgtactgcaaccaccggaacagaagcaagcggagccggctgctgcac | |
| | agcgactacatgaacatgaccccaagacggcctggccccacccggaagcactaccagccttacgcccctcccagag | |
| | acttcgccgcctaccggtccagagtgaagttcagcagatccgccgacgcccctgcctaccagcagggacagaaccag | |
| | ctgtacaacgagctgaacctgggcagacgggaagagtacgacgtgctggacaagcggagaggccgggaccccgag | |
| | atgggcggaaagcccagacggaagaacccccaggaaggcctgtataacgaactgcagaaagacaagatggccga | |
| | ggcctacagcgagatcggcatgaagggcgagcggaggcgcggcaagggccacgatggcctgtaccagggcctgag | |
| | caccgccaccaaggacacctacgacgccctgcacatgcaggccctgccccccagaggatccggcgctacaaattttt | |
| | cactgctgaaacaggcgggtgatgtggaggagaaccctggacccatgccacttggcctgctctggctgggcttggcat | |
| | tgctcggcgcgctccacgcccaggctgaactgatccgcgtggccatattgtggcatgagatgtggcatgagggattgg | |
| | aggaggcgagtaggctgtactttggggaaaggaatgttaaagggatgtttgaggtccttgaacccctccacgctatga | |
| | tggaaagaggacctcaaacgcttaaagagacgtcattcaatcaagcctatggacgggatcttatggaagctcaaga | |
| | atggtgtcgaaaatacatgaaaagcgggaatgttaaggacctcacgcaagcctgggatctgtattaccatgttttccg | |
| | acgcatttctaaacaaggaaaagatactatcccatggttggggcacttgctcgttgggctcagtggggcgtttggattc | |
| | atcatcctcgtatatctgttgattaattgtcggaacacaggtccctggcttaaaaaagttttgaagtgtaacaccccgg | |
| | atccttctaaattttttagtcaacttagttcagaacacgggggcgatgttcaaaagtggctgagttccccgtttcccagt | |
| | tcaagtttctcccctgggggtctcgcccccgagatatcacctcttgaagtgctcgagcgggacaaagttacacagcttc | |
| | ttttgcaacaggataaggttccggagccggcgtctctcagctctaaccattcactcacttcttgtttcaccaaccaaggg | |
| | tattttttcttccatctgcctgatgccttggagattgaggcttgtcaggtgtactttacctatgacccctatagtgaggaa | |
| | gaccctgacgaaggcgtagctggcgcccccactggctccagtccacagcctcttcagcctctgtcaggggaggacga | |
| | cgcatattgtacgttcccctcacgggacgaccttctgctgttttcaccctcactgctcggcggaccctccccgccaagca | |
| | cggcacctggggggagtggggcaggagaagaaaggatgcctcctagtttgcaggagcgggttcctcgcgactggga | |
| | tccgcaacccctcggaccacccacccctggcgtacctgatctggtcgacttccaaccacctccggagcttgtcctcaga | |
| | gaggccggagaggaagtcccagacgcggggccaagagagggtgtgtcatttccctggtcccgccctccgggacagg | |
| | gtgagtttcgggcgctgaatgcgaggctcccccttaataccgatgcgtacctgtcattgcaggaacttcagggccagg | |
| | atcctacccacctggtg | |
| 102 | ggttccggggagggccgagggtcattgctgacgtgtggagacgtggaggagaatcctggccccatggccctgcctgt | TRAC AAV |
| | gacagctctgctcctccctctggccctgctgctccatgccgccagacccgacatcgtgctgacccagagcccccccag | #5 |
| | cctggccatgtctctgggcaagagagccaccatcagctgccgggccagcgagagcgtgaccatcctgggcagccacc | cassette: |
| | tgatccactggtatcagcagaagcccggccagccccccaccctgctgatccagctcgccagcaatgtgcagaccggc | 2A- |
| | gtgcccgccagattcagcggcagcggcagcagaaccgacttcaccctgaccatcgaccccgtggaagaggacgacg | C11D5.3- |
| | tggccgtgtactactgcctgcagagccggaccatcccccggacctttggcggaggcaccaaactggaaatcaagggc | CD8-CD28- |
| | agcaccagcggctccggcaagcctggctctggcgagggcagcacaaagggacagattcagctggtgcagagcggc | CD3z- |
| | cctgagctgaagaaacccggcgagacagtgaagatcagctgcaaggcctccggctacaccttcaccgactacagca | CISCb |
| | tcaactgggtgaaaagagcccctggcaagggcctgaagtggatgggctggatcaacaccgagacaagagagcccg | |
| | cctacgcctacgacttccggggcagattcgccttcagcctggaaaccagcgccagcaccgcctacctgcagatcaac | |
| | aacctgaagtacgaggacaccgccacctacttttgcgccctggactacagctacgccatggactactggggccaggg | |
| | caccagcgtgaccgtgtccagcttcgtgcccgtgttcctgcccgccaaacctaccaccacccctgcccctagacctccc | |
| | accccagccccaacaatcgccagccagcctctgtctctgcggcccgaagcctgtagacctgctgccggcggagccgt | |
| | gcacaccagaggcctggacttcgcctgcgacatctacatctgggcccctctggccggcacctgtggcgtgctgctgct | |
| | gagcctggtgatcaccctgtactgcaaccaccggaacagaagcaagcggagccggctgctgcacagcgactacatg | |
| | aacatgaccccaagacggcctggccccacccggaagcactaccagccttacgcccctcccagagacttcgccgccta | |
| | ccggtccagagtgaagttcagcagatccgccgacgcccctgcctaccagcagggacagaaccagctgtacaacgag | |
| | ctgaacctgggcagacgggaagagtacgacgtgctggacaagcggagaggccgggaccccgagatgggcggaaa | |
| | gcccagacggaagaacccccaggaaggcctgtataacgaactgcagaaagacaagatggccgaggcctacagcg | |
| | agatcggcatgaagggcgagcggaggcgcggcaagggccacgatggcctgtaccagggcctgagcaccgccacca | |
| | aggacacctacgacgccctgcacatgcaggccctgccccccagaggatccggcgctacaaatttttcactgctgaaa | |
| | caggcgggtgatgtggaggagaaccctggacccatgccacttggcctgctctggctgggcttggcattgctcggcgcg | |
| | ctccacgcccaggctgaactgatccgcgtggccatattgtggcatgagatgtggcatgagggattggaggaggcgag | |
| | taggctgtactttggggaaaggaatgttaaagggatgtttgaggtccttgaacccctccacgctatgatggaaagagg | |
| | acctcaaacgcttaaagagacgtcattcaatcaagcctatggacgggatcttatggaagctcaagaatggtgtcgaa | |
| | aatacatgaaaagcgggaatgttaaggacctcacgcaagcctgggatctgtattaccatgttttccgacgcatttcta | |
| | aacaaggaaaagatactatcccatggttggggcacttgctcgttgggctcagtggggcgtttggattcatcatcctcgt | |
| | atatctgttgattaattgtcggaacacaggtccctggcttaaaaaagttttgaagtgtaacaccccggatccttctaaa | |
| | ttttttagtcaacttagttcagaacacgggggcgatgttcaaaagtggctgagttccccgtttcccagttcaagtttctc | |
| | ccctgggggtctcgcccccgagatatcacctcttgaagtgctcgagcgggacaaagttacacagcttcttttgcaaca | |
| | ggataaggttccggagccggcgtctctcagctctaaccattcactcacttcttgtttcaccaaccaagggtattttttctt | |
| | ccatctgcctgatgccttggagattgaggcttgtcaggtgtactttacctatgacccctatagtgaggaagaccctgac | |
| | gaaggcgtagctggcgcccccactggctccagtccacagcctcttcagcctctgtcaggggaggacgacgcatattgt | |
| | acgttcccctcacgggacgaccttctgctgttttcaccctcactgctcggcggaccctccccgccaagcacggcacctg | |
| | gggggagtggggcaggagaagaaaggatgcctcctagtttgcaggagcgggttcctcgcgactgggatccgcaacc | |
| | cctcggaccacccacccctggcgtacctgatctggtcgacttccaaccacctccggagcttgtcctcagagaggccgg | |
| | agaggaagtcccagacgcggggccaagagagggtgtgtcatttccctggtcccgccctccgggacagggtgagtttc | |
| | gggcgctgaatgcgaggctcccccttaataccgatgcgtacctgtcattgcaggaacttcagggccaggatcctaccc | |
| | acctggtg | |
| 103 | atggccctgcctgtgacagctctgctcctccctctggccctgctgctccatgccgccagacccgacatcgtgctgaccc | TRAC AAV |
| | agagcccccccagcctggccatgtctctgggcaagagagccaccatcagctgccgggccagcgagagcgtgaccat | #6 |
| | cctgggcagccacctgatccactggtatcagcagaagcccggccagccccccaccctgctgatccagctcgccagca | cassette: |
| | atgtgcagaccggcgtgcccgccagattcagcggcagcggcagcagaaccgacttcaccctgaccatcgaccccgtg | C11D5.3- |
| | gaagaggacgacgtggccgtgtactactgcctgcagagccggaccatcccccggacctttggcggaggcaccaaac | CD8-4166- |
| | tggaaatcaagggcagcaccagcggctccggcaagcctggctctggcgagggcagcacaaagggacagattcagc | CD3z-P2A- |
| | tggtgcagagcggccctgagctgaagaaacccggcgagacagtgaagatcagctgcaaggcctccggctacacctt | CISCb |
| | caccgactacagcatcaactgggtgaaaagagcccctggcaagggcctgaagtggatgggctggatcaacaccgag | |
| | acaagagagcccgcctacgcctacgacttccggggcagattcgccttcagcctggaaaccagcgccagcaccgccta | |
| | cctgcagatcaacaacctgaagtacgaggacaccgccacctacttttgcgccctggactacagctacgccatggact | |
| | actggggccagggcaccagcgtgaccgtgtccagcgccgccgccttcgtgcccgtgttcctgcccgccaaacctacca | |
| | ccacccctgcccctagacctcccaccccagccccaacaatcgccagccagcctctgtctctgcggcccgaagcctgta | |
| | gacctgctgccggcggagccgtgcacaccagaggcctggacttcgcctgcgacatctacatctgggcccctctggccg | |
| | gcacctgtggcgtgctgctgctgagcctggtgatcaccctgtactgcaaccaccggaacagattcagcgtcgtgaagc | |
| | ggggcagaaagaagctgctgtacatcttcaagcagcccttcatgcggcccgtgcagaccacacaagaggaagatgg | |
| | ctgctcctgcagattccctgaggaagaagaaggcggctgcgagctgagagtgaagttcagcagatccgccgacgcc | |
| | cctgcctaccagcagggacagaaccagctgtacaacgagctgaacctgggcagacgggaagagtacgacgtgctg | |
| | gacaagcggagaggccgggaccccgagatgggcggaaagcccagacggaagaacccccaggaaggcctgtataa | |
| | cgaactgcagaaagacaagatggccgaggcctacagcgagatcggcatgaagggcgagcggaggcgcggcaagg | |
| | gccacgatggcctgtaccagggcctgagcaccgccaccaaggacacctacgacgccctgcacatgcaggccctgcc | |
| | ccccagaggatccggcgctacaaatttttcactgctgaaacaggcgggtgatgtggaggagaaccctggacccatgc | |
| | cacttggcctgctctggctgggcttggcattgctcggcgcgctccacgcccaggctgaactgatccgcgtggccatatt | |
| | gtggcatgagatgtggcatgagggattggaggaggcgagtaggctgtactttggggaaaggaatgttaaagggatg | |
| | tttgaggtccttgaacccctccacgctatgatggaaagaggacctcaaacgcttaaagagacgtcattcaatcaagcc | |
| | tatggacgggatcttatggaagctcaagaatggtgtcgaaaatacatgaaaagcgggaatgttaaggacctcacgc | |
| | aagcctgggatctgtattaccatgttttccgacgcatttctaaacaaggaaaagatactatcccatggttggggcactt | |
| | gctcgttgggctcagtggggcgtttggattcatcatcctcgtatatctgttgattaattgtcggaacacaggtccctggc | |
| | ttaaaaaagttttgaagtgtaacaccccggatccttctaaattttttagtcaacttagttcagaacacgggggcgatgtt | |
| | caaaagtggctgagttccccgtttcccagttcaagtttctcccctgggggtctcgcccccgagatatcacctcttgaagt | |
| | gctcgagcgggacaaagttacacagcttcttttgcaacaggataaggttccggagccggcgtctctcagctctaacca | |
| | ttcactcacttcttgtttcaccaaccaagggtattttttcttccatctgcctgatgccttggagattgaggcttgtcaggtg | |
| | tactttacctatgacccctatagtgaggaagaccctgacgaaggcgtagctggcgcccccactggctccagtccacag | |
| | cctcttcagcctctgtcaggggaggacgacgcatattgtacgttcccctcacgggacgaccttctgctgttttcaccctc | |
| | actgctcggcggaccctccccgccaagcacggcacctggggggagtggggcaggagaagaaaggatgcctcctagt | |
| | ttgcaggagcgggttcctcgcgactgggatccgcaacccctcggaccacccacccctggcgtacctgatctggtcgac | |
| | ttccaaccacctccggagcttgtcctcagagaggccggagaggaagtcccagacgcggggccaagagagggtgtgt | |
| | catttccctggtcccgccctccgggacagggtgagtttcgggcgctgaatgcgaggctcccccttaataccgatgcgta | |
| | cctgtcattgcaggaacttcagggccaggatcctacccacctggtg | |
| 104 | ggttccggggagggccgagggtcattgctgacgtgtggagacgtggaggagaatcctggccccatggccctgcctgt | TRAC AAV |
| | gacagctctgctcctccctctggccctgctgctccatgccgccagacccgacatcgtgctgacccagagcccccccag | #7 |
| | cctggccatgtctctgggcaagagagccaccatcagctgccgggccagcgagagcgtgaccatcctgggcagccacc | cassette: |
| | tgatccactggtatcagcagaagcccggccagccccccaccctgctgatccagctcgccagcaatgtgcagaccggc | 2A- |
| | gtgcccgccagattcagcggcagcggcagcagaaccgacttcaccctgaccatcgaccccgtggaagaggacgacg | C11D5.3- |
| | tggccgtgtactactgcctgcagagccggaccatcccccggacctttggcggaggcaccaaactggaaatcaagggc | AAA-CD8- |
| | agcaccagcggctccggcaagcctggctctggcgagggcagcacaaagggacagattcagctggtgcagagcggc | 41BB- |
| | cctgagctgaagaaacccggcgagacagtgaagatcagctgcaaggcctccggctacaccttcaccgactacagca | CD3z-P2A- |
| | tcaactgggtgaaaagagcccctggcaagggcctgaagtggatgggctggatcaacaccgagacaagagagcccg | CISCb |
| | cctacgcctacgacttccggggcagattcgccttcagcctggaaaccagcgccagcaccgcctacctgcagatcaac | |
| | aacctgaagtacgaggacaccgccacctacttttgcgccctggactacagctacgccatggactactggggccaggg | |
| | caccagcgtgaccgtgtccagcgccgccgccttcgtgcccgtgttcctgcccgccaaacctaccaccacccctgcccct | |
| | agacctcccaccccagccccaacaatcgccagccagcctctgtctctgcggcccgaagcctgtagacctgctgccggc | |
| | ggagccgtgcacaccagaggcctggacttcgcctgcgacatctacatctgggcccctctggccggcacctgtggcgtg | |
| | ctgctgctgagcctggtgatcaccctgtactgcaaccaccggaacagattcagcgtcgtgaagcggggcagaaagaa | |
| | gctgctgtacatcttcaagcagcccttcatgcggcccgtgcagaccacacaagaggaagatggctgctcctgcagatt | |
| | ccctgaggaagaagaaggcggctgcgagctgagagtgaagttcagcagatccgccgacgcccctgcctaccagcag | |
| | ggacagaaccagctgtacaacgagctgaacctgggcagacgggaagagtacgacgtgctggacaagcggagagg | |
| | ccgggaccccgagatgggcggaaagcccagacggaagaacccccaggaaggcctgtataacgaactgcagaaag | |
| | acaagatggccgaggcctacagcgagatcggcatgaagggcgagcggaggcgcggcaagggccacgatggcctgt | |
| | accagggcctgagcaccgccaccaaggacacctacgacgccctgcacatgcaggccctgccccccagaggatccgg | |
| | cgctacaaatttttcactgctgaaacaggcgggtgatgtggaggagaaccctggacccatgccacttggcctgctctg | |
| | gctgggcttggcattgctcggcgcgctccacgcccaggctgaactgatccgcgtggccatattgtggcatgagatgtg | |
| | gcatgagggattggaggaggcgagtaggctgtactttggggaaaggaatgttaaagggatgtttgaggtccttgaac | |
| | ccctccacgctatgatggaaagaggacctcaaacgcttaaagagacgtcattcaatcaagcctatggacgggatctt | |
| | atggaagctcaagaatggtgtcgaaaatacatgaaaagcgggaatgttaaggacctcacgcaagcctgggatctgt | |
| | attaccatgttttccgacgcatttctaaacaaggaaaagatactatcccatggttggggcacttgctcgttgggctcag | |
| | tggggcgtttggattcatcatcctcgtatatctgttgattaattgtcggaacacaggtccctggcttaaaaaagttttga | |
| | agtgtaacaccccggatccttctaaattttttagtcaacttagttcagaacacgggggcgatgttcaaaagtggctgag | |
| | ttccccgtttcccagttcaagtttctcccctgggggtctcgcccccgagatatcacctcttgaagtgctcgagcgggaca | |
| | aagttacacagcttcttttgcaacaggataaggttccggagccggcgtctctcagctctaaccattcactcacttcttgt | |
| | ttcaccaaccaagggtattttttcttccatctgcctgatgccttggagattgaggcttgtcaggtgtactttacctatgac | |
| | ccctatagtgaggaagaccctgacgaaggcgtagctggcgcccccactggctccagtccacagcctcttcagcctctg | |
| | tcaggggaggacgacgcatattgtacgttcccctcacgggacgaccttctgctgttttcaccctcactgctcggcggac | |
| | cctccccgccaagcacggcacctggggggagtggggcaggagaagaaaggatgcctcctagtttgcaggagcgggt | |
| | tcctcgcgactgggatccgcaacccctcggaccacccacccctggcgtacctgatctggtcgacttccaaccacctccg | |
| | gagcttgtcctcagagaggccggagaggaagtcccagacgcggggccaagagagggtgtgtcatttccctggtcccg | |
| | ccctccgggacagggtgagtttcgggcgctgaatgcgaggctcccccttaataccgatgcgtacctgtcattgcagga | |
| | acttcagggccaggatcctacccacctggtg | |
| 105 | gtgtgaacagagaaacaggagaatatgggccaaacaggatatctgtggtaagcagttcctgccccggctcagggcc | IL2RG AAV |
| | aagaacagttggaacagcagaatatgggccaaacaggatatctgtggtaagcagttcctgccccggctcagggcca | #8 |
| | agaacagatggtccccagatgcggtcccgccctcagcagtttctagagaaccatcagatgtttccagggtgccccaag | cassette: |
| | gacctgaaatgaccctgtgccttatttgaactaaccaatcagttcgcttctcgcttctgttcgcgcgcttctgctccccga | MND- |
| | gctctatataagcagagctcgtttagtgaaccgtcagatcggtaccgccgccaccatggagatgtggcatgagggtct | nakedFRB- |
| | ggaagaagcgtctcgactgtactttggtgagcgcaatgtgaagggcatgtttgaagtcctcgaaccccttcatgccat | tLNGFR- |
| | gatggaacgcggaccccagaccttgaaggagacaagttttaaccaagcttacggaagagacctgatggaagcccag | CNb30- |
| | gaatggtgcaggaaatacatgaaaagcgggaatgtgaaggacttgctccaagcgtgggacctgtactatcacgtctt | CISCg |
| | taggcgcattagtaagggcagcggcgccacaaatttcagcctgctgaaacaggccggcgacgtggaagagaaccct | |
| | ggacccatgggtgctggcgcaactggacgcgctatggatggacctcgcttgctgcttcttctgcttctcggggtctcatt | |
| | gggtggtgctaaggaagcatgcccaacgggactttatacgcatagcggagagtgttgcaaagcttgtaacctgggcg | |
| | aaggcgtcgcgcaaccttgtggtgcaaatcaaaccgtctgcgagccatgtttggactctgttacgtttagtgacgtagt | |
| | atctgcgacagagccatgcaagccttgtacggaatgtgtaggattgcagagcatgtctgccccttgtgtagaagccga | |
| | cgatgcagtttgcaggtgcgcgtatggctattaccaagacgaaacaaccggacgatgtgaagcttgccgagtttgtga | |
| | agcgggttccgggcttgtattctcatgtcaggataagcagaacaccgtctgcgaagagtgccccgatggcacctacag | |
| | cgatgaagcgaaccatgtagacccctgcctgccttgcaccgtttgtgaagacacggaacgacagttgcgggagtgta | |
| | cccggtgggcagacgccgagtgcgaagagattccaggccgctggatcacgcgaagtaccccgccagaaggttccga | |
| | cagtactgcaccaagcacccaagaaccagaggcgccccccgagcaggacctgattgcctccaccgtggcgggtgttg | |
| | ttactacggttatgggctcatcccagcccgttgttacccgaggaactacagacaacctgattccggtatattgttctatc | |
| | ttggcggctgtagtagttggcttggtcgcctacatcgctttcaaaagaggttccggggagggccgagggtcattgctga | |
| | cgtgtggagacgtggaggagaatcctggccccatgggcaacgaggccagctaccctctggagatgtgctcccacttc | |
| | gacgccgacgagatcaagcggctgggcaagcgcttcaagaagctggacctggacaacagcggcagcctgagcgtg | |
| | gaggagtttatgtctctgcccgagctgcagcagaaccccctggtgcagcgcgtgatcgacatcttcgacaccgacggc | |
| | aacggcgaggtggacttcaaggagttcatcgagggcgtgagccagttcagcgtgaagggcgacaaggagcagaag | |
| | ctgcggttcgccttccggatctacgatatggataaagatggctatatttctaatggcgagctgttccaggtgctgaaga | |
| | tgatggtgggcaacaataccaagctggccgatacccagctgcagcagatcgtggacaagaccatcatcaacgccga | |
| | caaggacggcgacggcagaatcagcttcgaggagttctgtgccgtggtgggaggcctggatattcacaaaaaaatg | |
| | gtggtggacgtgggatccggcgctacaaatttttcactgctgaaacaggcgggtgacgtggaggagaaccctggacc | |
| | catgcctctgggcctgctgtggctgggcctggccctgctgggcgccctgcacgcccaggccggcgtgcaggtggagac | |
| | aatctccccaggcgacggacgcacattccctaagcggggccagacctgcgtggtgcactatacaggcatgctggagg | |
| | atggcaagaagtttgacagctcccgggatagaaacaagccattcaagtttatgctgggcaagcaggaagtgatcag | |
| | aggctgggaggagggcgtggcccagatgtctgtgggccagagggccaagctgaccatcagcccagactacgcctat | |
| | ggagcaacaggccacccaggaatcatcccacctcacgccaccctggtgttcgatgtggagctgctgaagctgggcga | |
| | gggcagcaacaccagcaaa | |
| 106 | gtgtgaacagagaaacaggagaatatgggccaaacaggatatctgtggtaagcagttcctgccccggctcagggcc | IL2RG AAV |
| | aagaacagttggaacagcagaatatgggccaaacaggatatctgtggtaagcagttcctgccccggctcagggcca | #9 |
| | agaacagatggtccccagatgcggtcccgccctcagcagtttctagagaaccatcagatgtttccagggtgccccaag | cassette: |
| | gacctgaaatgaccctgtgccttatttgaactaaccaatcagttcgcttctcgcttctgttcgcgcgcttctgctccccga | MND- |
| | gctctatataagcagagctcgtttagtgaaccgtcagatcggtaccgccgccaccatgagcaggtcagtggcgttggc | B2MCAR- |
| | ggttctggcgcttttgagtttgagcggactggaagccatccaacgaacgcctaagatccaggtatattcacgccaccc | nakedFRB- |
| | ggcggaaaacggcaaaagtaacttccttaattgttatgtgtctggcttccacccgtctgatattgaggtggacctcctt | tLNGFR- |
| | aaaaacggtgaacggatcgagaaagtggagcattccgatcttagtttcagtaaggattggagcttttaccttctctatt | CNb30- |
| | acactgagttcactccgactgaaaaggatgagtacgcctgtcgggtcaaccacgtcaccctgtctcaaccaaaaata | CISCg |
| | gtcaaatgggacagagatatgtcagatatttacatatgggcaccacttgcgggcacgtgtggcgtcctgcttctgagtc | |
| | tcgtcattacgctttattgtaaacggggtagaaaaaaactcctttatatatttaaacagccatttatgcggccagttcaa | |
| | acgacgcaggaagaagacggctgtagttgcagatttccagaggaagaggaaggtggatgcgagcttcgggtcaagt | |
| | ttagtaggtctgcagacgctcccgcctatcaacagggtcagaatcagctttataacgaactcaacctcggtcgccgag | |
| | aagagtacgacgtactcgataaaagaaggggtagagacccggaaatggggggcaaaccgcgccgcaaaaatcca | |
| | caagaggggctttataatgagcttcaaaaagacaaaatggccgaagcatacagtgagattgggatgaaaggtgaac | |
| | gcagaagaggtaagggtcacgacgggctgtaccagggtttgtcaactgccacaaaggatacttatgacgctctgcat | |
| | atgcaagctcttcccccacgcggcagcggcgaaggcagaggatccctgcttacatgtggcgacgtggaagagaacc | |
| | ctggccccatggagatgtggcatgagggtctggaagaagcgtctcgactgtactttggtgagcgcaatgtgaagggc | |
| | atgtttgaagtcctcgaaccccttcatgccatgatggaacgcggaccccagaccttgaaggagacaagttttaaccaa | |
| | gcttacggaagagacctgatggaagcccaggaatggtgcaggaaatacatgaaaagcgggaatgtgaaggacttg | |
| | ctccaagcgtgggacctgtactatcacgtctttaggcgcattagtaagggcagcggcgccacaaatttcagcctgctg | |
| | aaacaggccggcgacgtggaagagaaccctggacccatgggtgctggcgcaactggacgcgctatggatggacctc | |
| | gcttgctgcttcttctgcttctcggggtctcattgggtggtgctaaggaagcatgcccaacgggactttatacgcatagc | |
| | ggagagtgttgcaaagcttgtaacctgggcgaaggcgtcgcgcaaccttgtggtgcaaatcaaaccgtctgcgagcc | |
| | atgtttggactctgttacgtttagtgacgtagtatctgcgacagagccatgcaagccttgtacggaatgtgtaggattg | |
| | cagagcatgtctgccccttgtgtagaagccgacgatgcagtttgcaggtgcgcgtatggctattaccaagacgaaaca | |
| | accggacgatgtgaagcttgccgagtttgtgaagcgggttccgggcttgtattctcatgtcaggataagcagaacacc | |
| | gtctgcgaagagtgccccgatggcacctacagcgatgaagcgaaccatgtagacccctgcctgccttgcaccgtttgt | |
| | gaagacacggaacgacagttgcgggagtgtacccggtgggcagacgccgagtgcgaagagattccaggccgctgg | |
| | atcacgcgaagtaccccgccagaaggttccgacagtactgcaccaagcacccaagaaccagaggcgccccccgag | |
| | caggacctgattgcctccaccgtggcgggtgttgttactacggttatgggctcatcccagcccgttgttacccgaggaa | |
| | ctacagacaacctgattccggtatattgttctatcttggcggctgtagtagttggcttggtcgcctacatcgctttcaaa | |
| | agaggttccggggagggccgagggtcattgctgacgtgtggagacgtggaggagaatcctggccccatgggcaacg | |
| | aggccagctaccctctggagatgtgctcccacttcgacgccgacgagatcaagcggctgggcaagcgcttcaagaag | |
| | ctggacctggacaacagcggcagcctgagcgtggaggagtttatgtctctgcccgagctgcagcagaaccccctggt | |
| | gcagcgcgtgatcgacatcttcgacaccgacggcaacggcgaggtggacttcaaggagttcatcgagggcgtgagc | |
| | cagttcagcgtgaagggcgacaaggagcagaagctgcggttcgccttccggatctacgatatggataaagatggcta | |
| | tatttctaatggcgagctgttccaggtgctgaagatgatggtgggcaacaataccaagctggccgatacccagctgca | |
| | gcagatcgtggacaagaccatcatcaacgccgacaaggacggcgacggcagaatcagcttcgaggagttctgtgcc | |
| | gtggtgggaggcctggatattcacaaaaaaatggtggtggacgtgggatccggcgctacaaatttttcactgctgaaa | |
| | caggcgggtgacgtggaggagaaccctggacccatgcctctgggcctgctgtggctgggcctggccctgctgggcgc | |
| | cctgcacgcccaggccggcgtgcaggtggagacaatctccccaggcgacggacgcacattccctaagcggggccag | |
| | acctgcgtggtgcactatacaggcatgctggaggatggcaagaagtttgacagctcccgggatagaaacaagccatt | |
| | caagtttatgctgggcaagcaggaagtgatcagaggctgggaggagggcgtggcccagatgtctgtgggccagagg | |
| | gccaagctgaccatcagcccagactacgcctatggagcaacaggccacccaggaatcatcccacctcacgccaccct | |
| | ggtgttcgatgtggagctgctgaagctgggcgagggcagcaacaccagcaaa | |
| 107 | gtgtgaacagagaaacaggagaatatgggccaaacaggatatctgtggtaagcagttcctgccccggctcagggcc | IL2RG AAV |
| | aagaacagttggaacagcagaatatgggccaaacaggatatctgtggtaagcagttcctgccccggctcagggcca | #10 |
| | agaacagatggtccccagatgcggtcccgccctcagcagtttctagagaaccatcagatgtttccagggtgccccaag | cassette: |
| | gacctgaaatgaccctgtgccttatttgaactaaccaatcagttcgcttctcgcttctgttcgcgcgcttctgctccccga | MND- |
| | gctctatataagcagagctcgtttagtgaaccgtcagatcggtaccgccgccaccatggagatgtggcatgagggtct | nakedFRB- |
| | ggaagaagcgtctcgactgtactttggtgagcgcaatgtgaagggcatgtttgaagtcctcgaaccccttcatgccat | CNb30- |
| | gatggaacgcggaccccagaccttgaaggagacaagttttaaccaagcttacggaagagacctgatggaagcccag | CISCb- |
| | gaatggtgcaggaaatacatgaaaagcgggaatgtgaaggacttgctccaagcgtgggacctgtactatcacgtctt | CISCg |
| | taggcgcattagtaagggcagcggcgccacaaatttcagcctgctgaaacaggccggcgacgtggaagagaaccct | |
| | ggacccatgggcaacgaggccagctaccctctggagatgtgctcccacttcgacgccgacgagatcaagcggctggg | |
| | caagcgcttcaagaagctggacctggacaacagcggcagcctgagcgtggaggagtttatgtctctgcccgagctgc | |
| | agcagaaccccctggtgcagcgcgtgatcgacatcttcgacaccgacggcaacggcgaggtggacttcaaggagttc | |
| | atcgagggcgtgagccagttcagcgtgaagggcgacaaggagcagaagctgcggttcgccttccggatctacgatat | |
| | ggataaagatggctatatttctaatggcgagctgttccaggtgctgaagatgatggtgggcaacaataccaagctgg | |
| | ccgatacccagctgcagcagatcgtggacaagaccatcatcaacgccgacaaggacggcgacggcagaatcagctt | |
| | cgaggagttctgtgccgtggtgggaggcctggatattcacaaaaaaatggtggtggacgtgggcagcggcgaaggc | |
| | agaggatccctgcttacatgtggcgacgtggaagagaaccctggccccatgccacttggcctgctctggctgggcttg | |
| | gcattgctcggcgcgctccacgcccaggctgaactgatccgcgtggccatattgtggcacgagatgtggcacgaggg | |
| | attggaggaggcgagtaggctgtactttggggaaaggaatgttaaagggatgtttgaggtccttgaacccctccacgc | |
| | tatgatggaaagaggacctcaaacgcttaaagagacgtcattcaatcaagcctatggacgggatcttatggaagctc | |
| | aagaatggtgtcgaaaatacatgaaaagcgggaatgttaaggacctcacgcaagcctgggatctgtattaccatgtt | |
| | ttccgacgcatttctaaacaaggaaaagatactatcccatggttggggcacttgctcgttgggctcagtggggcgtttg | |
| | gattcatcatcctcgtatatctgttgattaattgtcggaacacaggtccctggcttaaaaaagttttgaagtgtaacacc | |
| | ccggatccttctaaattttttagtcaacttagttcagaacacgggggcgatgttcaaaagtggctgagttccccgtttcc | |
| | cagttcaagtttctcccctgggggtctcgcccccgagatatcacctcttgaagtgctcgagcgggacaaagttacaca | |
| | gcttcttttgcaacaggataaggttccggagccggcgtctctcagctctaaccattcactcacttcttgtttcaccaacc | |
| | aagggtattttttcttccatctgcctgatgccttggagattgaggcttgtcaggtgtactttacctatgacccctatagtg | |
| | aggaagaccctgacgaaggcgtagctggcgcccccactggctccagtccacagcctcttcagcctctgtcaggggag | |
| | gacgacgcatattgtacgttcccctcacgggacgaccttctgctgttttcaccctcactgctcggcggaccctccccgcc | |
| | aagcacggcacctggggggagtggggcaggagaagaaaggatgcctcctagtttgcaggagcgggttcctcgcgac | |
| | tgggatccgcaacccctcggaccacccacccctggcgtacctgatctggtcgacttccaaccacctccggagcttgtcc | |
| | tcagagaggccggagaggaagtcccagacgcggggccaagagagggtgtgtcatttccctggtcccgccctccggg | |
| | acagggtgagtttcgggcgctgaatgcgaggctcccccttaataccgatgcgtacctgtcattgcaggaacttcaggg | |
| | ccaggatcctacccacctggtgggatccggcgctacaaatttttcactgctgaaacaggcgggtgacgtggaggaga | |
| | accctggacccatgcctctgggcctgctgtggctgggcctggccctgctgggcgccctgcacgcccaggccggcgtgc | |
| | aggtggagacaatctccccaggcgacggacgcacattccctaagcggggccagacctgcgtggtgcactatacagg | |
| | catgctggaggatggcaagaagtttgacagctcccgggatagaaacaagccattcaagtttatgctgggcaagcagg | |
| | aagtgatcagaggctgggaggagggcgtggcccagatgtctgtgggccagagggccaagctgaccatcagcccag | |
| | actacgcctatggagcaacaggccacccaggaatcatcccacctcacgccaccctggtgttcgatgtggagctgctga | |
| | agctgggcgagggcagcaacaccagcaaa | |
| 108 | gtgtgaacagagaaacaggagaatatgggccaaacaggatatctgtggtaagcagttcctgccccggctcagggcc | IL2RG AAV |
| | aagaacagttggaacagcagaatatgggccaaacaggatatctgtggtaagcagttcctgccccggctcagggcca | #11 |
| | agaacagatggtccccagatgcggtcccgccctcagcagtttctagagaaccatcagatgtttccagggtgccccaag | cassette: |
| | gacctgaaatgaccctgtgccttatttgaactaaccaatcagttcgcttctcgcttctgttcgcgcgcttctgctccccga | MND- |
| | gctctatataagcagagctcgtttagtgaaccgtcagatcggtaccgccgccaccatgagcaggtcagtggcgttggc | B2MCAR- |
| | ggttctggcgcttttgagtttgagcggactggaagccatccaacgaacgcctaagatccaggtatattcacgccaccc | nakedFRB- |
| | ggcggaaaacggcaaaagtaacttccttaattgttatgtgtctggcttccacccgtctgatattgaggtggacctcctt | CISCb- |
| | aaaaacggtgaacggatcgagaaagtggagcattccgatcttagtttcagtaaggattggagcttttaccttctctatt | CISCg |
| | acactgagttcactccgactgaaaaggatgagtacgcctgtcgggtcaaccacgtcaccctgtctcaaccaaaaata | |
| | gtcaaatgggacagagatatgtcagatatttacatatgggcaccacttgcgggcacgtgtggcgtcctgcttctgagtc | |
| | tcgtcattacgctttattgtaaacggggtagaaaaaaactcctttatatatttaaacagccatttatgcggccagttcaa | |
| | acgacgcaggaagaagacggctgtagttgcagatttccagaggaagaggaaggtggatgcgagcttcgggtcaagt | |
| | ttagtaggtctgcagacgctcccgcctatcaacagggtcagaatcagctttataacgaactcaacctcggtcgccgag | |
| | aagagtacgacgtactcgataaaagaaggggtagagacccggaaatggggggcaaaccgcgccgcaaaaatcca | |
| | caagaggggctttataatgagcttcaaaaagacaaaatggccgaagcatacagtgagattgggatgaaaggtgaac | |
| | gcagaagaggtaagggtcacgacgggctgtaccagggtttgtcaactgccacaaaggatacttatgacgctctgcat | |
| | atgcaagctcttcccccacgcggcagcggcgaaggcagaggatccctgcttacatgtggcgacgtggaagagaacc | |
| | ctggccccatggagatgtggcatgagggtctggaagaagcgtctcgactgtactttggtgagcgcaatgtgaagggc | |
| | atgtttgaagtcctcgaaccccttcatgccatgatggaacgcggaccccagaccttgaaggagacaagttttaaccaa | |
| | gcttacggaagagacctgatggaagcccaggaatggtgcaggaaatacatgaaaagcgggaatgtgaaggacttg | |
| | ctccaagcgtgggacctgtactatcacgtctttaggcgcattagtaagggcagcggcgccacaaatttcagcctgctg | |
| | aaacaggccggcgacgtggaagagaaccctggacccatgccacttggcctgctctggctgggcttggcattgctcgg | |
| | cgcgctccacgcccaggctgaactgatccgcgtggccatattgtggcacgagatgtggcacgagggattggaggagg | |
| | cgagtaggctgtactttggggaaaggaatgttaaagggatgtttgaggtccttgaacccctccacgctatgatggaaa | |
| | gaggacctcaaacgcttaaagagacgtcattcaatcaagcctatggacgggatcttatggaagctcaagaatggtgt | |
| | cgaaaatacatgaaaagcgggaatgttaaggacctcacgcaagcctgggatctgtattaccatgttttccgacgcatt | |
| | tctaaacaaggaaaagatactatcccatggttggggcacttgctcgttgggctcagtggggcgtttggattcatcatcc | |
| | tcgtatatctgttgattaattgtcggaacacaggtccctggcttaaaaaagttttgaagtgtaacaccccggatccttct | |
| | aaattttttagtcaacttagttcagaacacgggggcgatgttcaaaagtggctgagttccccgtttcccagttcaagttt | |
| | ctcccctgggggtctcgcccccgagatatcacctcttgaagtgctcgagcgggacaaagttacacagcttcttttgcaa | |
| | caggataaggttccggagccggcgtctctcagctctaaccattcactcacttcttgtttcaccaaccaagggtatttttt | |
| | cttccatctgcctgatgccttggagattgaggcttgtcaggtgtactttacctatgacccctatagtgaggaagaccctg | |
| | acgaaggcgtagctggcgcccccactggctccagtccacagcctcttcagcctctgtcaggggaggacgacgcatatt | |
| | gtacgttcccctcacgggacgaccttctgctgttttcaccctcactgctcggcggaccctccccgccaagcacggcacc | |
| | tggggggagtggggcaggagaagaaaggatgcctcctagtttgcaggagcgggttcctcgcgactgggatccgcaa | |
| | cccctcggaccacccacccctggcgtacctgatctggtcgacttccaaccacctccggagcttgtcctcagagaggcc | |
| | ggagaggaagtcccagacgcggggccaagagagggtgtgtcatttccctggtcccgccctccgggacagggtgagtt | |
| | tcgggcgctgaatgcgaggctcccccttaataccgatgcgtacctgtcattgcaggaacttcagggccaggatcctac | |
| | ccacctggtgggatccggcgctacaaatttttcactgctgaaacaggcgggtgacgtggaggagaaccctggaccca | |
| | tgcctctgggcctgctgtggctgggcctggccctgctgggcgccctgcacgcccaggccggcgtgcaggtggagaca | |
| | atctccccaggcgacggacgcacattccctaagcggggccagacctgcgtggtgcactatacaggcatgctggagga | |
| | tggcaagaagtttgacagctcccgggatagaaacaagccattcaagtttatgctgggcaagcaggaagtgatcaga | |
| | ggctgggaggagggcgtggcccagatgtctgtgggccagagggccaagctgaccatcagcccagactacgcctatg | |
| | gagcaacaggccacccaggaatcatcccacctcacgccaccctggtgttcgatgtggagctgctgaagctgggcgag | |
| | ggcagcaacaccagcaaa | |

What is claimed is:

1. An engineered T cell comprising a) an endogenous T cell receptor alpha (TIM) gene modified to encode a non-functional T cell receptor alpha constant (TRAC) domain; and b) a nucleic acid encoding a chimeric antigen receptor (CAR) that can recognize B-cell maturation antigen (BCMA).

2. The cell of claim 1, wherein the CAR that can recognize BCMA comprises an extracellular BCMA recognition domain, a transmembrane domain, a co-stimulatory domain, and a cytoplasmic signaling domain.

3. The cell of claim 2, wherein the extracellular BCMA recognition domain is an antibody moiety that can specifically bind to BCMA.

4. The cell of claim 3, wherein the antibody moiety comprises a heavy chain variable domain (V_{H}) and a light chain variable domain (V_{L}) comprising heavy chain complementarity-determining region (HC-CDR)1, HC-CDR2, HC-CDR3, light chain complementarity-determining region (LC-CDR)1, LC-CDR2, and LC-CDR3 from SEQ ID NO: 55.

5. The cell of claim 3 or 4, wherein the antibody moiety is an scFv.

6. The cell of any one of claims 2-5, wherein the CAR transmembrane domain comprises a CD8 transmembrane domain, the CAR co-stimulatory domain comprises a 4-1BB and/or a CD28 co-stimulatory domain, and/or the CAR cytoplasmic signaling domain comprises a CD3-ζ cytoplasmic signaling domain.

7. The cell of any one of claims 1-6, wherein the b) nucleic acid encoding a CAR that can recognize BCMA is inserted into the region of the endogenous TIM gene encoding the TRAC domain or the b) nucleic acid encoding a CAR that can recognize BCMA is inserted into an endogenous IL2RG gene.

8. The cell of any one of claims 1-7, further comprising c) one or more nucleic acids encoding polypeptide components of a dimerization activatable chemical-induced signaling complex (CISC), wherein the polypeptide components of the CISC comprise
i) a first CISC component comprising a first extracellular binding domain or portion thereof, a hinge domain, a transmembrane domain, and a signaling domain or portion thereof; and
ii) a second CISC component comprising a second extracellular binding domain or portion thereof, a hinge domain, a transmembrane domain, and a signaling domain or portion thereof;
wherein the first CISC component and the second CISC component are configured such that when expressed, they dimerize in the presence of the ligand to create a signaling-competent CISC.

9. The cell of claim 8, wherein the signaling domain of the first CISC component comprises an IL-2 receptor subunit gamma (IL2Rγ) cytoplasmic signaling domain.

10. The cell of claim 9, wherein the IL2Rγ cytoplasmic signaling domain comprises the amino acid sequence of SEQ ID NO: 50 or a variant thereof having at least 85% homology to the amino acid sequence of SEQ ID NO: 50.

11. The cell of any one of claims 8-10, wherein the first extracellular binding domain or portion thereof comprises an FK506 binding protein (FKBP) domain or a portion thereof.

12. The cell of claim 11, wherein the FKBP domain comprises the amino acid sequence of SEQ ID NO: 47 or a variant thereof having at least 85% homology to the amino acid sequence of SEQ ID NO: 47.

13. The cell of any one of claims 8-12, wherein the signaling domain of the second CISC component comprises an IL-2 receptor subunit beta (IL2β) cytoplasmic signaling domain.

14. The cell of claim 13, wherein the IL2Rβ cytoplasmic signaling domain comprises the amino acid sequence of SEQ ID NO: 51 or a variant thereof having at least 85% homology to the amino acid sequence of SEQ ID NO: 51.

15. The cell of any one of claims 8-14, wherein the second extracellular binding domain or portion thereof comprises an FKBP rapamycin binding (FRB) domain or a portion thereof.

16. The cell of claim 15, wherein the FRB comprises the amino acid sequence of SEQ ID NO: 48 or a variant thereof having at least 85% homology to the amino acid sequence of SEQ ID NO: 48.

17. The cell of any one of claims 8-16, wherein the transmembrane domain of the first and second CISC components comprises, independently, an IL-2 receptor transmembrane domain.

18. The cell of any one of claims 8-17, wherein 1) the one or more nucleic acids encoding the first CISC component are inserted into an endogenous IL2RG gene and the one or more nucleic acids encoding the second CISC component are inserted into the region of the endogenous TIM gene encoding the TRAC domain; or 2) the one or more nucleic acids encoding the first CISC component are inserted into the region of the endogenous TIM gene encoding the TRAC domain and the one or more nucleic acids encoding the second CISC component are inserted into the endogenous IL2RG gene.

19. The cell of any one of claims 1-18, wherein the ligand is rapamycin or a rapamycin analog (rapalog).

20. The cell of claim 19, wherein the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

21. The cell of any one of claims 1-20, wherein the ligand is present or provided in an amount from 0.05 nM to 100 nM.

22. The cell of any one of claims 1-21, further comprising d) one or more nucleic acids encoding a chimeric receptor comprising an extracellular β2-microglobulin domain, a transmembrane domain, a co-stimulatory domain, and a cytoplasmic signaling domain.

23. The cell of claim 22, wherein the chimeric receptor transmembrane domain comprises a CD8 transmembrane domain, the chimeric receptor co-stimulatory domain comprises a 4-1BB co-stimulatory domain, and/or the chimeric receptor cytoplasmic signaling domain comprises a CD3-ζ cytoplasmic signaling domain.

24. The cell of claim 23, wherein the chimeric receptor comprises the amino acid sequence of SEQ ID NO: 65 or a variant thereof having at least 85% homology to the amino acid sequence of SEQ ID NO: 65

25. The cell of any one of claims 22-24, wherein the d) one or more nucleic acids encoding the chimeric receptor are inserted into the region of the endogenous TIM gene encoding the TRAC domain or the d) one or more nucleic acids encoding the chimeric receptor are inserted into an endogenous IL2RG gene.

26. The cell of any one of claims 1-25, further comprising g) a nucleic acid encoding a selectable marker.

27. The cell of claim 26, wherein the selectable marker is a truncated low-affinity nerve growth factor receptor (tLNGFR) polypeptide.

28. The cell of claim 27, wherein the tLNGFR polypeptide comprises the amino acid sequence of SEQ ID NO: 66.

29. The cell of any one of claims 26-28, wherein the nucleic acid encoding the selectable marker is inserted into the region of the endogenous TRA gene encoding the TRAC domain or the nucleic acid encoding the selectable marker is inserted into an endogenous IL2RG gene.

30. The cell of any one of claims 1-29, further comprising e) a nucleic acid encoding a polypeptide that confers resistance to one or more calcineurin inhibitors.

31. The cell of claim 30, wherein the polypeptide that confers resistance to one or more calcineurin inhibitors confers resistance to tacrolimus (FK506) and/or cyclosporin A (CsA).

32. The cell of claim 30 or 31, wherein the polypeptide that confers resistance to one or more calcineurin inhibitors is a mutant calcineurin (CN) polypeptide.

33. The cell of claim 32, wherein the mutant CN polypeptide confers resistance to tacrolimus (FK506) and cyclosporin A (CsA).

34. The cell of claim 32 or 33, wherein the mutant CN polypeptide is CNb30 (SEQ ID NO: 67).

35. The cell of any one of claims 30-34, wherein the nucleic acid encoding the polypeptide that confers resistance to one or more calcineurin inhibitors is inserted into the region of the endogenous TRA gene encoding the TRAC domain or the nucleic acid encoding the polypeptide that confers resistance to one or more calcineurin inhibitors is inserted into an endogenous IL2RG gene.

36. The cell of any one of claims 1-35, further comprising f) a nucleic acid encoding a FKBP-rapamycin binding (FRB) domain polypeptide of the mammalian target of rapamycin (mTOR) kinase.

37. The cell of claim 36, wherein the FRB domain polypeptide is expressed intracellularly.

38. The cell of claim 36 or 37, wherein the FRB domain polypeptide comprises the amino acid of SEQ ID NO: 68 or 69 or a variant having at least 90% sequence homology to the amino acid of SEQ ID NO: 68 or 69.

39. The cell of any one of claims 36-38, wherein the nucleic acid encoding the FRB domain polypeptide is inserted into the region of the endogenous TRA gene encoding the TRAC domain or the nucleic acid encoding the FRB domain polypeptide is inserted into an endogenous IL2RG gene.

40. A guide RNA (gRNA) comprising a sequence that is complementary to a sequence in an endogenous TRA gene within or near a region encoding the TRAC domain.

41. The gRNA of claim 40, wherein the gRNA comprises the polynucleotide sequence of any one of SEQ ID NOs: 1-3, or a variant thereof having at least 85% homology to any one of SEQ ID NOs: 1-3.

42. A guide RNA (gRNA) comprising a sequence that is complementary to a sequence within or near an endogenous IL2RG gene.

43. The gRNA of claim 42, wherein the gRNA comprises the polynucleotide sequence of any one of SEQ ID NOs: 4-18, or a variant thereof having at least 85% homology to any one of SEQ ID NOs: 4-18.

44. A system comprising a) a first gRNA and/or a second gRNA, wherein the first gRNA is the gRNA of claim 40 or 41 and the second gRNA is the gRNA of claim 42 or 43; and b) an RNA-guided endonuclease (RGEN) or a nucleic acid encoding the RGEN.

45. The system of claim 44, further comprising c) one or more donor templates comprising nucleic acid encoding:
i) a CAR that can recognize a B-cell maturation antigen (BCMA) polypeptide;
ii) a first CISC component comprising a first extracellular binding domain or portion thereof, a hinge domain, a transmembrane domain, and a signaling domain or portion thereof or functional derivative thereof; and
iii) a second CISC component comprising a second extracellular binding domain or portion thereof, a hinge domain, a transmembrane domain, and a signaling domain or portion thereof,
wherein the first CISC component and the second CISC component are configured such that when expressed by a T cell, they dimerize in the presence of a ligand to create a signaling competent CISC capable of promoting the survival and/or proliferation of the T cell.

46. The system of claim 45, wherein the CAR that can recognize BCMA comprises an extracellular BCMA recognition domain, a transmembrane domain, a co-stimulatory domain, and a cytoplasmic signaling domain.

47. The system of claim 46, wherein the extracellular BCMA recognition domain is an antibody moiety that can specifically bind to BCMA.

48. The system of claim 47, wherein the antibody moiety comprises a heavy chain variable domain (V_{H}) and a light chain variable domain (V_{L}) comprising heavy chain complementarity-determining region (HC-CDR)1, HC-CDR2, HC-CDR3, light chain complementarity-determining region (LC-CDR)1, LC-CDR2, and LC-CDR3 from SEQ ID NO: 55.

49. The system of claim 47 or 48, wherein the antibody moiety is an scFv.

50. The system of any one of claims 46-49, wherein the CAR transmembrane domain comprises a CD8 transmembrane domain, the CAR co-stimulatory domain comprises a 4-1BB and/or a CD28 co-stimulatory domain, and/or the CAR cytoplasmic signaling domain comprises a CD3-ζ cytoplasmic signaling domain.

51. The system of any one of claims 45-50, wherein the signaling domain of the first CISC component comprises an IL-2 receptor subunit gamma (IL2Rγ) domain.

52. The system of claim 51, wherein the IL2Rγ cytoplasmic signaling domain comprises the amino acid sequence of SEQ ID NO: 50 or a variant thereof having at least 85% homology to the amino acid sequence of SEQ ID NO: 50.

53. The system of any one of claims 45-52, wherein the first extracellular binding domain or portion thereof comprises an FK506 binding protein (FKBP) domain or a portion thereof.

54. The system of claim 53, wherein the FKBP domain comprises the amino acid sequence of SEQ ID NO: 47 or a variant thereof having at least 85% homology to the amino acid sequence of SEQ ID NO: 47.

55. The system of any one of claims 45-54, wherein the signaling domain of the second CISC component comprises an IL-2 receptor subunit beta (IL2Rβ) domain.

56. The system of claim 55, wherein the IL2Rβ cytoplasmic signaling domain comprises the amino acid sequence of SEQ ID NO: 51 or a variant thereof having at least 85% homology to the amino acid sequence of SEQ ID NO: 51.

57. The system of any one of claims 45-56, wherein the second extracellular binding domain or portion thereof comprises an FKBP rapamycin binding (FRB) domain or a portion thereof.

58. The system of claim 57, wherein the FRB comprises the amino acid sequence of SEQ ID NO: 48 or a variant thereof having at least 85% homology to the amino acid sequence of SEQ ID NO: 48.

59. The system of any one of claims 45-58, wherein the transmembrane domain of the first and second CISC components comprises, independently, an IL-2 receptor transmembrane domain.

60. The system of any one of claims 45-59, wherein the ligand is rapamycin or a rapalog.

61. The system of claim 60, wherein the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

62. The system of any one of claims 45-61, wherein the c) one or more donor templates further comprise nucleic acid encoding one or more of:
iv) a chimeric receptor comprising an extracellular β2-microglobulin domain, a transmembrane domain, a co-stimulatory domain, and a cytoplasmic signaling domain;
v) a selectable marker;
vi) a polypeptide that confers resistance to one or more calcineurin inhibitors; or
vii) an FKBP-rapamycin binding (FRB) domain polypeptide of the mammalian target of rapamycin (mTOR) kinase.

63. The system of claim 62, wherein the chimeric receptor transmembrane domain comprises a CD8 transmembrane domain polypeptide, the chimeric receptor co-stimulatory domain comprises a 4-1BB co-stimulatory domain, and/or the chimeric receptor cytoplasmic signaling domain comprises a CD3-ζ cytoplasmic signaling domain.

64. The system of claim 63, wherein the chimeric receptor comprises the amino acid sequence of SEQ ID NO: 65 or a variant thereof having at least 85% homology to the amino acid sequence of SEQ ID NO: 65.

65. The system of any one of claims 62-64, wherein the selectable marker is a truncated low-affinity nerve growth factor receptor (tLNGFR) polypeptide.

66. The system of claim 65, wherein the tLNGFR polypeptide comprises the amino acid sequence of SEQ ID NO: 66.

67. The system of any one of claims 62-66, wherein the polypeptide that confers resistance to one or more calcineurin inhibitors is a mutant calcineurin (CN) polypeptide.

68. The system of claim 67, wherein the mutant CN polypeptide is CNb30 (SEQ ID NO:
67).

69. The system of any one of claims 62-68, wherein the FRB domain polypeptide comprises the amino acid of SEQ ID NO: 68 or 69 or a variant having at least 90% sequence homology to the amino acid of SEQ ID NO: 68 or 69.

70. The system of any one of claims 44-69, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

71. The system of any one of claims 44-70, wherein the RGEN is Cas9.

72. The system of any one of claims 44-71, wherein the nucleic acid encoding the RGEN is a ribonucleic acid (RNA) sequence.

73. The system of claim 72, wherein the RNA sequence encoding the RGEN is linked to the first gRNA or the second gRNA via a covalent bond.

74. The system of any one of claims 45-73, comprising an Adeno-Associated Virus (AAV) vector comprising one of the one or more donor templates.

75. The system of claim 74, wherein the AAV vector comprises the polynucleotide sequence of any one of SEQ ID NOs: 19-46 and variants thereof having at least 85% homology to the polynucleotide sequence of any one of SEQ ID NOs: 19-46.

76. The system of claim 74 or 75, comprising the first gRNA and a first AAV vector and the second gRNA and a second AAV vector, wherein
(A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of any one of SEQ ID NOs: 28, 31, 34, and 37 and variants thereof having at least 85% homology to the polynucleotide sequence of any one of SEQ ID NOs: 28, 31, 34, and 37, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of any one of SEQ ID NOs: 40-44 or a variant thereof having at least 85% homology to the polynucleotide sequence of any one of SEQ ID NOs: 40-44;
(B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of any one of SEQ ID NOs: 29, 32, 35, and 38 and variants thereof having at least 85% homology to the polynucleotide sequence of any one of SEQ ID NOs: 29, 32, 35, and 38, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of any one of SEQ ID NOs: 40-44 or a variant thereof having at least 85% homology to the polynucleotide sequence of any one of SEQ ID NOs: 40-44; or
(C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of any one of SEQ ID NOs: 30, 33, 36, and 39 and variants thereof having at least 85% homology to the polynucleotide sequence of any one of SEQ ID NOs: 30, 33, 36, and 39, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of any one of SEQ ID NOs: 40-44 or a variant thereof having at least 85% homology to the polynucleotide sequence of any one of SEQ ID NOs: 40-44.

77. The system of claim 74 or 75, comprising:
(A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 19 or 22 or a variant thereof having at least 85% homology to the polynucleotide sequence of SEQ ID NO: 19 or 22, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45;
(B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 20 or 23 or a variant thereof having at least 85% homology to the polynucleotide sequence of SEQ ID NO: 20 or 23, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; or
(C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 21 or 24 or a variant thereof having at least 85% homology to the polynucleotide sequence of SEQ ID NO: 21 or 24, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45.

78. The system of claim 74 or 75, comprising:
(A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 25 or a variant thereof having at least 85% homology to the polynucleotide sequence of SEQ ID NO: 25, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46;
(B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 26 or a variant thereof having at least 85% homology to the polynucleotide sequence of SEQ ID NO: 26, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; or
(C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 27 or a variant thereof having at least 85% homology to the polynucleotide sequence of SEQ ID NO: 27, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46.

79. The system of any one of claims 44-78, comprising a ribonucleoprotein (RNP) complex comprising the RGEN and the first gRNA and/or the second gRNA.

80. The system of claim 79, wherein the RGEN is precomplexed with the first gRNA and/or the second gRNA at a molar ratio of gRNA to RGEN between 1:1 to 20:1, respectively, to form the RNP.

81. A vector comprising the nucleic acid sequence of any one of SEQ ID NOs: 19-46, or a variant thereof having at least 85% homology to any one of SEQ ID NOs: 19-46.

82. The vector of claim 81, wherein the vector is an Adeno Associated Virus (AAV) vector.

83. A method of editing the genome of a cell, the method comprising providing to the cell:
a) a first gRNA and/or a second gRNA, wherein the first gRNA is the gRNA of claim 40 or 41 and the second gRNA is the gRNA of claim 42 or 43;
b) an RGEN or a nucleic acid encoding the RGEN; and
c) one or more donor templates comprising nucleic acid encoding:
i) a CAR that can recognize a BCMA polypeptide;
ii) a first CISC component comprising a first extracellular binding domain or portion thereof, a hinge domain, a transmembrane domain, and a signaling domain or portion thereof or functional derivative thereof; and
iii) a second CISC component comprising a second extracellular binding domain or portion thereof, a hinge domain, a transmembrane domain, and a signaling domain or portion thereof,
wherein the first CISC component and the second CISC component are configured such that when expressed by a T cell, they dimerize in the presence of a ligand to create a signaling competent CISC capable of promoting the survival and/or proliferation of the T cell.

84. The method of claim 83, wherein the CAR that can recognize BCMA comprises an extracellular BCMA recognition domain, a transmembrane domain, a co-stimulatory domain, and a cytoplasmic signaling domain.

85. The method of claim 84, wherein the extracellular BCMA recognition domain is an antibody moiety that can specifically bind to BCMA.

86. The method of claim 85, wherein the antibody moiety comprises a heavy chain variable domain (V_{H}) and a light chain variable domain (V_{L}) comprising heavy chain complementarity-determining region (HC-CDR)1, HC-CDR2, HC-CDR3, light chain complementarity-determining region (LC-CDR)1, LC-CDR2, and LC-CDR3 from SEQ ID NO: 55.

87. The method of claim 85 or 86, wherein the antibody moiety is an scFv.

88. The method of any one of claims 84-87, wherein the CAR transmembrane domain comprises a CD8 transmembrane domain, the CAR co-stimulatory domain comprises a 4-1BB and/or a CD28 co-stimulatory domain, and/or the CAR cytoplasmic signaling domain comprises a CD3-ζ cytoplasmic signaling domain.

89. The method of any one of claims 83-88, wherein the signaling domain of the first CISC component comprises an IL-2 receptor subunit gamma (IL2Rγ) cytoplasmic signaling domain.

90. The method of claim 89, wherein the IL2Rγ cytoplasmic signaling domain comprises the amino acid sequence of SEQ ID NO: 50 or a variant thereof having at least 85% homology to the amino acid sequence of SEQ ID NO: 50.

91. The method of any one of claims 83-90, wherein the first extracellular binding domain or portion thereof comprises an FK506 binding protein (FKBP) domain or a portion thereof.

92. The method of claim 91, wherein the FKBP domain comprises the amino acid sequence of SEQ ID NO: 47 or a variant thereof having at least 85% homology to the amino acid sequence of SEQ ID NO: 47.

93. The method of any one of claims 83-92, wherein the signaling domain of the second CISC component comprises an IL-2 receptor subunit beta (IL2Rβ) cytoplasmic signaling domain.

94. The method of claim 93, wherein the IL2Rβ cytoplasmic signaling domain comprises the amino acid sequence of SEQ ID NO: 51 or a variant thereof having at least 85% homology to the amino acid sequence of SEQ ID NO: 51.

95. The method of any one of claims 83-94, wherein the second extracellular binding domain or portion thereof comprises an FKBP rapamycin binding (FRB) domain or a portion thereof.

96. The method of claim 95, wherein the FRB domain comprises the amino acid sequence of SEQ ID NO: 48 or a variant thereof having at least 85% homology to the amino acid sequence of SEQ ID NO: 48.

97. The method of any one of claims 83-96 wherein the transmembrane domain of the first and second CISC components comprises, independently, an IL-2 receptor transmembrane domain.

98. The method of any one of claims 83-97 wherein the ligand is rapamycin or a rapalog.

99. The method of claim 98, wherein the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

100. The method of any one of claims 83-99, wherein the c) one or more donor templates further comprise nucleic acid encoding one or more of:
iv) a chimeric receptor comprising an extracellular β2-microglobulin domain, a transmembrane domain, a co-stimulatory domain, and a cytoplasmic signaling domain;
v) a selectable marker;
vi) a polypeptide that confers resistance to one or more calcineurin inhibitors; or
vii) an FKBP-rapamycin binding (FRB) domain polypeptide of the mammalian target of rapamycin (mTOR) kinase.

101. The method of claim 100, wherein the chimeric receptor transmembrane domain comprises a CD8 transmembrane domain polypeptide, the chimeric receptor co-stimulatory domain comprises a 4-1BB co-stimulatory domain, and/or the chimeric receptor cytoplasmic signaling domain comprises a CD3-ζ cytoplasmic signaling domain.

102. The method of claim 101, wherein the chimeric receptor comprises the amino acid sequence of SEQ ID NO: 65 or a variant thereof having at least 85% homology to the amino acid sequence of SEQ ID NO: 65

103. The method of any one of claims 100-102, wherein the selectable marker is a truncated low-affinity nerve growth factor receptor (tLNGFR) polypeptide.

104. The method of claim 103, wherein the tLNGFR polypeptide comprises the amino acid sequence of SEQ ID NO: 66.

105. The method of any one of claims 100-104, wherein the polypeptide that confers resistance to one or more calcineurin inhibitors is a mutant calcineurin (CN) polypeptide.

106. The method of claim 105, wherein the mutant CN polypeptide is CNb30 (SEQ ID NO: 67).

107. The method of any one of claims 100-106, wherein the FRB domain polypeptide comprises the amino acid of SEQ ID NO: 68 or 69 or a variant having at least 90% sequence homology to the amino acid of SEQ ID NO: 68 or 69.

108. A method of editing the genome of a cell, the method comprising providing to the cell a first gRNA, a second gRNA, an RGEN or a nucleic acid encoding the RGEN, a first vector, and a second vector, wherein
(A) the first gRNA comprises the polynucleotide sequence of SEQ ID NO: 1 or a variant thereof having at least 85% homology to the polynucleotide sequence of SEQ ID NO: 1, the first vector comprises the polynucleotide sequence of any one of SEQ ID NOs: 28, 31, 34, and 37 and variants thereof having at least 85% homology to the polynucleotide sequence of any one of SEQ ID NOs: 28, 31, 34, and 37, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44;
(B) the first gRNA comprises the polynucleotide sequence of SEQ ID NO: 2 or a variant thereof having at least 85% homology to the polynucleotide sequence of SEQ ID NO: 2, the first vector comprises the polynucleotide sequence of any one of SEQ ID NOs: 29, 32, 35, and 38 and variants thereof having at least 85% homology to the polynucleotide sequence of any one of SEQ ID NOs: 29, 32, 35, and 38, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44; or
(C) the first gRNA comprises the polynucleotide sequence of SEQ ID NO: 3 or a variant thereof having at least 85% homology to the polynucleotide sequence of SEQ ID NO: 3, the first vector comprises the polynucleotide sequence of any one of SEQ ID NOs: 30, 33, 36, and 39 and variants thereof having at least 85% homology to the polynucleotide sequence of any one of SEQ ID NOs: 30, 33, 36, and 39, the second gRNA comprises the polynucleotide sequence of any one of SEQ ID NOs: 4-18 and variants thereof having at least 85% homology to the polynucleotide sequence of any one of SEQ ID NOs: 4-18, and the second vector comprises the polynucleotide sequence of any one of SEQ ID NOs: 40-44 or a variant thereof having at least 85% homology to the polynucleotide sequence of any one of SEQ ID NOs: 40-44.

109. A method of editing the genome of a cell, the method comprising providing to the cell a first gRNA, a second gRNA, an RGEN or a nucleic acid encoding the RGEN, a first vector, and a second vector, wherein
(A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 19 or 22 or a variant thereof having at least 85% homology to the polynucleotide sequence of SEQ ID NO: 19 or 22, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45;
(B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 20 or 23 or a variant thereof having at least 85% homology to the polynucleotide sequence of SEQ ID NO: 20 or 23, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45; or
(C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 21 or 24 or a variant thereof having at least 85% homology to the polynucleotide sequence of SEQ ID NO: 21 or 24, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 45 or a variant thereof having at least 85% homology to the polynucleotide sequence of SEQ ID NO: 45.

110. A method of editing the genome of a cell, the method comprising providing to the cell a first gRNA, a second gRNA, an RGEN or a nucleic acid encoding the RGEN, a first vector, and a second vector, wherein
(A) the first gRNA comprises the polynucleotide sequence of SEQ ID NO: 1 or a variant thereof having at least 85% homology to the polynucleotide sequence of SEQ ID NO: 1, the first AAV vector comprises the polynucleotide sequence of SEQ ID NO: 25 or a variant thereof having at least 85% homology to the polynucleotide sequence of SEQ ID NO: 25, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46;
(B) the first gRNA comprises the polynucleotide sequence of SEQ ID NO: 2 or a variant thereof having at least 85% homology to the polynucleotide sequence of SEQ ID NO: 2, the first AAV vector comprises the polynucleotide sequence of SEQ ID NO: 26 or a variant thereof having at least 85% homology to the polynucleotide sequence of SEQ ID NO: 26, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46; or
(C) the first gRNA comprises the polynucleotide sequence of SEQ ID NO: 3 or a variant thereof having at least 85% homology to the polynucleotide sequence of SEQ ID NO: 3, the first AAV vector comprises the polynucleotide sequence of SEQ ID NO: 27 or a variant thereof having at least 85% homology to the polynucleotide sequence of SEQ ID NO: 27, the second gRNA comprises the polynucleotide sequence of any one of SEQ ID NOs: 4-18 and variants thereof having at least 85% homology to the polynucleotide sequence of any one of SEQ ID NOs: 4-18, and the second AAV vector comprises the polynucleotide sequence of SEQ ID NO: 46 or a variant thereof having at least 85% homology to the polynucleotide sequence of SEQ ID NO: 46.

111. The method of any one of claims 83-110, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

112. The method of any one of claims 83-111, wherein the RGEN is Cas9.

113. The method of any one of claims 83-112, wherein the nucleic acid encoding the RGEN is a ribonucleic acid (RNA) sequence.

114. The method of claim 113, wherein the RNA sequence encoding the RGEN is linked to the first gRNA or the second gRNA via a covalent bond.

115. The method of any one of claims 83-114, wherein the donor template is contained in an AAV vector.

116. The method of any one of claims 83-115, wherein the RGEN is precomplexed with the first gRNA and/or the second gRNA, forming an RNP complex, prior to the provision to the cell.

117. The method of claim 116, wherein the RGEN is precomplexed with the first gRNA and/or the second gRNA at a molar ratio of gRNA to RGEN between 1:1 to 20:1, respectively.

118. The method of any one of claims 83-117, wherein the one or more donor templates are, independently, inserted into the genome of the cell.

119. The method of claim 118, wherein a first donor template is inserted at, within, or near a TIM gene or gene regulatory element and/or a second donor template is inserted at, within, or near an IL2RG gene or gene regulatory element.

120. The method of claim 118 or 119, wherein nucleic acid encoding i) the first CISC component is inserted into an endogenous IL2RG gene, and/or nucleic acid encoding ii) the second CISC component is inserted into the region of the endogenous TIM gene encoding the TRAC domain; or nucleic acid encoding i) the first CISC component is inserted into the region of the endogenous TIM gene encoding the TRAC domain, and/or nucleic acid encoding ii) the second CISC component is inserted into the endogenous IL2RG gene.

121. The method of any one of claims 83-120, wherein the cell is a T cell.

122. The method of claim 121, wherein the T cell is a CD8+ cytotoxic T lymphocyte or a CD3+ pan T cell.

123. The method of claim 121 or 122, wherein the T cell is a member of a pool of T cells derived from multiple donors.

124. The method of claim 123, wherein the multiple donors are human donors.

125. The method of any one of claims 83-124, wherein the cell is cytotoxic to plasma cells.

126. An engineered cell produced by the method of any one of claims 83-125.

127. The engineered cell of any one of claims 1-39 and 126, wherein the engineered cell is cytotoxic to plasma cells.

128. A method of treating graft vs host disease (GvHD) or an autoimmune disease in a subject in need thereof, the method comprising: administering the engineered cell of any one of claim 1-39 or 126 to the subject.

129. A method of treating a disease or condition in a subject in need thereof, wherein the disease or condition is characterized by adverse antibody production, the method comprising:
a) editing the genome of T cells according to the method of any one of claims 83-120, thereby producing engineered T cells; and
b) administering the engineered T cells to the subject.

130. The method of claim 129, wherein the T cells are autologous to the subject.

131. The method of claim 120, wherein the T cells are allogenic to the subject.

132. The method of claim 131, wherein the T cells comprise a pool of T cells derived from multiple donors.

133. The method of claim 132, wherein the multiple donors are human donors.

134. A method of treating a disease or condition in a subject in need thereof, wherein the disease or condition is characterized by adverse antibody production, the method comprising editing the genome of a T cell in the subject according to the method of any one of claims 83-120.

135. The method of any one of claims 129-134, wherein the T cells comprise CD8+ cytotoxic T cells or CD3+ pan T cells.

136. The method of any one of claims 128-135, wherein the subject is human.

137. The method of any one of claims 128-136, further comprising administering rapamycin or a rapalog to the subject.

138. The method of claim 137, wherein the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

139. The method of any one of claims 137-138, wherein the rapamycin or the rapalog is administered in a concentration from 0.05 nM to 100 nM.

140. The method of any one of claims 129-139, wherein the disease or condition is graft-versus-host disease (GvHD), antibody-mediated autoimmunity, or light-chain amyloidosis.

141. The method of claim 140, wherein the disease or condition is GvHD, and the subject has previously received an allogeneic transplant.

142. A kit comprising instructions for use and a) the engineered cell of any one of claim 1-39 or 126 and/or one or more components of the system of any one of claims 44-80; and/or b) rapamycin or a rapalog.

143. The kit of claim 142, wherein the rapalog is selected from the group consisting of everolimus, CCI-779, C20-methallylrapamycin, C16-(S)-3-methylindolerapamycin, C16-iRap, AP21967, sodium mycophenolic acid, benidipine hydrochloride, AP1903, or AP23573, or metabolites, derivatives, and/or combinations thereof.

144. A syringe comprising the engineered cell of any one of claim 1-39 or 126 or a composition comprising one or more components of the system of any one of claims 44-80.

145. A catheter comprising the engineered cell of any one of claim 1-39 or 126 or a composition comprising one or more components of the system of any one of claims 44-80.
